SEQUENCE LISTING (94 sequences)

SEQ ID NO: 1 - DNA, 1037 bp, Pichia pastoris
aaatgcgtacctcttctacgagattcaagcgaatgagaataatgtaatatgcaagatcagaaagaatgaaaggagttgaaaaaaaaaaccgttgcgttttgaccttgaatggggtggaggtttccattcaaagtaaagcctgtgtcttggtattttcggcggcacaagaaatcgtaattttcatcttctaaacgatgaagatcgcagcccaacctgtatgtagttaaccggtcggaattataagaaagattttcgatcaacaaaccctagcaaatagaaagcagggttacaactttaaaccgaagtcacaaacgataaaccactcagctcccacccaaattcattcccactagcagaaaggaattatttaatccctcaggaaacctcgatgattctcccgttcttccatgggcgggtatcgcaaaatgaggaatttttcaaatttctctattgtcaagactgtttattatctaagaaatagcccaatccgaagctcagttttgaaaaaatcacttccgcgtttcttttttacagcccgatgaatatccaaatttggaatatggattactctatcgggactgcagataatatgacaacaacgcagattacattttaggtaaggcataaacaccagccagaaatgaaacgcccactagccatggtcgaatagtccaatgaattcagatagctatggtctaaaagctgatgttttttattgggtaatggcgaagagtccagtacgacttccagcagagctgagatggccatttttgggggtattagtaactttttgagctcttttcacttcgatgaagtgtcccattcgggatataatcggatcgcgtcgttttctcgaaaatacagcttagcgtcgtccgcttgttgtaaaagcagcaccacattcctaatctcttatataaacaaaacaacccaaattatcagtgctgttttcccaccagatataagtttcttttctcttccgctttttgattttttatctctttcctttaaaaacttctttaccttaaagggcggcc

SEQ ID NO: 2 - DNA, 934 bp, Pichia pastoris
aacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 3 - DNA, 293 bp, Saccharomyces cerevisiae
acaggccccttttcctttgtcgatatcatgtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacattatactgaaaaccttgcttgagaaggttttgggacgctcgaaggctttaatttgcaagctgccggctcttaag

SEQ ID NO: 4 - DNA, 600 bp, Pichia pastoris
gttcttcgcttggtcttgtatctccttacactgtatcttcccatttgcgtttaggtggttatcaaaaactaaaaggaaaaatttcagatgtttatctctaaggttttttctttttacagtataacacgtgatgcgtcacgtggtactagattacgtaagttattttggtccggtgggtaagtgggtaagaatagaaagcatgaaggtttacaaaaacgcagtcacgaattattgctacttcgagcttggaaccaccccaaagattatattgtactgatgcactaccttctcgattttgctcctccaagaacctacgaaaaacatttcttgagccttttcaacctagactacacatcaagttatttaaggtatgttccgttaacatgtaagaaaaggagaggatagatcgtttatggggtacgtcgcctgattcaagcgtgaccattcgaagaataggccttcgaaagctgaataaagcaaatgtcagttgcgattggtatgctgacaaattagcataaaaagcaatagactttctaaccacctgtttttttccttttactttatttatattttgccaccgtactaacaagttcagacaaa

SEQ ID NO: 5 - DNA, 486 bp, Pichia pastoris
tttttgtagaaatgtcttggtgtcctcgtccaatcaggtagccatctctgaaatatctggctccgttgcaactccgaacgacctgctggcaacgtaaaattctccggggtaaaacttaaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactatcaaaacaca

SEQ ID NO: 6 - DNA, 600 bp, Pichia pastoris
ttaaggtttggaacaacactaaactaccttgcggtactaccattgacactacacatccttaattccaatcctgtctggcctccttcaccttttaaccatcttgcccattccaactcgtgtcagattgcgtatcaagtgaaaaaaaaaaaattttaaatctttaacccaatcaggtaataactgtcgcctcttttatctgccgcactgcatgaggtgtccccttagtgggaaagagtactgagccaaccctggaggacagcaagggaaaaatacctacaacttgcttcataatggtcgtaaaaacaatccttgtcggatataagtgttgtagactgtcccttatcctctgcgatgttcttcctctcaaagtttgcgatttctctctatcagaattgccatcaagagactcaggactaatttcgcagtcccacacgcactcgtacatgattggctgaaatttccctaaagaatttctttttcacgaaaattttttttttacacaagattttcagcagatataaaatggagagcaggacctccgctgtgactcttcttttttttcttttattctcactacatacattttagttattcgccaac

SEQ ID NO: 7 - DNA, 301 bp, Pichia pastoris
attgcttgaagctttaatttattttattaacataataataatacaagcatgatatatttgtattttgttcgttaacattgatgttttcttcatttactgttattgtttgtaactttgatcgatttatcttttctactttactgtaatatggctggcgggtgagccttgaactccctgtattactttaccttgctattacttaatctattgactagcagcgacctcttcaaccgaagggcaagtacacagcaagttcatgtctccgtaagtgtcatcaaccctggaaacagtgggccatgtc

SEQ ID NO: 8 - DNA, 376 bp, Pichia pastoris
atttacaattagtaatattaaggtggtaaaaacattcgtagaattgaaatgaattaatatagtatgacaatggttcatgtctataaatctccggcttcggtaccttctccccaattgaatacattgtcaaaatgaatggttgaactattaggttcgccagtttcgttattaagaaaactgttaaaatcaaattccatatcatcggttccagtgggaggaccagttccatcgccaaaatcctgtaagaatccattgtcagaacctgtaaagtcagtttgagatgaaatttttccggtctttgttgacttggaagcttcgttaaggttaggtgaaacagtttgatcaaccagcggctcccgttttcgtcgcttagtag

SEQ ID NO: 9 - DNA, 672 bp, Pichia pastoris
gcggaaacggcagtaaacaatggagcttcattagtgggtgttattatggtccctggccgggaacgaacggtgaaacaagaggttgcgagggaaatttcgcagatggtgcgggaaaagagaatttcaaagggctcaaaatacttggattccagacaactgaggaaagagtgggacgactgtcctctggaagactggtttgagtacaacgtgaaagaaataaacagcagtggtccatttttagttggagtttttcgtaatcaaagtatagatgaaatccagcaagctatccacactcatggtttggatttcgtccaactacatgggtctgaggattttgattcgtatatacgcaatatcccagttcctgtgattaccagatacacagataatgccgtcgatggtcttaccggagaagacctcgctataaatagggccctggtgctactggacagcgagcaaggaggtgaaggaaaaaccatcgattgggctcgtgcacaaaaatttggagaacgtagaggaaaatatttactagccggaggtttgacacctgataatgttgctcatgctcgatctcatactggctgtattggtgttgacgtctctggtggggtagaaacaaatgcctcaaaagatatggacaagatcacacaatttatcagaaacgctacataa

SEQ ID NO: 10 - DNA, 834 bp, Pichia pastoris
aagtcaattaaatacacgcttgaaaggacattacatagctttcgatttaagcagaaccagaaatgtagaaccacttgtcaatagattggtcaatcttagcaggagcggctgggctagcagttggaacagcagaggttgctgaaggtgagaaggatggagtggattgcaaagtggtgttggttaagtcaatctcaccagggctggttttgccaaaaatcaacttctcccaggcttcacggcattcttgaatgacctcttctgcatacttcttgttcttgcattcaccagagaaagcaaactggttctcaggttttccatcagggatcttgtaaattctgaaccattcgttggtagctctcaacaagcccggcatgtgcttttcaacatcctcgatgtcattgagcttaggagccaatgggtcgttgatgtcgatgacgatgaccttccagtcagtctctccctcatccaacaaagccataacaccgaggaccttgacttgcttgacctgtccagtgtaacctacggcttcaccaatttcgcaaacgtccaatggatcattgtcacccttggccttggtctctggatgagtgacgttagggtcttcccatgtctgagggaaggcaccgtagttgtgaatgtatccgtggtgagggaaacagttacgaacgaaacgaagttttcccttctttgtgtcctgaagaattgggttcagtttctcctccttggaaatctccaacttggcgttggtccaacgggggacttcaacaaccatgttgagaaccttcttggattcgtcagcataaagtgggatgtcgtggaaaggagatacgactt

SEQ ID NO: 11 - DNA, 1215 bp, Saccharomyces cerevisiae
atgtcagaagatcaaaaaagtgaaaattccgtaccttctaaggttaatatggtgaatcgcaccgatatactgactacgatcaagtcattgtcatggcttgacttgatgttgccatttactataattctctccataatcattgcagtaataatttctgtctatgtgccttcttcccgtcacacttttgacgctgaaggtcatcccaatctaatgggagtgtccattcctttgactgttggtatgattgtaatgatgattcccccgatctgcaaagtttcctgggagtctattcacaagtacttctacaggagctatataaggaagcaactagccctctcgttatttttgaattgggtcatcggtcctttgttgatgacagcattggcgtggatggcgctattcgattataaggaataccgtcaaggcattattatgatcggagtagctagatgcattgccatggtgctaatttggaatcagattgctggaggagacaatgatctctgcgtcgtgcttgttattacaaactcgcttttacagatggtattatatgcaccattgcagatattttactgttatgttatttctcatgaccacctgaatacttcaaatagggtattattcgaagaggttgcaaagtctgtcggagtttttctcggcataccactgggaattggcattatcatacgtttgggaagtcttaccatagctggtaaaagtaattatgaaaaatacattttgagatttatttctccatgggcaatgatcggatttcattacactttatttgttatttttattagtagaggttatcaatttatccacgaaattggttctgcaatattgtgctttgtcccattggtgctttacttctttattgcatggtttttgaccttcgcattaatgaggtacttatcaatatctaggagtgatacacaaagagaatgtagctgtgaccaagaactacttttaaagagggtctggggaagaaagtcttgtgaagctagcttttctattacgatgacgcaatgtttcactatggcttcaaataattttgaactatccctggcaattgctatttccttatatggtaacaatagcaagcaagcaatagctgcaacatttgggccgttgctagaagttccaattttattgattttggcaatagtcgcgagaatccttaaaccatattatatatggaacaatagaaattaa

SEQ ID NO: 12 - DNA, 1144 bp, Pichia pastoris
caaatgcaagaggacattagaaatgtgtttggtaagaacatgaagccggaggcatacaaacgattcacagatttgaaggaggaaaacaaactgcatccaccggaagtgccagcagccgtgtatgccaaccttgctctcaaaggcattcctacggatctgagtgggaaatatctgagattcacagacccactattggaacagtaccaaacctagtttggccgatccatgattatgtaatgcatatagtttttgtcgatgctcacccgtttcgagtctgtctcgtatcgtcttacgtataagttcaagcatgtttaccaggtctgttagaaactcctttgtgagggcaggacctattcgtctcggtcccgttgtttctaagagactgtacagccaagcgcagaatggtggcattaaccataagaggattctgatcggacttggtctattggctattggaaccaccctttacgggacaaccaaccctaccaagactcctattgcatttgtggaaccagccacggaaagagcgtttaaggacggagacgtctctgtgatttttgttctcggaggtccaggagctggaaaaggtacccaatgtgccaaactagtgagtaattacggatttgttcacctgtcagctggagacttgttacgtgcagaacagaagagggaggggtctaagtatggagagatgatttcccagtatatcagagatggactgatagtacctcaagaggtcaccattgcgctcttggagcaggccatgaaggaaaacttcgagaaagggaagacacggttcttgattgatggattccctcgtaagatggaccaggccaaaacttttgaggaaaaagtcgcaaagtccaaggtgacacttttctttgattgtcccgaatcagtgctccttgagagattacttaaaagaggacagacaagcggaagagaggatgataatgcggagagtatcaaaaaaagattcaaaacattcgtggaaacttcgatgcctgtggtggactatttcgggaagcaaggacgcgttttgaaggtatcttgtgaccaccctgtggatcaagtgtattcacaggttgtgtcggtgctaaaagagaaggggatctttgccgataacgagacggagaataaataa

SEQ ID NO: 13 - DNA, 1201 bp, Artificial Sequence
  NatR expression cassette
tgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaccactcttgacgacacggcttaccggtaccgcaccagtgtcccgggggacgccgaggccatcgaggcactggatgggtccttcaccaccgacaccgtcttccgcgtcaccgccaccggggacggcttcaccctgcgggaggtgccggtggacccgcccctgaccaaggtgttccccgacgacgaatcggacgacgaatcggacgacggggaggacggcgacccggactcccggacgttcgtcgcgtacggggacgacggcgacctggcgggcttcgtggtcgtctcgtactccggctggaaccgccggctgaccgtcgaggacatcgaggtcgccccggagcaccgggggcacggggtcgggcgcgcgttgatggggctcgcgacggagttcgcccgcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcaggcgctctacatgagcatgccctgcccctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaac

SEQ ID NO: 14 - DNA, 375 bp, Artificial Sequence
  Sh ble ORF
atggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactga

SEQ ID NO: 15 - DNA, 260 bp, Pichia pastoris
tcaagaggatgtcagaatgccatttgcctgagagatgcaggcttcattttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagacgttcgtttgtgca

SEQ ID NO: 16 - DNA, 427 bp, Saccharomyces cerevisiae
gatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaaa

SEQ ID NO: 17 - DNA, 3029 bp, Saccharomyces cerevisiae
  CDS (909)..(2507)
aggcctcgcaacaacctataattgagttaagtgcctttccaagctaaaaagtttgaggttataggggcttagcatccacacgtcacaatctcgggtatcgagtatagtatgtagaattacggcaggaggtttcccaatgaacaaaggacaggggcacggtgagctgtcgaaggtatccattttatcatgtttcgtttgtacaagcacgacatactaagacatttaccgtatgggagttgttgtcctagcgtagttctcgctcccccagcaaagctcaaaaaagtacgtcatttagaatagtttgtgagcaaattaccagtcggtatgctacgttagaaaggcccacagtattcttctaccaaaggcgtgcctttgttgaactcgatccattatgagggcttccattattccccgcatttttattactctgaacaggaataaaaagaaaaaacccagtttaggaaattatccgggggcgaagaaatacgcgtagcgttaatcgaccccacgtccagggtttttccatggaggtttctggaaaaactgacgaggaatgtgattataaatccctttatgtgatgtctaagacttttaaggtacgcccgatgtttgcctattaccatcatagagacgtttcttttcgaggaatgcttaaacgactttgtttgacaaaaatgttgcctaagggctctatagtaaaccatttggaagaaagatttgacgactttttttttttggatttcgatcctataatccttcctcctgaaaagaaacatataaatagatatgtattattcttcaaaacattctcttgttcttgtgctttttttttaccatatatcttacttttttttttctctcagagaaacaagcaaaacaaaaagcttttcttttcactaacgtatatgatgcttttgcaagctttccttttccttttggctggttttgcagccaaaatatctgcatcaatgacaaacgaaactagcgatagacctttggtccacttcacacccaacaagggctggatgaatgacccaaatgggttgtggtacgatgaaaaagatgccaaatggcatctgtactttcaatacaacccaaatgacaccgtatggggtacgccattgttttggggccatgctacttccgatgatttgactaattgggaagatcaacccattgctatcgctcccaagcgtaacgattcaggtgctttctctggctccatggtggttgattacaacaacacgagtgggtttttcaatgatactattgatccaagacaaagatgcgttgcgatttggacttataacactcctgaaagtgaagagcaatacattagctattctcttgatggtggttacacttttactgaataccaaaagaaccctgttttagctgccaactccactcaattcagagatccaaaggtgttctggtatgaaccttctcaaaaatggattatgacggctgccaaatcacaagactacaaaattgaaatttactcctctgatgacttgaagtcctggaagctagaatctgcatttgccaatgaaggtttcttaggctaccaatacgaatgtccaggtttgattgaagtcccaactgagcaagatccttccaaatcttattgggtcatgtttatttctatcaacccaggtgcacctgctggcggttccttcaaccaatattttgttggatccttcaatggtactcattttgaagcgtttgacaatcaatctagagtggtagattttggtaaggactactatgccttgcaaactttcttcaacactgacccaacctacggttcagcattaggtattgcctgggcttcaaactgggagtacagtgcctttgtcccaactaacccatggagatcatccatgtctttggtccgcaagttttctttgaacactgaatatcaagctaatccagagactgaattgatcaatttgaaagccgaaccaatattgaacattagtaatgctggtccctggtctcgttttgctactaacacaactctaactaaggccaattcttacaatgtcgatttgagcaactcgactggtaccctagagtttgagttggtttacgctgttaacaccacacaaaccatatccaaatccgtctttgccgacttatcactttggttcaagggtttagaagatcctgaagaatatttgagaatgggttttgaagtcagtgcttcttccttctttttggaccgtggtaactctaaggtcaagtttgtcaaggagaacccatatttcacaaacagaatgtctgtcaacaaccaaccattcaagtctgagaacgacctaagttactataaagtgtacggcctactggatcaaaacatcttggaattgtacttcaacgatggagatgtggtttctacaaatacctacttcatgaccaccggtaacgctctaggatctgtgaacatgaccactggtgtcgataatttgttctacattgacaagttccaagtaagggaagtaaaatagaggttataaaacttattgtcttttttatttttttcaaaagccattctaaagggctttagctaacgagtgacgaatgtaaaactttatgatttcaaagaatacctccaaaccattgaaaatgtatttttatttttattttctcccgaccccagttacctggaatttgttctttatgtactttatataagtataattctcttaaaaatttttactactttgcaatagacatcattttttcacgtaataaacccacaatcgtaatgtagttgccttacactactaggatggacctttttgcctttatctgttttgttactgacacaatgaaaccgggtaaagtattagttatgtgaaaatttaaaagcattaagtagaagtataccatattgtaaaaaaaaaaagcgttgtcttctacgtaaaagtgttctcaaaaagaagtagtgagggaaatggataccaagctatctgtaacaggagctaaaaaatctcagggaaaagcttctggtttgggaaacggtcgac

SEQ ID NO: 18 - DNA, 898 bp, Pichia pastoris
atcggcctttgttgatgcaagttttacgtggatcatggactaaggagttttatttggaccaagttcatcgtcctagacattacggaaagggttctgctcctctttttggaaactttttggaacctctgagtatgacagcttggtggattgtacccatggtatggcttcctgtgaatttctattttttctacattggattcaccaatcaaaacaaattagtcgccatggctttttggcttttgggtctatttgtttggaccttcttggaatatgctttgcatagatttttgttccacttggactactatcttccagagaatcaaattgcatttaccattcatttcttattgcatgggatacaccactatttaccaatggataaatacagattggtgatgccacctacacttttcattgtactttgctacccaatcaagacgctcgtcttttctgttctaccatattacatggcttgttctggatttgcaggtggattcctgggctatatcatgtatgatgtcactcattacgttctgcatcactccaagctgcctcgttatttccaagagttgaagaaatatcatttggaacatcactacaagaattacgagttaggctttggtgtcacttccaaattctgggacaaagtctttgggacttatctgggtccagacgatgtgtatcaaaagacaaattagagtatttataaagttatgtaagcaaataggggctaatagggaaagaaaaattttggttctttatcagagctggctcgcgcgcagtgtttttcgtgctcctttgtaatagtcatttttgactactgttcagattgaaatcacattgaagatgtcactcgaggggtaccaaaaaaggtttttggatgctgcagtggcttcgc

SEQ ID NO: 19 - DNA, 1060 bp, Pichia pastoris
ggtcttttcaacaaagctccattagtgagtcagctggctgaatcttatgcacaggccatcattaacagcaacctggagatagacgttgtatttggaccagcttataaaggtattcctttggctgctattaccgtgttgaagttgtacgagctcggcggcaaaaaatacgaaaatgtcggatatgcgttcaatagaaaagaaaagaaagaccacggagaaggtggaagcatcgttggagaaagtctaaagaataaaagagtactgattatcgatgatgtgatgactgcaggtactgctatcaacgaagcatttgctataattggagctgaaggtgggagagttgaaggtagtattattgccctagatagaatggagactacaggagatgactcaaataccagtgctacccaggctgttagtcagagatatggtacccctgtcttgagtatagtgacattggaccatattgtggcccatttgggcgaaactttcacagcagacgagaaatctcaaatggaaacgtatagaaaaaagtatttgcccaaataagtatgaatctgcttcgaatgaatgaattaatccaattatcttctcaccattattttcttctgtttcggagctttgggcacggcggcgggtggtgcgggctcaggttccctttcataaacagatttagtacttggatgcttaatagtgaatggcgaatgcaaaggaacaatttcgttcatctttaaccctttcactcggggtacacgttctggaatgtacccgccctgttgcaactcaggtggaccgggcaattcttgaactttctgtaacgttgttggatgttcaaccagaaattgtcctaccaactgtattagtttccttttggtcttatattgttcatcgagatacttcccactctccttgatagccactctcactcttcctggattaccaaaatcttgaggatgagtcttttcaggctccaggatgcaaggtatatccaagtacctgcaagcatctaatattgtctttgccagggggttctccacaccatactccttttggcgcatgc

SEQ ID NO: 20 - DNA, 957 bp, Pichia pastoris
tctagagggacttatctgggtccagacgatgtgtatcaaaagacaaattagagtatttataaagttatgtaagcaaataggggctaatagggaaagaaaaattttggttctttatcagagctggctcgcgcgcagtgtttttcgtgctcctttgtaatagtcatttttgactactgttcagattgaaatcacattgaagatgtcactggaggggtaccaaaaaaggtttttggatgctgcagtggcttcgcaggccttgaagtttggaactttcaccttgaaaagtggaagacagtctccatacttctttaacatgggtcttttcaacaaagctccattagtgagtcagctggctgaatcttatgctcaggccatcattaacagcaacctggagatagacgttgtatttggaccagcttataaaggtattcctttggctgctattaccgtgttgaagttgtacgagctgggcggcaaaaaatacgaaaatgtcggatatgcgttcaatagaaaagaaaagaaagaccacggagaaggtggaagcatcgttggagaaagtctaaagaataaaagagtactgattatcgatgatgtgatgactgcaggtactgctatcaacgaagcatttgctataattggagctgaaggtgggagagttgaaggttgtattattgccctagatagaatggagactacaggagatgactcaaataccagtgctacccaggctgttagtcagagatatggtacccctgtcttgagtatagtgacattggaccatattgtggcccatttgggcgaaactttcacagcagacgagaaatctcaaatggaaacgtatagaaaaaagtatttgcccaaataagtatgaatctgcttcgaatgaatgaattaatccaattatcttctcaccattattttcttctgtttcggagctttgggcacggcggcggatcc

SEQ ID NO: 21 - DNA, 709 bp, Pichia pastoris
cctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccag

SEQ ID NO: 22 - DNA, 2875 bp, Pichia pastoris
aaaaccttttttcctattcaaacacaaggcattgcttcaacacgtgtgcgtatccttaacacagatactccatacttctaataatgtgatagacgaatacaaagatgttcactctgtgttgtgtctacaagcatttcttattctgattggggatattctagttacagcactaaacaactggcgatacaaacttaaattaaataatccgaatctagaaaatgaacttttggatggtccgcctgttggttggataaatcaataccgattaaatggattctattccaatgagagagtaatccaagacactctgatgtcaataatcatttgcttgcaacaacaaacccgtcatctaatcaaagggtttgatgaggcttaccttcaattgcagataaactcattgctgtccactgctgtattatgtgagaatatgggtgatgaatctggtcttctccactcagctaacatggctgtttgggcaaaggtggtacaattatacggagatcaggcaatagtgaaattgttgaatatggctactggacgatgcttcaaggatgtacgtctagtaggagccgtgggaagattgctggcagaaccagttggcacgtcgcaacaatccccaagaaatgaaataagtgaaaacgtaacgtcaaagacagcaatggagtcaatattgataacaccactggcagagcggttcgtacgtcgttttggagccgatatgaggctcagcgtgctaacagcacgattgacaagaagactctcgagtgacagtaggttgagtaaagtattcgcttagattcccaaccttcgttttattctttcgtagacaaagaagctgcatgcgaacatagggacaacttttataaatccaattgtcaaaccaacgtaaaaccctctggcaccattttcaacatatatttgtgaagcagtacgcaatatcgataaatactcaccgttgtttgtaacagccccaacttgcatacgccttctaatgacctcaaatggataagccgcagcttgtgctaacataccagcagcaccgcccgcggtcagctgcgcccacacatataaaggcaatctacgatcatgggaggaattagttttgaccgtcaggtcttcaagagttttgaactcttcttcttgaactgtgtaaccttttaaatgacgggatctaaatacgtcatggatgagatcatgtgtgtaaaaactgactccagcatatggaatcattccaaagattgtaggagcgaacccacgataaaagtttcccaaccttgccaaagtgtctaatgctgtgacttgaaatctgggttcctcgttgaagaccctgcgtactatgcccaaaaactttcctccacgagccctattaacttctctatgagtttcaaatgccaaacggacacggattaggtccaatgggtaagtgaaaaacacagagcaaaccccagctaatgagccggccagtaaccgtcttggagctgtttcataagagtcattagggatcaataacgttctaatctgttcataacatacaaattttatggctgcatagggaaaaattctcaacagggtagccgaatgaccctgatatagacctgcgacaccatcatacccatagatctgcctgacagccttaaagagcccgctaaaagacccggaaaaccgagagaactctggattagcagtctgaaaaagaatcttcactctgtctagtggagcaattaatgtcttagcggcacttcctgctactccgccagctactcctgaatagatcacatactgcaaagactgcttgtcgatgaccttggggttatttagcttcaagggcaatttttgggacattttggacacaggagactcagaaacagacacagagcgttctgagtcctggtgctcctgacgtaggcctagaacaggaattattggctttatttgtttgtccatttcataggcttggggtaatagatagatgacagagaaatagagaagacctaatattttttgttcatggcaaatcgcgggttcgcggtcgggtcacacacggagaagtaatgagaagagctggtaatctggggtaaaagggttcaaaagaaggtcgcctggtagggatgcaatacaaggttgtcttggagtttacattgaccagatgatttggctttttctctgttcaattcacatttttcagcgagaatcggattgacggagaaatggcggggtgtggggtggatagatggcagaaatgctcgcaatcaccgcgaaagaaagactttatggaatagaactactgggtggtgtaaggattacatagctagtccaatggagtccgttggaaaggtaagaagaagctaaaaccggctaagtaactagggaagaatgatcagactttgatttgatgaggtctgaaaatactctgctgctttttcagttgctttttccctgcaacctatcattttccttttcataagcctgccttttctgttttcacttatatgagttccgccgagacttccccaaattctctcctggaacattctctatcgctctccttccaagttgcgccccctggcactgcctagtaatattaccacgcgacttatattcagttccacaatttccagtgttcgtagcaaatatcatcagccatggcgaaggcagatggcagtttgctctactataatcctcacaatccacccagaaggtattacttctacatggctatattcgccgtttctgtcatttgcgttttgtacggaccctcacaacaattatcatctccaaaaatagactatgatccattgacgctccgatcacttgatttgaagactttggaagctccttcacagttgagtccaggcaccgtagaagataatcttcg

SEQ ID NO: 23 - DNA, 997 bp, Pichia pastoris
aaagctagagtaaaatagatatagcgagattagagaatgaataccttcttctaagcgatcgtccgtcatcatagaatatcatggactgtatagttttttttttgtacatataatgattaaacggtcatccaacatctcgttgacagatctctcagtacgcgaaatccctgactatcaaagcaagaaccgatgaagaaaaaaacaacagtaacccaaacaccacaacaaacactttatcttctcccccccaacaccaatcatcaaagagatgtcggaaccaaacaccaagaagcaaaaactaaccccatataaaaacatcctggtagataatgctggtaacccgctctccttccatattctgggctacttcacgaagtctgaccggtctcagttgatcaacatgatcctcgaaatgggtggcaagatcgttccagacctgcctcctctggtagatggagtgttgtttttgacaggggattacaagtctattgatgaagataccctaaagcaactgggggacgttccaatatacagagactccttcatctaccagtgttttgtgcacaagacatctcttcccattgacactttccgaattgacaagaacgtcgacttggctcaagatttgatcaatagggcccttcaagagtctgtggatcatgtcacttctgccagcacagctgcagctgctgctgttgttgtcgctaccaacggcctgtcttctaaaccagacgctcgtactagcaaaatacagttcactcccgaagaagatcgttttattcttgactttgttaggagaaatcctaaacgaagaaacacacatcaactgtacactgagctcgctcagcacatgaaaaaccatacgaatcattctatccgccacagatttcgtcgtaatctttccgctcaacttgattgggtttatgatatcgatccattgaccaaccaacctcgaaaagatgaaaacgggaactacatcaaggtacaaggccttcca

SEQ ID NO: 24 - DNA, 2159 bp, Kluyveromyces lactis
  CDS (1024)..(2010)
aaacgtaacgcctggcactctattttctcaaacttctgggacggaagagctaaatattgtgttgcttgaacaaacccaaaaaaacaaaaaaatgaacaaactaaaactacacctaaataaaccgtgtgtaaaacgtagtaccatattactagaaaagatcacaagtgtatcacacatgtgcatctcatattacatcttttatccaatccattctctctatcccgtctgttcctgtcagattctttttccataaaaagaagaagaccccgaatctcaccggtacaatgcaaaactgctgaaaaaaaaagaaagttcactggatacgggaacagtgccagtaggcttcaccacatggacaaaacaattgacgataaaataagcaggtgagcttctttttcaagtcacgatccctttatgtctcagaaacaatatatacaagctaaacccttttgaaccagttctctcttcatagttatgttcacataaattgcgggaacaagactccgctggctgtcaggtacacgttgtaacgttttcgtccgcccaattattagcacaacattggcaaaaagaaaaactgctcgttttctctacaggtaaattacaatttttttcagtaattttcgctgaaaaatttaaagggcaggaaaaaaagacgatctcgactttgcatagatgcaagaactgtggtcaaaacttgaaatagtaattttgctgtgcgtgaactaataaatatatatatatatatatatatatatttgtgtattttgtatatgtaattgtgcacgtcttggctattggatataagattttcgcgggttgatgacatagagcgtgtactactgtaatagttgtatattcaaaagctgctgcgtggagaaagactaaaatagataaaaagcacacattttgacttcggtaccgtcaacttagtgggacagtcttttatatttggtgtaagctcatttctggtactattcgaaacagaacagtgttttctgtattaccgtccaatcgtttgtcatgagttttgtattgattttgtcgttagtgttcggaggatgttgttccaatgtgattagtttcgagcacatggtgcaaggcagcaatataaatttgggaaatattgttacattcactcaattcgtgtctgtgacgctaattcagttgcccaatgctttggacttctctcactttccgtttaggttgcgacctagacacattcctcttaagatccatatgttagctgtgtttttgttctttaccagttcagtcgccaataacagtgtgtttaaatttgacatttccgttccgattcatattatcattagattttcaggtaccactttgacgatgataataggttgggctgtttgtaataagaggtactccaaacttcaggtgcaatctgccatcattatgacgcttggtgcgattgtcgcatcattataccgtgacaaagaattttcaatggacagtttaaagttgaatacggattcagtgggtatgacccaaaaatctatgtttggtatctttgttgtgctagtggccactgccttgatgtcattgttgtcgttgctcaacgaatggacgtataacaagtacgggaaacattggaaagaaactttgttctattcgcatttcttggctctaccgttgtttatgttggggtacacaaggctcagagacgaattcagagacctcttaatttcctcagactcaatggatattcctattgttaaattaccaattgctacgaaacttttcatgctaatagcaaataacgtgacccagttcatttgtatcaaaggtgttaacatgctagctagtaacacggatgctttgacactttctgtcgtgcttctagtgcgtaaatttgttagtcttttactcagtgtctacatctacaagaacgtcctatccgtgactgcatacctagggaccatcaccgtgttcctgggagctggtttgtattcatatggttcggtcaaaactgcactgcctcgctgaaacaatccacgtctgtatgatactcgtttcagaatttttttgattttctgccggatatggtttctcatctttacaatcgcattcttaattataccagaacgtaattcaatgatcccagtgactcgtaactcttatatgtcaatttaagc

SEQ ID NO: 25 - DNA, 870 bp, Pichia pastoris
ggccgagcgggcctagattttcactacaaatttcaaaactacgcggatttattgtctcagagagcaatttggcatttctgagcgtagcaggaggcttcataagattgtataggaccgtaccaacaaattgccgaggcacaacacggtatgctgtgcacttatgtggctacttccctacaacggaatgaaaccttcctctttccgcttaaacgagaaagtgtgtcgcaattgaatgcaggtgcctgtgcgccttggtgtattgtttttgagggcccaatttatcaggcgccttttttcttggttgttttcccttagcctcaagcaaggttggtctatttcatctccgcttctataccgtgcctgatactgttggatgagaacacgactcaacttcctgctgctctgtattgccagtgttttgtctgtgatttggatcggagtcctccttacttggaatgataataatcttggcggaatctccctaaacggaggcaaggattctgcctatgatgatctgctatcattgggaagcttcaacgacatggaggtcgactcctatgtcaccaacatctacgacaatgctccagtgctaggatgtacggatttgtcttatcatggattgttgaaagtcaccccaaagcatgacttagcttgcgatttggagttcataagagctcagattttggacattgacgtttactccgccataaaagacttagaagataaagccttgactgtaaaacaaaaggttgaaaaacactggtttacgttttatggtagttcagtctttctgcccgaacacgatgtgcattacctggttagacgagtcatcttttcggctgaaggaaaggcgaactctccagtaacatc

SEQ ID NO: 26 - DNA, 1733 bp, Pichia pastoris
ccatatgatgggtgtttgctcactcgtatggatcaaaattccatggtttcttctgtacaacttgtacacttatttggacttttctaacggtttttctggtgatttgagaagtccttattttggtgttcgcagcttatccgtgattgaaccatcagaaatactgcagctcgttatctagtttcagaatgtgttgtagaatacaatcaattctgagtctagtttgggtgggtcttggcgacgggaccgttatatgcatctatgcagtgttaaggtacatagaatgaaaatgtaggggttaatcgaaagcatcgttaatttcagtagaacgtagttctattccctacccaaataatttgccaagaatgcttcgtatccacatacgcagtggacgtagcaaatttcactttggactgtgacctcaagtcgttatcttctacttggacattgatggtcattacgtaatccacaaagaattggatagcctctcgttttatctagtgcacagcctaatagcacttaagtaagagcaatggacaaatttgcatagacattgagctagatacgtaactcagatcttgttcactcatggtgtactcgaagtactgctggaaccgttacctcttatcatttcgctactggctcgtgaaactactggatgaaaaaaaaaaaagagctgaaagcgagatcatcccattttgtcatcatacaaattcacgcttgcagttttgcttcgttaacaagacaagatgtctttatcaaagacccgttttttcttcttgaagaatacttccctgttgagcacatgcaaaccatatttatctcagatttcactcaacttgggtgcttccaagagaagtaaaattcttcccactgcatcaacttccaagaaacccgtagaccagtttctcttcagccaaaagaagttgctcgccgatcaccgcggtaacagaggagtcagaaggtttcacacccttccatcccgatttcaaagtcaaagtgctgcgttgaaccaaggttttcaggttgccaaagcccagtctgcaaaaactagttccaaatggcctattaattcccataaaagtgttggctacgtatgtatcggtacctccattctggtatttgctattgttgtcgttggtgggttgactagactgaccgaatccggtctttccataacggagtggaaacctatcactggttcggttcccccactgactgaggaagactggaagttggaatttgaaaaatacaaacaaagccctgagtttcaggaactaaattctcacataacattggaagagttcaagtttatattttccatggaatggggacatagattgttgggaagggtcatcggcctgtcgtttgttcttcccacgttttacttcattgcccgtcgaaagtgttccaaagatgttgcattgaaactgcttgcaatatgctctatgataggattccaaggtttcatcggctggtggatggtgtattccggattggacaaacagcaattggctgaacgtaactccaaaccaactgtgtctccatatcgcttaactacccatcttggaactgcatttgttatttactgttacatgatttacacagggcttcaagttttgaagaactataagatcatgaaacagcctgaagcgtatgttcaaattttcaagcaaattgcgtctccaaaattgaaaactttcaagagactctcttcagttctattaggcctggtg

SEQ ID NO: 27 - DNA, 981 bp, Mus musculus
atgtctgccaacctaaaatatctttccttgggaattttggtgtttcagactaccagtctggttctaacgatgcggtattctaggactttaaaagaggaggggcctcgttatctgtcttctacagcagtggttgtggctgaatttttgaagataatggcctgcatctttttagtctacaaagacagtaagtgtagtgtgagagcactgaatagagtactgcatgatgaaattcttaataagcccatggaaaccctgaagctcgctatcccgtcagggatatatactcttcagaacaacttactctatgtggcactgtcaaacctagatgcagccacttaccaggttacatatcagttgaaaatacttacaacagcattattttctgtgtctatgcttggtaaaaaattaggtgtgtaccagtggctctccctagtaattctgatggcaggagttgcttttgtacagtggccttcagattctcaagagctgaactctaaggacctttcaacaggctcacagtttgtaggcctcatggcagttctcacagcctgtttttcaagtggctttgctggagtttattttgagaaaatcttaaaagaaacaaaacagtcagtatggataaggaacattcaacttggtttctttggaagtatatttggattaatgggtgtatacgtttatgatggagaattggtctcaaagaatggattttttcagggatataatcaactgacgtggatagttgttgctctgcaggcacttggaggccttgtaatagctgctgtcatcaaatatgcagataacattttaaaaggatttgcgacctccttatccataatattgtcaacaataatatcttatttttggttgcaagattttgtgccaaccagtgtctttttccttggagccatccttgtaatagcagctactttcttgtatggttacgatcccaaacctgcaggaaatcccactaaagcatag

SEQ ID NO: 28 - DNA, 1128 bp, Pichia pastoris
gatctggccattgtgaaacttgacactaaagacaaaactcttagagtttccaatcacttaggagacgatgtttcctacaacgagtacgatccctcattgatcatgagcaatttgtatgtgaaaaaagtcatcgaccttgacaccttggataaaagggctggaggaggtggaaccacctgtgcaggcggtctgaaagtgttcaagtacggatctactaccaaatatacatctggtaacctgaacggcgtcaggttagtatactggaacgaaggaaagttgcaaagctccaaatttgtggttcgatcctctaattactctcaaaagcttggaggaaacagcaacgccgaatcaattgacaacaatggtgtgggttttgcctcagctggagactcaggcgcatggattctttccaagctacaagatgttagggagtaccagtcattcactgaaaagctaggtgaagctacgatgagcattttcgatttccacggtcttaaacaggagacttctactacagggcttggggtagttggtatgattcattcttacgacggtgagttcaaacagtttggtttgttcactccaatgacatctattctacaaagacttcaacgagtgaccaatgtagaatggtgtgtagcgggttgcgaagatggggatgtggacactgaaggagaacacgaattgagtgatttggaacaactgcatatgcatagtgattccgactagtcaggcaagagagagccctcaaatttacctctctgcccctcctcactccttttggtacgcataattgcagtataaagaacttgctgccagccagtaatcttatttcatacgcagttctatatagcacataatcttgcttgtatgtatgaaatttaccgcgttttagttgaaattgtttatgttgtgtgccttgcatgaaatctctcgttagccctatccttacatttaactggtctcaaaacctctaccaattccattgctgtacaacaatatgaggcggcattactgtagggttggaaaaaaattgtcattccagctagagatcacacgacttcatcacgcttattgctcctcattgctaaatcatttactcttgacttcgacccagaaaagttcgcc

SEQ ID NO: 29 - DNA, 1231 bp, Pichia pastoris
gcatgtcaaacttgaacacaacgactagatagttgttttttctatataaaacgaaacgttatcatctttaataatcattgaggtttacccttatagttccgtattttcgtttccaaacttagtaatcttttggaaatatcatcaaagctggtgccaatcttcttgtttgaagtttcaaactgctccaccaagctacttagagactgttctaggtctgaagcaacttcgaacacagagacagctgccgccgattgttcttttttgtgtttttcttctggaagaggggcatcatcttgtatgtccaatgcccgtatcctttctgagttgtccgacacattgtccttcgaagagtttcctgacattgggcttcttctatccgtgtattaattttgggttaagttcctcgtttgcatagcagtggatacctcgatttttttggctcctatttacctgacataatattctactataatccaacttggacgcgtcatctatgataactaggctctcctttgttcaaaggggacgtcttcataatccactggcacgaagtaagtctgcaacgaggcggcttttgcaacagaacgatagtgtcgtttcgtacttggactatgctaaacaaaaggatctgtcaaacatttcaaccgtgtttcaaggcactctttacgaattatcgaccaagaccttcctagacgaacatttcaacatatccaggctactgcttcaaggtggtgcaaatgataaaggtatagatattagatgtgtttgggacctaaaacagttcttgcctgaagattcccttgagcaacaggcttcaatagccaagttagagaagcagtaccaaatcggtaacaaaagggggaagcatataaaacctttactattgcgacaaaatccatccttgaaagtaaagctgtttgttcaatgtaaagcatacgaaacgaaggaggtagatcctaagatggttagagaacttaacgggacatactccagctgcatcccatattacgatcgctggaagacttttttcatgtacgtatcgcccaccaacctttcaaagcaagctaggtatgattttgacagttctcacaatccattggttttcatgcaacttgaaaaaacccaactcaaacttcatggggatccatacaatgtaaatcattacgagagggcgaggttgaaaagtttccattgcaatcacgtcgcatcatggctactgaaaggccttaac

SEQ ID NO: 30 - DNA, 937 bp, Pichia pastoris
tcattctatatgttcaagaaaagggtagtgaaaggaaagaaaaggcatataggcgagggagagttagctagcatacaagataatgaaggatcaatagcggtagttaaagtgcacaagaaaagagcacctgttgaggctgatgataaagctccaattacattgccacagagaaacacagtaacagaaataggaggggatgcaccacgagaagagcattcagtgaacaactttgccaaattcataaccccaagcgctaataagccaatgtcaaagtcggctactaacattaatagtacaacaactatcgattttcaaccagatgtttgcaaggactacaaacagacaggttactgcggatatggtgacacttgtaagtttttgcacctgagggatgatttcaaacagggatggaaattagatagggagtgggaaaatgtccaaaagaagaagcataatactctcaaaggggttaaggagatccaaatgtttaatgaagatgagctcaaagatatcccgtttaaatgcattatatgcaaaggagattacaaatcacccgtgaaaacttcttgcaatcattatttttgcgaacaatgtttcctgcaacggtcaagaagaaaaccaaattgtattatatgtggcagagacactttaggagttgctttaccagcaaagaagttgtcccaatttctggctaagatacataataatgaaagtaataaagtttagtaattgcattgcgttgactattgattgcattgatgtcgtgtgatactttcaccgaaaaaaaacacgaagcgcaataggagcggttgcatattagtccccaaagctatttaattgtgcctgaaactgttttttaagctcatcaagcataattgtatgcattgcgacgtaaccaacgtttaggcgcagtttaatcatagcccactgctaagcc

SEQ ID NO: 31 - DNA, 1906 bp, Pichia pastoris
cggaggaatgcaaataataatctccttaattacccactgataagctcaagagacgcggtttgaaaacgatataatgaatcatttggattttataataaaccctgacagtttttccactgtattgttttaacactcattggaagctgtattgattctaagaagctagaaatcaatacggccatacaaaagatgacattgaataagcaccggcttttttgattagcatataccttaaagcatgcattcatggctacatagttgttaaagggcttcttccattatcagtataatgaattacataatcatgcacttatatttgcccatctctgttctctcactcttgcctgggtatattctatgaaattgcgtatagcgtgtctccagttgaaccccaagcttggcgagtttgaagagaatgctaaccttgcgtattccttgcttcaggaaacattcaaggagaaacaggtcaagaagccaaacattttgatccttcccgagttagcattgactggctacaattttcaaagccagcagcggatagagccttttttggaggaaacaaccaagggagctagtacccaatgggctcaaaaagtatccaagacgtgggattgctttactttaataggatacccagaaaaaagtttagagagccctccccgtatttacaacagtgcggtacttgtatcgcctcagggaaaagtaatgaacaactacagaaagtccttcttgtatgaagctgatgaacattggggatgttcggaatcttctgatgggtttcaaacagtagatttattaattgaaggaaagactgtaaagacatcatttggaatttgcatggatttgaatccttataaatttgaagctccattcacagacttcgagttcagtggccattgcttgaaaaccggtacaagactcattttgtgcccaatggcctggttgtcccctctatcgccttccattaaaaaggatcttagtgatatagagaaaagcagacttcaaaagttctaccttgaaaaaatagataccccggaatttgacgttaattacgaattgaaaaaagatgaagtattgcccacccgtatgaatgaaacgttggaaacaattgactttgagccttcaaaaccggactactctaatataaattattggatactaaggttttttccctttctgactcatgtctataaacgagatgtgctcaaagagaatgcagttgcagtcttatgcaaccgagttggcattgagagtgatgtcttgtacggaggatcaaccacgattctaaacttcaatggtaagttagcatcgacacaagaggagctggagttgtacgggcagactaatagtctcaaccccagtgtggaagtattgggggcccttggcatgggtcaacagggaattctagtacgagacattgaattaacataatatacaatatacaataaacacaaataaagaatacaagcctgacaaaaattcacaaattattgcctagacttgtcgttatcagcagcgacctttttccaatgctcaatttcacgatatgccttttctagctctgctttaagcttctcattggaattggctaactcgttgactgcttggtcagtgatgagtttctccaaggtccatttctcgatgttgttgttttcgttttcctttaatctcttgatataatcaacagccttctttaatatctgagccttgttcgagtcccctgttggcaacagagcggccagttcctttattccgtggtttatattttctcttctacgcctttctacttctttgtgattctctttacgcatcttatgccattcttcagaaccagtggctggcttaaccgaatagccagagcctgaagaagccgcactagaagaagcagtggcattgttgactatgg

SEQ ID NO: 32 - DNA, 1224 bp, Artificial Sequence
  GnTI
tcagtcagtgctcttgatggtgacccagcaagtttgaccagagaagtgattagattggcccaagacgcagaggtggagttggagagacaacgtggactgctgcagcaaatcggagatgcattgtctagtcaaagaggtagggtgcctaccgcagctcctccagcacagcctagagtgcatgtgacccctgcaccagctgtgattcctatcttggtcatcgcctgtgacagatctactgttagaagatgtctggacaagctgttgcattacagaccatctgctgagttgttccctatcatcgttagtcaagactgtggtcacgaggagactgcccaagccatcgcctcctacggatctgctgtcactcacatcagacagcctgacctgtcatctattgctgtgccaccagaccacagaaagttccaaggttactacaagatcgctagacactacagatgggcattgggtcaagtcttcagacagtttagattccctgctgctgtggtggtggaggatgacttggaggtggctcctgacttctttgagtactttagagcaacctatccattgctgaaggcagacccatccctgtggtgtgtctctgcctggaatgacaacggtaaggagcaaatggtggacgcttctaggcctgagctgttgtacagaaccgacttctttcctggtctgggatggttgctgttggctgagttgtgggctgagttggagcctaagtggccaaaggcattctgggacgactggatgagaagacctgagcaaagacagggtagagcctgtatcagacctgagatctcaagaaccatgacctttggtagaaagggagtgtctcacggtcaattctttgaccaacacttgaagtttatcaagctgaaccagcaatttgtgcacttcacccaactggacctgtcttacttgcagagagaggcctatgacagagatttcctagctagagtctacggagctcctcaactgcaagtggagaaagtgaggaccaatgacagaaaggagttgggagaggtgagagtgcagtacactggtagggactcctttaaggctttcgctaaggctctgggtgtcatggatgaccttaagtctggagttcctagagctggttacagaggtattgtcacctttcaattcagaggtagaagagtccacttggctcctccacctacttgggagggttatgatccttcttggaattag

SEQ ID NO: 33 - DNA, 99 bp, Pichia pastoris
atgcccagaaaaatatttaactacttcattttgactgtattcatggcaattcttgctattgttttacaatggtctatagagaatggacatgggcgcgcc

SEQ ID NO: 34 - DNA, 435 bp, Pichia pastoris
gaagtaaagttggcgaaactttgggaacctttggttaaaactttgtaatttttgtcgctacccattaggcagaatctgcatcttgggagggggatgtggtggcgttctgagatgtacgcgaagaatgaagagccagtggtaacaacaggcctagagagatacgggcataatgggtataacctacaagttaagaatgtagcagccctggaaaccagattgaaacgaaaaacgaaatcatttaaactgtaggatgttttggctcattgtctggaaggctggctgtttattgccctgttctttgcatgggaataagctattatatccctcacataatcccagaaaatagattgaagcaacgcgaaatccttacgtatcgaagtagccttcttacacattcacgttgtacggataagaaaactactcaaacgaacaatc

SEQ ID NO: 35 - DNA, 404 bp, Pichia pastoris
aatagatatagcgagattagagaatgaataccttcttctaagcgatcgtccgtcatcatagaatatcatggactgtatagttttttttttgtacatataatgattaaacggtcatccaacatctcgttgacagatctctcagtacgcgaaatccctgactatcaaagcaagaaccgatgaagaaaaaaacaacagtaacccaaacaccacaacaaacactttatcttctcccccccaacaccaatcatcaaagagatgtcggaacacaaacaccaagaagcaaaaactaaccccatataaaaacatcctggtagataatgctggtaacccgctctccttccatattctgggctacttcacgaagtctgaccggtctcagttgatcaacatgatcctcgaaatgg

SEQ ID NO: 36 - DNA, 1407 bp, Mus musculus
gagcccgctgacgccaccatccgtgagaagagggcaaagatcaaagagatgatgacccatgcttggaataattataaacgctatgcgtggggcttgaacgaactgaaacctatatcaaaagaaggccattcaagcagtttgtttggcaacatcaaaggagctacaatagtagatgccctggatacccttttcattatgggcatgaagactgaatttcaagaagctaaatcgtggattaaaaaatatttagattttaatgtgaatgctgaagtttctgtttttgaagtcaacatacgcttcgtcggtggactgctgtcagcctactatttgtccggagaggagatatttcgaaagaaagcagtggaacttggggtaaaattgctacctgcatttcatactccctctggaataccttgggcattgctgaatatgaaaagtgggatcgggcggaactggccctgggcctctggaggcagcagtatcctggccgaatttggaactctgcatttagagtttatgcacttgtcccacttatcaggagacccagtctttgccgaaaaggttatgaaaattcgaacagtgttgaacaaactggacaaaccagaaggcctttatcctaactatctgaaccccagtagtggacagtggggtcaacatcatgtgtcggttggaggacttggagacagcttttatgaatatttgcttaaggcgtggttaatgtctgacaagacagatctcgaagccaagaagatgtattttgatgctgttcaggccatcgagactcacttgatccgcaagtcaagtgggggactaacgtacatcgcagagtggaaggggggcctcctggaacacaagatgggccacctgacgtgctttgcaggaggcatgtttgcacttggggcagatggagctccggaagcccgggcccaacactaccttgaactcggagctgaaattgcccgcacttgtcatgaatcttataatcgtacatatgtgaagttgggaccggaagcgtttcgatttgatggcggtgtggaagctattgccacgaggcaaaatgaaaagtattacatcttacggcccgaggtcatcgagacatacatgtacatgtggcgactgactcacgaccccaagtacaggacctgggcctgggaagccgtggaggctctagaaagtcactgcagagtgaacggaggctactcaggcttacgggatgtttacattgcccgtgagagttatgacgatgtccagcaaagtttcttcctggcagagacactgaagtatttgtacttgatattttccgatgatgaccttcttccactagaacactggatcttcaacaccgaggctcatcctttccctatactccgtgaacagaagaaggaaattgatggcaaagagaaatga

SEQ ID NO: 37 - DNA, 318 bp, Saccharomyces cerevisiae
atgaacactatccacataataaaattaccgcttaactacgccaactacacctcaatgaaacaaaaaatctctaaatttttcaccaacttcatccttattgtgctgctttcttacattttacagttctcctataagcacaatttgcattccatgcttttcaattacgcgaaggacaattttctaacgaaaagagacaccatctcttcgccctacgtagttgatgaagacttacatcaaacaactttgtttggcaaccacggtacaaaaacatctgtacctagcgtagattccataaaagtgcatggcgtggggcgcgcc

SEQ ID NO: 38 - DNA, 1250 bp, Pichia pastoris
gagtcggccaagagatgataactgttactaagcttctccgtaattagtggtattttgtaacttttaccaataatcgtttatgaatacggatatttttcgaccttatccagtgccaaatcacgtaacttaatcatggtttaaatactccacttgaacgattcattattcagaaaaaagtcaggttggcagaaacacttgggcgctttgaagagtataagagtattaagcattaaacatctgaactttcaccgccccaatatactactctaggaaactcgaaaaattcctttccatgtgtcatcgcttccaacacactttgctgtatccttccaagtatgtccattgtgaacactgatctggacggaatcctacctttaatcgccaaaggaaaggttagagacatttatgcagtcgatgagaacaacttgctgttcgtcgcaactgaccgtatctccgcttacgatgtgattatgacaaacggtattcctgataagggaaagattttgactcagctctcagttttctggtttgattttttggcaccctacataaagaatcatttggttgcttctaatgacaaggaagtctttgctttactaccatcaaaactgtctgaagaaaaatacaaatctcaattagagggacgatccttgatagtaaaaaagcacagactgatacctttggaagccattgtcagaggttacatcactggaagtgcatggaaagagtacaagaactcaaaaactgtccatggagtcaaggttgaaaacgagaaccttcaagagagcgacgcctttccaactccgattttcacaccttcaacgaaagctgaacagggtgaacacgatgaaaacatctctattgaacaagctgctgagattgtaggtaaagacatttgtgagaaggtcgctgtcaaggcggtcgagttgtattctgctgcaaaaaacctcgcccttttgaaggggatcattattgctgatacgaaattcgaatttggactggacgaaaacaatgaattggtactagtagatgaagttttaactccagattcttctagattttggaatcaaaagacttaccaagtgggtaaatcgcaagagagttacgataagcagtttctcagagattggttgacggccaacggattgaatggcaaagagggcgtagccatggatgcagaaattgctatcaagagtaaagaaaagtatattgaagcttatgaagcaattactggcaagaaatgggcttga

SEQ ID NO: 39 - DNA, 882 bp, Pichia pastoris
atgattagtaccctcctcgcctttttcagacatctgaaatttcccttattcttccaattccatataaaatcctatttaggtaattagtaaacaatgatcataaagtgaaatcattcaagtaaccattccgtttatcgttgatttaaaatcaataacgaatgaatgtcggtctgagtagtcaatttgttgccttggagctcattggcagggggtcttttggctcagtatggaaggttgaaaggaaaacagatggaaagtggttcgtcagaaaagaggtatcctacatgaagatgaatgccaaagagatatctcaagtgatagctgagttcagaattcttagtgagttaagccatcccaacattgtgaagtaccttcatcacgaacatatttctgagaataaaactgtcaatttatacatggaatactgtgatggtggagatctctccaagctgattcgaacacatagaaggaacaaagagtacatttcagaagaaaaaatatggagtatttttacgcaggttttattagcattgtatcgttgtcattatggaactgatttcacggcttcaaaggagtttgaatcgctcaataaaggtaatagacgaacccagaatccttcgtgggtagactcgacaagagttattattcacagggatataaaacccgacaacatctttctgatgaacaattcaaaccttgtcaaactgggagattttggattagcaaaaattctggaccaagaaaacgattttgccaaaacatacgtcggtacgccgtattacatgtctcctgaagtgctgttggaccaaccctactcaccattatgtgatatatggtctcttgggtgcgtcatgtatgagctatgtgcattgaggcctcctt

SEQ ID NO: 40 - DNA, 2100 bp, Saccharomyces cerevisiae
atgacagctcagttacaaagtgaaagtacttctaaaattgttttggttacaggtggtgctggatacattggttcacacactgtggtagagctaattgagaatggatatgactgtgttgttgctgataacctgtcgaattcaacttatgattctgtagccaggttagaggtcttgaccaagcatcacattcccttctatgaggttgatttgtgtgaccgaaaaggtctggaaaaggttttcaaagaatataaaattgattcggtaattcactttgctggtttaaaggctgtaggtgaatctacacaaatcccgctgagatactatcacaataacattttgggaactgtcgttttattagagttaatgcaacaatacaacgtttccaaatttgttttttcatcttctgctactgtctatggtgatgctacgagattcccaaatatgattcctatcccagaagaatgtcccttagggcctactaatccgtatggtcatacgaaatacgccattgagaatatcttgaatgatctttacaatagcgacaaaaaaagttggaagtttgctatcttgcgttattttaacccaattggcgcacatccctctggattaatcggagaagatccgctaggtataccaaacaatttgttgccatatatggctcaagtagctgttggtaggcgcgagaagctttacatcttcggagacgattatgattccagagatggtaccccgatcagggattatatccacgtagttgatctagcaaaaggtcatattgcagccctgcaatacctagaggcctacaatgaaaatgaaggtttgtgtcgtgagtggaacttgggttccggtaaaggttctacagtttttgaagtttatcatgcattctgcaaagcttctggtattgatcttccatacaaagttacgggcagaagagcaggtgatgttttgaacttgacggctaaaccagatagggccaaacgcgaactgaaatggcagaccgagttgcaggttgaagactcctgcaaggatttatggaaatggactactgagaatccttttggttaccagttaaggggtgtcgaggccagattttccgctgaagatatgcgttatgacgcaagatttgtgactattggtgccggcaccagatttcaagccacgtttgccaatttgggcgccagcattgttgacctgaaagtgaacggacaatcagttgttcttggctatgaaaatgaggaagggtatttgaatcctgatagtgcttatataggcgccacgatcggcaggtatgctaatcgtatttcgaagggtaagtttagtttatgcaacaaagactatcagttaaccgttaataacggcgttaatgcgaatcatagtagtatcggttctttccacagaaaaagatttttgggacccatcattcaaaatccttcaaaggatgtttttaccgccgagtacatgctgatagataatgagaaggacaccgaatttccaggtgatctattggtaaccatacagtatactgtgaacgttgcccaaaaaagtttggaaatggtatataaaggtaaattgactgctggtgaagcgacgccaataaatttaacaaatcatagttatttcaatctgaacaagccatatggagacactattgagggtacggagattatggtgcgttcaaaaaaatctgttgatgtcgacaaaaacatgattcctacgggtaatatcgtcgatagagaaattgctacctttaactctacaaagccaacggtcttaggccccaaaaatccccagtttgattgttgttttgtggtggatgaaaatgctaagccaagtcaaatcaatactctaaacaatgaattgacgcttattgtcaaggcttttcatcccgattccaatattacattagaagttttaagtacagagccaacttatcaattttataccggtgatttcttgtctgctggttacgaagcaagacaaggttttgcaattgagcctggtagatacattgatgctatcaatcaagagaactggaaagattgtgtaaccttgaaaaacggtgaaacttacgggtccaagattgtctacagattttcctga

SEQ ID NO: 41 - DNA, 512 bp, Pichia pastoris
taagcttcacgatttgtgttccagtttatcccccctttatataccgttaaccctttccctgttgagctgactgttgttgtattaccgcaatttttccaagtttgccatgcttttcgtgttatttgaccgatgtcttttttcccaaatcaaactatatttgttaccatttaaaccaagttatcttttgtattaagagtctaagtttgttcccaggcttcatgtgagagtgataaccatccagactatgattcttgttttttattgggtttgtttgtgtgatacatctgagttgtgattcgtaaagtatgtcagtctatctagatttttaatagttaattggtaatcaatgacttgtttgttttaacttttaaattgtgggtcgtatccacgcgtttagtatagctgttcatggctgttagaggagggcgatgtttatatacagaggacaagaatgaggaggcggcgtgtatttttaaaatggagacgcgactcctgtacaccttatcggttgg

SEQ ID NO: 42 - DNA, 1068 bp, Artificial Sequence
  Ga1T
ggtagagatttgtctagattgccacagttggttggtgtttccactccattgcaaggaggttctaactctgctgctgctattggtcaatcttccggtgagttgagaactggtggagctagaccacctccaccattgggagcttcctctcaaccaagaccaggtggtgattcttctccagttgttgactctggtccaggtccagcttctaacttgacttccgttccagttccacacactactgctttgtccttgccagcttgtccagaagaatccccattgttggttggtccaatgttgatcgagttcaacatgccagttgacttggagttggttgctaagcagaacccaaacgttaagatgggtggtagatacgctccaagagactgtgtttccccacacaaagttgctatcatcatcccattcagaaacagacaggagcacttgaagtactggttgtactacttgcacccagttttgcaaagacagcagttggactacggtatctacgttatcaaccaggctggtgacactattttcaacagagctaagttgttgaatgttggtttccaggaggctttgaaggattacgactacacttgtttcgttttctccgacgttgacttgattccaatgaacgaccacaacgcttacagatgtttctcccagccaagacacatttctgttgctatggacaagttcggtttctccttgccatacgttcaatacttcggtggtgtttccgctttgtccaagcagcagttcttgactatcaacggtttcccaaacaattactggggatggggtggtgaagatgacgacatctttaacagattggttttcagaggaatgtccatctctagaccaaacgctgttgttggtagatgtagaatgatcagacactccagagacaagaagaacgagccaaacccacaaagattcgacagaatcgctcacactaaggaaactatgttgtccgacggattgaactccttgacttaccaggttttggacgttcagagatacccattgtacactcagatcactgttgacatcggtactccatcctag

SEQ ID NO: 43 - DNA, 183 bp, Saccharomyces cerevisiae
atggccctctttctcagtaagagactgttgagatttaccgtcattgcaggtgcggttattgttctcctcctaacattgaattccaacagtagaactcagcaatatattccgagttccatctccgctgcatttgattttacctcaggatctatatcccctgaacaacaagtcatcgggcgcgcc

SEQ ID NO: 44 - DNA, 1074 bp, Drosophila melanogaster
atgaatagcatacacatgaacgccaatacgctgaagtacatcagcctgctgacgctgaccctgcagaatgccatcctgggcctcagcatgcgctacgcccgcacccggccaggcgacatcttcctcagctccacggccgtactcatggcagagttcgccaaactgatcacgtgcctgttcctggtcttcaacgaggagggcaaggatgcccagaagtttgtacgctcgctgcacaagaccatcattgcgaatcccatggacacgctgaaggtgtgcgtcccctcgctggtctatatcgttcaaaacaatctgctgtacgtctctgcctcccatttggatgcggccacctaccaggtgacgtaccagctgaagattctcaccacggccatgttcgcggttgtcattctgcgccgcaagctgctgaacacgcagtggggtgcgctgctgctcctggtgatgggcatcgtcctggtgcagttggcccaaacggagggtccgacgagtggctcagccggtggtgccgcagctgcagccacggccgcctcctctggcggtgctcccgagcagaacaggatgctcggactgtgggccgcactgggcgcctgcttcctctccggattcgcgggcatctactttgagaagatcctcaagggtgccgagatctccgtgtggatgcggaatgtgcagttgagtctgctcagcattcccttcggcctgctcacctgtttcgttaacgacggcagtaggatcttcgaccagggattcttcaagggctacgatctgtttgtctggtacctggtcctgctgcaggccggcggtggattgatcgttgccgtggtggtcaagtacgcggataacattctcaagggcttcgccacctcgctggccatcatcatctcgtgcgtggcctccatatacatcttcgacttcaatctcacgctgcagttcagcttcggagctggcctggtcatcgcctccatatttctctacggctacgatccggccaggtcggcgccgaagccaactatgcatggtcctggcggcgatgaggagaagctgctgccgcgcgtctag

SEQ ID NO: 45 - DNA, 798 bp, Pichia pastoris
tggacacaggagactcagaaacagacacagagcgttctgagtcctggtgctcctgacgtaggcctagaacaggaattattggctttatttgtttgtccatttcataggcttggggtaatagatagatgacagagaaatagagaagacctaatattttttgttcatggcaaatcgcgggttcgcggtcgggtcacacacggagaagtaatgagaagagctggtaatctggggtaaaagggttcaaaagaaggtcgcctggtagggatgcaatacaaggttgtcttggagtttacattgaccagatgatttggctttttctctgttcaattcacatttttcagcgagaatcggattgacggagaaatggcggggtgtggggtggatagatggcagaaatgctcgcaatcaccgcgaaagaaagactttatggaatagaactactgggtggtgtaaggattacatagctagtccaatggagtccgttggaaaggtaagaagaagctaaaaccggctaagtaactagggaagaatgatcagactttgatttgatgaggtctgaaaatactctgctgctttttcagttgctttttccctgcaacctatcattttccttttcataagcctgccttttctgttttcacttatatgagttccgccgagacttccccaaattctctcctggaacattctctatcgctctccttccaagttgcgccccctggcactgcctagtaatattaccacgcgacttatattcagttccacaatttccagtgttcgtagcaaatatcatcagcc

SEQ ID NO: 46 - DNA, 302 bp, Pichia pastoris
aatatatacctcatttgttcaatttggtgtaaagagtgtggcggatagacttcttgtaaatcaggaaagctacaattccaattgctgcaaaaaataccaatgcccataaaccagtatgagcggtgccttcgacggattgcttactttccgaccctttgtcgtttgattcttctgcctttggtgagtcagtttgtttcgactttatatctgactcatcaacttcctttacggttgcgtttttaatcataattttagccgttggcttattatcccttgagttggtaggagttttgatgatgctg

SEQ ID NO: 47 - DNA, 461 bp, Pichia pastoris
taactggccctttgacgtttctgacaatagttctagaggagtcgtccaaaaactcaactctgacttgggtgacaccaccacgggatccggttcttccgaggaccttgatgaccttggctaatgtaactggagttttagtatccattttaagatgtgtgtttctgtaggttctgggttggaaaaaaattttagacaccagaagagaggagtgaactggtttgcgtgggtttagactgtgtaaggcactactctgtcgaagttttagataggggttacccgctccgatgcatgggaagcgattagcccggctgttgcccgtttggtttttgaagggtaattttcaatatctctgtttgagtcatcaatttcatattcaaagattcaaaaacaaaatctggtccaaggagcgcatttaggattatggagttggcgaatcacttgaacgatagactattatttgc

SEQ ID NO: 48 - DNA, 1841 bp, Pichia pastoris
gtgacattcttgtctttgagatcagtaattgtagagcatagatagaataatattcaagaccaacggcttctcttcggaagctccaagtagcttatagtgatgagtaccggcatatatttataggcttaaaatttcgagggttcactatattcgtttagtgggaagagttcctttcactcttgttatctatattgtcagcgtggactgtttataactgtaccaacttagtttctttcaactccaggttaagagacataaatgtcctttgatgctgacaataatcagtggaattcaaggaaggacaatcccgacctcaatctgttcattaatgaagagttcgaatcgtccttaaatcaagcgctagactcaattgtcaatgagaaccctttctttgaccaagaaactataaatagatcgaatgacaaagttggaaatgagtccattagcttacatgatattgagcaggcagaccaaaataaaccgtcctttgagagcgatattgatggttcggcgccgttgataagagacgacaaattgccaaagaaacaaagctgggggctgagcaattttttttcaagaagaaatagcatatgtttaccactacatgaaaatgattcaagtgttgttaagaccgaaagatctattgcagtgggaacaccccatcttcaatactgcttcaatggaatctccaatgccaagtacaatgcatttacctttttcccagtcatcctatacgagcaattcaaattttttttcaatttatactttactttagtggctctctctcaagcgataccgcaacttcgcattggatatctttcttcgtatgtcgtcccacttttgtttgtactcatagtgaccatgtcaaaagaggcgatggatgatattcaacgccgaagaagggatagagaacagaacaatgaaccatatgaggttctgtccagcccatcaccagttttgtccaaaaacttaaaatgtggtcacttggttcgattgcataagggaatgagagtgcccgcagatatggttcttgtccagtcaagcgaatccaccggagagtcatttatcaagacagatcagctggatggtgagactgattggaagcttcggattgtttctccagttacacaatcgttaccaatgactgaacttcaaaatgtcgccatcactgcaagcgcaccctcaaaatcaattcactcctttcttggaagattgacctacaatgggcaatcatatggtcttacgatagacaacacaatgtggtgtaatactgtattagcttctggttcagcaattggttgtataatttacacaggtaaagatactcgacaatcgatgaacacaactcagcccaaactgaaaacgggcttgttagaactggaaatcaatagtttgtccaagatcttatgtgtttgtgtgtttgcattatctgtcatcttagtgctattccaaggaatagctgatgattggtacgtcgatatcatgcggtttctcattctattctccactattatcccagtgtctctgagagttaaccttgatcttggaaagtcagtccatgctcatcaaatagaaactgatagctcaatacctgaaaccgttgttagaactagtacaataccggaagacctgggaagaattgaatacctattaagtgacaaaactggaactcttactcaaaatgatatggaaatgaaaaaactacacctaggaacagtctcttatgctggtgataccatggatattatttctgatcatgttaaaggtcttaataacgctaaaacatcgaggaaagatcttggtatgagaataagagatttggttacaactctggccatctg

SEQ ID NO: 49 - DNA, 3105 bp, Artificial Sequence
  Drosophila melanogaster ManII
agagacgatccaattagacctccattgaaggttgctagatccccaagaccaggtcaatgtcaagatgttgttcaggacgtcccaaacgttgatgtccagatgttggagttgtacgatagaatgtccttcaaggacattgatggtggtgtttggaagcagggttggaacattaagtacgatccattgaagtacaacgctcatcacaagttgaaggtcttcgttgtcccacactcccacaacgatcctggttggattcagaccttcgaggaatactaccagcacgacaccaagcacatcttgtccaacgctttgagacatttgcacgacaacccagagatgaagttcatctgggctgaaatctcctacttcgctagattctaccacgatttgggtgagaacaagaagttgcagatgaagtccatcgtcaagaacggtcagttggaattcgtcactggtggatgggtcatgccagacgaggctaactcccactggagaaacgttttgttgcagttgaccgaaggtcaaacttggttgaagcaattcatgaacgtcactccaactgcttcctgggctatcgatccattcggacactctccaactatgccatacattttgcagaagtctggtttcaagaatatgttgatccagagaacccactactccgttaagaaggagttggctcaacagagacagttggagttcttgtggagacagatctgggacaacaaaggtgacactgctttgttcacccacatgatgccattctactcttacgacattcctcatacctgtggtccagatccaaaggtttgttgtcagttcgatttcaaaagaatgggttccttcggtttgtcttgtccatggaaggttccacctagaactatctctgatcaaaatgttgctgctagatccgatttgttggttgatcagtggaagaagaaggctgagttgtacagaaccaacgtcttgttgattccattgggtgacgacttcagattcaagcagaacaccgagtgggatgttcagagagtcaactacgaaagattgttcgaacacatcaactctcaggctcacttcaatgtccaggctcagttcggtactttgcaggaatacttcgatgctgttcaccaggctgaaagagctggacaagctgagttcccaaccttgtctggtgacttcttcacttacgctgatagatctgataactactggtctggttactacacttccagaccataccataagagaatggacagagtcttgatgcactacgttagagctgctgaaatgttgtccgcttggcactcctgggacggtatggctagaatcgaggaaagattggagcaggctagaagagagttgtccttgttccagcaccacgacggtattactggtactgctaaaactcacgttgtcgtcgactacgagcaaagaatgcaggaagctttgaaagcttgtcaaatggtcatgcaacagtctgtctacagattgttgactaagccatccatctactctccagacttctccttctcctacttcactttggacgactccagatggccaggttctggtgttgaggactctagaactaccatcatcttgggtgaggatatcttgccatccaagcatgttgtcatgcacaacaccttgccacactggagagagcagttggttgacttctacgtctcctctccattcgtttctgttaccgacttggctaacaatccagttgaggctcaggtttctccagtttggtcttggcaccacgacactttgactaagactatccacccacaaggttccaccaccaagtacagaatcatcttcaaggctagagttccaccaatgggtttggctacctacgttttgaccatctccgattccaagccagagcacacctcctacgcttccaatttgttgcttagaaagaacccaacttccttgccattgggtcaatacccagaggatgtcaagttcggtgatccaagagagatctccttgagagttggtaacggtccaaccttggctttctctgagcagggtttgttgaagtccattcagttgactcaggattctccacatgttccagttcacttcaagttcttgaagtacggtgttagatctcatggtgatagatctggtgcttacttgttcttgccaaatggtccagcttctccagtcgagttgggtcagccagttgtcttggtcactaagggtaaattggagtcttccgtttctgttggtttgccatctgtcgttcaccagaccatcatgagaggtggtgctccagagattagaaatttggtcgatattggttctttggacaacactgagatcgtcatgagattggagactcatatcgactctggtgatatcttctacactgatttgaatggattgcaattcatcaagaggagaagattggacaagttgccattgcaggctaactactacccaattccatctggtatgttcattgaggatgctaataccagattgactttgttgaccggtcaaccattgggtggatcttctttggcttctggtgagttggagattatgcaagatagaagattggcttctgatgatgaaagaggtttgggtcagggtgttttggacaacaagccagttttgcatatttacagattggtcttggagaaggttaacaactgtgtcagaccatctaagttgcatccagctggttacttgacttctgctgctcacaaagcttctcagtctttgttggatccattggacaagttcatcttcgctgaaaatgagtggatcggtgctcagggtcaattcggtggtgatcatccatctgctagagaggatttggatgtctctgtcatgagaagattgaccaagtcttctgctaaaacccagagagttggttacgttttgcacagaaccaatttgatgcaatgtggtactccagaggagcatactcagaagttggatgtctgtcacttgttgccaaatgttgctagatgtgagagaactaccttgactttcttgcagaatttggagcacttggatggtatggttgctccagaagtttgtccaatggaaaccgctgcttacgtctcttctcactcttcttga

SEQ ID NO: 50 - DNA, 108 bp, Saccharomyces cerevisiae
atgctgcttaccaaaaggttttcaaagctgttcaagctgacgttcatagttttgatattgtgcgggctgttcgtcattacaaacaaatacatggatgagaacacgtcg

SEQ ID NO: 51 - DNA, 1729 bp, Pichia pastoris
caagttgcgtccggtatacgtaacgtctcacgatgatcaaagataatacttaatcttcatggtctactgaataactcatttaaacaattgactaattgtacattatattgaacttatgcatcctattaacgtaatcttctggcttctctctcagactccatcagacacagaatatcgttctctctaactggtcctttgacgtttctgacaatagttctagaggagtcgtccaaaaactcaactctgacttgggtgacaccaccacgggatccggttcttccgaggaccttgatgaccttggctaatgtaactggagttttagtatccattttaagatgtgtgtttctgtaggttctgggttggaaaaaaattttagacaccagaagagaggagtgaactggtttgcgtgggtttagactgtgtaaggcactactctgtcgaagttttagataggggttacccgctccgatgcatgggaagcgattagcccggctgttgcccgtttggtttttgaagggtaattttcaatatctctgtttgagtcatcaatttcatattcaaagattcaaaaacaaaatctggtccaaggagcgcatttaggattatggagttggcgaatcacttgaacgatagactattatttgctgttcctaaagagggcagattgtatgagaaatgcgttgaattacttaggggatcagatattcagtttcgaagatccagtagattggatatagctttgtgcactaacctgcccctggcattggttttccttccagctgctgacattcccacgtttgtaggagagggtaaatgtgatttgggtataactggtattgaccaggttcaggaaagtgacgtagatgtcatacctttattagacttgaatttcggtaagtgcaagttgcagattcaagttcccgagaatggtgacttgaaagaacctaaacagctaattggtaaagaaattgtttcctcctttactagcttaaccaccaggtactttgaacaactggaaggagttaagcctggtgagccactaaagacaaaaatcaaatatgttggagggtctgttgaggcctcttgtgccctaggagttgccgatgctattgtggatcttgttgagagtggagaaaccatgaaagcggcagggctgatcgatattgaaactgttctttctacttccgcttacctgatctcttcgaagcatcctcaacacccagaactgatggatactatcaaggagagaattgaaggtgtactgactgctcagaagtatgtcttgtgtaattacaacgcacctagaggtaaccttcctcagctgctaaaactgactccaggcaagagagctgctaccgtttctccattagatgaagaagattgggtgggagtgtcctcgatggtagagaagaaagatgttggaagaatcatggacgaattaaagaaacaaggtgccagtgacattcttgtctttgagatcagtaattgtagagcatagatagaataatattcaagaccaacggcttctcttcggaagctccaagtagcttatagtgatgagtaccggcatatatttataggcttaaaatttcgagggttcactatattcgtttagtgggaagagttcctttcactcttgttatctatattgtcagcgtggactgtttataactgtaccaacttagtttctttcaactccaggttaagagacataaatgtcctttgatgc

SEQ ID NO: 52 - DNA, 1068 bp, Artificial Sequence
  Rattus norvegicus GnT II
tccttggtttaccaattgaacttcgaccagatgttgagaaacgttgacaaggacggtacttggtctcctggtgagttggttttggttgttcaggttcacaacagaccagagtacttgagattgttgatcgactccttgagaaaggctcaaggtatcagagaggttttggttatcttctcccacgatttctggtctgctgagatcaactccttgatctcctccgttgacttctgtccagttttgcaggttttcttcccattctccatccaattgtacccatctgagttcccaggttctgatccaagagactgtccaagagacttgaagaagaacgctgctttgaagttgggttgtatcaacgctgaatacccagattctttcggtcactacagagaggctaagttctcccaaactaagcatcattggtggtggaagttgcactttgtttgggagagagttaaggttttgcaggactacactggattgatcttgttcttggaggaggatcattacttggctccagacttctaccacgttttcaagaagatgtggaagttgaagcaacaagagtgtccaggttgtgacgttttgtccttgggaacttacactactatcagatccttctacggtatcgctgacaaggttgacgttaagacttggaagtccactgaacacaacatgggattggctttgactagagatgcttaccagaagttgatcgagtgtactgacactttctgtacttacgacgactacaactgggactggactttgcagtacttgactttggcttgtttgccaaaagtttggaaggttttggttccacaggctccaagaattttccacgctggtgactgtggaatgcaccacaagaaaacttgtagaccatccactcagtccgctcaaattgagtccttgttgaacaacaacaagcagtacttgttcccagagactttggttatcggagagaagtttccaatggctgctatttccccaccaagaaagaatggtggatggggtgatattagagaccacgagttgtgtaaatcctacagaagattgcagtag

SEQ ID NO: 53 - DNA, 300 bp, Saccharomyces cerevisiae
atgctgcttaccaaaaggttttcaaagctgttcaagctgacgttcatagttttgatattgtgcgggctgttcgtcattacaaacaaatacatggatgagaacacgtcggtcaaggagtacaaggagtacttagacagatatgtccagagttactccaataagtattcatcttcctcagacgccgccagcgctgacgattcaaccccattgagggacaatgatgaggcaggcaatgaaaagttgaaaagcttctacaacaacgttttcaactttctaatggttgattcgcccgggcgcgcc

SEQ ID NO: 54 - DNA, 1373 bp, Pichia pastoris
gatctggccttccctgaatttttacgtccagctatacgatccgttgtgactgtatttcctgaaatgaagtttcaacctaaagttttggttgtacttgctccacctaccacggaaactaatatcgaaaccaatgaaaaagtagaactggaatcgtcaatcgaaattcgcaaccaagtggaacccaaagacttgaatctttctaaagtctattctagtgacactaatggcaacagaagatttgagctgacttttcaaatgaatctcaataatgcaatatcaacatcagacaatcaatgggctttgtctagtgacacaggatcaattatagtagtgtcttctgcaggaagaataacttccccgatcctagaagtcggggcatccgtctgtgtcttaagatcgtacaacgaacaccttttggcaataacttgtgaaggaacatgcttttcatggaatttaaagaagcaagaatgtgttctaaacagcatttcattagcacctatagtcaattcacacatgctagttaagaaagttggagatgcaaggaactattctattgtatctgccgaaggagacaacaatccgttaccccagattctagactgcgaactttccaaaaatggcgctccaattgtggctcttagcacgaaagacatctactcttattcaaagaaaatgaaatgctggatccatttgattgattcgaaatactttgaattgttgggtgctgacaatgcactgtttgagtgtgtggaagcgctagaaggtccaattggaatgctaattcatagattggtagatgagttcttccatgaaaacactgccggtaaaaaactcaaactttacaacaagcgagtactggaggacctttcaaattcacttgaagaactaggtgaaaatgcgtctcaattaagagagaaacttgacaaactctatggtgatgaggttgaggcttcttgacctcttctctctatctgcgtttctttttttttttttttttttttttttttcagttgagccagaccgcgctaaacgcataccaattgccaaatcaggcaattgtgagacagtggtaaaaaagatgcctgcaaagttagattcacacagtaagagagatcctactcataaatgaggcgcttatttagtagctagtgatagccactgcggttctgctttatgctatttgttgtatgccttactatctttgtttggctcctttttcttgacgttttccgttggagggactccctattctgagtcatgagccgcacagattatcgcccaaaattgacaaaatcttctggcgaaaaaagtataaaaggagaaaaaagctcacccttttccagcgtagaaagtatatatcagtcattgaagac

SEQ ID NO: 55 - DNA, 1470 bp, Pichia pastoris
gggactttaactcaagtaaaaggatagttgtacaattatatatacgaagaataaatcattacaaaaagtattcgtttctttgattcttaacaggattcattttctgggtgtcatcaggtacagcgctgaatatcttgaagttaacatcgagctcatcatcgacgttcatcacactagccacgtttccgcaacggtagcaataattaggagcggaccacacagtgacgacatctttctctttgaaatggtatctgaagccttccatgaccaattgatgggctctagcgatgagttgcaagttattaatgtggttgaactcacgtgctactcgagcaccgaataaccagccagctccacgaggagaaacagcccaactgtcgacttcatctgggtcagaccaaaccaagtcacaaaatcctccttcatgagggacctcttgcgctcggctgagaactctgatttgatctaacatgcgaatatcgggagagagaccaccatggatacataatattttaccatcaatgatggcactaagggttaaaaagtcgaacacctggcaacagtacttccagacagtggtggaaccatatttattgagacattcctcataaaatccataaacctgagtgatctgtctggattcatgatttccccttaccaatgtgatatgttgaggaaacttaatttttaaaatcatgagtaacgtgaacgtctccaacgagaaatagcctctatccacatagtctcctaggaagatatagttctgttttattccattagaggaggatccgggaaacccaccactaatcttgaaaagttccagtagatcgtgaaattggccgtgaatatctccgcatactgtcactggactctgcactggctgtatattggattcctccatcagcaaatccttcacccgttcgcaaagatgcttcatatcattttcacttaaagccttgcagcttttgacttcttcaaaccactgatctggtcctctttctggcatgattaaggtctataatatttctgagctgagatgtaaaaaaaaataataaaaatggggagtgaaaaagtgtgtagcttttaggagtttgggattgataccccaaaatgatctttatgagaattaaaaggtagatacgcttttaataagaacacctatctatagtactttgtggtcttgagtaattgagatgttcagcttctgaggtttgccgttattctgggatagtagtgcgcgaccaaacaacccgccaggcaaagtgtgttgtgctcgaagacgattgccagaagagtaagtccgtcctgcctcagatgttacacactttcttccctagacagtcgatgcatcatcggatttaaacctgaaactttgatgccatgatacgcctagtcacgtcgactgagattttagataagccccgatccctttagtacattcctgttatccatggatggaatggcctgata

SEQ ID NO: 56 - DNA, 1043 bp, Pichia pastoris
aagcttgttcaccgttgggacttttccgtggacaatgttgactactccaggagggattccagctttctctactagctcagcaataatcaatgcagccccaggcgcccgttctgatggcttgatgaccgttgtattgcctgtcactatagccaggggtagggtccataaaggaatcatagcagggaaattaaaagggcatattgatgcaatcactcccaatggctctcttgccattgaagtctccatatcagcactaacttccaagaaggaccccttcaagtctgacgtgatagagcacgcttgctctgccacctgtagtcctctcaaaacgtcaccttgtgcatcagcaaagactttaccttgctccaatactatgacggaggcaattctgtcaaaattctctctcagcaattcaaccaacttgaaagcaaattgctgtctcttgatgatggagacttttttccaagattgaaatgcaatgtgggacgactcaattgcttcttccagctcctcttcggttgattgaggaacttttgaaaccacaaaattggtcgttgggtcatgtacatcaaaccattctgtagatttagattcgacgaaagcgttgttgatgaaggaaaaggttggatacggtttgtcggtctctttggtatggccggtggggtatgcaattgcagtagaagataattggacagccattgttgaaggtagagaaaaggtcagggaacttgggggttatttataccattttaccccacaaataacaactgaaaagtacccattccatagtgagaggtaaccgacggaaaaagacgggcccatgttctgggaccaatagaactgtgtaatccattgggactaatcaacagacgattggcaatataatgaaatagttcgttgaaaagccacgtcagctgtcttttcattaactttggtcggacacaacattttctactgttgtatctgtcctactttgcttatcatctgccacagggcaagtggatttccttctcgcgcggctgggtgaaaacggttaacgtgaa

SEQ ID NO: 57 - DNA, 695 bp, Pichia pastoris
gccttgggggacttcaagtctttgctagaaactagatgaggtcaggccctcttatggttgtgtcccaattgggcaatttcactcacctaaaaagcatgacaattatttagcgaaataggtagtatattttccctcatctcccaagcagtttcgtttttgcatccatatctctcaaatgagcagctacgactcattagaaccagagtcaagtaggggtgagctcagtcatcagccttcgtttctaaaacgattgagttcttttgttgctacaggaagcgccctagggaactttcgcactttggaaatagattttgatgaccaagagcgggagttgatattagagaggctgtccaaagtacatgggatcaggccggccaaattgattggtgtgactaaaccattgtgtacttggacactctattacaaaagcgaagatgatttgaagtattacaagtcccgaagtgttagaggattctatcgagcccagaatgaaatcatcaaccgttatcagcagattgataaactcttggaaagcggtatcccattttcattattgaagaactacgataatgaagatgtgagagacggcgaccctctgaacgtagacgaagaaacaaatctacttttggggtacaatagagaaagtgaatcaagggaggtatttgtggccataatactcaactctatcattaatg

SEQ ID NO: 58 - DNA, 411 bp, Pichia pastoris
catatggtgagagccgttctgcacaactagatgttttcgagcttcgcattgtttcctgcagctcgactattgaattaagatttccggatatctccaatctcacaaaaacttatgttgaccacgtgctttcctgaggcgaggtgttttatatgcaagctgccaaaaatggaaaacgaatggccatttttcgcccaggcaaattattcgattactgctgtcataaagacagtgttgcaaggctcacatttttttttaggatccgagataaagtgaatacaggacagcttatctctatatcttgtaccattcgtgaatcttaagagttcggttagggggactctagttgagggttggcactcacgtatggctgggcgcagaaataaaattcaggcgcagcagcacttatcgatg

SEQ ID NO: 59 - DNA, 692 bp, Pichia pastoris
gaattcacagttataaataaaaacaaaaactcaaaaagtttgggctccacaaaataacttaatttaaatttttgtctaataaatgaatgtaattccaagattatgtgatgcaagcacagtatgcttcagccctatgcagctactaatgtcaatctcgcctgcgagcgggcctagattttcactacaaatttcaaaactacgcggatttattgtctcagagagcaatttggcatttctgagcgtagcaggaggcttcataagattgtataggaccgtaccaacaaattgccgaggcacaacacggtatgctgtgcacttatgtggctacttccctacaacggaatgaaaccttcctctttccgcttaaacgagaaagtgtgtcgcaattgaatgcaggtgcctgtgcgccttggtgtattgtttttgagggcccaatttatcaggcgccttttttcttggttgttttcccttagcctcaagcaaggttggtctatttcatctccgcttctataccgtgcctgatactgttggatgagaacacgactcaacttcctgctgctctgtattgccagtgttttgtctgtgatttggatcggagtcctccttacttggaatgataataatcttggcggaatctccctaaacggaggcaaggattctgcctatgatgatctgctatcattgggaagctt

SEQ ID NO: 60 - DNA, 546 bp, Pichia pastoris
gatatctccctggggacaatatgtgttgcaactgttcgttgttggtgccccagtcccccaaccggtactaatcggtctatgttcccgtaactcatattcggttagaactagaacaataagtgcatcattgttcaacattgtggttcaattgtcgaacattgctggtgcttatatctacagggaagacgataagcctttgtacaagagaggtaacagacagttaattggtatttctttgggagtcgttgccctctacgttgtctccaagacatactacattctgagaaacagatggaagactcaaaaatgggagaagcttagtgaagaagagaaagttgcctacttggacagagctgagaaggagaacctgggttctaagaggctggactttttgttcgagagttaaactgcataattttttctaagtaaatttcatagttatgaaatttctgcagcttagtgtttactgcatcgtttactgcatcaccctgtaaataatgtgagcttttttccttccattgcttggtatcttccttgctgctgttt

SEQ ID NO: 61 - DNA, 378 bp, Pichia pastoris
acaaaacagtcatgtacagaactaacgcctttaagatgcagaccactgaaaagaattgggtcccatttttcttgaaagacgaccaggaatctgtccattttgtttactcgttcaatcctctgagagtactcaactgcagtcttgataacggtgcatgtgatgttctatttgagttaccacatgattttggcatgtcttccgagctacgtggtgccactcctatgctcaatcttcctcaggcaatcccgatggcagacgacaaagaaatttgggtttcattcccaagaacgagaatatcagattgcgggtgttctgaaacaatgtacaggccaatgttaatgctttttgttagagaaggaacaaacttttttgctgagc

SEQ ID NO: 62 - DNA, 1302 bp, Pichia pastoris
actgggcctttagagggtgctgaagttgaccccttggtgcttctggaaaaagaactgaagggcaccagacaagcgcaacttcctggtattcctcgtctaagtggtggtgccataggatacatctcgtacgattgtattaagtactttgaaccaaaaactgaaagaaaactgaaagatgttttgcaacttccggaagcagctttgatgttgttcgacacgatcgtggcttttgacaatgtttatcaaagattccaggtaattggaaacgtttctctatccgttgatgactcggacgaagctattcttgagaaatattataagacaagagaagaagtggaaaagatcagtaaagtggtatttgacaataaaactgttccctactatgaacagaaagatattattcaaggccaaacgttcacctctaatattggtcaggaagggtatgaaaaccatgttcgcaagctgaaagaacatattctgaaaggagacatcttccaagctgttccctctcaaagggtagccaggccgacctcattgcaccctttcaacatctatcgtcatttgagaactgtcaatccttctccatacatgttctatattgactatctagacttccaagttgttggtgcttcacctgaattactagttaaatccgacaacaacaacaaaatcatcacacatcctattgctggaactcttcccagaggtaaaactatcgaagaggacgacaattatgctaagcaattgaagtcgtctttgaaagacagggccgagcacgtcatgctggtagatttggccagaaatgatattaaccgtgtgtgtgagcccaccagtaccacggttgatcgtttattgactgtggagagattttctcatgtgatgcatcttgtgtcagaagtcagtggaacattgagaccaaacaagactcgcttcgatgctttcagatccattttcccagcaggtaccgtctccggtgctccgaaggtaagagcaatgcaactcataggagaattggaaggagaaaagagaggtgtttatgcgggggccgtaggacactggtcgtacgatggaaaatcgatggacacatgtattgccttaagaacaatggtcgtcaaggacggtgtcgcttaccttcaagccggaggtggaattgtctacgattctgacccctatgacgagtacatcgaaaccatgaacaaaatgagatccaacaataacaccatcttggaggctgagaaaatctggaccgataggttggccagagacgagaatcaaagtgaatccgaagaaaacgatcaatga

SEQ ID NO: 63 - DNA, 1085 bp, Pichia pastoris
acggaggacgtaagtaggaatttatgtaatcatgccaatacatctttagatttcttcctcttctttttaacgaaagacctccagttttgcactctcgactctctagtatcttcccatttctgttgctgcaacctcttgccttctgtttccttcaattgttcttctttcttctgttgcacttggccttcttcctccatctttcgttttttttcaagccttttcagcagttcttcttccaagagcagttctttgattttctctctccaatccaccaaaaaactggatgaattcaaccgggcatcatcaatgttccactttctttctcttatcaataatctacgtgcttcggcatacgaggaatccagttgctccctaatcgagtcatccacaaggttagcatgggcctttttcagggtgtcaaaagcatctggagctcgtttattcggagtcttgtctggatggatcagcaaagactttttgcggaaagtctttcttatatcttccggagaacaacctggtttcaaatccaagatggcatagctgtccaatttgaaagtggaaagaatcctgccaatttccttctctcgtgtcagctcgttctcctccttttgcaacaggtccacttcatctggcatttttctttatgttaactttaattattattaattataaagttgattatcgttatcaaaataatcatattcgagaaataatccgtccatgcaatatataaataagaattcataataatgtaatgataacagtacctctgatgacctttgatgaaccgcaattttctttccaatgacaagacatccctataatacaattatacagtttatatatcacaaataatcacctttttataagaaaaccgtcctctccgtaacagaacttattatccgcacgttatggttaacacactactaataccgatatagtgtatgaagtcgctacgagatagccatccaggaaacttaccaattcatcagcactttcatgatccgattgttggctttattctttgcgagacagatacttgccaatgaaataactgatcccacagatgagaatccggtgctcgt

SEQ ID NO: 64 - DNA, 1014 bp, Artificial Sequence
  MmCST
atggctccagctagagaaaacgtttccttgttcttcaagttgtactgtttggctgttatgactttggttgctgctgcttacactgttgctttgagatacactagaactactgctgaggagttgtacttctccactactgctgtttgtatcactgaggttatcaagttgttgatctccgttggtttgttggctaaggagactggttctttgggaagattcaaggcttccttgtccgaaaacgttttgggttccccaaaggagttggctaagttgtctgttccatccttggtttacgctgttcagaacaacatggctttcttggctttgtctaacttggacgctgctgtttaccaagttacttaccagttgaagatcccatgtactgctttgtgtactgttttgatgttgaacagaacattgtccaagttgcagtggatctccgttttcatgttgtgtggtggtgttactttggttcagtggaagccagctcaagcttccaaagttgttgttgctcagaacccattgttgggtttcggtgctattgctatcgctgttttgtgttccggtttcgctggtgtttacttcgagaaggttttgaagtcctccgacacttctttgtgggttagaaacatccagatgtacttgtccggtatcgttgttactttggctggtacttacttgtctgacggtgctgagattcaagagaagggattcttctacggttacacttactatgtttggttcgttatcttcttggcttccgttggtggtttgtacacttccgttgttgttaagtacactgacaacatcatgaagggattctctgctgctgctgctattgttttgtccactatcgcttccgttttgttgttcggattgcagatcacattgtcctttgctttgggagctttgttggtttgtgtttccatctacttgtacggattgccaagacaagacactacttccattcagcaagaggctacttccaaggagagaatcatcggtgtttagtag

SEQ ID NO: 65 - DNA, 2172 bp, Artificial Sequence
  HsGNE
atggaaaagaacggtaacaacagaaagttgagagtttgtgttgctacttgtaacagagctgactactccaagttggctccaatcatgttcggtatcaagactgagccagagttcttcgagttggacgttgttgttttgggttcccacttgattgatgactacggtaacacttacagaatgatcgagcaggacgacttcgacatcaacactagattgcacactattgttagaggagaggacgaagctgctatggttgaatctgttggattggctttggttaagttgccagacgttttgaacagattgaagccagacatcatgattgttcacggtgacagattcgatgctttggctttggctacttccgctgctttgatgaacattagaatcttgcacatcgagggtggtgaagtttctggtactatcgacgactccatcagacacgctatcactaagttggctcactaccatgtttgttgtactagatccgctgagcaacacttgatttccatgtgtgaggaccacgacagaattttgttggctggttgtccatcttacgacaagttgttgtccgctaagaacaaggactacatgtccatcatcagaatgtggttgggtgacgacgttaagtctaaggactacatcgttgctttgcagcacccagttactactgacatcaagcactccatcaagatgttcgagttgactttggacgctttgatctccttcaacaagagaactttggttttgttcccaaacattgacgctggttccaaagagatggttagagttatgagaaagaagggtatcgaacaccacccaaacttcagagctgttaagcacgttccattcgaccaattcatccagttggttgctcatgctggttgtatgatcggtaactcctcctgtggtgttagagaagttggtgctttcggtactccagttatcaacttgggtactagacagatcggtagagagactggagaaaacgttttgcatgttagagatgctgacactcaggacaagattttgcaggctttgcacttgcaattcggaaagcagtacccatgttccaaaatctacggtgacggtaacgctgttccaagaatcttgaagtttttgaagtccatcgacttgcaagagccattgcagaagaagttctgtttcccaccagttaaggagaacatctcccaggacattgaccacatcttggagacattgtccgctttggctgttgatttgggtggaactaacttgagagttgctatcgtttccatgaagggagagatcgttaagaagtacactcagttcaacccaaagacttacgaggagagaatcaacttgatcttgcagatgtgtgttgaagctgctgctgaggctgttaagttgaactgtagaatcttgggtgttggtatctctactggtggtagagttaatccaagagagggtatcgttttgcactccactaagttgattcaggagtggaactccgttgatttgagaactccattgtccgacacattgcacttgccagtttgggttgacaacgacggtaattgtgctgctttggctgagagaaagttcggtcaaggaaagggattggagaacttcgttactttgatcactggtactggtattggtggtggtatcattcaccagcacgagttgattcacggttcttccttctgtgctgctgaattgggacacttggttgtttctttggacggtccagactgttcttgtggttcccacggttgtattgaagcttacgcatcaggaatggcattgcagagagaggctaagaagttgcacgacgaggacttgttgttggttgagggaatgtctgttccaaaggacgaggctgttggtgctttgcatttgatccaggctgctaagttgggtaatgctaaggctcagtccatcttgagaactgctggtactgctttgggattgggtgttgttaatatcttgcacactatgaacccatccttggttatcttgtccggtgttttggcttctcactacatccacatcgttaaggacgttatcagacagcaagctttgtcctccgttcaagacgttgatgttgttgtttccgacttggttgacccagctttgttgggtgctgcttccatggttttggactacactactagaagaatctactaatag

SEQ ID NO: 66 - DNA, 1854 bp, Pichia pastoris
cagttgagccagaccgcgctaaacgcataccaattgccaaatcaggcaattgtgagacagtggtaaaaaagatgcctgcaaagttagattcacacagtaagagagatcctactcataaatgaggcgcttatttagtagctagtgatagccactgcggttctgctttatgctatttgttgtatgccttactatctttgtttggctcctttttcttgacgttttccgttggagggactccctattctgagtcatgagccgcacagattatcgcccaaaattgacaaaatcttctggcgaaaaaagtataaaaggagaaaaaagctcacccttttccagcgtagaaagtatatatcagtcattgaagactattatttaaataacacaatgtctaaaggaaaagtttgtttggcctactccggtggtttggatacctccatcatcctagcttggttgttggagcagggatacgaagtcgttgcctttttagccaacattggtcaagaggaagactttgaggctgctagagagaaagctctgaagatcggtgctaccaagtttatcgtcagtgacgttaggaaggaatttgttgaggaagttttgttcccagcagtccaagttaacgctatctacgagaacgtctacttactgggtacctctttggccagaccagtcattgccaaggcccaaatagaggttgctgaacaagaaggttgttttgctgttgcccacggttgtaccggaaagggtaacgatcaggttagatttgagctttccttttatgctctgaagcctgacgttgtctgtatcgccccatggagagacccagaattcttcgaaagattcgctggtagaaatgacttgctgaattacgctgctgagaaggatattccagttgctcagactaaagccaagccatggtctactgatgagaacatggctcacatctccttcgaggctggtattctagaagatccaaacactactcctccaaaggacatgtggaagctcactgttgacccagaagatgcaccagacaagccagagttctttgacgtccactttgagaagggtaagccagttaaattagttctcgagaacaaaactgaggtcaccgatccggttgagatctttttgactgctaacgccattgctagaagaaacggtgttggtagaattgacattgtcgagaacagattcatcggaatcaagtccagaggttgttatgaaactccaggtttgactctactgagaaccactcacatcgacttggaaggtcttaccgttgaccgtgaagttagatcgatcagagacacttttgttaccccaacctactctaagttgttatacaacgggttgtactttaccccagaaggtgagtacgtcagaactatgattcagccttctcaaaacaccgtcaacggtgttgttagagccaaggcctacaaaggtaatgtgtataacctaggaagatactctgaaaccgagaaattgtacgatgctaccgaatcttccatggatgagttgaccggattccaccctcaagaagctggaggatttatcacaacacaagccatcagaatcaagaagtacggagaaagtgtcagagagaagggaaagtttttgggactttaactcaagtaaaaggatagttgtacaattatatatacgaagaataaatcattacaaaaagtattcgtttctttgattcttaacaggattcattttctgggtgtcatcaggtacagcgctgaatatcttgaagttaacatcgagctcatcatcgacgttcatcacactagccacgtttccgcaacggtagcaataattaggagcggaccacacagtgacgacatc

SEQ ID NO: 67 - DNA, 1308 bp, Artificial Sequence
  HSaccharomy ces cerevisiae SS
atggactctgttgaaaagggtgctgctacttctgtttccaacccaagaggtagaccatccagaggtagacctcctaagttgcagagaaactccagaggtggtcaaggtagaggtgttgaaaagccaccacacttggctgctttgatcttggctagaggaggttctaagggtatcccattgaagaacatcaagcacttggctggtgttccattgattggatgggttttgagagctgctttggactctggtgctttccaatctgtttgggtttccactgaccacgacgagattgagaacgttgctaagcaattcggtgctcaggttcacagaagatcctctgaggtttccaaggactcttctacttccttggacgctatcatcgagttcttgaactaccacaacgaggttgacatcgttggtaacatccaagctacttccccatgtttgcacccaactgacttgcaaaaagttgctgagatgatcagagaagagggttacgactccgttttctccgttgttagaaggcaccagttcagatggtccgagattcagaagggtgttagagaggttacagagccattgaacttgaacccagctaaaagaccaagaaggcaggattgggacggtgaattgtacgaaaacggttccttctacttcgctaagagacacttgatcgagatgggatacttgcaaggtggaaagatggcttactacgagatgagagctgaacactccgttgacatcgacgttgatatcgactggccaattgctgagcagagagttttgagatacggttacttcggaaaggagaagttgaaggagatcaagttgttggtttgtaacatcgacggttgtttgactaacggtcacatctacgtttctggtgaccagaaggagattatctcctacgacgttaaggacgctattggtatctccttgttgaagaagtccggtatcgaagttagattgatctccgagagagcttgttccaagcaaacattgtcctctttgaagttggactgtaagatggaggtttccgtttctgacaagttggctgttgttgacgaatggagaaaggagatgggtttgtgttggaaggaagttgcttacttgggtaacgaagtttctgacgaggagtgtttgaagagagttggtttgtctggtgctccagctgatgcttgttccactgctcaaaaggctgttggttacatctgtaagtgtaacggtggtagaggtgctattagagagttcgctgagcacatctgtttgttgatggagaaagttaataactcctgtcagaagtagtag

SEQ ID NO: 68 - DNA, 1080 bp, Artificial Sequence
  HsSPS
atgccattggaattggagttgtgtcctggtagatgggttggtggtcaacacccatgtttcatcatcgctgagatcggtcaaaaccaccaaggagacttggacgttgctaagagaatgatcagaatggctaaggaatgtggtgctgactgtgctaagttccagaagtccgagttggagttcaagttcaacagaaaggctttggaaagaccatacacttccaagcactcttggggaaagacttacggagaacacaagagacacttggagttctctcacgaccaatacagagagttgcagagatacgctgaggaagttggtatcttcttcactgcttctggaatggacgaaatggctgttgagttcttgcacgagttgaacgttccattcttcaaagttggttccggtgacactaacaacttcccatacttggaaaagactgctaagaaaggtagaccaatggttatctcctctggaatgcagtctatggacactatgaagcaggtttaccagatcgttaagccattgaacccaaacttttgtttcttgcagtgtacttccgcttacccattgcaaccagaggacgttaatttgagagttatctccgagtaccagaagttgttcccagacatcccaattggttactctggtcacgagactggtattgctatttccgttgctgctgttgctttgggtgctaaggttttggagagacacatcactttggacaagacttggaagggttctgatcactctgcttctttggaacctggtgagttggctgaacttgttagatcagttagattggttgagagagctttgggttccccaactaagcaattgttgccatgtgagatggcttgtaacgagaagttgggaaagtccgttgttgctaaggttaagatcccagagggtactatcttgactatggacatgttgactgttaaagttggagagccaaagggttacccaccagaggacatctttaacttggttggtaaaaaggttttggttactgttgaggaggacgacactattatggaggagttggttgacaaccacggaaagaagatcaagtcctag

SEQ ID NO: 69 - DNA, 1092 bp, Artificial Sequence
  MmmST6
gtttttcaaatgccaaagtcccaggagaaagttgctgttggtccagctccacaagctgttttctccaactccaagcaagatccaaaggagggtgttcaaatcttgtcctacccaagagttactgctaaggttaagccacaaccatccttgcaagtttgggacaaggactccacttactccaagttgaacccaagattgttgaagatttggagaaactacttgaacatgaacaagtacaaggtttcctacaagggtccaggtccaggtgttaagttctccgttgaggctttgagatgtcacttgagagaccacgttaacgtttccatgatcgaggctactgacttcccattcaacactactgaatgggagggatacttgccaaaggagaacttcagaactaaggctggtccatggcataagtgtgctgttgtttcttctgctggttccttgaagaactcccagttgggtagagaaattgacaaccacgacgctgttttgagattcaacggtgctccaactgacaacttccagcaggatgttggtactaagactactatcagattggttaactcccaattggttactactgagaagagattcttgaaggactccttgtacactgagggaatcttgattttgtgggacccatctgtttaccacgctgacattccacaatggtatcagaagccagactacaacttcttcgagacttacaagtcctacagaagattgcacccatcccagccattctacatcttgaagccacaaatgccatgggaattgtgggacatcatccaggaaatttccccagacttgatccaaccaaacccaccatcttctggaatgttgggtatcatcatcatgatgactttgtgtgaccaggttgacatctacgagttcttgccatccaagagaaagactgatgtttgttactaccaccagaagttcttcgactccgcttgtactatgggagcttaccacccattgttgttcgagaagaacatggttaagcacttgaacgaaggtactgacgaggacatctacttgttcggaaaggctactttgtccggtttcagaaacaacagatgttag

SEQ ID NO: 70 - DNA, 54 bp, Artificial Sequence
  HSA signal peptide
atgaagtgggttacctttatctctttgttgtttcttttctcttctgcttactct

SEQ ID NO: 71 - protein, 18 aa, Homo sapiens
MKWVTFISLLFLFSSAYS

SEQ ID NO: 72 - DNA, 1401 bp, Artificial Sequence
  TNFRII-Fc-fragment
ctgccagctcaagttgcttttactccatacgctccagaaccaggttctacttgtagattgagagagtactacgaccaaactgctcagatgtgttgttccaagtgttctccaggtcaacacgctaaggttttctgtactaagacttccgacactgtttgtgactcttgtgaggactccacttacactcaattgtggaactgggttccagaatgtttgtcctgtggttccagatgttcttccgaccaagttgagactcaggcttgtactagagagcagaacagaatctgtacttgtagacctggttggtactgtgctttgtccaagcaagagggttgtagattgtgtgctccattgagaaagtgtagaccaggtttcggtgttgctagaccaggtacagaaacttccgacgttgtttgtaagccatgtgctccaggaactttctccaacactacttcctccactgacatctgtagaccacaccaaatctgtaacgttgttgctatcccaggtaacgcttctatggacgctgtttgtacttctacttccccaactagatccatggctccaggtgctgttcatttgccacagccagtttccactagatcccaacacactcaaccaactccagaaccatctactgctccatccacttcctttttgttgccaatgggaccatctccacctgctgaaggttctactggtgacgagccaaagtcctgtgacaagacacatacttgtccaccatgtccagctccagaattgttgggtggtccatccgttttcttgttcccaccaaagccaaaggacactttgatgatctccagaactccagaggttacatgtgttgttgttgacgtttctcacgaggacccagaggttaagttcaactggtacgttgacggtgttgaagttcacaacgctaagactaagccaagagaagagcagtacaactccacttacagagttgtttccgttttgactgttttgcaccaggattggttgaacggtaaagaatacaagtgtaaggtttccaacaaggctttgccagctccaatcgaaaagacaatctccaaggctaagggtcaaccaagagagccacaggtttacactttgccaccatccagagaagagatgactaagaaccaggtttccttgacttgtttggttaaaggattctacccatccgacattgctgttgaatgggaatctaacggtcaaccagagaacaactacaagactactccaccagttttggattctgacggttccttcttcttgtactccaagttgactgttgacaagtccagatggcaacagggtaacgttttctcctgttccgttatgcatgaggctttgcacaaccactacactcaaaagtccttgtctttgtccccaggttag

SEQ ID NO: 73 - protein, 466 aa, Artificial Sequence
  TNFRII-Vc-fragment
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 74 - DNA, 1404 bp, Artificial Sequence
  TNFRII-Fc fragment
ctgccagctcaagttgcttttactccatacgctccagaaccaggttctacttgtagattgagagagtactacgaccaaactgctcagatgtgttgttccaagtgttctccaggtcaacacgctaaggttttctgtactaagacttccgacactgtttgtgactcttgtgaggactccacttacactcaattgtggaactgggttccagaatgtttgtcctgtggttccagatgttcttccgaccaagttgagactcaggcttgtactagagagcagaacagaatctgtacttgtagacctggttggtactgtgctttgtccaagcaagagggttgtagattgtgtgctccattgagaaagtgtagaccaggtttcggtgttgctagaccaggtacagaaacttccgacgttgtttgtaagccatgtgctccaggaactttctccaacactacttcctccactgacatctgtagaccacaccaaatctgtaacgttgttgctatcccaggtaacgcttctatggacgctgtttgtacttctacttccccaactagatccatggctccaggtgctgttcatttgccacagccagtttccactagatcccaacacactcaaccaactccagaaccatctactgctccatccacttcctttttgttgccaatgggaccatctccacctgctgaaggttctactggtgacgagccaaagtcctgtgacaagacacatacttgtccaccatgtccagctccagaattgttgggtggtccatccgttttcttgttcccaccaaagccaaaggacactttgatgatctccagaactccagaggttacatgtgttgttgttgacgtttctcacgaggacccagaggttaagttcaactggtacgttgacggtgttgaagttcacaacgctaagactaagccaagagaagagcagtacaactccacttacagagttgtttccgttttgactgttttgcaccaggattggttgaacggtaaagaatacaagtgtaaggtttccaacaaggctttgccagctccaatcgaaaagacaatctccaaggctaagggtcaaccaagagagccacaggtttacactttgccaccatccagagaagagatgactaagaaccaggtttccttgacttgtttggttaaaggattctacccatccgacattgctgttgaatgggaatctaacggtcaaccagagaacaactacaagactactccaccagttttggattctgacggttccttcttcttgtactccaagttgactgttgacaagtccagatggcaacagggtaacgttttctcctgttccgttatgcatgaggctttgcacaaccactacactcaaaagtccttgtctttgtccccaggtaagtag

SEQ ID NO: 75 - protein, 467 aa, Artificial Sequence
  TNFRII-Fc fragment
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - DNA, 1804 bp, Artificial Sequence
  POMGnT I
cgcgccatttctgaagctaacgaggaccctgaaccagaacaagattacgacgaggctttgggaagattggaatccccaagaagaagaggatcctcccctagaagagttttggacgttgaggtttactcttccagatccaaggtttacgttgctgttgacggtactactgttttggaggacgaggctagagaacaaggtagaggtatccacgttatcgttttgaaccaggctactggtcatgttatggctaagagagttttcgacacttactctccacacgaagatgaggctatggttttgttcttgaacatggttgctccaggtagagttttgatttgtactgttaaggacgagggatccttccatttgaaggacactgctaaggctttgttgagatccttgggttctcaagctggtccagctttgggatggagagatacttgggctttcgttggtagaaagggtggtccagttttgggtgaaaagcactctaagtccccagctttgtcctcttggggtgacccagttttgttgaaaactgacgttccattgtcctctgctgaagaggctgaatgtcactgggctgacactgagttgaacagaagaagaagaagattctgttccaaggttgagggttacggttctgtttgttcctgtaaggacccaactccaattgaattctccccagacccattgccagataacaaggttttgaacgttccagttgctgttatcgctggtaacagaccaaactacttgtacagaatgttgagatctttgttgtccgctcagggagtttctccacagatgatcactgttttcatcgacggttactacgaagaaccaatggacgttgttgctttgttcggattgagaggtattcagcacactccaatctccatcaagaacgctagagtttcccaacactacaaggcttccttgactgctactttcaacttgttcccagaggctaagttcgctgttgttttggaagaggacttggacattgctgttgatttcttctccttcttgtcccaatccatccacttgttggaagaggatgactccttgtactgtatctctgcttggaacgaccaaggttacgaacacactgctgaggatccagctttgttgtacagagttgagactatgccaggattgggatgggttttgagaaagtccttgtacaaagaggagttggagccaaagtggccaactccagaaaagttgtgggattgggacatgtggatgagaatgccagagcagagaagaggtagagagtgtatcatcccagacgtttccagatcttaccacttcggtattgttggattgaacatgaacggttacttccacgaggcttacttcaagaagcacaagttcaacactgttccaggtgttcagttgagaaacgttgactccttgaagaaagaggcttacgaggttgagatccacagattgttgtctgaggctgaggttttggatcactccaaggatccatgtgaggactcattcttgccagatactgagggtcatacttacgttgctttcatcagaatggaaactgacgacgactttgctacttggactcagttggctaagtgtttgcacatttgggacttggatgttagaggtaaccacagaggattgtggagattgttcagaaagaagaaccacttcttggttgttggtgttccagcttctccatactccgttaagaagccaccatccgttactccaattttcttggagccaccaccaaaggaagaaggtgctcctggagctgctgaacaaacttagtagttaa

SEQ ID NO: 77 - DNA, 99 bp, Saccharomyces cerevisiae
atgcacgtactgctgagcaaaaaaatagcacgctttctgttgatttcgtttgttttcgtgcttgcgctaatggtgacaataaatcatccagggcgcgcc

SEQ ID NO: 78 - DNA, 114 bp, Saccharomyces cerevisiae
atgctgattaggttaaagaagagaaaaatcctgcaggtcatcgtgagcgcagtagtgctaattttatttttttgttctgtgcataatgatgtgtcttctagttgggggcgcgcc

SEQ ID NO: 79 - DNA, 1666 bp, Artificial Sequence
  HYGr
gatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcggacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagct

SEQ ID NO: 80 - DNA, 57 bp, Saccharomyces cerevisiae
atgagattcccatccatcttcactgctgttttgttcgctgcttcttctgctttggct

SEQ ID NO: 81 - DNA, 1494 bp, Trichoderma reesei
cgcgccggatctcccaaccctacgagggcggcagcagtcaaggccgcattccagacgtcgtggaacgcttaccaccattttgcctttccccatgacgacctccacccggtcagcaacagctttgatgatgagagaaacggctggggctcgtcggcaatcgatggcttggacacggctatcctcatgggggatgccgacattgtgaacacgatccttcagtatgtaccgcagatcaacttcaccacgactgcggttgccaaccaaggcatctccgtgttcgagaccaacattcggtacctcggtggcctgctttctgcctatgacctgttgcgaggtcctttcagctccttggcgacaaaccagaccctggtaaacagccttctgaggcaggctcaaacactggccaacggcctcaaggttgcgttcaccactcccagcggtgtcccggaccctaccgtcttcttcaaccctactgtccggagaagtggtgcatctagcaacaacgtcgctgaaattggaagcctggtgctcgagtggacacggttgagcgacctgacgggaaacccgcagtatgcccagcttgcgcagaagggcgagtcgtatctcctgaatccaaagggaagcccggaggcatggcctggcctgattggaacgtttgtcagcacgagcaacggtacctttcaggatagcagcggcagctggtccggcctcatggacagcttctacgagtacctgatcaagatgtacctgtacgacccggttgcgtttgcacactacaaggatcgctgggtccttgctgccgactcgaccattgcgcatctcgcctctcacccgtcgacgcgcaaggacttgacctttttgtcttcgtacaacggacagtctacgtcgccaaactcaggacatttggccagttttgccggtggcaacttcatcttgggaggcattctcctgaacgagcaaaagtacattgactttggaatcaagcttgccagctcgtactttgccacgtacaaccagacggcttctggaatcggccccgaaggcttcgcgtgggtggacagcgtgacgggcgccggcggctcgccgccctcgtcccagtccgggttctactcgtcggcaggattctgggtgacggcaccgtattacatcctgcggccggagacgctggagagcttgtactacgcataccgcgtcacgggcgactccaagtggcaggacctggcgtgggaagcgttcagtgccattgaggacgcatgccgcgccggcagcgcgtactcgtccatcaacgacgtgacgcaggccaacggcgggggtgcctctgacgatatggagagcttctggtttgccgaggcgctcaagtatgcgtacctgatctttgcggaggagtcggatgtgcaggtgcaggccaacggcgggaacaaatttgtctttaacacggaggcgcacccctttagcatccgttcatcatcacgacggggcggccaccttgcttaa

SEQ ID NO: 82 - DNA, 747 bp, Pichia pastoris
ttgggggcctccaggacttgctgaaatttgctgactcatcttcgccatccaaggataatgagttagctaatgtgacagttaatgagtcgtcttgactaacggggaacatttcattatttatatccagagtcaatttgatagcagagtttgtggttgaaatacctatgattcgggagactttgttgtaacgaccattatccacagtttggaccgtgaaaatgtcatcgaagagagcagacgacatattatctattgtggtaagtgatagttggaagtccgactaaggcatgaaaatgagaagactgaaaatttaaagtttttgaaaacactaatcgggtaataacttggaaattacgtttacgtgcctttagctcttgtccttacccctgataatctatccatttcccgagagacaatgacatctcggacagctgagaacccgttcgatatagagcttcaagagaatctaagtccacgttcttccaattcgtccatattggaaaacattaatgagtatgctagaagacatcgcaatgattcgctttcccaagaatgtgataatgaagatgagaacgaaaatctcaattatactgataacttggccaagttttcaaagtctggagtatcaagaaagagctgtatgctaatatttggtatttgctttgttatctggctgtttctctttgccttgtatgcgagggacaatcgattttccaatttgaacgagtacgttccagattcaaacag

SEQ ID NO: 83 - DNA, 924 bp, Pichia pastoris
ctactgggaaccacgagacatcactgcagtagtttccaagtggatttcagatcactcatttgtgaatcctgacaaaactgcgatatgggggtggtcttacggtgggttcactacgcttaagacattggaatatgattctggagaggttttcaaatatggtatggctgttgctccagtaactaattggcttttgtatgactccatctacactgaaagatacatgaaccttccaaaggacaatgttgaaggctacagtgaacacagcgtcattaagaaggtttccaattttaagaatgtaaaccgattcttggtttgtcacgggactactgatgataacgtgcattttcagaacacactaaccttactggaccagttcaatattaatggtgttgtgaattacgatcttcaggtgtatcccgacagtgaacatagcattgcccatcacaacgcaaataaagtgatctacgagaggttattcaagtggttagagcgggcatttaacgatagatttttgtaacattccgtacttcatgccatactatatatcctgcaaggtttccctttcagacacaataattgctttgcaattttacataccaccaattggcaaaaataatctcttcagtaagttgaatgcttttcaagccagcaccgtgagaaattgctacagcgcgcattctaacatcactttaaaattccctcgccggtgctcactggagtttccaacccttagcttatcaaaatcgggtgataactctgagttttttttttcacttctattcctaaaccttcgcccaatgctaccacctccaatcaacatcccgaaatggatagaagagaatggacatctcttgcaacctccggttaataattactgtctccacagaggaggatttacggtaatgattgtaggtgggcctaatg

SEQ ID NO: 84 - DNA, 980 bp, Pichia pastoris
cacctgggcctgttgctgctggtactgctgttggaactgttggtattgttgctgatctaaggccgcctgttccacaccgtgtgtatcgaatgcttgggcaaaatcatcgcctgccggaggccccactaccgcttgttcctcctgctcttgtttgttttgctcattgatgatatcggcgtcaatgaattgatcctcaatcgtgtggtggtggtgtcgtgattcctcttctttcttgagtgccttatccatattcctatcttagtgtaccaataattttgttaaacacacgctgttgtttatgaaaagtcgtcaaaaggttaaaaattctacttggtgtgtgtcagagaaagtagtgcagacccccagtttgttgactagttgagaaggcggctcactattgcgcgaatagcatgagaaatttgcaaacatctggcaaagtggtcaatacctgccaacctgccaatcttcgcgacggaggctgttaagcgggttgggttcccaaagtgaatggatattacgggcaggaaaaacagccccttccacactagtctttgctactgacatcttccctctcatgtatcccgaacacaagtatcgggagtatcaacggagggtgcccttatggcagtactccctgttggtgattgtactgctatacgggtctcatttgcttatcagcaccatcaacttgatacactataaccacaaaaattatcatgcacacccagtcaatagtggtatcgttcttaatgagtttgctgatgacgattcattctctttgaatggcactctgaacttggagaactggagaaatggtaccttttcccctaaatttcattccattcagtggaccgaaataggtcaggaagatgaccagggatattacattctctcttccaattcctcttacatagtaaagtctttatccgacccagactttgaatctgttctattcaacgagtctacaatcacttacaacg

SEQ ID NO: 85 - DNA, 1117 bp, Pichia pastoris
ggcagcaaagccttacgttgatgagaatagactggccatttggggttggtcttatggaggttacatgacgctaaaggttttagaacaggataaaggtgaaacattcaaatatggaatgtctgttgcccctgtgacgaattggaaattctatgattctatctacacagaaagatacatgcacactcctcaggacaatccaaactattataattcgtcaatccatgagattgataatttgaagggagtgaagaggttcttgctaatgcacggaactggtgacgacaatgttcacttccaaaatacactcaaagttctagatttatttgatttacatggtcttgaaaactatgatatccacgtgttccctgatagtgatcacagtattagatatcacaacggtaatgttatagtgtatgataagctattccattggattaggcgtgcattcaaggctggcaaataaataggtgcaaaaatattattagactttttttttcgttcgcaagttattactgtgtaccataccgatccaatccgtattgtaattcatgttctagatccaaaatttgggactctaattcatgaggtctaggaagatgatcatctctatagttttcagcggggggctcgatttgcggttggtcaaagctaacatcaaaatgtttgtcaggttcagtgaatggtaactgctgctcttgaattggtcgtctgacaaattctctaagtgatagcacttcatctacaatcatttgcttcatcgtttctatatcgtccacgacctcaaacgagaaatcgaatttggaagaacagacgggctcatcgttaggatcatgccaaaccttgagatatggatgctctaaagcctcagtaactgtaattctgtgagtgggatctaccgtgagcattcgatccagtaagtctatcgcttcagggttggcaccgggaaataactggctgaatgggatcttgggcatgaatggcagggagcgaacataatcctgggcacgctctgatctgatagactgaagtgtctcttccgaaacagtacccagcgtactcaaaatcaagttcaattgatccacatagtctcttcctctaaaaatgggtcggccaccta

SEQ ID NO: 86 - DNA, 1936 bp, Pichia pastoris
ggccagcccatcaccatgaatgcttaaaacgccaactccttccatctcattttcgtaccagattatgactcttaggcggggagaatcccgtccagcatagcgaacatttcttttttttttttttttcgtttcgcatctctctatcgcattcagaaaaaaatacatataattcttccagtttccgtcattcattacgtttaaaactacgaaagttttagctctcttttgtttttgtttcctagattcgaaatattttctttattgagtttaatttgtgtggcagacaatggttagatctttcaccatcaaagtgcctgcttcctcagcaaatataggaccggggtttgacgttctgggaattggtctcaacctttacttggaactacaagtcaccattgatcccaaaattgatacctcaagcgatccagaaaatgtgttattgtcgtatgaaggtgagggggctgatgaggtgtcattgaaaagtgacgaaaacttgattacgcgcacagctctctatgttctacgttgtgacgacgtcaggactttccctaagggaaccaagattcacgtcattaaccctattcctctaggaagaggcttgggatcttcgggtgctgcagttgtcgccggtgcattgctcggaaattccatcggacagcttggatactccaaacaacgtttactggattactgtttgatgatagaacgtcatccagataacatcaccgcagctatggtgggtggtttcgttggatcttatcttagagatctttcaccagaagacacccagagaaaagagattccattagcagaagtcctgccagaacctcaaggtggtattaacaccggtctcaacccaccagtgcctccaaaaaacattgggcaccacatcaaatacggctgggcaaaagagatcaaatgtattgccattattccagactttgaagtatcaaccgcttcatctagaggcgttcttccaaccacttacgagagacatgacattattttcaacctgcaaaggatagccgttcttaccactgccctgacacaatctccaccagatccaagcttgatatacccagctatgcaggacaggattcaccaaccttacaggaaaactttgatccacggactgactgaaatactgtcttcattcaccccagaattacacaaaggtttgttgggaatctgtctttccggtgctgggcccacaatattagccctcgcaactgaaaacttcgatcagattgctaaggacatcattgccagatttgctgtcgaagacatcacctgtagttggaaactcttgaccccagctcttgaaggttctgttgttgaggagcttgcttaatagaaattagaacatcctctttagattatgataatacgtttttaacttttcccctaactgtagtgatggtatctgaccctcttagaccttaggttggaccttctcgaatttcctgcctctatcaaaaatccgaccctcgacatcgtttacgtactttgcaaccaattaactagtaccggcagacgttcagtgatcatggctctctatacaaataccctgataacgtttgcattcctgacagtcggaggatgtacgtgcttattttcttgctagtcccaaatgttttgagattgctccaatcgttttttcaacaatactaactgccaacaaatagatcttttattcaacggaaatggggaacaattcaacgtgggtgactttttggagactacatctccctatatgtgggcaaatctgggtatagcaagttgcattggattctcggtcattggtgctgcatggggaattttcataacaggttcttcgatcatcggtgcaggtgtcaaagctcccagaatcacaacaaaaaatttaatctccatcattttctgtgaggtggtggctatttatgggcttattatggcc

SEQ ID NO: 87 - DNA, 588 bp, Pichia pastoris
cctgtgagtctggctcaatcacttttcaaagataaggactattctgcagaacatgcagcccaggcaacatcatcccagttcatctctgtgaacacaggaataggattcctggaccatatgttacacgcacttgctaagcacggcggctggtctgtcattatcgaatgtgtaggtgatttgcacattgatgaccatcattcagcagaagatactggaatcgcattggggatggcattcaaagaagccttgggccatgttcgtggtatcaaaagattcgggtccggatttgctccactagacgaagctctcagtcgggctgttattgatatgtctaacaggccctatgctgttgtcgatctgggtttgaaaagagagaagattggagacctatcgtgtgagatgattccccatgttttggaaagttttgcccaaggagcccatgtaaccatgcacgtagattgtttgcgaggtttcaacgaccatcatcgtgccgagagtgcattcaaagctttggctatagctatcaaagaggccatttcaagcaacggcacggacgacattccaagtacgaagggtgttcttttctga

SEQ ID NO: 88 - DNA, 1049 bp, Pichia pastoris
gtctggaaggtgtctacatctgtgaaatccgtatttatttaagtaaaacaatcagtaatataagatcttagttggtttaccacatagtcggtaccggtcgtgtgaacaatagttcaatgcctccgattgtgccttattgttgtggtctgcattttcgcggcgaaatttctacttcagatcggggctgagatgaccttagtactcacatcaaccagctcgttgaaagttcccacatgaccactcaatgtttaatagcttggcacccatgaggttgaagaaactacttaaggtgttttgtgcctcagtagtgctgttagcggcgacatctgtggtgttatttttccactttggaggtcagatcataatccccataccggaacgcactgtgaccttaagtactcctcccgcaaacgatacttggcagtttcaacagttcttcaacggctatttagacgccctgttagagaataacctgtcgtatccgataccagaaaggtggaatcatgaagttacaaatgtaagattcttcaatcgcataggtgaattgctctcggagagtaggctacaggagctgattcattttagtcctgagttcatagaggataccagtgacaaattcgacaatattgttgaacaaattccagcaaaatggccttacgaaaacatgtacagaggagatggatacgttattgttggtggtggcagacacacctttttggcactgctgaatatcaacgctttgagaagagcaggcaataaactgccagttgaggtcgtgttgccaacttacgacgactatgaggaagatttctgtgaaaatcattttccacttttgaatgcaagatgcgtaatcttagaagaacgatttggtgaccaagtttatccccggttacaactaggaggctaccagtttaaaatatttgcgatagcagcaagttcattcaaaaactgctttttgttagattcagataatatacccttgcgaaagatggataagatattctcaagcgaactatacaagaataagacaatgattacttggccagact

SEQ ID NO: 89 - DNA, 631 bp, Pichia pastoris
cgagtcggccagcccatcaccatgaatgcttaaaacgccaactccttccatctcattttcgtaccagattatgactcttaggcggggagaatcccgtccagcatagcgaacatttcttttttttttttttttcgtttcgcatctctctatcgcattcagaaaaaaatacatataattcttccagtttccgtcattcattacgtttaaaactacgaaagttttagctctcttttgtttttgtttcctagattcgaaatattttctttattgagtttaatttgtgtggcagacaatggttagatctttcaccatcaaagtgcctgcttcctcagcaaatataggaccggggtttgacgttctgggaattggtctcaacctttacttggaactacaagtcaccattgatcccaaaattgatacctcaagcgatccagaaaatgtgttattgtcgtatgaaggtgagggggctgatgaggtgtcattgaaaagtgacgaaaacttgattacgcgcacagctctctatgttctacgttgtgacgacgtcaggactttccctaagggaaccaagattcacgtcattaaccctattcctctaggaagaggcttgggatcttcgggtgctgcagttgtc

SEQ ID NO: 90 - DNA, 590 bp, Pichia pastoris
tagaaattagaacatcctctttagattatgataatacgtttttaacttttcccctaactgtagtgatggtatctgaccctcttagaccttaggttggaccttctcgaatttcctgcctctatcaaaaatccgaccctcgacatcgtttacgtactttgcaaccaattaactagtaccggcagacgttcagtgatcatggctctctatacaaataccctgataacgtttgcattcctgacagtcggaggatgtacgtgcttattttcttgctagtcccaaatgttttgagattgctccaatcgttttttcaacaatactaactgccaacaaatagatcttttattcaacggaaatggggaacaattcaacgtgggtgactttttggagactacatctccctatatgtgggcaaatctgggtatagcaagttgcattggattctcggtcattggtgctgcatggggaattttcataacaggttcttcgatcatcggtgcaggtgtcaaagctcccagaatcacaacaaaaaatttaatctccatcattttctgtgaggtggtggctatttatgggcttattatggccattgt

SEQ ID NO: 91 - DNA, 1634 bp, Pichia pastoris
aagtgggccagattatataaatatggatcaacatgaagccttgaaagatttcaaggacaggcttaggaattacgaaaaagtttacgagactattgacgaccaggaggaagaggagaacgaacggtacaatattcagtatctgaagataatcaacgcaggaaagaagatagtcagttataacataaatgggtatttatcgtcccacaccgttttttatctcctgaatttcaatcttgcagaacgtcaaatatggttgacgacgaatggagagacagagtataaccttcaaaataggattggaggtgattccaaattaagcaatgagggatggaaatttgccaaagcattgcccaagtttatagcacagaaaagaaaagagtttcaacttagacagttgaccaaacactatatcgagactcaaacgcccattgaagacgtaccgttggaggagcacaccaagccagtcaaatattctgatctgcatttccatgtttggtcatcggctttaaagagatctactcaatcaacaacattttttccatcggaaaattactctctgaagcaattcagaacgttgaatgatctctgttgcggatcactggatggtttgactgaacaagagttcaaaagtaaatacaaagaagaataccagaattctcagactgataaactgagtttcagtttccctggtatcggtggggagtcttatttggacgtgatcaaccgtttgagaccactaatagttgaactagaaaggttgccagaacatgtcctggtcattacccaccgggtcatagtaaggattttactaggatatttcatgaatttggatagaaatctgttgacagatttggaaattttgcatgggtatgtttattgtattgagccgaaaccttatggtttagacttaaagatctggcagtatgatgaggcggacaacgagtttaatgaagttgataagctggaattcatgaaaagaagaagaaaatcgatcaacgtcaacacgacagatttcagaatgcagttaaacaaagagttgcaacaggacgctctcaataatagtcctggtaataatagtccgggcgtatcatctctatcttcatactcgtcgtcctcttccctttccgctgacgggagcgagggagaaacattaataccacaagtatcccaggcggagagctacaactttgaatttaactctctttcatcatcagtttcatcgttgaaaaggacgacatcttcttcccaacatttgagctccaatcctagttgtctgagcatgcataatgcctcattggacgagaatgacgacgaacatttaatagacccggcttctacagacgacaagctaaacatggtattacaggacaaaacgctaattaaaaagctcaaaagtttactacttgacgaggccgaaggctagacaatccacagttaattttgatactgtactttataacgagtaacatacatatcttatgtaatcatctatgtcacgtcacgtgcgcgcgacattattccgagaacttgcgccctgctagctccactgtcagagtgataacttccccaaaataggatccaactgtttccaattgcttttggaaatgtggattgaaagaaacctcatagcgtaa

SEQ ID NO: 92 - DNA, 1211 bp, Pichia pastoris
gacgacgaggagaatatcaattttgattcccggtagatagctcacccacggtcacacacacaaacacacatacacattaacacacagagttattagttaacagagaaaactctaacaaagtatttattttcgttacgtaatccgacttttctttttaccgttttctattgctcctctcatttgcccctaaaagttgctcctcattactaaaatcaccacaccatgctcgaatatgatgttactaaatgcaaattgtagtcgtgcctcttgtggtaatactatagggaatatctctcgattactcgattctggttaattttttctttttttataggggaagtttttttttcttcccctttctctccagtttatttatttactaagaaaatccaacagataccaaccacccaaaaagatcctaaacagcctgtttttgaggagtttttcagcagctaagcttcatcagttttttaatacttaatttattgcccttcactttgtttcttgtggcttttaaggctctccggaacagcggtttcaaaatcaaatctcagttatttgtttgctccgctttgtcagttcaaagatcatggtttccgaaaacaagaatcaatcttcgattttgatggacaactccaagaagctctctccgaagcccattttgaataacaagaatgaaccgtttggcatcggcgtcgatggacttcaacatcctcaaccgactttatgccgcacagaatcggaactcttgttcaacttgagccaagtcaataaatcccaaataactttggacggtgcagttactccacctgctgatggtaatgggaatgaagcaaaaagagcaaatctcatctcttttgatgttccatcgtctcaagtgaaacatagagggtctattagtgcaaggccctcggcagtgaatgtgtcccaaattaccggggccctttctcaatccggatcttctagaaatccctacgatcaaacacagtcacctccacctagcacttacgcctccaggcagaactccacccatggaaataatatcgatagcttgcaatatttggcaacaagagatcttagtgctttaaggctggaaagagatgcttccgcacgagaagctacctcttctgcagtgtccactcctgttcagttcgatgtacccaaacaacatcatctccttcatttagaacaagacccgacaaggcccatcc

SEQ ID NO: 93 - DNA, 365 bp, Pichia pastoris
acgacggccaaattcatgatacacactctgtttcagctggtttggactaccctggagttggtcctgaattggctgcctggaaagcaaatggtagagcccaattttccgctgtaactgatgcccaagcattagagggattcaaaatcctgtctcaattggaagggatcattccagcactagagtctagtcatgcaatctacggcgcattgcaaattgcaaagactatgtcttcggaccagtccttagttattaatgtatctggaaggggtgataaggacgtccagagtgtagctgagattttacctaaattgggacctcaaattggatgggatttgcgtttcagcgaagacattactaaagagtga

SEQ ID NO: 94 - DNA, 613 bp, Pichia pastoris
tcgatagcacaatattcaacttgactgggtgttaagaactaagagctctgggaaactttgtatttattactaccaacacagtcaaattattggatgtgtttttttttccagtacatttcactgagcagtttgttatactcggtctttaatctccatatacatgcagattgtaatacagatctgaacagtttgattctgattgatcttgccaccaatattctatttttgtatcaagtaacagagtcaatgatcattggtaacgtaacggttttcgtgtatagtagttagagcccatcttgtaacctcatttcctcccatattaaagtatcagtgattcgctggaacgattaactaagaaaaaaaaaatatctgcacatactcatcagtctgtaaatctaagtcaaaactgctgtatccaatagaaatcgggatatacctggatgttttttccacataaacaaacgggagttcagcttacttatggtgttgatgcaattcagtatgatcctaccaataaaacgaaactttgggattttggctgtttgagggatcaaaagctgcacctttacaagattgacggatcgaccattagaccaaagcaaatggccaccaa

(19) United States
(12) Patent Application Publication
(10) Pub. No.: US 2013/0330340 A1
(43) Pub. Date: Dec. 12, 2013

(54) PRODUCTION OF N- AND O-SIALYLATED TNFRII-FC FUSION PROTEIN IN YEAST

(71) Applicants: Stephen R. Hamilton, Enfield, NH (US); W. James Cook, Hanover, NH (US); Sujatha Gomathinayagam, Hanover, NH (US)

(72) Inventors: Stephen R. Hamilton, Enfield, NH (US); W. James Cook, Hanover, NH (US); Sujatha Gomathinayagam, Hanover, NH (US)

(21) Appl. No.: 13/985,130

(22) PCT Filed: Feb. 20, 2012
(86) PCT No.: PCT/US2012/025812

§ 371 Date: Aug. 13, 2013

Related U.S. Application Data
(60) Provisional application No. 61/446,853, filed on Feb. 25, 2011.

(51) Int. Cl. C07K 14/705; C07K 16/46

(52) U.S. Cl. CPC C07K 14/70578; C07K 16/46
USPC 424/134.1; 435/69.6; 530/387.3

(57) ABSTRACT

Production of recombinant Tumor Necrosis Factor Receptor fused to the Fc region of an antibody (TNFRII-Fc fragment fusion protein) in a glycoengineered yeast strain that is capable of producing sialylated N-glycans and O-glycans is described. Compositions of TNFRII-Fc fragment fusion protein comprising dystroglycan type O-glycans and sialylated N- and O-glycans with only terminal N-acetylneuraminic acid (NANA) residues in an α2,6-linkage are provided. In particular aspects, methods are provided for modulating the in vivo pharmacokinetics of the TNFRII-Fc fragment fusion protein by altering the O-glycan structure on the molecule.

### BACKGROUND OF THE INVENTION

(1) Field of the Invention

The present invention relates to the production of recombinant soluble tumor necrosis factor receptor II (TNFRII) fused to the Fc region of an antibody (TNFRII-Fc fragment fusion protein) in a glycoengineered yeast strain that is capable of producing sialylated N-glycans and O-glycans. In particular aspects, the present invention further relates to compositions of TNFRII-Fc fragment fusion protein comprising dystroglycan type O-glycans and sialylated N- and O-glycans with only terminal N-acetylneuraminic acid (NANA) residues in an α2,6-linkage. In particular aspects, the present invention relates to methods for modulating the in vivo pharmacokinetics of the TNFRII-Fc fragment fusion protein by altering the sialylation state of the molecule.

(2) Background of the Invention

Tumor necrosis factor receptor II (TNFRII) is a type I membrane glycoprotein belonging to the tumor necrosis factor (TNF) receptor superfamily and has an important role in independent signaling in chronic inflammatory conditions. Several inflammatory diseases and cancers display an increased and/or unregulated level of soluble TNFRII or polymorphisms. These observations have suggested that TNFRII might be an important target in treatments for these inflammatory diseases and cancers. Currently, TNFRII is used in therapies for treating rheumatoid arthritis. By binding TNFα, a cytokine, and blocking its interactions with receptors. Etanercept is a commercially available product marketed under the tradename ENBREL that is approved for treating moderate to severe rheumatoid arthritis; psoriatic arthritis; ankylosing spondylitis; chronic, moderate to severe psoriasis; and moderate to severe active polyarticular juvenile idiopathic arthritis. Etanercept is produced in Chinese hamster ovary (CHO) cells as a fusion protein consisting of the soluble domain of the TNFRII fused to the Fc region of an antibody (TNFRII-Fc). Soluble TNFRII-Fc fusion proteins and methods for producing them have been disclosed in Scallon et al., Cytokine 7: 759-770 (1995); Olsen & Stein, N. Engl. J. Med. 350: 2167-2179 (2004), Davis et al., Biotechnol. Prog. 16: 736-743 (2000), U.S. Pat. No. 5,605,690, U.S. Pat. No. 7,476,722, and U.S. Pat. No. 7,157,557.

Soluble TNFRII-Fc contains several N-glycosylation sites and multiple O-glycosylation sites. The extent and type of glycosylation is important as it conveys many desirable properties to the glycoprotein, including but not limited regulation of serum half-life and regulation of biological activity. In general, TNFRII-Fc produced in mammalian cells such as CHO cells has a glycosylation pattern that is similar to but not identical to the glycosylation pattern that would be produced in human cells. (See Wilson et al., Apollo Cytokine Research Pty., (2006); Jiang et al. Apollo Cytokine Research Pty.; Flossier et al., Glycobiol. 19: 936-949 (2009)). In addition, sialic acid on glycoproteins obtained from human cells is primarily of the N-acetylneuraminic acid (NANA) type. In contrast, the sialic acid on glycoproteins obtained from non-human cells such as CHO cells can include mixtures of NANA and N-glycolylneuraminic acid (NGNA). The ratio of NANA to NGNA is variable and depends on culturing conditions and cell line (Raju et al., Glycobiol. 10: 477-486 (2000); Baker et al., Biotechnol. Bioeng. 73: 188-202 (2001)). High levels NGNA has been shown to elicit an immune response (Noguchi et al., J. Biochem. 117: 59-62 (1995)) and can cause the rapid removal of glycoproteins from serum (Flesher et al., Biotechnol. Bioeng. 46: 309-407 (1995)).

Commercially available soluble TNFRII-Fc has been shown to be a useful product for treating a variety of inflammatory conditions and cancers. However, in light of the difference in glycosylation pattern between TNFRII-Fc produced in human cells verses TNFRII-Fc produced in non-human mammalian cell lines and the general observation that varying the glycosylation profile of a therapeutic glycoprotein can affect the pharmacokinetics and/or pharmacodynamics of the therapeutic glycoprotein, there remains a need for providing TNFRII-Fc with other glycosylation patterns. For example, it would be desirable to provide a TNFRII-Fc wherein the sialic acid is of only the NANA type.

### SUMMARY OF THE INVENTION

The present invention provides a soluble recombinant tumor necrosis factor receptor II (TNFRII) fused to the Fc region of an antibody (TNFRII-Fc fragment fusion protein) produced in a glycoengineered yeast strain. The soluble TNFRII-Fc fragment fusion protein has sialylated N-glycans and O-glycans comprising sialic acid of only the NANA type, which further aspects are linked to the N-glycan or O-glycan in an α2,6 or α2,3 linkage. By modulating the amount and sialylation of the O-glycan structure on the molecule, the present invention enables the in vivo half-life of the TNFRII-Fc to be regulated.

Therefore, the present invention provides a composition comprising or consisting essentially of a recombinant fragment of human tumor necrosis factor receptor fused to the constant region of an antibody (TNFRII-Fc) wherein the TNFRII-Fc has N-glycans and O-glycans and wherein the O-glycans are of the dystroglycan-type, and pharmaceutically acceptable salts thereof.

In further aspects of the invention, the N-glycans and O-glycans on the TNFRII-Fc are predominantly sialylated with α-2,6 sialic acid residues. In other aspects of the invention, the N-glycans and O-glycans on the TNFRII-Fc are predominantly sialylated with α-2,3 sialic acid residues. In further still aspects, the N-glycans on the TNFRII-Fc lack fucose residues. In further still aspects, the N-glycans and O-glycans on the TNFRII-Fc, which are sialylated, comprise N-acetylneuraminic acid (NANA) and no N-glycolylneuraminic acid (NGNA).

In further still aspects, a ratio of mole sialic acid to mole of the TNFRII-Fc is at least 10. In further still aspects, a ratio of mole sialic acid to mole of the TNFRII-Fc is about 10 to 21. In further still aspects, a ratio of mole sialic acid to mole of the TNFRII-Fc is greater than 21.

In particular aspects, at least 50%, 60%, 70%, 80%, 90%, or 100% of the N-glycans are sialylated. In further still aspects, the N-glycans on the TNFRII-Fc comprise or consist of predominantly mono-, bi-, tri-, or tetra-sialylated N-glycans. In further still aspects, the N-glycans on the TNFRII-Fc comprise or consist of predominantly mono-sialylated N-glycans. In further still aspects, the N-glycans on the TNFRII-Fc comprise or consist of predominantly bi-sialylated N-glycans. In further still aspects, the N-glycans on the TNFRII-Fc comprise or consist of predominantly tri-sialylated N-glycans. In further still aspects, the N-glycans on the TNFRII-Fc comprise or consist of predominantly tetra-sialylated N-glycans.

In further still aspects, the O-glycans on the TNFRII-Fc comprise or consist of predominantly sialylated O-glycans. In further still aspects, greater than 10%, 20%, 30%, 40%, or 50% of the O-glycans on the TNFRII-Fc comprise or consist of sialylated O-glycans. In further still aspects, less than 10%, 20%, 40% or 50% of the O-glycans on the TNFRII-Fc terminate in mannose.

In further still aspects, the TNFRII domain of the TNFRII-Fc comprises or consists of an amino acid sequence with at least 90%, 91%, 92%, 93%, 94%, 95%, 96%, 97%, 98%, or 99% identity to the amino acid sequence for the TNFRII domain set forth in SEQ ID NO:73 or 75. The receptor domain includes amino acids 1 to 235 of SEQ ID NO:73 or 75 and is encoded by nucleotides 1-705 of SEQ ID NO:72 or 74.

Further provided is a method for producing a recombinant human tumor necrosis factor fused to the constant region of an antibody (TNFRII-Fc) having sialylated N-glycans and O-glycans comprising or consisting of (a) providing a recombinant yeast host cell genetically engineered to produce glycoproteins having sialylated N-glycans and further comprising (i) a nucleic acid molecule encoding the TNFRII-Fc; (ii) a nucleic acid molecule encoding an α1,2-mannosidase activity linked to a heterologous targeting or signaling peptide that targets the mannosidase activity to the secretory pathway; and (iii) a nucleic acid molecule encoding an O-linked mannose β1,2-N-acetylglucosaminyltransferase 1 (POMGnT1); (b) culturing the host cell under conditions suitable for producing the TNFRII-Fc; and (c) recovering the TNFRII-Fc from the culture fluid to produce the TNFRII-Fc having sialylated N-glycans and O-glycans.

In further aspects, the POMGnT1 is provided as a fusion protein comprising the receptor domain of the POMGnT1 fused to a heterologous cellular targeting or signaling (or leader) peptide that targets the POMGnT1 to the secretory pathway, e.g., the ER or Golgi apparatus. Particular heterologous targeting or signal peptides include but are not limited to the Saccharomyces cerevisiae MNN2, MNN5 or MNN6 targeting or signal peptide.

In further aspects of the method, the N-glycans and O-glycans on the TNFRII-Fc are predominantly sialylated with α-2,6 sialic acid residues. In further still aspects, the N-glycans on the TNFRII-Fc lack fucose residues. In further still aspects, the N-glycans and O-glycans on the TNFRII-Fc, which are sialylated, comprise N-acetylneuraminic acid (NANA) and no N-glycolylneuraminic acid (NGNA).

In further still aspects, a ratio of mole sialic acid to a mole of the TNFRII-Fc is at least 10. In further still aspects, a ratio of mole sialic acid to mole of the TNFRII-Fc is about 10 to 21. In further still aspects, a ratio of mole sialic acid to mole of the TNFRII-Fc is greater than 21.

In particular aspects, at least 50%, 60%, 70%, 80%, 90%, or 100% of the N-glycans are sialylated. In further still aspects, the N-glycans on the TNFRII-Fc comprise or consist of predominantly mono-, bi-, tri-, or tetra-sialylated N-glycans. In further still aspects, the N-glycans on the TNFRII-Fc comprise or consist of predominantly mono-sialylated N-glycans. In further still aspects, the N-glycans on the TNFRII-Fc comprise or consist of predominantly bi-sialylated N-glycans. In further still aspects, the N-glycans on the TNFRII-Fc comprise or consist of predominantly tri-sialylated N-glycans. In further still aspects, the N-glycans on the TNFRII-Fc comprise or consist of predominantly tetra-sialylated N-glycans.

In further still aspects, the O-glycans on the TNFRII-Fc comprise or consist of predominantly sialylated O-glycans. In further still aspects, greater than 10%, 20%, 30%, 40%, or 50% of the O-glycans on the TNFRII-Fc comprise or consist of sialylated O-glycans. In further still aspects, less than 10%, 20%, 40% or 50% of the O-glycans on the TNFRII-Fc terminate in mannose.

In further still aspects, the TNFRII domain of the TNFRII-Fc comprises or consists of an amino acid sequence with at least 90%, 91%, 92%, 93%, 94%, 95%, 96%, 97%, 98%, or 99% identity to the amino acid sequence for the TNFRII domain set forth in SEQ ID NO:73 or 75. The receptor domain includes amino acids 1 to 235 of SEQ ID NO:73 or 75 and is encoded by nucleotides 1-705 of SEQ ID NO:72 or 74.

In further aspects of the method, the TNFRII-Fc is recovered from the culture fluid in a process comprising a hydroxyapatite or aminophenyl borate chromatography step. In further aspects of the method, the TNFRII-Fc is recovered from the culture fluid in a process comprising an affinity capture chromatography step and a hydroxyapatite or aminophenyl borate chromatography step. In further aspects of the method, the TNFRII-Fc is recovered from the culture fluid in a process comprising the steps of an affinity capture chromatography step, a hydrophobic interaction chromatography step, a hydroxyapatite or aminophenyl borate chromatography step, and a cation exchange chromatography step.

Further provided is a composition comprising or consisting essentially of a recombinant fragment of human tumor necrosis factor receptor fused to the constant region of an antibody (TNFRII-Fc) wherein the TNFRII-Fc has N-glycans and O-glycans and wherein the O-glycans are O-mannose reduced glycans, and pharmaceutically acceptable salts thereof. An O-mannose reduced glycan is an O-glycan in which the predominant O-glycan consists predominantly of a single mannose (mannose type) or mannobiose type (two mannose residues). In further aspects of the composition, the TNFRII domain of the TNFRII-Fc comprises or consists of an amino acid sequence with at least 90%, 91%, 92%, 93%, 94%, 95%, 96%, 97%, 98%, or 99% identity to the amino acid sequence for the TNFRII domain set forth in SEQ ID NO:73 or 75. The receptor domain includes amino acids 1 to 235 of SEQ ID NO:73 or 75 and is encoded by nucleotides 1-705 of SEQ ID NO:72 or 74.

Further provided is a method for producing a recombinant human tumor necrosis factor fused to the constant region of an antibody (TNFRII-Fc) having sialylated N-glycans and O-mannose reduced glycans comprising or consisting of (a) providing a recombinant lower eukaryote host cell genetically engineered to produce glycoproteins having sialylated N-glycans and further comprising (i) a nucleic acid molecule encoding the TNFRII-Fc; and (ii) a nucleic acid molecule encoding an α1,2-mannosidase activity linked to a heterologous targeting or signaling peptide that targets the mannosidase activity to the secretory pathway; (b) culturing the host cell under conditions suitable for producing the TNFRII-Fc; and (c) recovering the TNFRII-Fc from the culture fluid to produce the TNFRII-Fc having sialylated N-glycans and O-mannose reduced glycans.

In further aspects of the method, the TNFRII domain of the TNFRII-Fc comprises or consists of an amino acid sequence with at least 90%, 91%, 92%, 93%, 94%, 95%, 96%, 97%, 98%, or 99% identity to the amino acid sequence for the TNFRII domain set forth in SEQ ID NO:73 or 75. The receptor domain includes amino acids 1 to 235 of SEQ ID NO:73 or 75 and is encoded by nucleotides 1-705 of SEQ ID NO:72 or 74.

In further aspects of the method, the host cells are cultured in the presence of a PMT inhibitor which reduces the number of sites on the TNFRII-Fc that are O-glycosylated.

Further provided is a pharmaceutical composition comprising or consisting of the polypeptide of any one of aspects above and a pharmaceutically suitable carrier.

Further provided is the use of the above pharmaceutical composition in the manufacture of a medicament for inflammatory diseases and cancers that display an increased and/or unregulated level of soluble TNFRII or polymorphisms or the use of the pharmaceutical composition of claim 25 in the manufacture of a medicament for treating rheumatoid arthritis.

### DEFINITIONS

As used herein, the terms “N-glycan” and “glycoform” are used interchangeably and refer to an N-linked oligosaccharide, for example, one that is attached by an asparagine-N-acetylglucosamine linkage to an asparagine residue of a polypeptide. N-linked glycoproteins contain an N-acetylglucosamine residue linked to the amide nitrogen of an asparagine residue in the protein. The predominant sugars found on glycoproteins are glucose, galactose, mannose, fucose, N-acetylgalactosamine (GalNAc), N-acetylglucosamine (GlcNAc) and sialic acid (e.g., N-acetyl-neuraminic acid (NANA)). The processing of the sugar groups occurs co-translationally in the lumen of the ER and continues post-translationally in the Golgi apparatus for N-linked glycoproteins.

N-glycans have a common pentasaccharide core of Man₃GlcNAc₂ (“Man” refers to mannose; “Glc” refers to glucose; and “NAc” refers to N-acetyl; GlcNAc refers to N-acetylglucosamine). Usually, N-glycan structures are presented with the non-reducing end to the left and the reducing end to the right. The reducing end of the N-glycan is the end that is attached to the Asn residue comprising the glycosylation site on the protein. N-glycans differ with respect to the number of branches (antennae) comprising peripheral sugars (e.g., GlcNAc, galactose, fucose and sialic acid) that are added to the Man₃GlcNAc₂ (“Man3”) core structure which is also referred to as the “trimannose core”, the “pentasaccharide core” or the “paucimannose core”. N-glycans are classified according to their branched constituents (e.g., high mannose, complex or hybrid). A “high mannose” type N-glycan has five or more mannose residues. A “complex” type N-glycan typically has at least one GlcNAc attached to the 1,3 mannose arm and at least one GlcNAc attached to the 1,6 mannose arm of a “trimannose” core. Complex N-glycans may also have galactose (“Gal”) or N-acetylgalactosamine (“GalNAc”) residues that are optionally modified with sialic acid or derivatives (e.g., “NANA” or “NeuAc”, where “Neu” refers to neuraminic acid and “Ac” refers to acetyl). Complex N-glycans may also have intrachain substitutions comprising “bisecting” GlcNAc and core fucose (“Fuc”). Complex N-glycans may also have multiple antennae on the “trimannose core,” often referred to as “multiple antennary glycans.” A “hybrid” N-glycan has at least one GlcNAc on the terminal of the 1,3 mannose arm of the trimannose core and zero or more mannoses on the 1,6 mannose arm of the trimannose core. The various N-glycans are also referred to as “glycoforms.”

With respect to complex N-glycans, the terms “G-2”, “G-1”, “G0”, “G1”, “G2”, “A1”, and “A2” mean the following. “G-2” refers to an N-glycan structure that can be characterized as Man₃GlcNAc₂; the term “G-1” refers to an N-glycan structure that can be characterized as GlcNAcMan₃GlcNAc₂; the term “G0” refers to an N-glycan structure that can be characterized as GlcNAc₂Man₃GlcNAc₂; the term “G1” refers to an N-glycan structure that can be characterized as GalGlcNAc₂Man₃GlcNAc₂; the term “G2” refers to an N-glycan structure that can be characterized as Gal₂GlcNAc₂Man₃GlcNAc₂; the term “A1” refers to an N-glycan structure that can be characterized as NANAGal₂GlcNAc₂Man₃GlcNAc₂; and, the term “A2” refers to an N-glycan structure that can be characterized as NANA₂Gal₂GlcNAc₂Man₃GlcNAc₂. Unless otherwise indicated, the terms G-2″, “G-1”, “G0”, “G1”, “G2”, “A 1”, and “A2” refer to N-glycan species that lack fucose attached to the GlcNAc residue at the reducing end of the N-glycan. When the term includes an “F”, the “F” indicates that the N-glycan species contains a fucose residue on the GlcNAc residue at the reducing end of the N-glycan. For example, G0F, G1F, G2F, A1F, and A2F all indicate that the N-glycan further includes a fucose residue attached to the GlcNAc residue at the reducing end of the N-glycan. Lower eukaryotes such as yeast and filamentous fungi do not normally produce N-glycans that produce fucose.

With respect to multiantennary N-glycans, the term “multiantennary N-glycan” refers to N-glycans that further comprise a GlcNAc residue on the mannose residue comprising the non-reducing end of the 1,6 arm or the 1,3 arm of the N-glycan or a GlcNAc residue on each of the mannose residues comprising the non-reducing end of the 1,6 arm and the 1,3 arm of the N-glycan. Thus, multiantennary N-glycans can be characterized by the formulas GlcNAc₍₂₋₄₎Man3GlcNAc2, Gal₍₁₋₄₎GlcNAc₍₂₋₄₎Man₃GlcNAc₂, or NANA₍₁₋₄₎Gal₍₁₋₄₎GlcNAc₍₂₋₄₎Man₃GlcNAc₂. The term “1-4” refers to 1, 2, 3, or 4 residues.

With respect to bisected N-glycans, the term “bisected N-glycan” refers to N-glycans in which a GlcNAc residue is linked to the mannose residue at the reducing end of the N-glycan. A bisected N-glycan can be characterized by the formula GlcNAc₃Man₃GlcNAc₂ wherein each mannose residue is linked at its non-reducing end to a GlcNAc residue. In contrast, when a multiantennary N-glycan is characterized as GlcNAc₃Man₃GlcNAc₂, the formula indicates that two GlcNAc residues are linked to the mannose residue at the non-reducing end of one of the two arms of the N-glycans and one GlcNAc residue is linked to the mannose residue at the non-reducing end of the other arm of the N-glycan.

Abbreviations used herein are of common usage in the art, see, e.g., abbreviations of sugars, above. Other common abbreviations include “PNGase”, or “glycanase” or “glucosidase” which all refer to peptide N-glycosidase F (EC 3.2.2.18).

The term “recombinant host cell” (“expression host cell”, “expression host system”, “expression system” or simply “host cell”), as used herein, is intended to refer to a cell into which a recombinant vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A recombinant host cell may be an isolated cell or cell line grown in culture or may be a cell which resides in a living tissue or organism. Preferred host cells are yeasts and fungi.

When referring to “mole percent” of a glycan present in a preparation of a glycoprotein, the term means the molar percent of a particular glycan present in the pool of N-linked oligosaccharides released when the protein preparation is treated with PNGase and then quantified by a method that is not affected by glycoform composition, (for instance, labeling a PNGase released glycan pool with a fluorescent tag such as 2-aminobenzamide and then separating by high performance liquid chromatography or capillary electrophoresis and then quantifying glycans by fluorescence intensity). For example, 50 mole percent NANA₂Gal₂GlcNAc₂Man₃GlcNAc₂ means that 50 percent of the released glycans are NANA₂ Gal₂GlcNAc₂Man₃GlcNAc₂ and the remaining 50 percent are comprised of other N-linked oligosaccharides. In embodiments, the mole percent of a particular glycan in a preparation of glycoprotein will be between 20% and 100%, preferably above 25%, 30%, 35%, 40% or 45%, more preferably above 50%, 55%, 60%, 65% or 70% and most preferably above 75%, 80% 85%, 90% or 95%.

The term “operably linked” expression control sequences refers to a linkage in which the expression control sequence is contiguous with the gene of interest to control the gene of interest, as well as expression control sequences that act in trans or at a distance to control the gene of interest.

The term “expression control sequence” or “regulatory sequences” are used interchangeably and as used herein refer to polynucleotide sequences which are necessary to affect the expression of coding sequences to which they are operably linked. Expression control sequences are sequences which control the transcription, post-transcriptional events and translation of nucleic acid sequences. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to include, at a minimum, all components whose presence is essential for expression, and can also include additional components whose presence is advantageous, for example, leader sequences and fusion partner sequences.

The term “transfect”, transfection”, “transfecting” and the like refer to the introduction of a heterologous nucleic acid into eukaryote cells, both higher and lower eukaryote cells. Historically, the term “transformation” has been used to describe the introduction of a nucleic acid into a yeast or fungal cell; however, herein the term “transfection” is used to refer to the introduction of a nucleic acid into any eukaryote cell, including yeast and fungal cells.

The term “eukaryotic” refers to a nucleated cell or organism, and includes insect cells, plant cells, mammalian cells, animal cells and lower eukaryotic cells.

The term “lower eukaryotic cells” includes yeast and filamentous fungi. Yeast and filamentous fungi include, but are not limited to Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Physcomitrella patens and Neurospora crassa. Pichia sp., any Saccharomyces sp., Hansenula polymorpha, any Kluyveromyces sp., Candida albicans, any Aspergillus sp., Trichoderma reesei, Chrysosporium lucknowense, any Fusarium sp. and Neurospora crassa.

As used herein, the terms “antibody,” “immunoglobulin,” “immunoglobulins” and “immunoglobulin molecule” are used interchangeably. Each immunoglobulin molecule has a unique structure that allows it to bind its specific antigen, but all immunoglobulins have the same overall structure as described herein. The basic immunoglobulin structural unit is known to comprise a tetramer of subunits. Each tetramer has two identical pairs of polypeptide chains, each pair having one “light” chain (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Light chains are classified as either kappa or lambda. Heavy chains are classified as gamma, mu, alpha, delta, or epsilon, and define the antibody's isotype as IgG, IgM, IgA, IgD, and IgE, respectively.

The term “Fc fragment” refers to the ‘fragment crystallized’ C-terminal region of the antibody containing the CH2 and CH3 domains.

As used herein, the term “consisting essentially of” will be understood to imply the inclusion of a stated integer or group of integers; while excluding modifications or other integers which would materially affect or alter the stated integer. With respect to species of N-glycans, the term “consisting essentially of” a stated N-glycan will be understood to include the N-glycan whether or not that N-glycan is fucosylated at the N-acetylglucosamine (GlcNAc) which is directly linked to the asparagine residue of the glycoprotein.

As used herein, the term “predominantly” or variations such as “the predominant” or “which is predominant” will be understood to mean the glycan species that has the highest mole percent (%) of total N-glycans after the glycoprotein has been treated with PNGase and released glycans analyzed by mass spectroscopy, for example, MALDI-TOF MS or HPLC. In other words, the phrase “predominantly” is defined as an individual entity, such as a specific glycoform, is present in greater mole percent than any other individual entity. For example, if a composition consists of species A at 40 mole percent, species B at 35 mole percent and species C at 25 mole percent, the composition comprises predominantly species A, and species B would be the next most predominant species. Some host cells may produce compositions comprising neutral N-glycans and charged N-glycans such as mannosylphosphate or sialic acid. Therefore, a composition of glycoproteins can include a plurality of charged and uncharged or neutral N-glycans. In the present invention, it is within the context of the total plurality of N-glycans in the composition in which the predominant N-glycan determined. Thus, as used herein, “predominant N-glycan” means that of the total plurality of N-glycans in the composition, the predominant N-glycan is of a particular structure.

As used herein, the term “essentially free of” a particular sugar residue, such as fucose, or galactose and the like, is used to indicate that the glycoprotein composition is substantially devoid of N-glycans which contain such residues. Expressed in terms of purity, essentially free means that the amount of N-glycan structures containing such sugar residues does not exceed 10%, and preferably is below 5%, more preferably below 1%, most preferably below 0.5%, wherein the percentages are by weight or by mole percent. Thus, substantially all of the N-glycan structures in a glycoprotein composition according to the present invention are free of, for example, fucose, or galactose, or both.

As used herein, a glycoprotein composition “lacks” or “is lacking” a particular sugar residue, such as fucose or galactose, when no detectable amount of such sugar residue is present on the N-glycan structures at any time. For example, in preferred embodiments of the present invention, the glycoprotein compositions are produced by lower eukaryotic organisms, as defined above, including yeast (for example, Pichia sp.; Saccharomyces sp.; Kluyveromyces sp.; Aspergillus sp.), and will “lack fucose,” because the cells of these organisms do not have the enzymes needed to produce fucosylated N-glycan structures. Thus, the term “essentially free of fucose” encompasses the term “lacking fucose.” However, a composition may be “essentially free of fucose” even if the composition at one time contained fucosylated N-glycan structures or contains limited, but detectable amounts of fucosylated N-glycan structures as described above.

### BRIEF DESCRIPTION OF THE DRAWINGS

FIGS. 1A-G are flow-diagrams showing the construction of strains YGLY11731, YGLY10299, and YGLY13571, each strain capable of producing a TNFRII-Fc fragment fusion protein comprising sialylated N-glycans.

FIGS. 2A-B show the construction of YGLY12680, a strain capable of producing a TNFRII-Fc fragment fusion protein comprising sialylated N-glycans and O-glycans.

FIG. 3 shows the construction of strain YGLY14252, a strain capable of producing a TNFRII-Fc fragment fusion protein comprising sialylated N-glycans and O-glycans.

FIG. 4 shows the construction of strains YGLY14954 and YGLY14927, each strain capable of producing a TNFRII-Fc fragment fusion protein comprising sialylated N-glycans and O-glycans.

FIG. 5 shows a map of plasmid pGLY6. Plasmid pGLY6 is an integration vector that targets the URA5 locus and contains a nucleic acid molecule comprising the S. cerevisiae invertase gene or transcription unit (ScSUC2) flanked on one side by a nucleic acid molecule comprising a nucleotide sequence from the 5′ region of the P. pastoris URA5 gene (PpURA5-5′) and on the other side by a nucleic acid molecule comprising the a nucleotide sequence from the 3′ region of the P. pastoris URA5 gene (PpURA5-3′).

FIG. 6 shows a map of plasmid pGLY40. Plasmid pGLY40 is an integration vector that targets the OCH1 locus and contains a nucleic acid molecule comprising the P. pastoris URA5 gene or transcription unit (PpURA5) flanked by nucleic acid molecules comprising lacZ repeats (lacZ repeat) which in turn is flanked on one side by a nucleic acid molecule comprising a nucleotide sequence from the 5′ region of the OCH1 gene (PpOCH1-5′) and on the other side by a nucleic acid molecule comprising a nucleotide sequence from the 3′ region of the OCH1 gene (PpOCH1-3′).

FIG. 7 shows a map of plasmid pGLY43a. Plasmid pGLY43a is an integration vector that targets the BMT2 locus and contains a nucleic acid molecule comprising the K. lactis UDP-N-acetylglucosamine (UDP-GlcNAc) transporter gene or transcription unit (KlGlcNAc Transp.) adjacent to a nucleic acid molecule comprising the P. pastoris URA5 gene or transcription unit (PpURA5) flanked by nucleic acid molecules comprising lacZ repeats (lacZ repeat). The adjacent genes are flanked on one side by a nucleic acid molecule comprising a nucleotide sequence from the 5′ region of the BMT2 gene (PpPBS2-5′) and on the other side by a nucleic acid molecule comprising a nucleotide sequence from the 3′ region of the BMT2 gene (PpPBS2-3′).

FIG. 8 shows a map of plasmid pGLY48. Plasmid pGLY48 is an integration vector that targets the MNN4 L1 locus and contains an expression cassette comprising a nucleic acid molecule encoding the mouse homologue of the UDP-GlcNAc transporter (MmGlcNAc Transp.) open reading frame (ORF) operably linked at the 5′ end to a nucleic acid molecule comprising the P. pastoris GAPDH promoter (PpGAPDH Prom) and at the 3′ end to a nucleic acid molecule comprising the S. cerevisiae CYC termination sequence (ScCYC TT) adjacent to a nucleic acid molecule comprising the P. pastoris URA5 gene or transcription unit (PpURA5) flanked by lacZ repeats (lacZ repeat) and in which the expression cassettes together are flanked on one side by a nucleic acid molecule comprising a nucleotide sequence from the 5′ region of the P. pastoris MNN4 L1 gene (PpMNN4 L1-5′) and on the other side by a nucleic acid molecule comprising a nucleotide sequence from the 3′ region of the MNN4 L1 gene (PpMNN4 L1-3′).

FIG. 9 shows as map of plasmid pGLY45. Plasmid pGLY45 is an integration vector that targets the PNO1/MNN4 loci contains a nucleic acid molecule comprising the P. pastoris URA5 gene or transcription unit (PpURA5) flanked by nucleic acid molecules comprising lacZ repeats (lacZ repeat) which in turn is flanked on one side by a nucleic acid molecule comprising a nucleotide sequence from the 5′ region of the PNO1 gene (PpPNO1-5′) and on the other side by a nucleic acid molecule comprising a nucleotide sequence from the 3′ region of the MNN4 gene (PpMNN4-3′).

FIG. 10 shows a map of plasmid pGLY1430. Plasmid pGLY1430 is a KINKO integration vector that targets the ADE1 locus without disrupting expression of the locus and contains in tandem four expression cassettes encoding (1) the human GlcNAc transferase I catalytic domain (codon optimized) fused at the N-terminus to P. pastoris SEC12 leader peptide (CO-NA10), (2) mouse homologue of the UDP-GlcNAc transporter (MmTr), (3) the mouse mannosidase IA catalytic domain (FB) fused at the N-terminus to S. cerevisiae SEC12 leader peptide (FBS), and (4) the P. pastoris URA5 gene or transcription unit (PpURA5) flanked by lacZ repeats (lacZ). All flanked by the 5′ region of the ADE1 gene and ORF (ADE1 5′ and ORF) and the 3′ region of the ADE1 gene (PpADE1-3′). PpPMA1 prom is the P. pastoris PMA1 promoter; PpPMA1 TT is the P. pastoris PMA1 termination sequence; SEC4 is the P. pastoris SEC4 promoter; OCH1 TT is the P. pastoris OCH1 termination sequence; ScCYC TT is the S. cerevisiae CYC termination sequence; PpOCH 1 Prom is the P. pastoris OCH1 promoter; PpALG3 TT is the P. pastoris ALG3 termination sequence; and PpGAPDH is the P. pastoris GADPH promoter.

FIG. 11 shows a map of plasmid pGLY582. Plasmid pGLY582 is an integration vector that targets the HIS1 locus and contains in tandem four expression cassettes encoding (1) the S. cerevisiae UDP-glucose epimerase (ScGAL10), (2) the human galactosyltransferase I (hGalT) catalytic domain fused at the N-terminus to the S. cerevisiae KRE2-s leader peptide (33), (3) the P. pastoris URA5 gene or transcription unit (PpURA5) flanked by lacZ repeats (lacZ repeat), and (4) the D. melanogaster UDP-galactose transporter (DmUGT). All flanked by the 5′ region of the HIS1 gene (PpHIS1-5′) and the 3′ region of the HIS1 gene (PpHIS1-3′). PMA1 is the P. pastoris PMA1 promoter; PpPMA1 TT is the P. pastoris PMA1 termination sequence; GAPDH is the P. pastoris GADPH promoter and ScCYC TT is the S. cerevisiae CYC termination sequence; PpOCH1 Prom is the P. pastoris OCH1 promoter and PpALG12 TT is the P. pastoris ALG12 termination sequence.

FIG. 12 shows a map of plasmid pGLY167b. Plasmid pGLY167b is an integration vector that targets the ARG1 locus and contains in tandem three expression cassettes encoding (1) the D. melanogaster mannosidase II catalytic domain (codon optimized) fused at the N-terminus to S. cerevisiae MNN2 leader peptide (CO-KD53), (2) the P. pastoris HIS1 gene or transcription unit, and (3) the rat N-acetylglucosamine (GlcNAc) transferase II catalytic domain (codon optimized) fused at the N-terminus to S. cerevisiae MNN2 leader peptide (CO-TC54). All flanked by the 5′ region of the ARG1 gene (PpARG1-5′) and the 3′ region of the ARG1 gene (PpARG1-3′). PpPMA1 prom is the P. pastoris PMA1 promoter; PpPMA1 TT is the P. pastoris PMA1 termination sequence; PpGAPDH is the P. pastoris GADPH promoter; ScCYC TT is the S. cerevisiae CYC termination sequence; PpOCH1 Prom is the P. pastoris OCH1 promoter; and PpALG12 TT is the P. pastoris ALG12 termination sequence.

FIG. 13 shows a map of plasmid pGLY3411 (pSH1092). Plasmid pGLY3411 (pSH1092) is an integration vector that contains the expression cassette comprising the P. pastoris URA5 gene or transcription unit (PpURA5) flanked by lacZ repeats (lacZ repeat) flanked on one side with the 5′ nucleotide sequence of the P. pastoris BMT4 gene (PpPBS4 5′) and on the other side with the 3′ nucleotide sequence of the P. pastoris BMT4 gene (PpPBS4 3′).

FIG. 14 shows a map of plasmid pGLY3419 (pSH1110). Plasmid pGLY3419 (pSH1110) is an integration vector that contains an expression cassette comprising the P. pastoris URA5 gene or transcription unit (PpURA5) flanked by lacZ repeats (lacZ repeat) flanked on one side with the 5′ nucleotide sequence of the P. pastoris BMT1 gene (PBS1 5′) and on the other side with the 3′ nucleotide sequence of the P. pastoris BMT1 gene (PBS 1 3′)

FIG. 15 shows a map of plasmid pGLY3421 (pSH1106). Plasmid pGLY3421 (pSH1106) contains an expression cassette comprising the P. pastoris URA5 gene or transcription unit (PpURA5) flanked by lacZ repeats (lacZ repeat) flanked on one side with the 5′ nucleotide sequence of the P. pastoris BMT3 gene (PpPBS3 5′) and on the other side with the 3′ nucleotide sequence of the P. pastoris BMT3 gene (PpPBS3 3′).

FIG. 16 shows a map of plasmid pGLY2456. Plasmid pGLY2456 is a KINKO integration vector that targets the TRP2 locus without disrupting expression of the locus and contains six expression cassettes encoding (1) the mouse CMP-sialic acid transporter codon optimized (CO mCMP-Sia Transp), (2) the human UDP-GlcNAc 2-epimerase/N-acetylmannosamine kinase codon optimized (CO hGNE), (3) the Pichia pastoris ARG1 gene or transcription unit, (4) the human CMP-sialic acid synthase codon optimized (CO hCMP-NANA S), (5) the human N-acetylneuraminate-9-phosphate synthase codon optimized (CO hSIAP S), and, (6) the mouse α-2,6-sialyltransferase catalytic domain codon optimized fused at the N-terminus to S. cerevisiae KRE2 leader peptide (comST6-33). All flanked by the 5′ region of the TRP2 gene and ORF (PpTRP2 5′) and the 3′ region of the TRP2 gene (PpTRP2-3′). PpPMA1 prom is the P. pastoris PMA1 promoter; PpPMA1 TT is the P. pastoris PMA1 termination sequence; CYC TT is the S. cerevisiae CYC termination sequence; PpTEF Prom is the P. pastoris TEF1 promoter; PpTEF TT is the P. pastoris TEF1 termination sequence; PpALG3 TT is the P. pastoris ALG3 termination sequence; and pGAP is the P. pastoris GAPDH promoter.

FIG. 17 shows a map of plasmid pGLY5048. Plasmid pGLY5048 is an integration vector that targets the STE13 locus and contains expression cassettes encoding (1) the T. reesei α-1,2-mannosidase catalytic domain fused at the N-terminus to S. cerevisiae αMATpre signal peptide (αMATTrMan) to target the chimeric protein to the secretory pathway and secretion from the cell and (2) the P. pastoris URA5 gene or transcription unit.

FIG. 18 shows a map of plasmid pGLY5019. Plasmid pGLY5019 is an integration vector that targets the DAP2 locus and contains an expression cassette comprising a nucleic acid molecule encoding the Nourseothricin resistance (NAT^{R}) ORF operably linked to the Ashbya gossypii TEF1 promoter and A. gossypii TEF1 termination sequences flanked one side with the 5′ nucleotide sequence of the P. pastoris DAP2 gene and on the other side with the 3′ nucleotide sequence of the P. pastoris DAP2 gene.

FIG. 19 is a map of plasmid pGLY5045. Plasmid pGLY5045 is a roll-in integration vector that targets the URA6 locus and contains an expression cassette encoding the TNFRII-Fc fragment fusion protein. The plasmid contains two expression cassettes, each comprising a nucleic acid molecule encoding the TNFRII-Fc fragment fusion protein fused at the 5′ end to a nucleic acid molecule encoding the human serum albumin signal peptide, which is operably linked at the 5′ end to a nucleic acid molecule comprising the P. pastoris AOX1 promoter and at the 3′ end to a nucleic acid molecule comprising the S. cerevisiae CYC transcription termination sequence. The plasmid also includes a ZeocinR expression cassette comprising a nucleic acid molecule encoding the Sh ble ORF operably linked at the 5′ end to the S. cerevisiae TEF1 promoter and at the 3′ end to the S. cerevisiae CYC termination sequence.

FIG. 20 shows a plasmid map of pGLY6391. Plasmid pGLY6391 is a roll-in integration vector that targets the THR1 locus and contains an expression cassette encoding the TNFRII-Fc fragment fusion protein. The plasmid contains two expression cassettes, each comprising a nucleic acid molecule encoding the TNFRII-Fc fragment fusion protein without the C-terminal lysine residue fused at the 5′ end to a nucleic acid molecule encoding the human serum albumin signal peptide, which is operably linked at the 5′ end to a nucleic acid molecule comprising the P. pastoris AOX1 promoter and at the 3′ end to a nucleic acid molecule comprising the S. cerevisiae CYC transcription termination sequence. The plasmid also includes a ZeocinR expression cassette comprising a nucleic acid molecule encoding the Sh hie ORF operably linked at the 5′ end to the S. cerevisiae TEF1 promoter and at the 3′ end to the S. cerevisiae CYC termination sequence.

FIG. 21 shows a plasmid map of pGLY5085. Plasmid pGLY5085 is a KINKO plasmid for introducing a second set of the genes involved in producing sialylated N-glycans into P. pastoris. The plasmid is similar to plasmid YGLY2456 except that the P. pastoris ARG1 gene has been replaced with an expression cassette encoding hygromycin resistance (HygR) and the plasmid targets the P. pastoris TRP5 locus. The six tandem cassettes are flanked on one side by a nucleic acid molecule comprising a nucleotide sequence from the 5′ region and ORF of the TRP5 gene ending at the stop codon followed by a P. pastoris ALG3 termination sequence and on the other side by a nucleic acid molecule comprising a nucleotide sequence from the 3′ region of the TRP5 gene.

FIG. 22 shows a plasmid map of pGLY5755. Plasmid pGLY5755 is a KINKO integration plasmid that encodes a chimeric mouse POMGnT I and targets the HIS3 locus in P. pastoris. The expression cassette encoding the chimeric mouse POMGnT I comprises a nucleic acid molecule encoding the catalytic domain of the mouse POMGnT I ORF codon-optimized for effective expression in P. pastoris ligated in-frame with a nucleic acid molecule encoding S. cerevisiae MNN2-s signal peptide (53) operably linked at the 5′ end to a nucleic acid molecule that has the inducible P. pastoris AOX1 promoter sequence and at the 3′ end to a nucleic acid molecule that has the S. cerevisiae CYC transcription termination sequence. For selecting transformants, the plasmid comprises an expression cassette encoding the S. cerevisiae ARR3 ORF in which the nucleic acid molecule encoding the ORF is operably linked at the 5′ end to a nucleic acid molecule having the P. pastoris RPL10 promoter sequence and at the 3′ end to a nucleic acid molecule having the S. cerevisiae CYC transcription termination sequence.

FIG. 23 shows a plasmid map of pGLY5086. Plasmid pGLY5086 is a KINKO plasmid for introducing a second set of the genes involved in producing sialylated N-glycans into P. pastoris. The plasmid is similar to plasmid YGLY5085 except that the plasmid targets the P. pastoris THR1 locus.

FIG. 24 shows a plasmid map of pGLY5219. Plasmid pGLY5219 (FIG. 24) is an integration plasmid that encodes a chimeric mouse POMGnT I and targets the VPS10-1 locus in P. pastoris. The expression cassette encoding the chimeric mouse POMGnT I comprises a nucleic acid molecule encoding the catalytic domain of the mouse POMGnT I ORF ORF codon-optimized for effective expression in P. pastoris ligated in-frame with a nucleic acid molecule encoding S. cerevisiae Mnn6-s signal peptide (65) operably linked at the 5′ end to a nucleic acid molecule that has the constitutive P. pastoris GAPDH promoter sequence (SEQ ID NO:5) and at the 3′ end to a nucleic acid molecule that has the S. cerevisiae CYC transcription termination sequence. For selecting transformants, the plasmid comprises an expression cassette comprising the URA5 gene flanked by lacZ repeats.

FIG. 25 shows a map of pGLY5192. Plasmid pGLY5192 is an integration plasmid that targets the VPS10-1 locus. The plasmid comprises an expression cassette comprising the URA5 gene flanked by lacZ repeats flanked on one side by a nucleic acid molecule comprising a nucleotide sequence from the 5′ region of the VPS10-1 gene and on the other side by a nucleic acid molecule comprising a nucleotide sequence from the 3′ region of the VPS10-1 gene.

FIG. 26 shows a map of pGLY7087cv, Plasmid pGLY7087cv is a KINKO integration plasmid that encodes a chimeric mouse POMGnT I and targets the HIS3 locus in P. pastoris. The expression cassette encoding the chimeric mouse POMGnT I comprises a nucleic acid molecule encoding the catalytic domain of the mouse POMGnT I ORF codon-optimized for effective expression in P. pastoris ligated in-frame with a nucleic acid molecule encoding S. cerevisiae Mnn5-s signal peptide (56) operably linked at the 5′ end to a nucleic acid molecule that has the constitutive P. pastoris GAPDH promoter sequence and at the 3′ end to a nucleic acid molecule that has the S. cerevisiae CYC transcription termination sequence. For selecting transformants, the plasmid comprises an expression cassette encoding the S. cerevisiae ARR3 ORF in which the nucleic acid molecule encoding the ORF is operably linked at the 5′ end to a nucleic acid molecule having the P. pastoris RPL10 promoter sequence and at the 3′ end to a nucleic acid molecule having the S. cerevisiae CYC transcription termination sequence.

FIG. 27 shows the amino acid sequence of TNFRII-Fc (SEQ ID NO:75). Represented are the features: TNFRII ectodomain (in bold); IgG1 Fc domain (regular text): cysteine-rich subdomains of TNFRII domain (outlined by arrows): N-linked glycosylation sites (“N” residues encircled); and, optional C-terminal lysine (in brackets).

FIG. 28 shows a comparison of mucin-type O-glycosylation and dystroglycan-type O-glycosylation.

FIG. 29 shows a schematic representation of the O-glycosylation engineering strategy for TNFRII-Fc. Form 1: mannose-reduced O-glycans (strain YGLY10299); Form 2: mannose-reduced O-glycans and enhanced sialylation of N-glycans (strain YGLY11731); Form 3: sialylated O-glycans (strain YGLY12680). Forms 5A, 5B & 5C: sialylated O-glycans (strain YGLY14252). Form 7A: sialylated O-glycans (strain YGLY14954).

FIG. 30 shows a schematic representation of a purification strategy for recovering TNFRII-Fc produced in recombinant strains.

FIG. 31 shows a composite of gradient SDS-PAGE analyses of TNFRII-Fc purified using the method shown in FIG. 30. Purified TNFRII-Fc samples were resolved on 4-20% Tris-HCl BIORAD gels loaded with 3 μg/mL of reduced (R) or non-reduced (NR) TNFRII-Fc. Form 1: mannose-reduced O-glycans (strain YGLY10299); Form 2: mannose-reduced O-glycans and enhanced sialylation of N-glycans (strain YGLY11731); Form 3: sialylated O-glycans (strain YGLY12680). The control was commercial ENBREL.

FIG. 32 shows a table comparing the glycans composition of Form 1, Form 2, and Form 3 TNFRII-Fc. Form 1: mannose-reduced O-glycans (strain YGLY10299); Form 2: mannose-reduced O-glycans and enhanced sialylation of N-glycans (strain YGLY11731); Form 3: sialylated O-glycans (strain YGLY12680).

FIG. 33 shows the results of in vitro TNFRII-Fc-induced cell killing of L929 cells. Experimental design: L929 cells seeded overnight in 96-well plate (1×10⁴/well); cells treated with human recombinant TNFα (0.25 ng/mL) +/−TNFRII-Fc and incubated for 24 hours; and cell viability measured by ATPlite (luminescence readout). Form 1: mannose-reduced O-glycans (strain YGLY10299); Form 2: mannose-reduced O-glycans and enhanced sialylation of N-glycans (strain YGLY11731); Form 3: sialylated O-glycans (strain YGLY12680). The control was commercial ENBREL.

FIG. 34 shows the results of in vitro TNFRII-Fc-stimulated release of IL-6 in A549 cells. Experimental design: A549 cells seeded at 5×10⁴ per well in a 96 well plate and allowed to recover overnight; TNFRII-Fc samples titrated in triplicate; cells stimulated with 3 ng/mL human recombinant TNFα overnight at 37° C.; and IL6 production determined by AlphaLISA assay. Form 1: mannose-reduced O-glycans (strain YGLY10299); Form 2: mannose-reduced O-glycans and enhanced sialylation of N-glycans (strain YGLY11731); Form 3: sialylated O-glycans (strain YGLY12680). The control was commercial ENBREL,

FIG. 35 shows the results of in vivo rat pharmacokinetic analysis of TNFRII-Fc. Sprague Dawley (SD) rats were dosed SC at 1 mg/kg and serum samples collected at 4, 24, 48, 72, 96, 120, 144 and 168 hr. Serum TNFRII-Fc concentration was determined with a Gyro immunoassay using anti-TNFRII antibody for capture and labeled-anti-Fc antibody for detection. Form 1: mannose-reduced O-glycans (strain YGLY10299); Form 2: mannose-reduced O-glycans and enhanced sialylation of N-glycans (strain YGLY11731); Form 3: sialylated O-glycans (strain YGLY12680). The control was commercial ENBREL.

FIG. 36 shows a schematic representation of a purification strategy for recovering TNFRII-Fc from strain YGLY14252. Form 5A, hydroxyl apatite (HA) unbound wash purified. Form 5C, HA bound TNFRII-Fc eluted and purified. Form B, a 1:1 mix of Form 5A and 5C. The control was commercial ENBREL.

FIG. 37 shows a composite of gradient SDS-PAGE analyses of TNFRII-Fc purified using the method shown in FIG. 36. Purified TNFRII-Fc samples were resolved on 4-20% Tris-HCl BIORAD gels loaded with 2.5 μg/lane of non-reduced (NR) TNFRII-Fc. YGLY14252. The control was commercial ENBREL.

FIG. 38 shows a table comparing the glycans composition of TNFRII-Fc in Form 5A, Form 5B, and Form 5C.

FIG. 39 shows a table comparing the in vitro TNFRII-Fc-induced cell killing of L929 cells and the in vitro TNFRII-Fc fragment fusion protein-stimulated release of IL-6 in A549 cells of TNFRII-Fc Form 5A, Form 5B, and Form 5C. Assays were performed as in FIGS. 33 and 34. The control was commercial ENBREL.

FIG. 40 shows the results of in vivo rat pharmacokinetic analysis of TNFRII-Fc fragment fusion protein. SD rats were dosed SC at 1 mg/kg and serum samples collected at 4, 24, 48, 72, 96, 120, 144 and 168 hr. Serum TNFRII-Fc fragment fusion protein concentration was determined with a Gyro immunoassay using anti-TNFRII as capture and anti-Fc as detection. The control was commercial ENBREL.

FIG. 41 shows the results of in vivo mouse pharmacokinetic analysis of TNFRII-Fc fragment fusion protein. Mice were dosed with TNFRII-Fc fragment fusion protein SC at varying doses (0.1, 1, 5, 10 and 20 mg/kg) and the serum harvested at 48 hours post-inoculation. Serum TNFRII-Fc fusion protein concentration was determined with a Gyro immunoassay using anti-TNFRII as capture and anti-Fc as detection. The control was commercial ENBREL.

FIG. 42 shows the results of the in vivo mouse chronic rheumatoid arthritic model. Transgenic mice were separated into 7 groups consisting of 8 gender and age-matched mice each, which received intraperitoneally 10 μl of test compounds per gram of body weight, twice weekly. The groups received different test materials and dose levels, as follows: Vehicle, Pichia TNFRII-Fc at 30, 10 and 3 mg/kg; commercial Enbrel at 30, 10 and 3 mg/kg. Treatment was initiated at the onset of arthritis (three weeks of age) and continued over 8 weeks; the study was concluded at 10 weeks of age.

FIG. 43 shows a schematic representation of an alternative purification strategy for recovering TNFRII-Fc with enriched sialic acid content.

FIG. 44 shows a composite of gradient SDS-PAGE analyses of TNFRII-Fc purified isolated from strain YGLY14954, using the method shown in FIG. 43. Purified TNFRII-Fc samples were resolved on 4-20% Tris-HCl BIORAD gels loaded with 2.5 μg/Lane of non-reduced TNFRII-Fc. The control was commercial ENBREL.

FIG. 45 shows a table comparing the glycans composition of TNFRII-Fc in Form 7A and commercial ENBREL.

FIG. 46 shows the results of in vivo rat pharmacokinetic analysis of TNFRII-Fc fragment fusion protein purified by the Prosep-PB strategy compared to commercial ENBREL. SD rats were dosed SC at 1 mg/kg and serum samples collected at 4, 24, 48, 72, 96, 120, 144 and 168 hours. Serum TNFRII-Fc fragment fusion protein concentration was determined with a Gyro immunoassay using anti-TNFRII as capture and anti-Fc as detection. The control was commercial ENBREL.

### DETAILED DESCRIPTION OF THE INVENTION

The present invention provides compositions comprising a recombinant human tumor necrosis factor fused to the constant region of an antibody (TNFRII-Fc fragment fusion protein) wherein the recombinant TNFRII-Fc fragment fusion protein comprises sialylated, afucosylated N-glycans and O-glycans. The sialylated O-glycans are of the dystroglycan type and not the mucin type. The sialic acid residue comprising the N-glycans and O-glycans consist only of N-acetylneuraminic acid (NANA) residues. In addition, the sialic acid residues are linked to the non-reducing end of the oligosaccharide comprising the N-glycan and O-glycans in an α-2,6 linkage. Further provided are host cells for making the a recombinant TNFRII-Fc fragment fusion protein.

N-linked and O-linked are two major types of glycosylation. N-linked glycosylation (N-glycosylation) is characterized by the β-glycosylamine linkage of N-acetylglucosamine (GlcNac) to asparagine (Asn) (Spiro, Glycobiol. 12: 43R-56R (2002)). It has been well established that the consensus sequence motif Asn-X-Ser/Thr is essential in N-glycosylation (Blom et al., Proteomics 4: 1633-1649 (2004)). The most abundant form of O-linked glycosylation (O-glycosylation) is of the mucin-type, which is characterized by α-N-acetylgalactosamine (GalNAc) attached to the hydroxyl group of serine/threonine (Ser/Thr) side chains by the enzyme UDP-N-acetyl-D-galactosamine:polypeptide N-acetylgalactosaminyltransferase (Hang & Bertozzi, Bioorg. Med. Chem. 13: 5021-5034 (2005); Julenius et al., Glycobiol. 15: 153-164 (2005)). Mucin-type O-glycans can further include galactose and sialic acid residues. Mucin-type O-glycosylation is commonly found in many secreted and membrane-bound mucins in mammal, although it also exists in other higher eukaryotes (Hanish, Biol. Chem. 382: 143-149 (2001)). As the main component of mucus, a gel playing crucial role in defending epithelial surface against pathogens and environmental injury, mucins are in charge of organizing the framework and conferring the rheological property of mucus. Beyond the above properties exhibited by mucins, mucin-type O-glycosylation is also known to modulate various protein functions in vivo (Hang & Bertozzi, Bioorg. Med. Chem. 13: 5021-5034 (2005)). For instance, mucin-like glycans can serve as receptor-binding ligands during an inflammatory response (McEver & Cummings, J. Chin. Invest. 100: 485-491 (1997

Another form of O-glycosylation is that of the O-mannose-type glycosylation (T. Endo, BBA 1473: 237-246 (1999)). In mammalian organisms this form of glycosylation can be sub-divided into two forms. The first form is the addition of a single mannose to a serine or threonine residue of a protein. This is a rare occurrence and has been demonstrated on very few proteins, including IgG2 light chain (Martinez et al, J. Chromatogr. A. 1156: 183-187 (2007)). A more common form of O-mannose-type glycosylation in mammalian systems is that of the dystroglycan-type, which is characterized by β-N-acetylglucosamine (GlcNAc) attached to a mannose residue attached to the hydroxyl group of serine/threonine side chains in an α1 linkage by an O-linked mannose β1,2-N-acetylglucosaminyltransferase 1 (POMGnT1) (T. Endo, BBA 1473: 237-246 (1999)). Dystroglycan-type O-glycans can further include galactose and sialic acid residues. Unlike N-glycosylation, the consensus motif has not been identified in the sequence context of mucin or dystroglycan O-glycosylation sites.

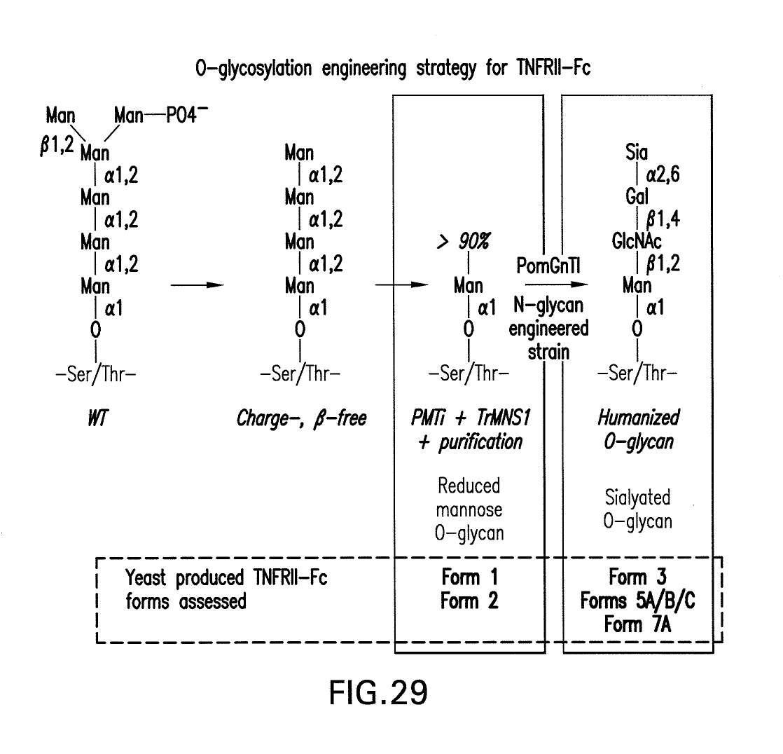

In fungi such as Pichia pastor's, O-glycosylation produces O-glycans that can include up to five or six mannose residues (See for example, Tanner & Lehle, Biochim. Biophys. Acta 906: 81-89 (1987); Herscovics & Orlean, FASEB J. 7: 540-550 (1993); Trimble et al., GlycoBiol. 14: 265-274 (2004); Lommel & Strahl, Glycobiol. 19: 816-828 (2009). Wild-type Pichia pastoris as shown in FIG. 29 can produce O-mannose-type O-glycans consisting of up to six mannose residues in which the terminal mannose residue can be phosphorylated. By abrogating phosphomannosyltransferase activity and β-mannosyltransferase activity in the Pichia pastoris, which results in charge-free O-glycans without β-linked mannose residues, and cultivating the Pichia pastoris lacking phosphomannosyltransferase activity and β-mannosyltransferase activity in the presence of a protein PMT inhibitor, which reduces O-glycosylation site occupancy, and a secreted α-1,2-mannosidase, which reduces the chain length of the charge-free O-glycans, O-mannose reduced glycans (or mannose-reduced O-glycans) can be produced (See U.S. Published Application No. 20090170159 and U.S. patent No.). The consensus motif has not been identified in the sequence context of fungal O-glycosylation sites.

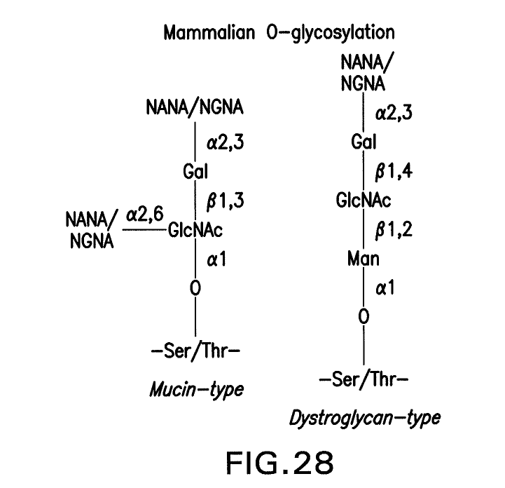

Mucin-type O-glycosylation is primarily found on cell surface proteins and secreted proteins. Dystroglycan-type O-glycosylation is primarily associated with proteins comprising the extracellular matrix. Both mucin- and dystroglycan-type O-glycans may possess terminal sialic acid residues. As shown in FIG. 28, the terminal sialic acid residues are in α-2,3 linkage with the preceding galactose residue. In some instances, as shown in FIG. 28, mucin-type O-glycans can also possess a branched α-2,6 sialic acid residue. The sialic acid present on each type of structure on glycoproteins obtained from recombinant non-human cell lines can include mixtures of N-acetylneuraminic acid (NANA) and N-glycolylneuraminic acid (NGNA). However, in contrast to glycoproteins obtained from mammalian cells, the sialic acid present on each type of structure on glycoproteins obtained from human cells is primarily composed of NANA. Thus, glycoprotein compositions obtained from mammalian cell culture include sialylated N-glycans that have a structure primarily associated to glycoproteins produced in non-human mammalian cells. ENBREL (etanercept) is a commercially provided TNFRII-Fc fragment fusion protein composition that is produced in Chinese Hamster Ovary (CHO) cells. U.S. Pat. No. 5,459,031 discloses that the level of NONA in a glycoprotein produced by a mammalian recombinant host cell can be controlled by monitoring and adjusting the levels of CO₂ during production of the glycoprotein in the host cell. The method was shown to reduce but not eliminate the presence of NGNA in the glycoprotein. In contrast, the present invention provides methods for producing TNFRII-Fc fusion protein compositions wherein the NANA is the only sialic acid species on the glycoprotein.

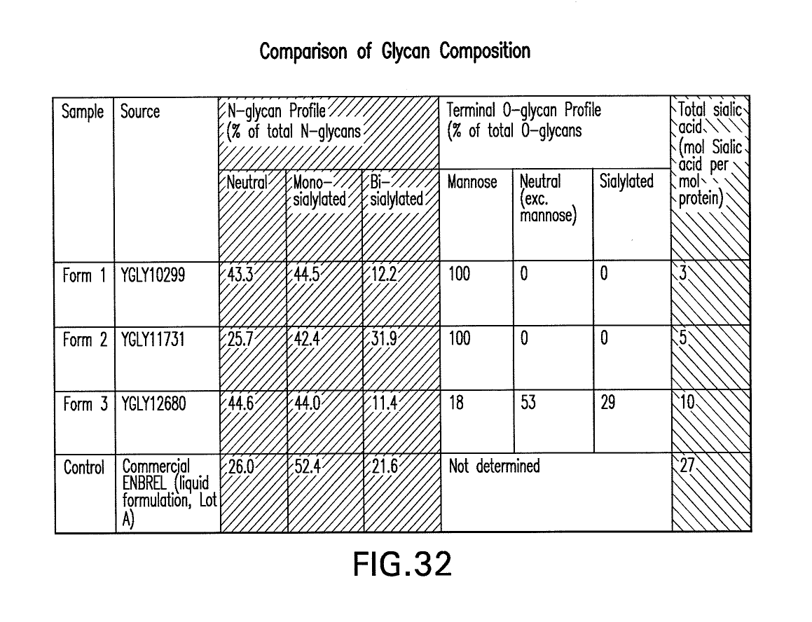

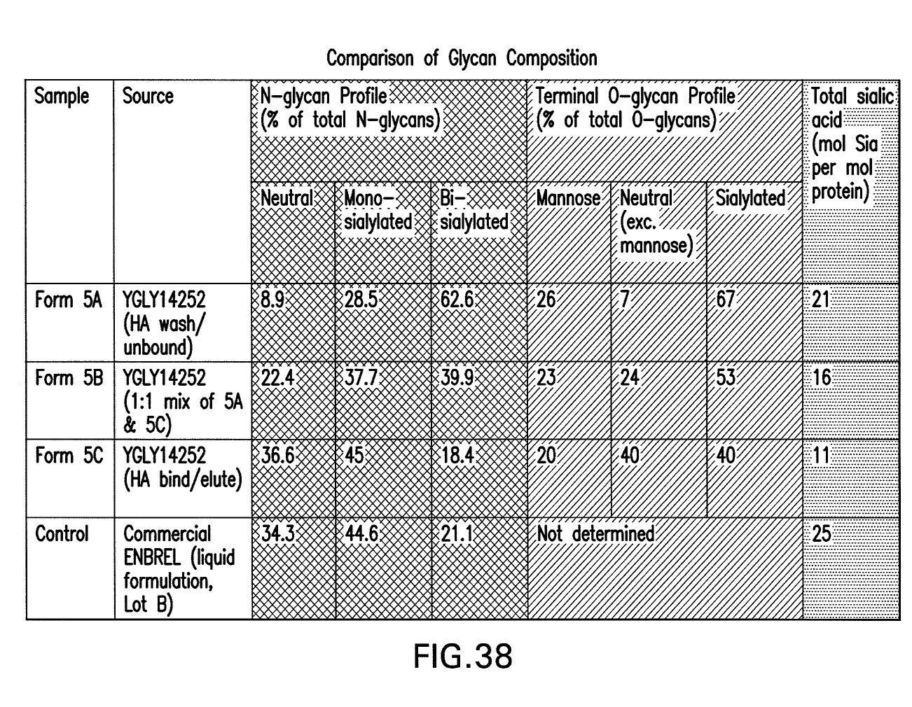

The N-glycan and O-glycan profiles of the several compositions of TNFRII-Fc fragment fusion protein of the present invention are shown in FIGS. 32 and 38. FIG. 32 shows the glycosylation profiles for TNFRII-Fc fragment fusion protein produced in strain YGLY12680, a Pichia pastoris strain genetically engineered to produce sialylated N-glycans and O-glycans, compared to the profile of a TNFRII-Fc fragment fusion protein produced in strains that lacks the ability to produce sialylated O-glycans. Strain YGLY12680 is a genetically engineered strain that includes a chimeric POMGnT I comprising the catalytic domain of POMGnT I fused to a heterologous targeting or signaling peptide that targets the chimeric POMGnT to the endoplasmic reticulum (ER) or Golgi apparatus, which transfers a GlcNAc residue to the O-linked mannose residue of an O-glycan, and a duplication of the nucleic acid molecules encoding a chimeric α-2,6-sialyltransferase (α-2,6ST) comprising the catalytic domain of an α-2,6ST fused to a heterologous targeting or signaling peptide that targets the chimeric α-2,6ST to the ER or Golgi apparatus, and the enzymes involved in making the CMP-sialic acid substrate for the chimeric α-2,6ST. Because yeast do not include an endogenous sialic acid pathway, the sialylated N-glycans and O-glycans produced by the strain are only of the NANA type. Thus, the strains herein produce sialylated N-glycans and O-glycans that include only the NANA type, similar to the N-glycans and O-glycans produced in human cells. This is in contrast to mammalian cells that produce N-glycans and O-glycans in a mixture of NANA and NGNA types. In general, the mole of sialic acid per mole of protein produced in strain YGLY12680 was about 10. Sialylated N-glycans were the predominant species in the strain of which the predominant subspecies was mono-sialylated. Neutral O-glycans were the predominant species in the strain and were of the dystroglycan type. Neutral N-glycans in either glycoform include galactose-, GlcNAc-, or mannose-terminated oligosaccharide chains.

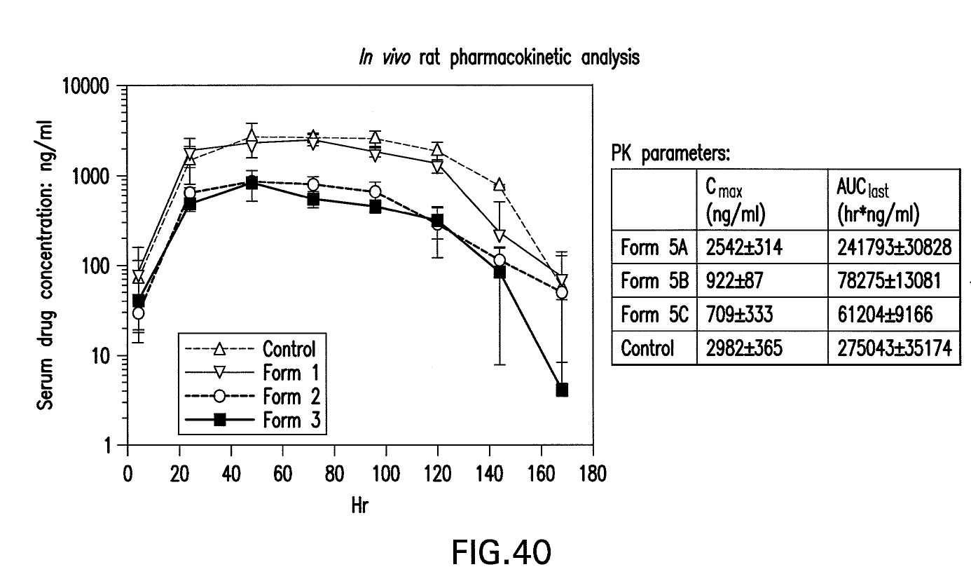

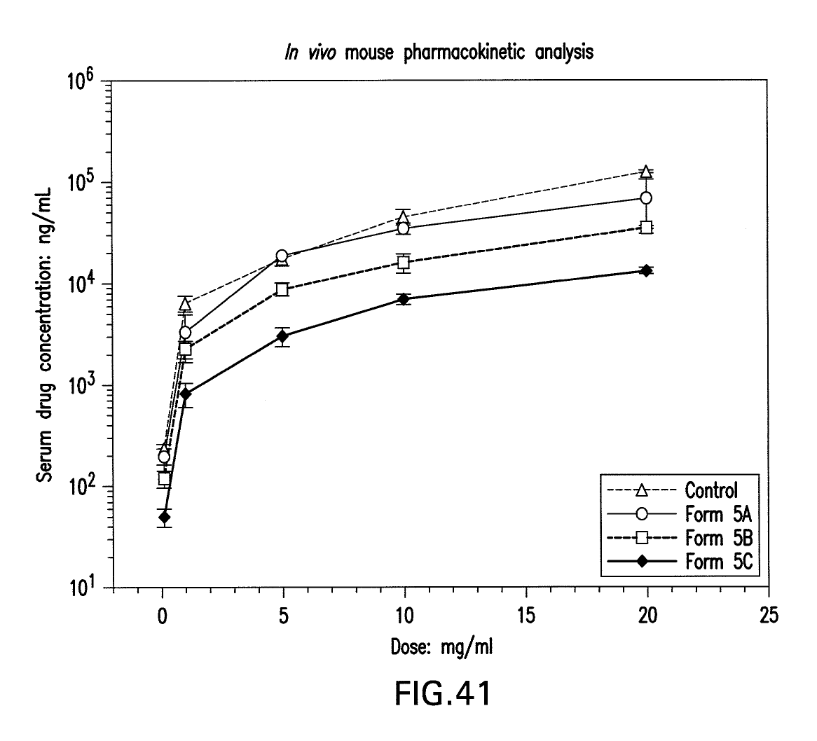

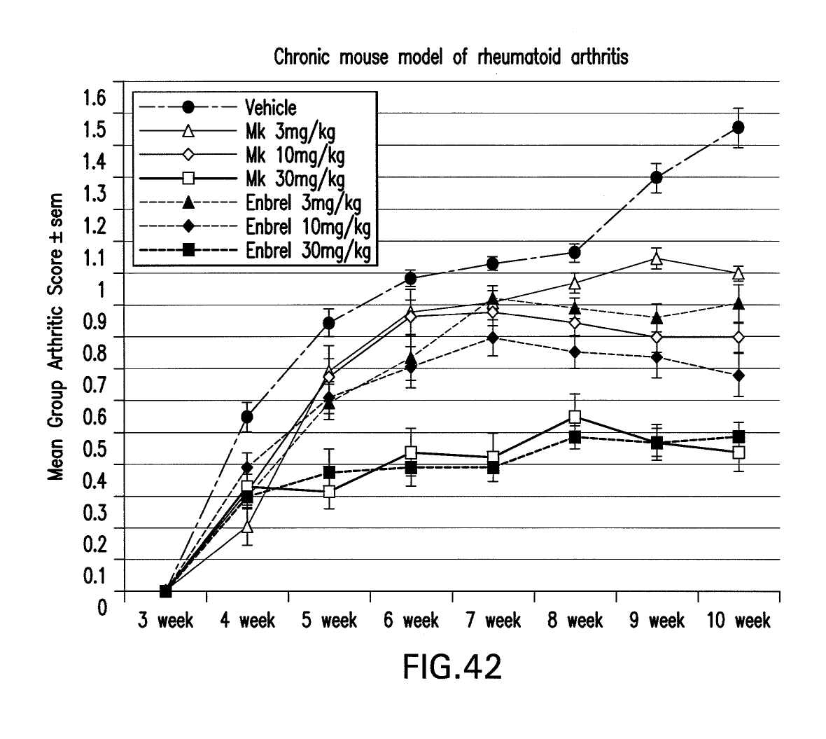

FIG. 38 shows the glycosylation profiles for TNFRII-Fc fragment fusion protein produced in strain YGLY14252. The TNFRII-Fc fragment fusion protein was fractionated into three fractions, and the glycosylation profiles determined for each fraction. The mole of sialic acid per mole of protein ranged from about 11 to 21 depending on the fraction. For Form 5A, the sialylated N-glycan and O-glycan glycoforms comprised the predominant species. As shown in FIGS. 40-41, Form 5A pharmacokinetics was similar to commercially available ENBREL where as the less sialylated forms (Form 5B and 5C) had reduced pharmacokinetics compared to ENBREL. The sialylated N-glycans and O-glycans produced by the strain are only of the NANA type. The TNFRII-Fc produced in the recombinant Pichia pastoris strains when compared to commercial Enbrel in the mouse chronic rheumatoid arthritic model demonstrated a dose dependent potency similar to commercial Enbrel (FIG. 42).

Therefore, the present invention provides a composition comprising or consisting essentially of a recombinant fragment of human tumor necrosis factor receptor fused to the constant region of an antibody (TNFRII-Fc) wherein the TNFRII-Fc has N-glycans and O-glycans and wherein the O-glycans are of the dystroglycan- or O-man type, and pharmaceutically acceptable salts thereof.

In further aspects of the composition, the N-glycans and O-glycans on the TNFRII-Fc are predominantly sialylated with α-2,6 or α-2,3 sialic acid residues. In further still aspects of the composition, the N-glycans on the TNFRII-Fc lack fucose residues; however, in particular aspects of the composition, one or more of the N-glycans on the TNFRII-Fc are fucosylated. In further still aspects, the N-glycans and O-glycans on the TNFRII-Fc, which are sialylated, comprise N-acetylneuraminic acid (NANA) and no N-glycolylneuraminic acid (NGNA).

In further still aspects of the composition, a ratio of mole sialic acid to mole of the TNFRII-Fc is at least 10. In further still aspects of the composition, a ratio of mole sialic acid to mole of the TNFRII-Fc is about 10 to 21. In further still aspects of the composition, a ratio of mole sialic acid to mole of the TNFRII-Fc is greater than 21.

In further aspects of the composition, at least 50%, 60%, 70%, 80%, 90%, or 100% of the N-glycans are sialylated. In further still aspects, the N-glycans on the TNFRII-Fc comprise or consist of predominantly mono-, bi-, tri-, or tetra-sialylated N-glycans. In further still aspects, the N-glycans on the TNFRII-Fc comprise or consist of predominantly mono-sialylated N-glycans. In further still aspects of the composition, the N-glycans on the TNFRII-Fc comprise or consist of predominantly bi-sialylated N-glycans. In further still aspects, the N-glycans on the TNFRII-Fc comprise or consist of predominantly tri-sialylated N-glycans. In further still aspects of the composition, the N-glycans on the TNFRII-Fc comprise or consist of predominantly tetra-sialylated N-glycans.

In further still aspects of the composition, the O-glycans on the TNFRII-Fc comprise or consist of predominantly sialylated O-glycans. In further still aspects, greater than 10%, 20%, 30%, 40%, or 50% of the O-glycans on the TNFRII-Fc comprise or consist of sialylated O-glycans. In further still aspects, less than 10%, 20%, 40% or 50% of the O-glycans on the TNFRII-Fc terminate in mannose.

In further still aspects of the composition, the TNFRII domain of the TNFRII-Fc comprises or consists of an amino acid sequence with at least 90%, 91%, 92%, 93%, 94%, 95%, 96%, 97%, 98%, or 99% identity to the amino acid sequence for the TNFRII domain set forth in SEQ ID NO:73 or 75. The receptor domain includes amino acids 1 to 235 of SEQ ID NO:73 or 75 and is encoded by nucleotides 1-705 of SEQ ID NO:72 or 74. The receptor domain includes amino acids 1 to 235 of SEQ ID NO:73 or 75 and is encoded by nucleotides 1-705 of SEQ ID NO:72 or 74.

Further provided is a composition comprising or consisting essentially of a recombinant fragment of human tumor necrosis factor receptor fused to the constant region of an antibody (TNFRII-Fc) wherein the TNFRII-Fc has N-glycans and O-glycans and wherein the O-glycans are O-mannose reduced glycans, and pharmaceutically acceptable salts thereof. An O-mannose reduced glycan is an O-glycan in which the predominant O-glycan consists of a single mannose (mannose type) or mannobiose type (two mannose residues). In further aspects of the composition, the TNFRII domain of the TNFRII-Fc comprises or consists of an amino acid sequence with at least 90%, 91%, 92%, 93%, 94%, 95%, 96%, 97%, 98%, or 99% identity to the amino acid sequence for the TNFRII domain set forth in SEQ ID NO:73 or 75. The receptor domain includes amino acids 1 to 235 of SEQ ID NO:73 or 75 and is encoded by nucleotides 1-705 of SEQ ID NO:72 or 74.

Lower eukaryotes such as yeast or filamentous fungi are often used for expression of recombinant glycoproteins because they can be economically cultured, give high yields, and when appropriately modified are capable of suitable glycosylation. Yeast in particular offers established genetics allowing for rapid transfections, tested protein localization strategies and facile gene knock-out techniques. Suitable vectors have expression control sequences, such as promoters, including 3-phosphoglycerate kinase or other glycolytic enzymes, and an origin of replication, termination sequences, and the like as desired. These glycoengineered host cells enable the production of the TNFRII-Fc comprising the compositions disclosed herein.

Therefore, further provided is a method for producing a recombinant human tumor necrosis factor fused to the constant region of an antibody (TNFRII-Fc) having sialylated N-glycans and O-glycans comprising or consisting of (a) providing a recombinant lower eukaryote host cell genetically engineered to produce glycoproteins having sialylated N-glycans and further comprising (i) a nucleic acid molecule encoding the TNFRII-Fc; (ii) a nucleic acid molecule encoding an α1,2-mannosidase activity linked to a heterologous targeting or signaling peptide that targets the mannosidase activity to the secretory pathway; and (iii) a nucleic acid molecule encoding an O-linked mannose β1,2-N-acetylglucosaminyltransferase 1 (POMGnT1); (b) culturing the host cell under conditions suitable for producing the TNFRII-Fc; and (c) recovering the TNFRII-Fc from the culture fluid to produce the TNFRII-Fc having sialylated N-glycans and O-glycans.

In further aspects, the POMGnT1 is provided as a fusion protein comprising the catalytic domain of the POMGnT1 fused to a heterologous targeting or signaling peptide that targets the POMGnT1 to the secretory pathway, e.g., the ER or Golgi apparatus. Examples of heterologous targeting or signaling peptides include but are not limited to the MNN2, MNN5 and MNN6 targeting or signaling peptides.

In further aspects of the method, the N-glycans and O-glycans on the TNFRII-Fc are predominantly sialylated with α-2,6 or α-2,3 sialic acid residues. In further still aspects, the N-glycans on the TNFRII-Fc lack fucose residues. In further still aspects of the method, the N-glycans and O-glycans on the TNFRII-Fc, which are sialylated, comprise N-acetylneuraminic acid (NANA) and no N-glycolylneuraminic acid (NGNA).

In further still aspects of the method, a ratio of mole sialic acid to the mole of the TNFRII-Fc is at least 10. In further still aspects, a ratio of mole sialic acid to mole of the TNFRII-Fc is about 10 to 21. In further still aspects of the method, a ratio of mole sialic acid to mole of the TNFRII-Fc is greater than 21.

In further aspects of the method, at least 50%, 60%, 70%, 80%, 90%, or 100% of the N-glycans are sialylated. In further still aspects, the NV glycans on the TNFRII-Fc comprise or consist of predominantly mono-, bi-, tri-, or tetra-sialylated N-glycans. In further still aspects of the method, the N-glycans on the TNFRII-Fc comprise or consist of predominantly mono-sialylated N-glycans. In further still aspects, the N-glycans on the TNFRII-Fc comprise or consist of predominantly bi-sialylated N-glycans. In further still aspects of the method, the N-glycans on the TNFRII-Fc comprise or consist of predominantly tri-sialylated N-glycans. In further still aspects of the method, the N-glycans on the TNFRII-Fc comprise or consist of predominantly tetra-sialylated N-glycans.

In further still aspects of the method, the O-glycans on the TNFRII-Fc comprise or consist of predominantly sialylated O-glycans. In further still aspects, greater than 10%, 20%, 30%, 40%, or 50% of the O-glycans on the TNFRII-Fc comprise or consist of sialylated O-glycans. In further still aspects of the method, less than 10%, 20%, 40% or 50% of the O-glycans on the TNFRII-Fc terminate in mannose.

In further still aspects of the method, the TNFRII domain of the TNFRII-Fc comprises or consists of an amino acid sequence with at least 90%, 91%, 92%, 93%, 94%, 95%, 96%, 97%, 98%, or 99% identity to the amino acid sequence for the TNFRII domain set forth in SEQ ID NO:73 or 75. The receptor domain includes amino acids 1 to 235 of SEQ ID NO:73 or 75 and is encoded by nucleotides 1-705 of SEQ ID NO:72 or 74.

Further provided is a method for producing a recombinant human tumor necrosis factor fused to the constant region of an antibody (TNFRII-Fc) having sialylated N-glycans and O-mannose reduced glycans comprising or consisting of (a) providing a recombinant lower eukaryote host cell genetically engineered to produce glycoproteins having sialylated N-glycans and further comprising (i) a nucleic acid molecule encoding the TNFRII-Fc; and (ii) a nucleic acid molecule encoding an α-1,2-mannosidase activity linked to a heterologous targeting or signaling peptide that targets the mannosidase activity to the secretory pathway; (b) culturing the host cell under conditions suitable for producing the TNFRII-Fc; and (c) recovering the TNFRII-Fc from the culture fluid to produce the TNFRII-Fc having sialylated N-glycans and O-mannose reduced glycans.

In further aspects of the method, the TNFRII domain of the TNFRII-Fc comprises or consists of an amino acid sequence with at least 90%, 91%, 92%, 93%, 94%, 95%, 96%, 97%, 98%, or 99% identity to the amino acid sequence for the TNFRII domain set forth in SEQ ID NO:73 or 75. The receptor domain includes amino acids 1 to 235 of SEQ ID NO:73 or 75 and is encoded by nucleotides 1-705 of SEQ ID NO:72 or 74.

In further aspects, the host cells are cultured in the presence of a PMT inhibitor which reduces the number of sites on the TNFRII-Fc that is O-glycosylated.

### Host Cells

Useful lower eukaryote host cells for producing the TNFRII-Fc molecules disclosed herein are glycoengineered host cells that include but are not limited to Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum and Neurospora crassa. Various yeasts, such as K. lactis, Pichia pastoris, Pichia methanolica, and Hansenula polymorpha are particularly suitable for cell culture because they are able to grow to high cell densities and secrete large quantities of recombinant protein. Likewise, filamentous fungi, such as Aspergillus niger, Fusarium sp, Neurospora crassa and others can be used to produce glycoproteins of the invention at an industrial scale. In the case of lower eukaryotes, cells are routinely grown from between about one and a half to three days.

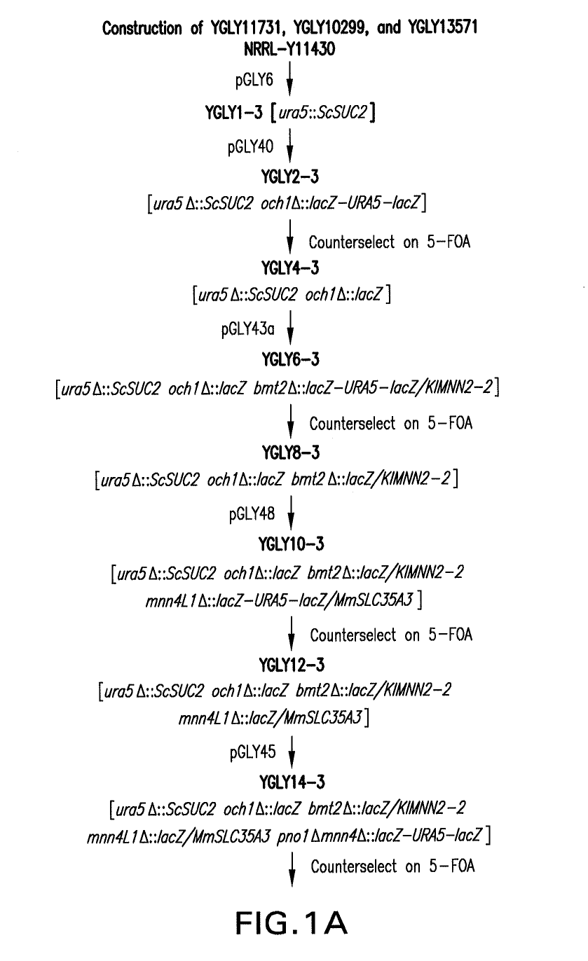

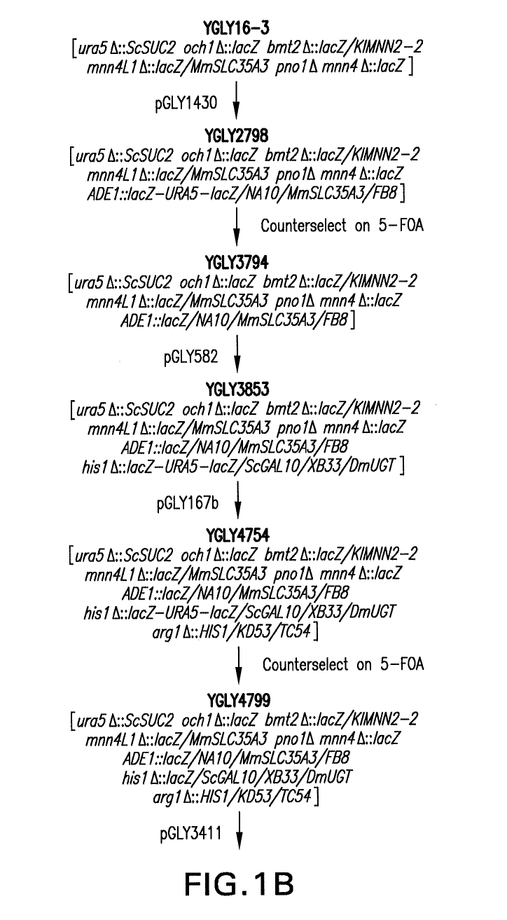

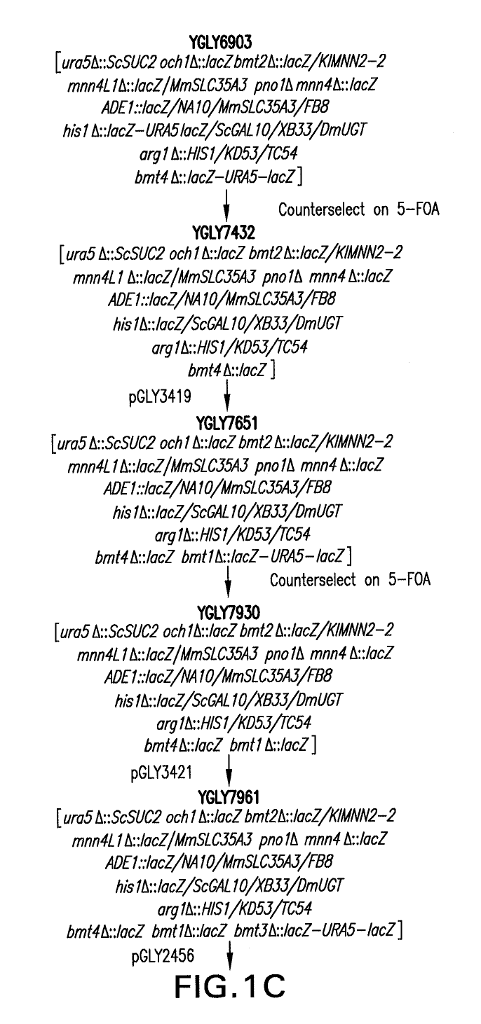

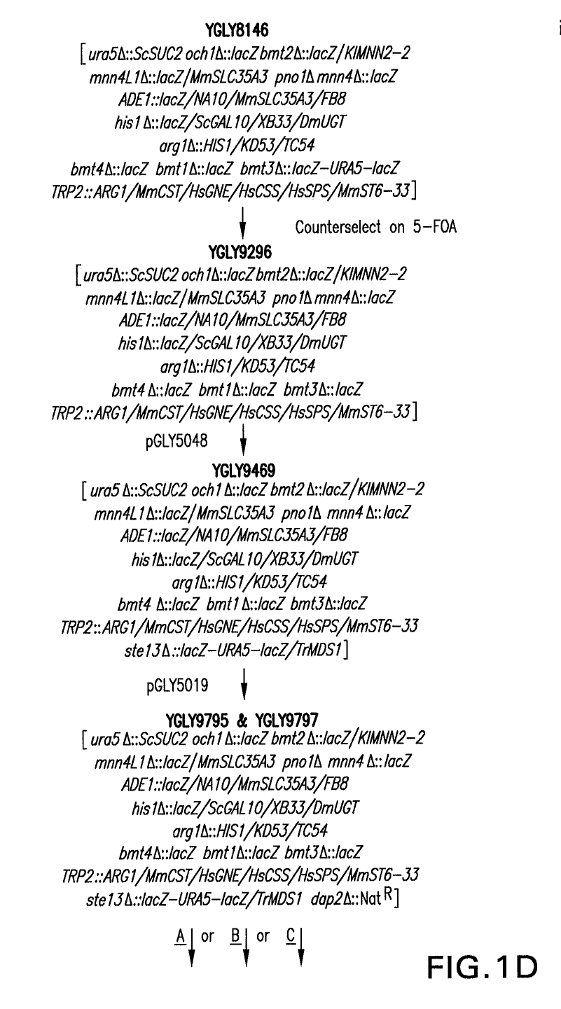

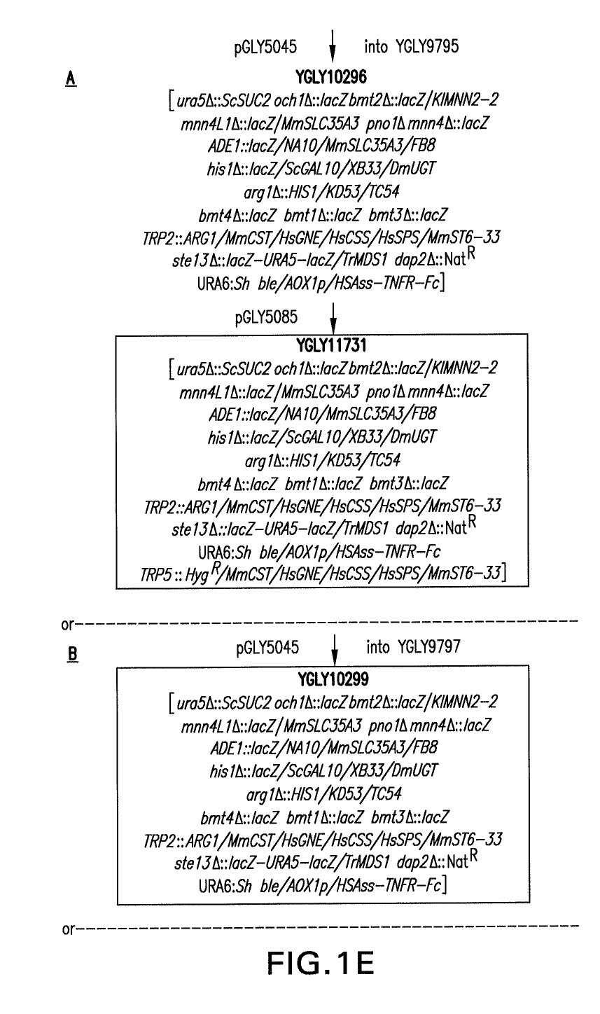

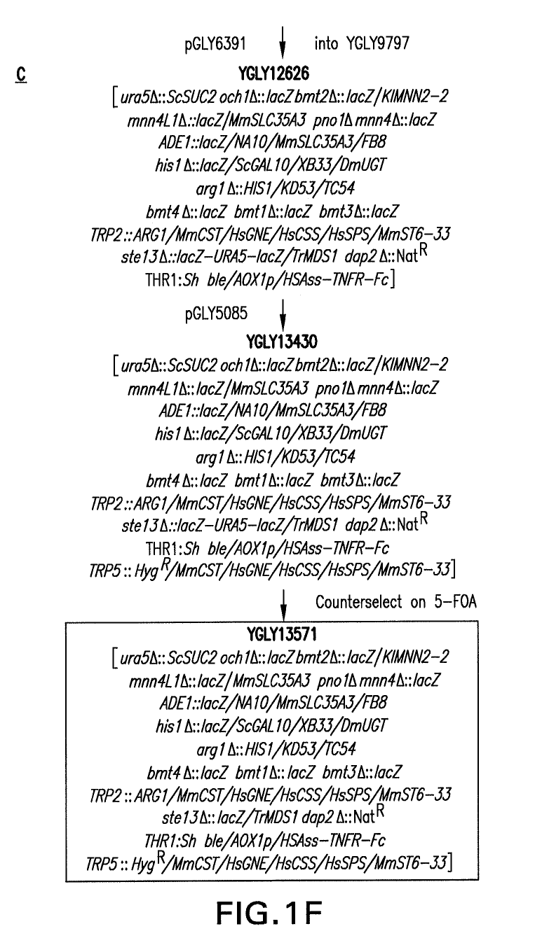

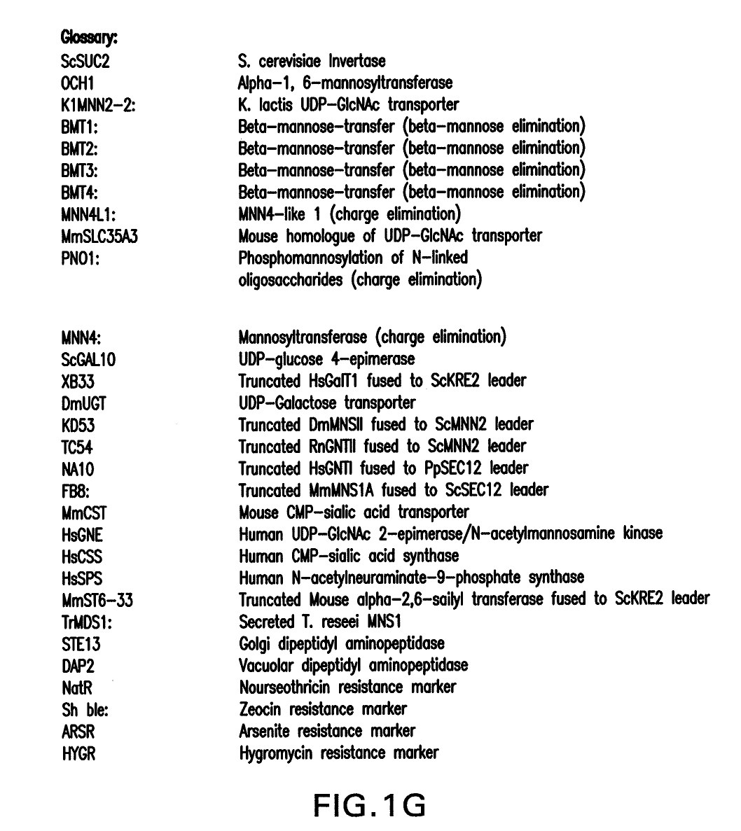

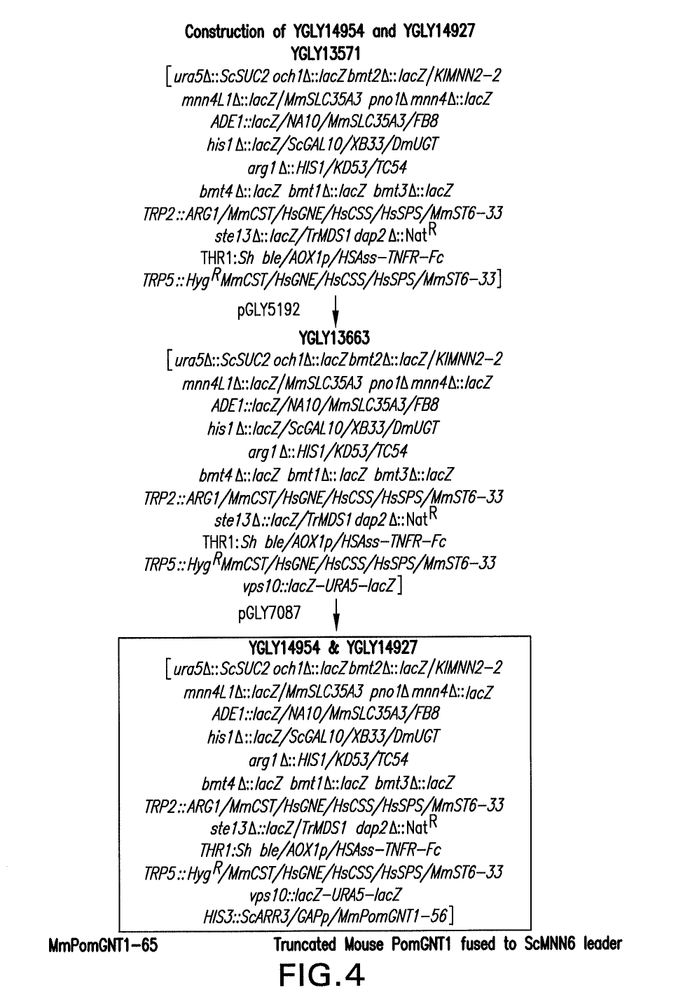

The Pichia pastoris strains YGLY11731, YGLY10299, YGLY13571, YGLY12680, and YGLY14252 shown in FIGS. 1A-G, 2A-B, and 3 and their construction are described in Examples 1-3. Example 4 describes the construction of strains YGLY14954 and YGLY14927, shown in FIG. 4. These strains are similar to strain YGLY14252 except that the chimeric POMGnT is fused to a different heterologous targeting or signaling peptide and it is inserted into a different locus in the Pichia pastoris genome. The methods for constructing the strains in Examples 1-4 can be used to construct other lower eukaryote host cells that express TNFRII-Fc fragment fusion protein with characteristics similar to the TNFRII-Fc fragment fusion protein described in Examples 1-4. In general, these lower eukaryote host cells can be achieved by eliminating selected endogenous glycosylation enzymes and/or supplying exogenous enzymes as described by Gerngross et al., U.S. Pat. No. 7,449,308, the disclosure of which is incorporated herein by reference. In particular aspects of the invention, the host cell is yeast, which in further aspects, a methylotrophic yeast such as Pichia pastoris or Ogataea minuta and mutants thereof. In general, the TNFRII-Fc fragment fusion protein produced in a lower eukaryote other than Pichia pastoris as exemplified in the examples or using variants or species of the enzymes and heterologous targeting or signaling peptides exemplified in the examples are expected to produce a TNFRII-Fc fragment fusion protein with general characteristics similar or the same as that for TNFRII-Fc fragment fusion protein produced as described in the examples. These general characteristics are that the O-glycans are of the dystroglycan type, the N-glycans are afucosylated, the N-glycans and O-glycans possess only NANA residues and no NGNA residues, and provided the sialyltransferase is an α-2,6 sialyltransferase, the sialic acid residues will linked via an α-2,6 linkage.

A general scheme for constructing a host cell that can produce the TNFRII-Fc fragment fusion protein disclosed herein can include the following. The host cell is selected that lacks in initiating 1,6-mannosyl transferase activity. Such host cells either naturally lack an endogenous initiating 1,6-mannosyl transferase activity or are genetically engineered to lack the initiating 1,6-mannosyl transferase activity. Then, the host cell further includes an α1,2-mannosidase catalytic domain fused to a heterologous targeting or signal peptide not normally associated with the catalytic domain and selected to target the α1,2-mannosidase activity to the ER or Golgi apparatus of the host cell. Passage of a recombinant glycoprotein through the ER or Golgi apparatus of the host cell produces a recombinant glycoprotein comprising a Man₅GlcNAc₂ glycoform, for example, a recombinant glycoprotein composition comprising predominantly a Man₅GlcNAc₂ glycoform. U.S. Pat. No. 7,029,872, U.S. Pat. No. 7,449,308, and U.S. Published Patent Application No. 2005/0170452, the disclosures of which are all incorporated herein by reference, disclose lower eukaryote host cells capable of producing a glycoprotein comprising a Man₅GlcNAc₂ glycoform.

The immediately preceding host cell further includes an N-netylglucosaminyltransferase I (GlcNAc transferase I or GnT I) catalytic domain fused to a heterologous targeting or signal peptide not normally associated with the catalytic domain and selected to target GlcNAc transferase I activity to the ER or Golgi apparatus of the host cell. Passage of the recombinant glycoprotein through the ER or Golgi apparatus of the host cell produces a recombinant glycoprotein comprising a GlcNAcMan₅GlcNAc₂ glycoform, for example a recombinant glycoprotein composition comprising predominantly a GlcNAcMan₅GlcNAc₂ glycoform. U.S. Pat. No. 7,029,872, U.S. Pat. No. 7,449,308, and U.S. Published Patent Application No. 2005/0170452, the disclosures of which are all incorporated herein by reference, disclose lower eukaryote host cells capable of producing a glycoprotein comprising a GlcNAcMan₅GlcNAc₂ glycoform.

The immediately preceding host cell further includes a mannosidase H catalytic domain fused to a heterologous targeting or signal peptide not normally associated with the catalytic domain and selected to target mannosidase II activity to the ER or Golgi apparatus of the host cell. Passage of the recombinant glycoprotein through the ER or Golgi apparatus of the host cell produces a recombinant glycoprotein comprising a GlcNAcMan₃GlcNAc₂ glycoform, for example a recombinant glycoprotein composition comprising predominantly a GlcNAcMan₃GlcNAc₂ glycoform. U.S. Pat. No. 7,029,872 and U.S. Pat. No. 7,625,756, the disclosures of which are all incorporated herein by reference, discloses lower eukaryote host cells that express mannosidase II enzymes and are capable of producing glycoproteins having predominantly a GlcNAcMan₃GlcNAc₂ glycoform.

The immediately preceding host cell further includes N-acetylglucosaminyltransferase II (GlcNAc transferase II or GnT II) catalytic domain fused to a heterologous targeting or signal peptide not normally associated with the catalytic domain and selected to target GlcNAc transferase II activity to the ER or Golgi apparatus of the host cell. Passage of the recombinant glycoprotein through the ER or Golgi apparatus of the host cell produces a recombinant glycoprotein comprising a GlcNAc₂Man₃GlcNAc₂ glycoform, for example a recombinant glycoprotein composition comprising predominantly a GlcNAc₂Man₃GlcNAc₂ glycoform. U.S. Pat. Nos. 7,029,872 and 7,449,308 and U.S. Published Patent Application No. 2005/0170452, the disclosures of which are all incorporated herein by reference, disclose lower eukaryote host cells capable of producing a glycoprotein comprising a GlcNAc₂Man₃GlcNAc₂ glycoform.

The immediately preceding host cell further includes a galactosyltransferase catalytic domain fused to a heterologous targeting or signal peptide not normally associated with the catalytic domain and selected to target galactosyltransferase activity to the ER or Golgi apparatus of the host cell. Passage of the recombinant glycoprotein through the ER or Golgi apparatus of the host cell produces a recombinant glycoprotein comprising a GalGlcNAc₂Man₃GlcNAc₂ or Gal₂GlcNAc₂Man₃GlcNAc₂ glycoform, or mixture thereof for example a recombinant glycoprotein composition comprising predominantly a GalGlcNAc₂Man₃GlcNAc₂ glycoform or Gal₂GlcNAc₂Man₃GlcNAc₂ glycoform or mixture thereof. U.S. Pat. No. 7,029,872 and U.S. Published Patent Application No. 2006/0040353, the disclosures of which are incorporated herein by reference, discloses lower eukaryote host cells capable of producing a glycoprotein comprising a Gal₂GlcNAc₂Man₃GlcNAc₂ glycoform.

The immediately preceding host cell further includes a sialyltransferase catalytic domain fused to a heterologous targeting or signal peptide not normally associated with the catalytic domain and selected to target sialyltransferase activity to the ER or Golgi apparatus of the host cell. The sialyltransferase can be an α-2,6-sialyltransferase or an α-2,3sialyltransferase. The type of sialyltransferase species will determine whether the sialic acid residue is attached in an α-2,6 linkage or an α-2,3 linkage. Passage of the recombinant glycoprotein through the ER or Golgi apparatus of the host cell produces a recombinant glycoprotein comprising predominantly a NANA₂Gal₂GlcNAc₂Man₃GlcNAc₂ glycoform or NANAGal₂GlcNAc₂Man₃GlcNAc₂ glycoform or mixture thereof. For lower eukaryote host cells such as yeast and filamentous fungi, the host cell further includes a means for providing CMP-sialic acid for transfer to the N-glycan. U.S. Published Patent Application No. 2005/0260729, the disclosure of which is incorporated herein by reference, discloses a method for genetically engineering lower eukaryotes to have a CMP-sialic acid synthesis pathway and U.S. Published Patent Application No. 2006/0286637, the disclosure of which is incorporated herein by reference, discloses a method for genetically engineering lower eukaryotes to produce sialylated glycoproteins. To enhance the amount of sialylation of the N-glycans and O-glycans, it can be advantageous to construct the host cell to include two or more copies of the CMP-sialic acid pathway and two ore more copies of the sialyltransferase.

Any one of the preceding host cells can further include one or more GlcNAc transferase selected from the group consisting of GnT III, GnT IV, GnT V, GnT VI, and GnT IX to produce glycoproteins having bisected (GnT III) and/or multiantennary (GnT IV, V, VI, and IX) N-glycan structures such as disclosed in U.S. Pat. No. 7,598,055 and U.S. Published Patent Application No. 2007/0037248, the disclosures of which are all incorporated herein by reference.

The above host cells are further genetically engineered to express a nucleic acid molecule encoding a protein O-mannose β-1,2-N-acetylglucosaminyltransferase I (POMGnT I) activity. In general, the POMGnT I catalytic domain is fused not normally associated with the catalytic domain and selected to target the fusion protein to a location in the ER or Golgi where it can then transfer a GlcNAc residue to O-linked mannose residues on the TNFRII-Fc fragment fusion protein as it traverses the secretory pathway. The human POMGnT and its expression in yeast have been disclosed in U.S. Pat. No. 7,217,548.

The host cells are also genetically modified to control the chain length of the O-glycans on the TNFRII-Fc fragment fusion protein so as to provide single-mannose O-glycans. The single-mannose O-glycans serve as a substrate for the POMGnT I to transfer a GlcNAc residue thereto. Control can be accomplished by growing the cells in the presence of Pmtp inhibitors that inhibit O-mannosyltransferase (PMT) protein activity or an alpha-mannosidase as disclosed in U.S. Published Application No. 20090170159, the disclosure of which is incorporated herein by reference), or both. Thus, in one aspect, controlling O-glycosylation includes expressing one or more secreted α-1,2-mannosidase enzymes in the host cell to produce the recombinant protein having reduced O-linked glycosylation, also referred to herein as O-mannose reduced glycans. In particular embodiments, the α1,2-mannosidase, which is capable of trimming multiple mannose residues from an O-linked glycan is produced by Trichoderma sp., Saccharomyces sp., or Aspergillus sp., Coccidiodes immitis, Coccidiodes posadasii, Penicillium citrinum, Magnaporthe grisea, Aspergillus saitoi, Aspergillus oryzae, or Chaetomiun globosum. For example, α-1,2-mannosidases can be obtained from Trichoderma reesei, Aspergillus niger, or Aspergillus oryzae. T. reesei is also known as Hypocrea jecorina. As shown in the examples, a transformed yeast comprising an expression cassette, which expresses the Trichoderma reesei α-1,2-mannosidase catalytic domain fused to the Saccharomyces cerevisiae αMAT pre signal sequence, was used to produce the TNFRII-Fc fragment fusion protein in which the O-glycans are trimmed to a single mannose residue, which can serve as a substrate for POMGnT1.

The Pmtp inhibitor reduces O-glycosylation occupancy (lowers the number of serines and threonine residues with O-mannose glycans on the TNFRII-Fc fragment fusion protein) from about 80 O-glycans to about 20 O-glycans per protein molecule. In the presence of the Pmtp inhibitor, the overall level of O-linked glycans on the TNFRII-Fc fragment fusion protein is significantly lowered. Thus, the Pmtp inhibitor and the secreted α-1,2-mannosidase results in a higher percentage of the O-glycans on the TNFRII-Fc fragment fusion protein being the desired sialylated O-glycan instead of the less desired O-linked mannobiose, mannotriose, and mannotetrose O-glycan structures or asialylated O-Man-GlcNAc or O-Man-GlcNAc-Gal. Thus, the control of O-glycosylation enables the overall levels of sialylated O-glycans to be increased while also reducing the level of asialylated or neutral charge O-glycans.

Pmtp inhibitors include but are not limited to a benzylidene thiazolidinediones. Examples of benzylidene thiazolidinediones that can be used are 5-[[3,4-bis(phenylmethoxy) phenyl]methylene]-4-oxo-2-thioxo-3-thiazolidineacetic Acid; 5-[[3-(1-Phenylethoxy)-4-(2-phenylethoxy)]phenyl]methylene]-4-oxo-2-thioxo-3-thiazolidineacetic Acid; and 5-[[3-(1-Phenyl-2-hydroxy)ethoxy)-4-(2-phenylethoxy)]phenyl]methylene]-4-oxo-2-thioxo-3-thiazolidineacetic Acid.

Pichia pastoris host cells further include strains that have been genetically engineered to eliminate glycoproteins having phosphomannose residues. This can be achieved by deleting or disrupting one or both of the phosphomannosyltransferase genes PNO1 and MNN4B (or MNN4 L1) (See for example, U.S. Pat. Nos. 7,198,921 and 7,259,007; the disclosures of which are all incorporated herein by reference), which in further aspects can also include deleting or disrupting the MNN4A (or MNN4) gene. Disruption includes disrupting the open reading frame encoding the particular enzymes or disrupting expression of the open reading frame or abrogating translation of RNAs encoding one or more of the β-mannosyltransferases and/or phosphomannosyltransferases using interfering RNA, antisense RNA, or the like. The host cells can further include any one of the aforementioned host cells modified to produce particular N-glycan structures.

To reduce or eliminate the likelihood of N-glycans and O-glycans with β-linked mannose residues, which are resistant to α-mannosidases, the recombinant glycoengineered Pichia pastoris host cells are genetically engineered to eliminate glycoproteins having α-mannosidase-resistant N-glycans by deleting or disrupting one or more of the 13-mannosyltransferase genes (e.g., BMT1, BMT2, BMT3, and BMT4)(See, U.S. Pat. No. 7,465,577 and U.S. Pat. No. 7,713,719). The deletion or disruption of BMT2 and one or more of BMT1, BMT3, and BMT4 also reduces or eliminates detectable cross reactivity to antibodies against host cell protein.

To reduce the risk of N-terminal clipping in Pichia pastoris host cells (LP diaminopeptidase activity), expression of the STE13 and DAP2 genes encoding the Ste13p and Dap2p proteases. Identification and deletion of the STE13 or DAP2 genes in Pichia pastoris has been described in Published PCT Application No. WO2007148345 and in Pabha et al., Protein Express. Purif. 64: 155-161 (2009).

Proteins that are destined for the vacuole are sorted from proteins destined for the cell surface in the late Golgi compartment. The sorting process is similar to the mammalian lysosomal sorting system; however, unlike the mammalian lysosomal sorting system where the sorting signal is a carbohydrate moiety, in yeast the sorting signal is contained within the polypeptide chains themselves. The most thoroughly studied vacuolar protein in S. cerevisiae is carboxypeptidase Y (CPY encoded by PRC1), which has a sorting signal at the N-terminus of its prosegment that is QRPL. This sorting signal sequence is recognized by the CPY sorting receptor Vps10p/Pep1p, which binds and directs the CPY to the vacuole. Mutational analysis of the sorting signal sequence by Van Voosrt et al., J. Biol. Chem. 271: 841-846 (1996) suggests that there may be cryptic sorting signals that if present in a recombinant protein such as TNFRII-Fc fragment fusion protein might direct the protein to the vacuole where it is degraded. To avoid potential sorting of the TNFRII-Fc fragment fusion protein to the vacuole, the Pichia pastoris host strain can further include a disruption or deletion of the expression of the VPS10-1 gene. The VPS10-1 gene in Pichia pastoris was identified and the gene deleted in the above glycoengineered Pichia pastoris to produce a Pichia pastoris strain that lacked CPY sorting mediated by the Vps10-1p.

Yield of glycoprotein can in some situations be improved by overexpressing nucleic acid molecules encoding mammalian or human chaperone proteins or replacing the genes encoding one or more endogenous chaperone proteins with nucleic acid molecules encoding one or more mammalian or human chaperone proteins. In addition, the expression of mammalian or human chaperone proteins in the host cell also appears to control O-glycosylation in the cell. Thus, further included are the host cells herein wherein the function of at least one endogenous gene encoding a chaperone protein has been reduced or eliminated, and a vector encoding at least one mammalian or human homolog of the chaperone protein is expressed in the host cell. Also included are host cells in which the endogenous host cell chaperones and the mammalian or human chaperone proteins are expressed. In further aspects, the lower eukaryotic host cell is a yeast or filamentous fungi host cell. Examples of the use of chaperones of host cells in which human chaperone proteins are introduced to improve the yield and reduce or control O-glycosylation of recombinant proteins has been disclosed in Published International Application No. WO 2009105357 and WO2010019487 (the disclosures of which are incorporated herein by reference).

The host cell can be further genetically engineered to include a nucleic acid molecule encoding a heterologous single-subunit oligosaccharyltransferase but wherein the endogenous host cell genes encoding the proteins comprising the oligosaccharyltransferase (OTase) complex are expressed. This includes expression of the endogenous STT3 gene, which in yeast is the STT3 gene. In general, in the above methods and host cells, the single-subunit oligosaccharyltransferase is capable of functionally suppressing the lethal phenotype of a mutation of at least one essential protein of the OTase complex. In further aspects, the essential protein of the OTase complex is encoded by the STT3 locus, WBP1 locus, OST1 locus, SWP1 locus, or OST2 locus, or homologue thereof. In further aspects, the for example single-subunit oligosaccharyltransferase is the Leishmania major STT3D protein.

Promoters are DNA sequence elements for controlling gene expression. In particular, promoters specify transcription initiation sites and can include a TATA box and upstream promoter elements. The promoters selected are those which would be expected to be operable in the particular host system selected. For example, yeast promoters are used when a yeast such as Saccharomyces cerevisiae, Kluyveromyces lactis, Ogataea minuta, or Pichia pastoris is the host cell whereas fungal promoters would be used in host cells such as Aspergillus niger, Neurospora crassa, or Tricoderma reesei. Examples of yeast promoters include but are not limited to the GAPDH, AOX1, SEC4, HH1, PMA1, OCH1, GAL1, PGK, GAP, TPI, CYC1, ADH2, PHO5, CUP1, MFα1, FLD1, PMA1, PDI, TEF, RPL10, and GUT1 promoters. Romanos et al., Yeast 8: 423-488 (1992) provide a review of yeast promoters and expression vectors. Hartner et al., Nuel. Acid Res. 36: e76 (pub on-line 6 Jun. 2008) describes a library of promoters for fine-tuned expression of heterologous proteins in Pichia pastoris.

The promoters that are operably linked to the nucleic acid molecules disclosed herein can be constitutive promoters or inducible promoters. An inducible promoter, for example the AOX1 promoter, is a promoter that directs transcription at an increased or decreased rate upon binding of a transcription factor in response to an inducer. Transcription factors as used herein include any factor that can bind to a regulatory or control region of a promoter and thereby affect transcription. The RNA synthesis or the promoter binding ability of a transcription factor within the host cell can be controlled by exposing the host to an inducer or removing an inducer from the host cell medium. Accordingly, to regulate expression of an inducible promoter, an inducer is added or removed from the growth medium of the host cell. Such inducers can include sugars, phosphate, alcohol, metal ions, hormones, heat, cold and the like. For example, commonly used inducers in yeast are glucose, galactose, alcohol, and the like.

Transcription termination sequences that are selected are those that are operable in the particular host cell selected. For example, yeast transcription termination sequences are used in expression vectors when a yeast host cell such as Saccharomyces cerevisiae, Kluyveromyces lactis, or Pichia pastoris is the host cell whereas fungal transcription termination sequences would be used in host cells such as Aspergillus niger, Neurospora crassa, or Tricoderma reesei. Transcription termination sequences include but are not limited to the Saccharomyces cerevisiae CYC transcription termination sequence (ScCYC TT), the Pichia pastoris ALG3 transcription termination sequence (ALG3 TT), the Pichia pastoris ALG6 transcription termination sequence (ALG6 TT), the Pichia pastoris ALG12 transcription termination sequence (ALG12 TT), the Pichia pastoris AOX1 transcription termination sequence (AOX1 TT), the Pichia pastoris OCH1 transcription termination sequence (OCH1 TT) and Pichia pastoris PMA1 transcription termination sequence (PMA1 TT). Other transcription termination sequences can be found in the examples and in the art.

For genetically engineering yeast, selectable markers can be used to construct the recombinant host cells include drug resistance markers and genetic functions which allow the yeast host cell to synthesize essential cellular nutrients, e.g. amino acids. Drug resistance markers which are commonly used in yeast include chloramphenicol, kanamycin, nourseothricin, hygromycin, methotrexate, G418 (geneticin), Zeocin, and the like. Genetic functions which allow the yeast host cell to synthesize essential cellular nutrients are used with available yeast strains having auxotrophic mutations in the corresponding genomic function. Common yeast selectable markers provide genetic functions for synthesizing leucine (LEU2), tryptophan (TRP1 and TRP2), proline (PRO1), uracil (URA3, URA5, URA6), histidine (HIS3), lysine (LYS2), adenine (ADE1 or ADE2), and the like. Other yeast selectable markers include the ARR3 gene from S. cerevisiae, which confers arsenite resistance to yeast cells that are grown in the presence of arsenite (Bobrowicz et al., Yeast, 13:819-828 (1997); Wysocki et al., J. Biol. Chem. 272:30061-30066 (1997)). A number of suitable integration sites include those enumerated in U.S. Pat. No. 7,479,389 (the disclosure of which is incorporated herein by reference) and include homologs to loci known for Saccharomyces cerevisiae and other yeast or fungi. Methods for integrating vectors into yeast are well known (See for example, U.S. Pat. No. 7,479,389, U.S. Pat. No. 7,514,253, U.S. Published Application No. 2009012400, and WO2009/085135; the disclosures of which are all incorporated herein by reference). Examples of insertion sites include, but are not limited to, Pichia ADE genes; Pichia TRP (including TRP1 through TRP2) genes; Pichia MCA genes; Pichia CYM genes; Pichia PEP genes; Pichia PRB genes; and Pichia LEU genes. The Pichia ADE1 and ARG4 genes have been described in Lin Cereghino et al., Gene 263:159-169 (2001) and U.S. Pat. No. 4,818,700 (the disclosure of which is incorporated herein by reference), the HIS3 and TRP1 genes have been described in Cosano et al., Yeast 14:861-867 (1998), HIS4 has been described in GenBank Accession No. X56180.

### Therapeutic Administration of the TNFRII-Fc Fragment Fusion Protein

The present invention provides methods of suppressing TNF-dependent inflammatory responses in humans comprising administering an effective amount of a composition comprising the TNFRII-Fc fragment fusion protein disclosed herein and a suitable diluent and carrier, for example, a pharmaceutical composition comprising a TNFRII-Fc fragment fusion protein in a pharmaceutically acceptable carrier.

For therapeutic use, a composition comprising the TNFRII-Fc fragment fusion protein is administered to a patient, preferably a human, for treatment of arthritis. Thus, for example, TNFRII-Fc fragment fusion protein compositions can be administered, for example, via intra-articular, intraperitoneal or subcutaneous routes by bolus injection, continuous infusion, sustained release from implants, or other suitable techniques. Typically, a composition comprising the TNFRII-Fc fragment fusion protein will be administered in the form of a composition comprising purified protein in conjunction with physiologically acceptable carriers, excipients or diluents. Such carriers will be nontoxic to recipients at the dosages and concentrations employed. Ordinarily, the preparation of such compositions entails combining the TNFRII-Fc fragment fusion protein with buffers, antioxidants such as ascorbic acid, low molecular weight (less than about 10 residues) polypeptides, proteins, amino acids, carbohydrates including glucose, sucrose or dextrins, chelating agents such as EDTA, glutathione and other stabilizers and excipients. Neutral buffered saline or saline mixed with conspecific serum albumin are exemplary appropriate diluents. Preferably, product is formulated as a lyophilizate using appropriate excipient solutions (e.g., sucrose) as diluents. Appropriate dosages can be determined in trials. In accordance with appropriate industry standards, preservatives may also be added, such as benzyl alcohol. The amount and frequency of administration will depend, of course, on such factors as the nature and severity of the indication being treated, the desired response, the condition of the patient, and so forth.

TNFRII-Fc fragment fusion protein compositions are administered to a mammal, preferably a human, for the purpose treating TNF-dependent inflammatory diseases, such as arthritis. For example, the TNFRII-Fc fragment fusion protein inhibits TNF-dependent arthritic responses. Because of the primary roles IL-1 and IL-2 play in the production of TNF, combination therapy using TNFR in combination with IL-1R and/or IL-2R may be used in the treatment of TNF-associated clinical indications. In the treatment of humans, the TNFRII-Fc fragment fusion proteins disclosed herein are preferred. Either Type I IL-1R or Type II IL-1R, or a combination thereof, may be used in accordance with the present invention to treat TNF-dependent inflammatory diseases, such as arthritis. Other types of TNF binding proteins may be similarly used.

For treatment of arthritis, the TNFRII-Fc fragment fusion protein composition is administered in systemic amounts ranging from about 0.1 mg/kg/week to about 100 mg/kg/week. In further aspects, the TNFRII-Fc fragment fusion protein is administered in amounts ranging from about 0.5 mg/kg/week to about 50 mg/kg/week. For local intra-articular administration, dosages preferably range from about 0.01 mg/kg to about 1.0 mg/kg per injection.

### Pharmaceutical Compositions

The TNFRII-Fc fragment fusion proteins disclosed herein may be provided as a pharmaceutical composition when combined with a pharmaceutically acceptable carrier. Such compositions comprise a therapeutically-effective amount of the TNFRII-Fc fragment fusion protein and a pharmaceutically acceptable carrier. Such a composition may also be comprised of (in addition to TNFRII-Fc fragment fusion protein and a carrier) diluents, fillers, salts, buffers, stabilizers, solubilizers, and other materials well known in the art and generally regarded as safe by pharmaceutical and biological regulatory agencies. Compositions comprising the TNFRII-Fc fragment fusion protein can be administered, if desired, in the form of salts provided the salts are pharmaceutically acceptable. Salts may be prepared using standard procedures known to those skilled in the art of synthetic organic chemistry.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. The term “pharmaceutically acceptable salt” further includes all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxynaphthoate, teoclate, iodide, tosylate, isothionate, triethiodide, lactate, panoate, valerate, and the like which can be used as a dosage form for modifying the solubility or hydrolysis characteristics or can be used in sustained release or pro-drug formulations. It will be understood that, as used herein, references to the TNFRII-Fc fragment fusion protein disclosed herein are meant to also include the pharmaceutically acceptable salts.

As utilized herein, the term “pharmaceutically acceptable” means a non-toxic material that does not interfere with the effectiveness of the biological activity of the active ingredient(s), approved by a regulatory agency of the Federal or a state government or listed in the U.S. Pharmacopoeia or other generally recognized pharmacopoeia for use in animals and, more particularly, in humans. The term “carrier” refers to a diluent, adjuvant, excipient, or vehicle with which the therapeutic is administered and includes, but is not limited to such sterile liquids as water and oils. The characteristics of the carrier will depend on the route of administration. The TNFRII-Fc fragment fusion protein disclosed herein may be in multimers (for example, heterodimers or homodimers) or complexes with itself or other peptides. As a result, pharmaceutical compositions of the invention may comprise one or more TNFRII-Fc fragment fusion protein molecules disclosed herein in such multimeric or complexed form.

As used herein, the term “therapeutically effective amount” means the total amount of each active component of the pharmaceutical composition or method that is sufficient to show a meaningful patient benefit, i.e., treatment, healing, prevention or amelioration of the relevant medical condition, or an increase in rate of treatment, healing, prevention or amelioration of such conditions. When applied to an individual active ingredient, administered alone, the term refers to that ingredient alone. When applied to a combination, the term refers to combined amounts of the active ingredients that result in the therapeutic effect, whether administered in combination, serially, or simultaneously.

The following examples are intended to promote a further understanding of the present invention.

### Example 1

This example shows the construction of Pichia pastoris strains YGLY10299, YGLY11731, and YGLY13571, each strain a GS6.0 strain capable of producing TNFRII-Fc fragment fusion protein comprising sialylated N-glycans. FIGS. 1A-G provide a flow-diagram illustrating construction of the strains.

All yeast transformations were as follows. P. pastoris strains were grown in 50 mL YPD media (yeast extract (1%), peptone (2%), dextrose (2%)) overnight to an optical density (“OD”) of between about 0.2 to 6. After incubation on ice for 30 minutes, cells were pelleted by centrifugation at 2500-3000 rpm for 5 minutes. Media was removed and the cells washed three times with ice cold sterile 1M sorbitol before resuspension in 0.5 ml ice cold sterile 1M sorbitol. Ten μL DNA (5-20 μg) and 100 μL cell suspension was combined in an electroporation cuvette and incubated for 5 minutes on ice. Electroporation was in a Bio-Rad GenePulser Xcell following the preset Pichia pastoris protocol (2 kV, 25 μF, 200Ω), immediately followed by the addition of 1 mL YPDS recovery media (YPD media plus 1 M sorbitol). The transformed cells were allowed to recover for four hours to overnight at room temperature (26° C.) before plating the cells on selective media.

The strain YGLY9469 was constructed from wild-type Pichia pastoris strain NRRL-Y 11430 using methods described earlier (See for example, U.S. Pat. No. 7,449,308; U.S. Pat. No. 7,479,389; U.S. Published Application No. 20090124000; Published PCT Application No. WO2009085135; Nett and Gerngross, Yeast 20:1279 (2003); Choi et al., Proc. Natl. Acad. Sci. USA 100:5022 (2003); Hamilton et al., Science 301:1244 (2003)). All plasmids were made in a pUC19 plasmid using standard molecular biology procedures. For nucleotide sequences that were optimized for expression in P. pastoris, the native nucleotide sequences were analyzed by the GENEOPTIMIZER software (GeneArt, Regensburg, Germany) and the results used to generate nucleotide sequences in which the codons were optimized for P. pastoris expression. Yeast strains were transformed by electroporation (using standard techniques as recommended by the manufacturer of the electroporator BioRad).

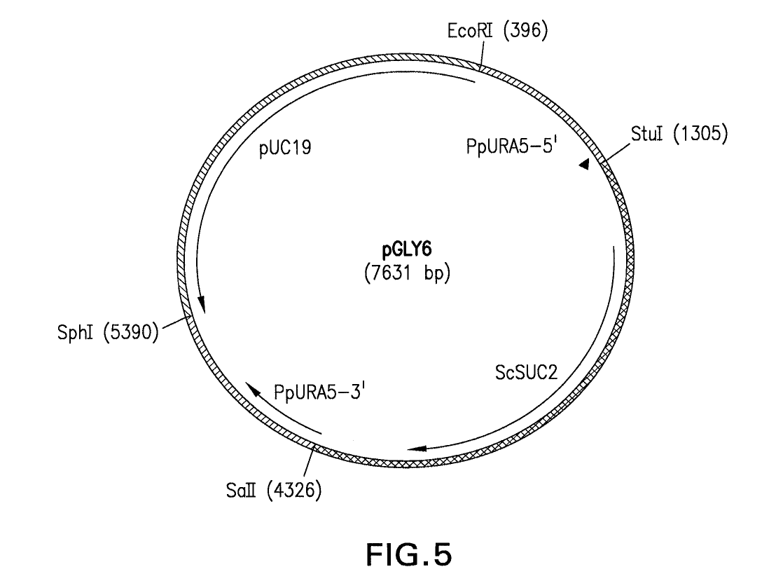

Plasmid pGLY6 (FIG. 5) is an integration vector that targets the URA5 locus. It contains a nucleic acid molecule comprising the S. cerevisiae invertase gene or transcription unit (ScSUC2; SEQ ID NO:17) flanked on one side by a nucleic acid molecule comprising a nucleotide sequence from the 5′ region of the P. pastoris URA5 gene (SEQ ID NO:18) and on the other side by a nucleic acid molecule comprising the nucleotide sequence from the 3′ region of the P. pastoris URA5 gene (SEQ ID NO:19). Plasmid pGLY6 was linearized and the linearized plasmid transformed into wild-type strain NRRL-Y 11430 to produce a number of strains in which the ScSUC2 gene was inserted into the URA5 locus by double-crossover homologous recombination. Strain YGLY1-3 was selected from the strains produced and is auxotrophic for uracil.

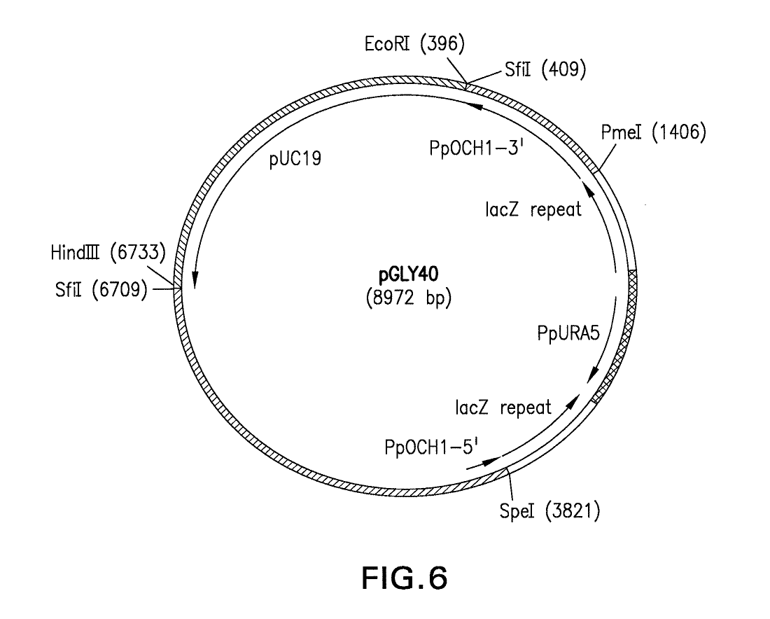

Plasmid pGLY40 (FIG. 6) is an integration vector that targets the OCH1 locus and contains a nucleic acid molecule comprising the P. pastoris URA5 gene or transcription unit (SEQ ID NO:20) flanked by nucleic acid molecules comprising lacZ repeats (SEQ ID NO:21) which in turn is flanked on one side by a nucleic acid molecule comprising a nucleotide sequence from the 5′ region of the OCH1 gene (SEQ ID NO:22) and on the other side by a nucleic acid molecule comprising a nucleotide sequence from the 3′ region of the OCH1 gene (SEQ ID NO:23). Plasmid pGLY40 was linearized with SfiI and the linearized plasmid transformed into strain YGLY1-3 to produce a number of strains in which the URA5 gene flanked by the lacZ repeats has been inserted into the OCH1 locus by double-crossover homologous recombination. Strain YGLY2-3 was selected from the strains produced and is prototrophic for URA5. Strain YGLY2-3 was counterselected in the presence of 5-fluoroorotic acid (5-FOA) to produce a number of strains in which the URA5 gene has been lost and only the lacZ repeats remain in the OCH1 locus. This renders the strain auxotrophic for uracil. Strain YGLY4-3 was selected.

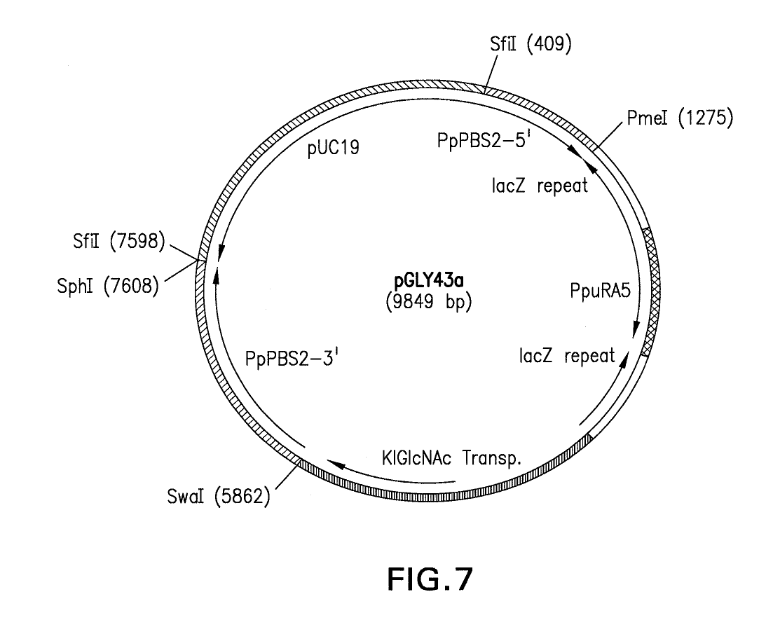

Plasmid pGLY43a (FIG. 7) is an integration vector that targets the BMT2 locus and contains a nucleic acid molecule comprising the K. lactis UDP-N-acetylglucosamine (UDP-GlcNAc) transporter gene or transcription unit (KlMNN2-2, SEQ ID NO:24) adjacent to a nucleic acid molecule comprising the P. pastoris URA5 gene or transcription unit flanked by nucleic acid molecules comprising lacZ repeats. The adjacent genes are flanked on one side by a nucleic acid molecule comprising a nucleotide sequence from the 5′ region of the BMT2 gene (SEQ ID NO: 25) and on the other side by a nucleic acid molecule comprising a nucleotide sequence from the 3′ region of the BMT2 gene (SEQ ID NO:26). Plasmid pGLY43a was linearized with SfiI and the linearized plasmid transformed into strain YGLY4-3 to produce to produce a number of strains in which the KlMNN2-2 gene and URA5 gene flanked by the lacZ repeats has been inserted into the BMT2 locus by double-crossover homologous recombination. The BMT2 gene has been disclosed in Mille et al., J. Biol. Chem. 283: 9724-9736 (2008) and U.S. Pat. No. 7,465,557. Strain YGLY6-3 was selected from the strains produced and is prototrophic for uracil. Strain YGLY6-3 was counterselected in the presence of 5-FOA to produce strains in which the URA5 gene has been lost and only the lacZ repeats remain. This renders the strain auxotrophic for uracil. Strain YGLY8-3 was selected.

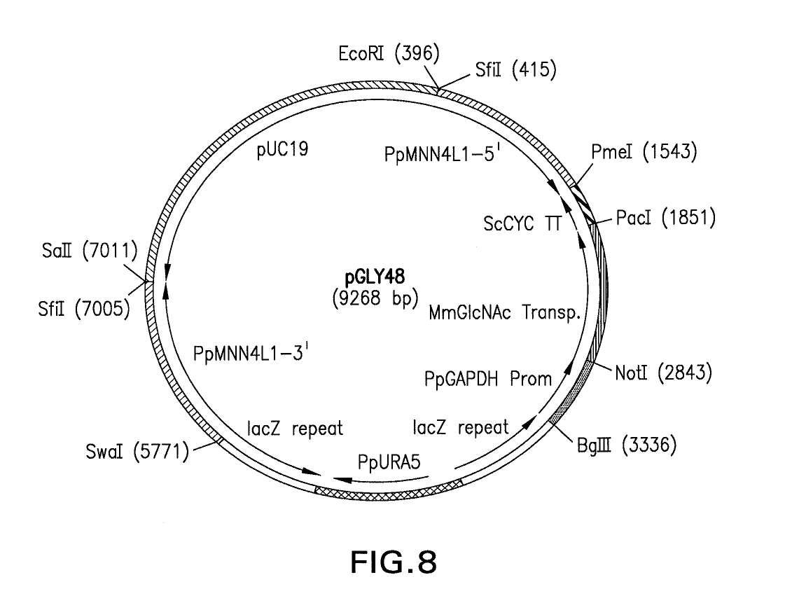

Plasmid pGLY48 (FIG. 8) is an integration vector that targets the MNN4 L1 locus and contains an expression cassette comprising a nucleic acid molecule encoding the mouse homologue of the UDP-GlcNAc transporter (SEQ ID NO:27) open reading frame (ORF) operably linked at the 5′ end to a nucleic acid molecule comprising the P. pastoris GAPDH promoter (SEQ ID NO:5) and at the 3′ end to a nucleic acid molecule comprising the S. cerevisiae CYC termination sequences (SEQ ID NO:3) adjacent to a nucleic acid molecule comprising the P. pastoris URA5 gene flanked by lacZ repeats and in which the expression cassettes together are flanked on one side by a nucleic acid molecule comprising a nucleotide sequence from the 5′ region of the P. pastoris MNN4 L1 gene (SEQ ID NO:28) and on the other side by a nucleic acid molecule comprising a nucleotide sequence from the 3′ region of the MNN4 L1 gene (SEQ ID NO:29). Plasmid pGLY48 was linearized with SfiI and the linearized plasmid transformed into strain YGLY8-3 to produce a number of strains in which the expression cassette encoding the mouse UDP-GlcNAc transporter and the URA5 gene have been inserted into the MNN4 L1 locus by double-crossover homologous recombination. The MNN4 L1 gene (also referred to as MNN4B) has been disclosed in U.S. Pat. No. 7,259,007. Strain YGLY10-3 was selected from the strains produced and then counterselected in the presence of 5-FOA to produce a number of strains in which the URA5 gene has been lost and only the lacZ repeats remain. Strain YGLY12-3 was selected.

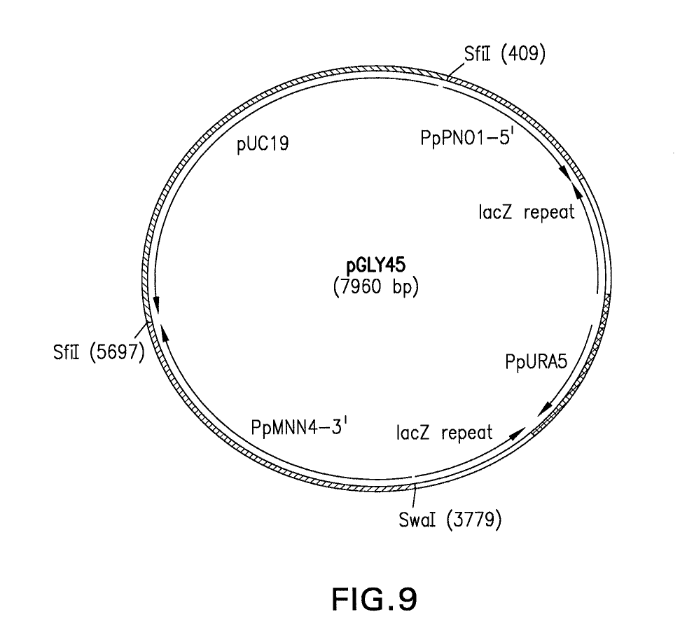

Plasmid pGLY45 (FIG. 9) is an integration vector that targets the PNO1/MNN4 loci and contains a nucleic acid molecule comprising the P. pastoris URA5 gene or transcription unit flanked by nucleic acid molecules comprising lacZ repeats which in turn is flanked on one side by a nucleic acid molecule comprising a nucleotide sequence from the 5′ region of the PNO1 gene (SEQ ID NO:30) and on the other side by a nucleic acid molecule comprising a nucleotide sequence from the 3′ region of the MNN4 gene (SEQ ID NO:31). Plasmid pGLY45 was linearized with SfiI and the linearized plasmid transformed into strain YGLY12-3 to produce a number of strains in which the URA5 gene flanked by the lacZ repeats has been inserted into the PNO1/MNN4 loci by double-crossover homologous recombination. The PNO1 gene has been disclosed in U.S. Pat. No. 7,198,921 and the MNN4 gene (also referred to as MNN4A) has been disclosed in U.S. Pat. No. 7,259,007. Strain YGLY14-3 was selected from the strains produced and then counterselected in the presence of 5-FOA to produce a number of strains in which the URA5 gene has been lost and only the lacZ repeats remain. Strain YGLY16-3 was selected.

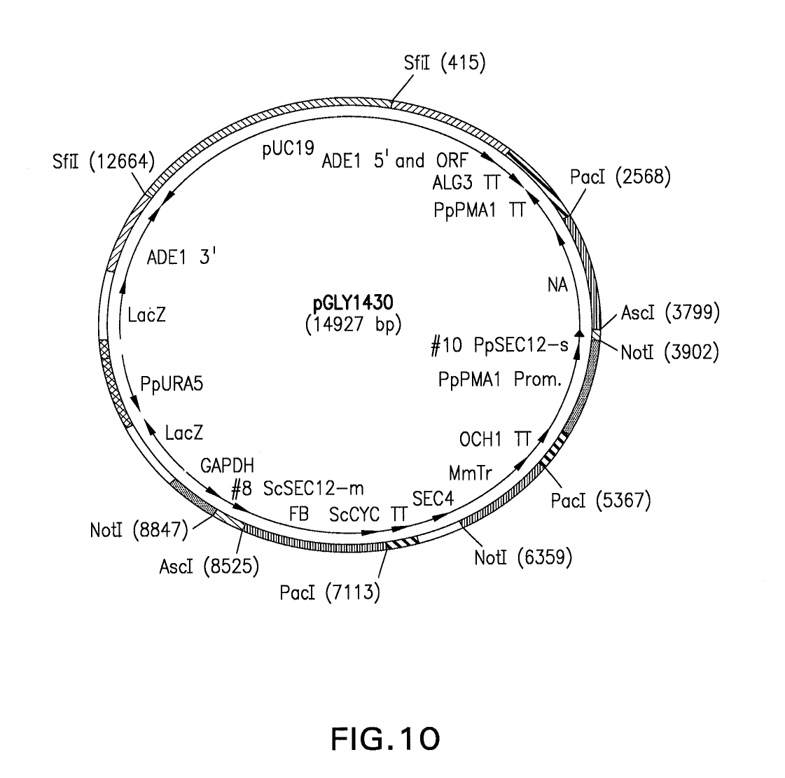

Plasmid pGLY1430 (FIG. 10) is a KINKO integration vector that targets the ADE1 locus without disrupting expression of the locus and contains in tandem four expression cassettes encoding (1) the human GlcNAc transferase I catalytic domain (NA) fused at the N-terminus to P. pastoris SEC12 leader peptide (10) to target the chimeric enzyme to the ER or Golgi, (2) mouse homologue of the UDP-GlcNAc transporter (MmTr), (3) the mouse mannosidase IA catalytic domain (FB) fused at the N-terminus to S. cerevisiae SEC12 leader peptide (8) to target the chimeric enzyme to the ER or Golgi, and (4) the P. pastoris URA5 gene or transcription unit. KINKO (Knock-In with little or No Knock-Out) integration vectors enable insertion of heterologous DNA into a targeted locus without disrupting expression of the gene at the targeted locus and have been described in U.S. Published Application No. 20090124000. The expression cassette encoding the NA 10 comprises a nucleic acid molecule encoding the human GlcNAc transferase I catalytic domain codon-optimized for expression in P. pastoris (SEQ ID NO:32) fused at the 5′ end to a nucleic acid molecule encoding the SEC12 leader 10 (SEQ ID NO:33), which is operably linked at the 5′ end to a nucleic acid molecule comprising the P. pastoris PMA1 promoter and at the 3′ end to a nucleic acid molecule comprising the P. pastoris PMA1 transcription termination sequence. The expression cassette encoding MmTr comprises a nucleic acid molecule encoding the mouse homologue of the UDP-GlcNAc transporter ORF operably linked at the 5′ end to a nucleic acid molecule comprising the P. pastoris SEC4 promoter (SEQ ID NO:34) and at the 3′ end to a nucleic acid molecule comprising the P. pastoris OCH1 termination sequences (SEQ ID NO:35). The expression cassette encoding the PBS comprises a nucleic acid molecule encoding the mouse mannosidase IA catalytic domain (SEQ ID NO:36) fused at the 5′ end to a nucleic acid molecule encoding the SEC12-m leader S (SEQ ID NO:37), which is operably linked at the 5′ end to a nucleic acid molecule comprising the P. pastoris GADPH promoter and at the 3′ end to a nucleic acid molecule comprising the S. cerevisiae CYC transcription termination sequence. The URA5 expression cassette comprises a nucleic acid molecule comprising the P. pastoris URA5 gene or transcription unit flanked by nucleic acid molecules comprising lacZ repeats. The four tandem cassettes are flanked on one side by a nucleic acid molecule comprising a nucleotide sequence from the 5′ region and complete ORF of the ADE1 gene (SEQ ID NO:38) followed by a P. pastoris ALG3 termination sequence (SEQ ID NO:8) and on the other side by a nucleic acid molecule comprising a nucleotide sequence from the 3′ region of the ADE1 gene (SEQ ID NO:39). Plasmid pGLY1430 was linearized with SfiI and the linearized plasmid transformed into strain YGLY16-3 to produce a number of strains in which the four tandem expression cassette have been inserted into the ADE1 locus immediately following the ADE1 ORF by double-crossover homologous recombination. The strain YGLY2798 was selected from the strains produced and is auxotrophic for arginine and now prototrophic for uridine, histidine, and adenine. The strain was then counterselected in the presence of 5-FOA to produce a number of strains now auxotrophic for uridine. Strain YGLY3794 was selected and is capable of making glycoproteins that have predominantly GlcNAcMan₅GlcNAc₂ terminated N-glycans.

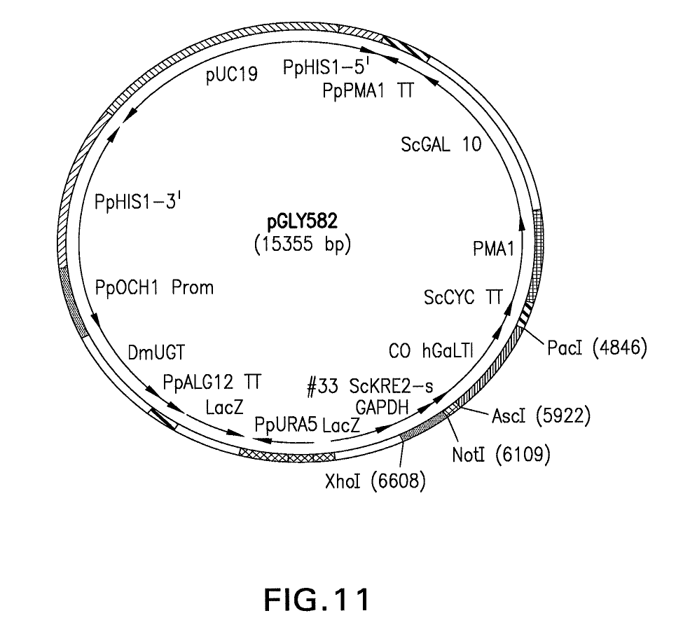

Plasmid pGLY582 (FIG. 11) is an integration vector that targets the HIS1 locus and contains in tandem four expression cassettes encoding (1) the S. cerevisiae UDP-glucose epimerase (ScGAL10), (2) the human galactosyltransferase I (hGalT) catalytic domain fused at the N-terminus to the S. cerevisiae KRE2-s leader peptide (33) to target the chimeric enzyme to the ER or Golgi, (3) the P. pastoris URA5 gene or transcription unit flanked by lacZ repeats, and (4) the D. melanogaster UDP-galactose transporter (DmUGT). The expression cassette encoding the ScGAL10 comprises a nucleic acid molecule encoding the ScGAL10 ORF (SEQ ID NO:40) operably linked at the 5′ end to a nucleic acid molecule comprising the P. pastoris PMA1 promoter (SEQ ID NO:1) and operably linked at the 3′ end to a nucleic acid molecule comprising the P. pastoris PMA1 transcription termination sequence (SEQ ID NO:41). The expression cassette encoding the chimeric galactosyltransferase I comprises a nucleic acid molecule encoding the hGalT catalytic domain codon optimized for expression in P. pastoris (SEQ ID NO:42) fused at the 5′ end to a nucleic acid molecule encoding the KRE2-s leader 33 (SEQ ID NO:43), which is operably linked at the 5′ end to a nucleic acid molecule comprising the P. pastoris GAPDH promoter and at the 3′ end to a nucleic acid molecule comprising the S. cerevisiae CYC transcription termination sequence. The URA5 expression cassette comprises a nucleic acid molecule comprising the P. pastoris URA5 gene or transcription unit flanked by nucleic acid molecules comprising lacZ repeats. The expression cassette encoding the DmUGT comprises a nucleic acid molecule encoding the DmUGT ORF (SEQ ID NO:44) operably linked at the 5′ end to a nucleic acid molecule comprising the P. pastoris OCH1 promoter (SEQ ID NO:45) and operably linked at the 3′ end to a nucleic acid molecule comprising the P. pastoris ALG12 transcription termination sequence (SEQ ID NO:46). The four tandem cassettes are flanked on one side by a nucleic acid molecule comprising a nucleotide sequence from the 5′ region of the HIS1 gene (SEQ ID NO:47) and on the other side by a nucleic acid molecule comprising a nucleotide sequence from the 3′ region of the HIS1 gene (SEQ ID NO:48). Plasmid pGLY582 was linearized and the linearized plasmid transformed into strain YGLY3794 to produce a number of strains in which the four tandem expression cassette have been inserted into the HIS1 locus by homologous recombination. Strain YGLY3853 was selected and is auxotrophic for histidine and prototrophic for uridine.

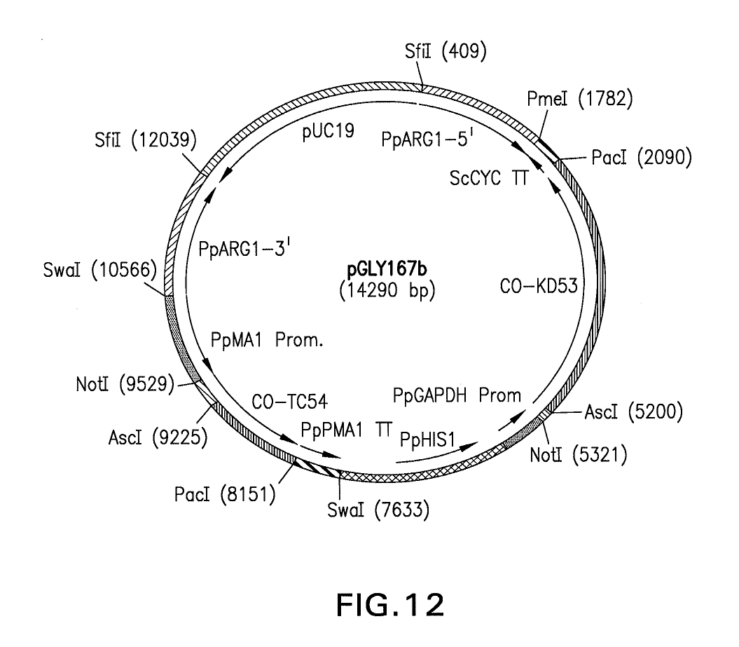

Plasmid pGLY167b (FIG. 12) is an integration vector that targets the ARG1 locus and contains in tandem three expression cassettes encoding (1) the D. melanogaster mannosidase II catalytic domain (KD) fused at the N-terminus to S. cerevisiae MNN2 leader peptide (53) to target the chimeric enzyme to the ER or Golgi, (2) the P. pastoris HIS1 gene or transcription unit, and (3) the rat N-acetylglucosamine (GlcNAc) transferase II catalytic domain (TC) fused at the N-terminus to S. cerevisiae MNN2 leader peptide (54) to target the chimeric enzyme to the ER or Golgi. The expression cassette encoding the KD53 comprises a nucleic acid molecule encoding the D. melanogaster mannosidase II catalytic domain codon-optimized for expression in P. pastoris (SEQ ID NO:49) fused at the 5′ end to a nucleic acid molecule encoding the MNN2 leader 53 (SEQ ID NO:50), which is operably linked at the 5′ end to a nucleic acid molecule comprising the P. pastoris GAPDH promoter and at the 3′ end to a nucleic acid molecule comprising the S. cerevisiae CYC transcription termination sequence. The HIS1 expression cassette comprises a nucleic acid molecule comprising the P. pastoris HIS1 gene or transcription unit (SEQ ID NO:51). The expression cassette encoding the TC54 comprises a nucleic acid molecule encoding the rat GlcNAc transferase II catalytic domain codon-optimized for expression in P. pastoris (SEQ ID NO:52) fused at the 5′ end to a nucleic acid molecule encoding the MNN2 leader 54 (SEQ ID NO:53), which is operably linked at the 5′ end to a nucleic acid molecule comprising the P. pastoris PMA1 promoter and at the 3′ end to a nucleic acid molecule comprising the P. pastoris PMA1 transcription termination sequence. The three tandem cassettes are flanked on one side by a nucleic acid molecule comprising a nucleotide sequence from the 5′ region of the ARG1 gene (SEQ ID NO:54) and on the other side by a nucleic acid molecule comprising a nucleotide sequence from the 3′ region of the ARG1 gene (SEQ ID NO:55). Plasmid pGLY167b was linearized with SfiI and the linearized plasmid transformed into strain YGLY3853 to produce a number of strains (in which the three tandem expression cassettes have been inserted into the ARG1 locus by double-crossover homologous recombination. The strain YGLY4754 was selected from the strains produced and is auxotrophic for arginine and prototrophic for uridine and histidine. The strain was then counterselected in the presence of 5-FOA to produce a number of strains now auxotrophic for uridine. Strain YGLY4799 was selected.

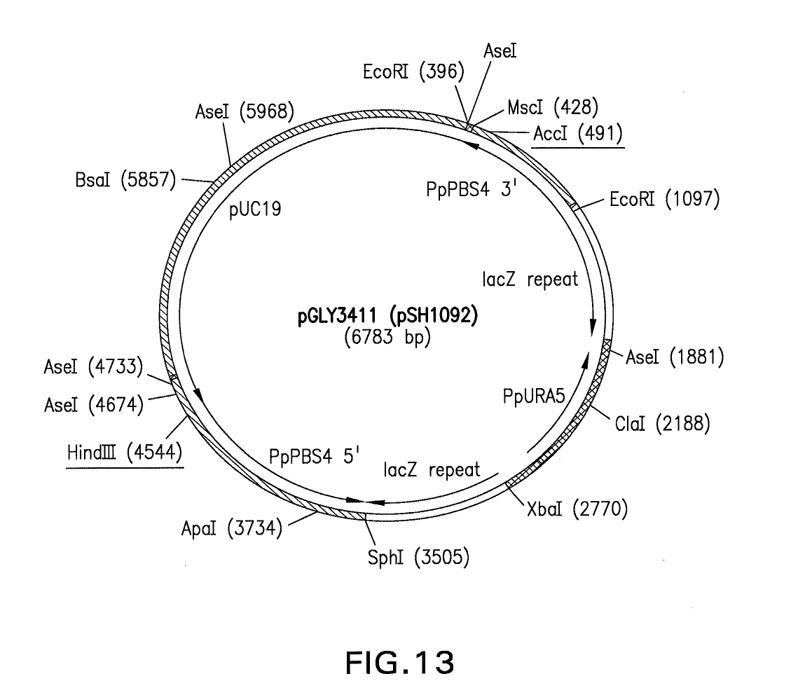

Plasmid pGLY3411 (FIG. 13) is an integration vector that contains the expression cassette comprising the P. pastoris URA5 gene flanked by lacZ repeats flanked on one side with the 5′ nucleotide sequence of the P. pastoris BMT4 gene (SEQ ID NO:56) and on the other side with the 3′ nucleotide sequence of the P. pastoris BMT4 gene (SEQ ID NO:57). Plasmid pGLY3411 was linearized and the linearized plasmid transformed into YGLY4799 to produce a number of strains in which the URA5 expression cassette has been inserted into the BMT4 locus by double-crossover homologous recombination. Strain YGLY6903 was selected from the strains produced and is prototrophic for uracil, adenine, histidine, proline, arginine, and tryptophan. The strain was then counterselected in the presence of 5-FOA to produce a number of strains now auxotrophic for uridine. Strain YGLY7432 was selected.

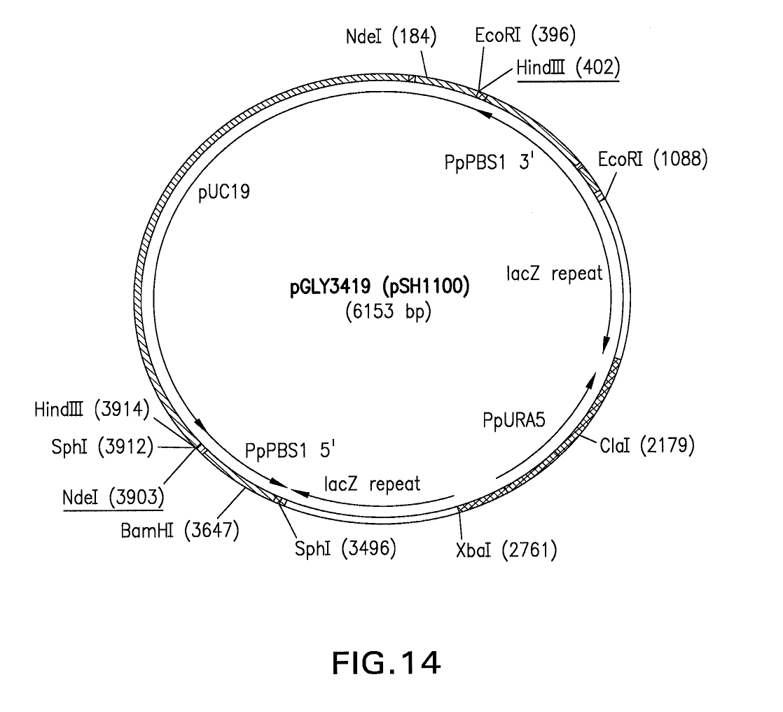

Plasmid pGLY3419 (FIG. 14) is an integration vector that contains an expression cassette comprising the P. pastoris URA5 gene flanked by lacZ repeats flanked on one side with the 5′ nucleotide sequence of the P. pastoris BMT1 gene (SEQ ID NO:58) and on the other side with the 3′ nucleotide sequence of the P. pastoris BMT1 gene (SEQ ID NO:59). Plasmid pGLY3419 was linearized and the linearized plasmid transformed into strain YGLY7432 to produce a number of strains in which the URA5 expression cassette has been inserted into the BMT1 locus by double-crossover homologous recombination. The strain YGLY7651 was selected from the strains produced and are prototrophic for uracil, adenine, histidine, proline, arginine, and tryptophan. The strains were then counterselected in the presence of 5-FOA to produce a number of strains now auxotrophic for uridine. Strain YGLY7930 was selected.

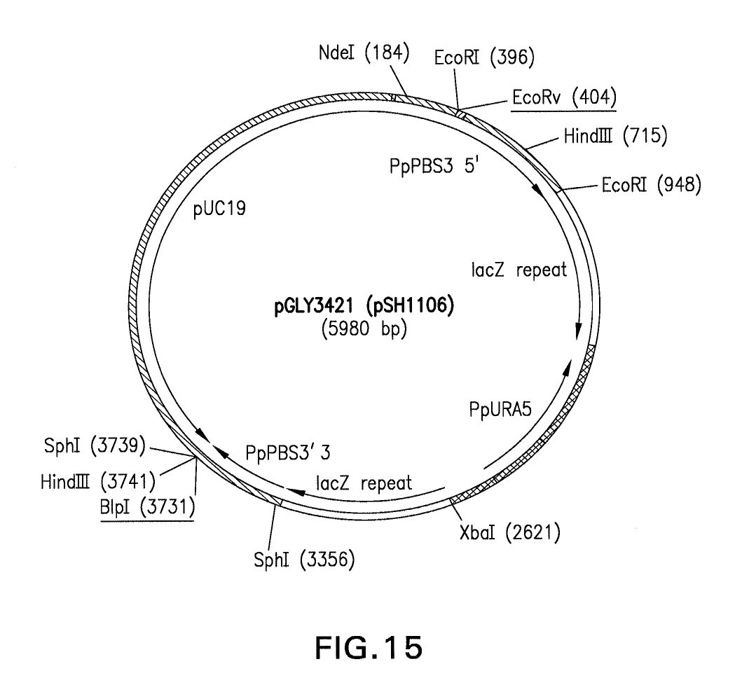

Plasmid pGLY3421 (FIG. 15) is an integration vector that contains an expression cassette comprising the P. pastoris URA5 gene flanked by lacZ repeats flanked on one side with the 5′ nucleotide sequence of the P. pastoris BMT3 gene (SEQ ID NO:60) and on the other side with the 3′ nucleotide sequence of the P. pastoris BMT3 gene (SEQ ID NO:61). Plasmid pGLY3419 was linearized and the linearized plasmid transformed into strain YGLY7930 to produce a number of strains in which the URA5 expression cassette has been inserted into the BMT1 locus by double-crossover homologous recombination. The strain YGLY7961 was selected from the strains produced and are prototrophic for uracil, adenine, histidine, proline, arginine, and tryptophan.

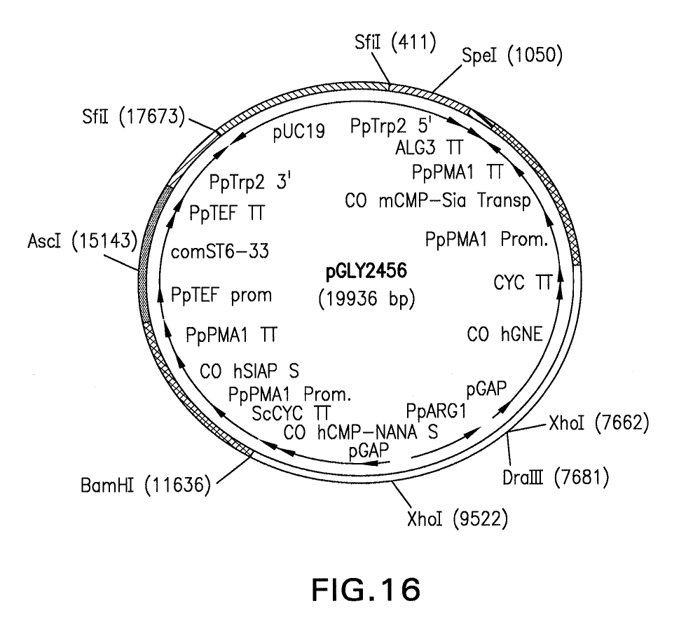

Plasmid pGLY2456 (FIG. 16) is a KINKO integration vector that targets the TRP2 locus without disrupting expression of the locus and contains six expression cassettes encoding (1) the mouse CMP-sialic acid transporter (mCMP-Sia Transp), (2) the human UDP-GlcNAc 2-epimerase/N-acetylmannosamine kinase (hGNE), (3) the Pichia pastoris ARG1 gene or transcription unit, (4) the human CMP-sialic acid synthase (hCSS), (5) the human N-acetylneuraminate-9-phosphate synthase (hSPS), (6) the mouse α-2,6-sialyltransferase catalytic domain (mST6) fused at the N-terminus to S. cerevisiae KRE2 leader peptide (33) to target the chimeric enzyme to the ER or Golgi, and the P. pastoris ARG1 gene or transcription unit. The expression cassette encoding the mouse CMP-sialic acid transporter comprises a nucleic acid molecule encoding the mCMP Sia Transp ORF codon optimized for expression in P. pastoris (SEQ ID NO:64), which is operably linked at the 5′ end to a nucleic acid molecule comprising the P. pastoris PMA1 promoter and at the 3′ end to a nucleic acid molecule comprising the P. pastoris PMA1 transcription termination sequence. The expression cassette encoding the human UDP-GlcNAc 2-epimerase/N-acetylmannosamine kinase comprises a nucleic acid molecule encoding the hGNE ORF codon optimized for expression in P. pastoris (SEQ ID NO:65), which is operably linked at the 5′ end to a nucleic acid molecule comprising the P. pastoris GAPDH promoter and at the 3′ end to a nucleic acid molecule comprising the S. cerevisiae CYC transcription termination sequence. The expression cassette encoding the P. pastoris ARG1 gene comprises (SEQ ID NO:66). The expression cassette encoding the human CMP-sialic acid synthase comprises a nucleic acid molecule encoding the hCSS ORF codon optimized for expression in P. pastoris (SEQ ID NO:67), which is operably linked at the 5′ end to a nucleic acid molecule comprising the P. pastoris GAPDH promoter and at the 3′ end to a nucleic acid molecule comprising the S. cerevisiae CYC transcription termination sequence. The expression cassette encoding the human N-acetylneuraminate-9-phosphate synthase comprises a nucleic acid molecule encoding the hSIAP S ORF codon optimized for expression in P. pastoris (SEQ ID NO:68), which is operably linked at the 5′ end to a nucleic acid molecule comprising the P. pastoris PMA1 promoter and at the 3′ end to a nucleic acid molecule comprising the P. pastoris PMA1 transcription termination sequence. The expression cassette encoding the chimeric mouse α-2,6-sialyltransferase comprises a nucleic acid molecule encoding the mST6 catalytic domain codon optimized for expression in P. pastoris (SEQ ID NO:69) fused at the 5′ end to a nucleic acid molecule encoding the S. cerevisiae KRE2 signal peptide, which is operably linked at the 5′ end to a nucleic acid molecule comprising the P. pastoris TEF promoter (SEQ ID NO:6) and at the 3′ end to a nucleic acid molecule comprising the P. pastoris TEF transcription termination sequence (SEQ ID NO:7). The six tandem cassettes are flanked on one side by a nucleic acid molecule comprising a nucleotide sequence from the 5′ region and ORF of the TRP2 gene ending at the stop codon (SEQ ID NO:62) followed by a P. pastoris ALG3 termination sequence and on the other side by a nucleic acid molecule comprising a nucleotide sequence from the 3′ region of the TRP2 gene (SEQ ID NO:63). Plasmid pGLY2456 was linearized with SfiI and the linearized plasmid transformed into strain YGLY7961 to produce a number of strains in which the six expression cassette have been inserted into the TRP2 locus immediately following the TRP2 ORF by double-crossover homologous recombination. The strain YGLY8146 was selected from the strains produced. The strain was then counterselected in the presence of 5-FOA to produce a number of strains now auxotrophic for uridine. Strain YGLY9296 was selected.

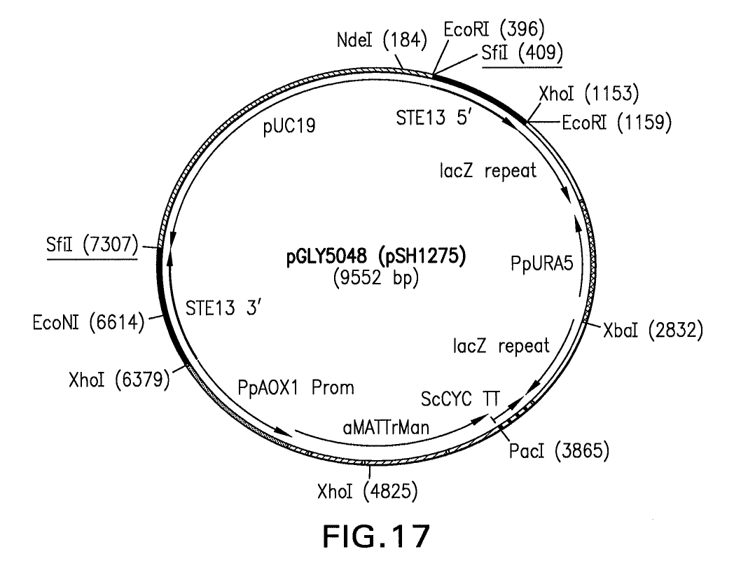

Plasmid pGLY5048 (FIG. 17) is an integration vector that targets the STE13 locus and contains expression cassettes encoding (1) the T. reesei α-1,2-mannosidase catalytic domain fused at the N-terminus to S. cerevisiae αMATpre signal peptide (aMATTrMan) to target the chimeric protein to the secretory pathway and secretion from the cell and (2) the P. pastoris URA5 gene or transcription unit. The expression cassette encoding the αMATTrMan comprises a nucleic acid molecule encoding the T. reesei catalytic domain (SEQ ID NO:81) fused at the 5′ end to a nucleic acid molecule encoding the S. cerevisiae αMATpre signal peptide (SEQ ID NO:80), which is operably linked at the 5′ end to a nucleic acid molecule comprising the P. pastoris AOX1 promoter and at the 3′ end to a nucleic acid molecule comprising the S. cerevisiae CYC transcription termination sequence. The URA5 expression cassette comprises a nucleic acid molecule comprising the P. pastoris URA5 gene or transcription unit flanked by nucleic acid molecules comprising lacZ repeats. The two tandem cassettes are flanked on one side by a nucleic acid molecule comprising a nucleotide sequence from the 5′ region of the STE13 gene (SEQ ID NO:82) and on the other side by a nucleic acid molecule comprising a nucleotide sequence from the 3′ region of the STE13 gene (SEQ ID NO:83). Plasmid pGLY5048 was linearized with SfiI and the linearized plasmid transformed into strain YGLY9296 to produce a number of strains. The strain YGLY9469 was selected from the strains produced. This strain is capable of producing glycoproteins that have single-mannose O-glycosylation (See Published U.S. Application No. 20090170159).

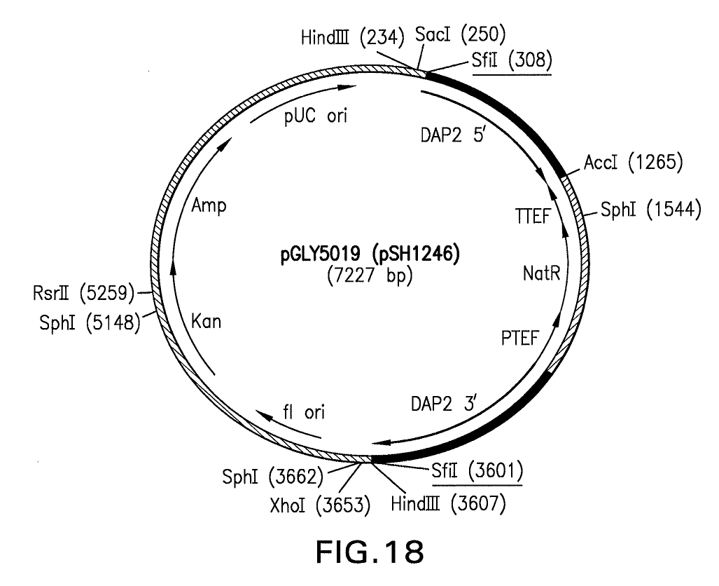

Plasmid pGLY5019 (FIG. 18) is an integration vector that targets the DAP2 locus and contains an expression cassette comprising a nucleic acid molecule encoding the Nourseothricin resistance (NATR) expression cassette (originally from pAG25 from EROSCARF, Scientific Research and Development GmbH, Daimlerstrasse 13a, D-61352 Bad Homburg, Germany, See Goldstein et al., Yeast 15: 1541 (1999)). The NAT^{R} expression cassette (SEQ ID NO:13) is operably regulated to the Ashbya gossypii TEF1 promoter and A. gossypii TEF1 termination sequences flanked one side with the 5′ nucleotide sequence of the P. pastoris DAP2 gene (SEQ ID NO:84) and on the other side with the 3′ nucleotide sequence of the P. pastoris DAP2 gene (SEQ ID NO:85). Plasmid pGLY5019 was linearized and the linearized plasmid transformed into strain YGLY9469 to produce a number of strains in which the NATR expression cassette has been inserted into the DAP2 locus by double-crossover homologous recombination. The strains YGLY9795 and YGLY9797 were selected from the strains produced.

Strain YGLY9795 was transformed with plasmids pGLY5045 to produce strain YGLY10296, and strain YGLY9797 was transformed with plasmid pGLY5045 or pGLY6391 to produce strains YGLY10299 and YGLY12626, respectively. Each strain can produce a TNFRII-Fc fragment fusion protein.

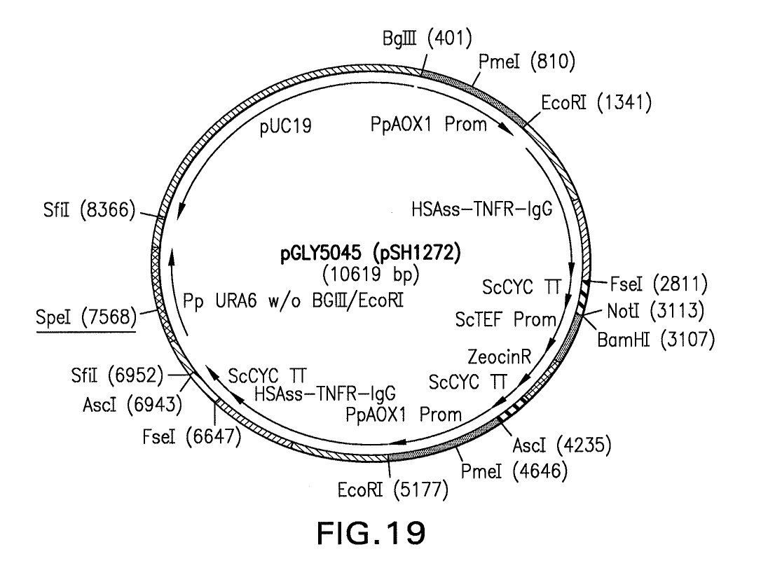

Plasmid pGLY5045 (FIG. 19) is a roll-in integration vector that targets the URA6 locus and contains an expression cassette encoding the TNFRII-Fc fragment fusion protein. The plasmid contains two expression cassettes, each comprising a nucleic acid molecule codon-optimized for expression in P. pastoris encoding the TNFRII-Fc fragment fusion protein (SEQ ID NO:74; encoding SEQ ID NO:75) fused at the 5′ end to a nucleic acid molecule encoding the human serum albumin signal peptide (SEQ ID NO:70; encoding SEQ ID NO:71), which is operably linked at the 5′ end to a nucleic acid molecule comprising the P. pastoris AOX1 promoter and at the 3′ end to a nucleic acid molecule comprising the S. cerevisiae CYC transcription termination sequence. The plasmid also includes a Zeocin^{R} expression cassette comprising a nucleic acid molecule encoding the Sh ble ORF (SEQ ID NO:14) operably linked at the 5′ end to the S. cerevisiae TEF1 promoter (SEQ ID NO:16) and at the 3′ end to the S. cerevisiae CYC termination sequence. The P. pastoris URA6 gene is shown in SEQ ID NO:12. Plasmid pGLY5045 was transformed into strains YGLY9795 and YGLY9797 to produce a number of strains of which strains YGLY10296 and YGLY10299 were selected.

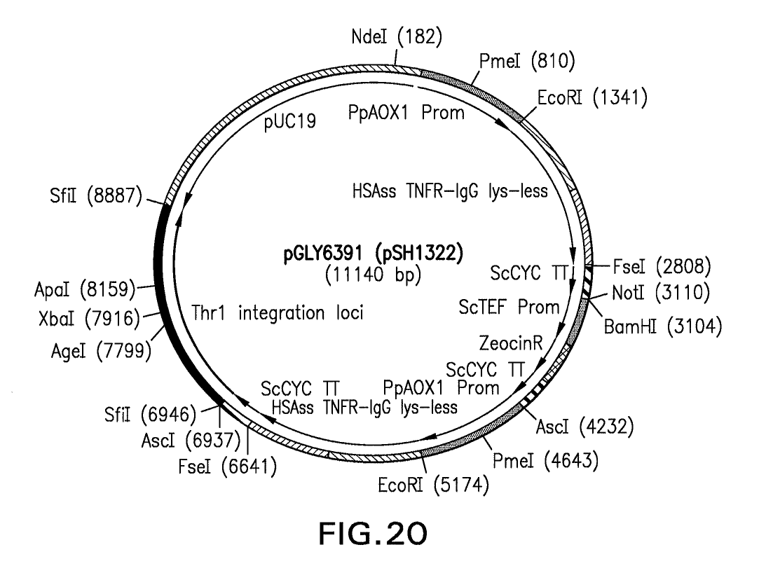

Plasmid pGLY6391 (FIG. 20) is a roll-in integration vector that targets the THR1 locus and contains an expression cassette encoding the TNFRII-Fc fragment fusion protein. The plasmid contains two expression cassettes, each comprising a nucleic acid molecule codon-optimized for expression in P. pastoris encoding the TNFRII-Fc fragment fusion protein without the C-terminal lysine residue (SEQ ID NO:72; encoding SEQ ID NO:73) fused at the 5′ end to a nucleic acid molecule encoding the human serum albumin signal peptide, which is operably linked at the 5′ end to a nucleic acid molecule comprising the P. pastoris AOX1 promoter and at the 3′ end to a nucleic acid molecule comprising the S. cerevisiae CYC transcription termination sequence. The plasmid also includes a Zeocin^{R} expression cassette comprising a nucleic acid molecule encoding the Sh ble ORF operably linked at the 5′ end to the S. cerevisiae TEF1 promoter and at the 3′ end to the S. cerevisiae CYC termination sequence. The P. pastoris THR1 gene is shown in SEQ ID NO:86. Plasmid pGLY6391 was transformed into strain YGLY9797 to produce a number of strains of which strain YGLY12626 was selected.

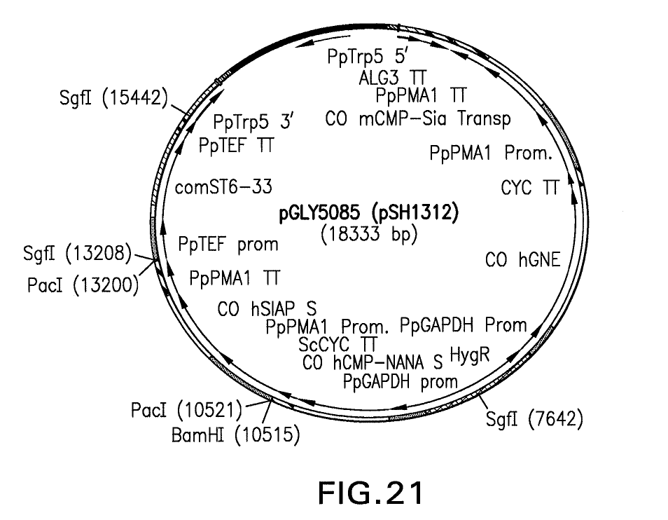

Plasmid pGLY5085 (FIG. 21) is a KINKO plasmid for introducing a second set of the genes involved in producing sialylated N-glycans into P. pastoris. The plasmid is similar to plasmid YGLY2456 except that the P. pastoris ARG1 gene has been replaced with an expression cassette encoding hygromycin resistance (HygR) and the plasmid targets the P. pastoris TRP5 locus. The HYG^{R} resistance cassette is SEQ ID NO:79. The HYG^{R} expression cassette (SEQ ID NO:79) is operably regulated to the Ashbya gossypii TEF1 promoter and A. gossypii TEF1 termination sequences (See Goldstein et al., Yeast 15: 1541 (1999)). The six tandem cassettes are flanked on one side by a nucleic acid molecule comprising a nucleotide sequence from the 5′ region and ORF of the TRP5 gene ending at the stop codon (SEQ ID NO:93) followed by a P. pastoris ALG3 termination sequence and on the other side by a nucleic acid molecule comprising a nucleotide sequence from the 3′ region of the TRP5 gene (SEQ ID NO:94). Plasmid pGLY5085 was transformed into strain YGLY10296 to produce a number of strains of which strain YGLY11731 was selected. Plasmid pGLY5085 was also transformed into strain YGLY12626 to produce a number of strains of which strain YGLY13430 was selected, YGLY13430 was then counterselected in the presence of 5-FOA to produce a number of strains now auxotrophic for uridine of which strain YGLY13571 was selected.

Thus, shown are the construction of Pichia pastoris strains YGLY10299, YGLY11731, and YGLY13571, each strain a GS6.0 strain capable of producing TNFRII-Fc fragment fusion protein comprising sialylated N-glycans.

### Example 2

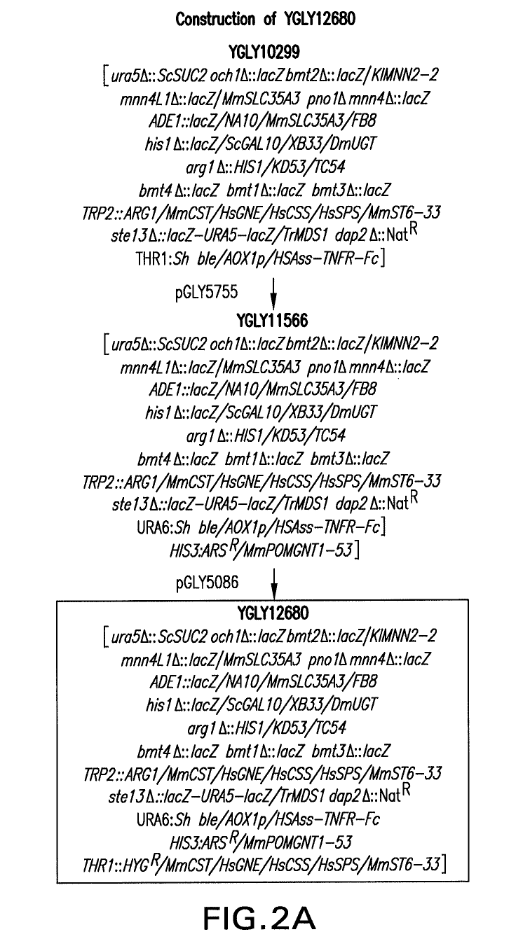

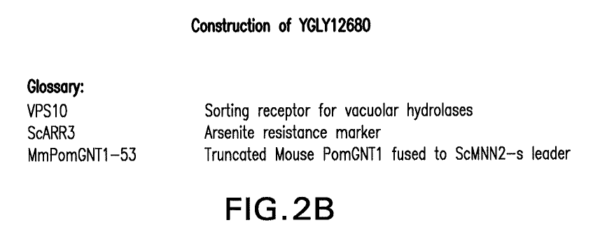

This example shows the construction of Pichia pastoris strains YGLY12680, a GS6.0 strain capable of producing TNFRII-Fc fragment fusion protein with sialylated N-glycans and O-glycans. FIGS. 2A-2B provide a flow-diagram illustrating construction of the strain. Strain YGLY10299 was transformed as follows to produce strain YGLY12680.

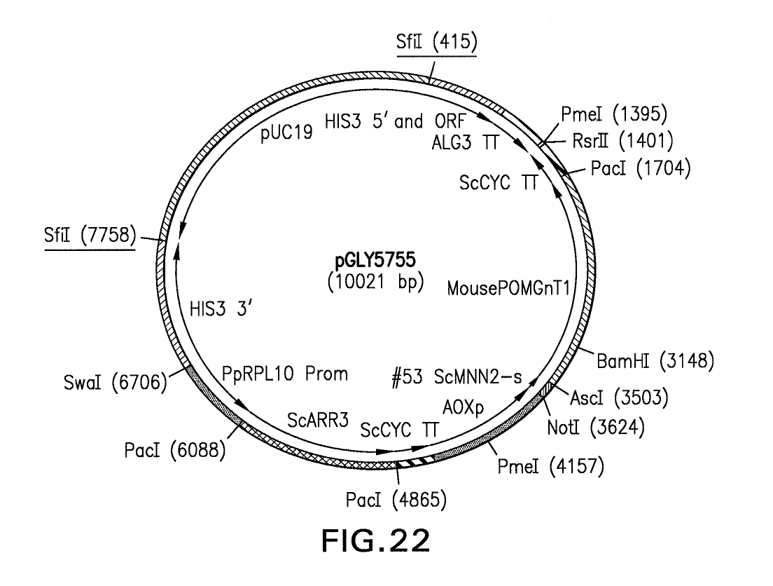

Plasmid pGLY5755 (FIG. 22) is a KINKO integration plasmid that encodes a chimeric mouse POMGnT I and targets the HIS3 locus in P. pastoris. The expression cassette encoding the chimeric mouse POMGnT I comprises a nucleic acid molecule encoding the catalytic domain of the mouse POMGnT I ORF codon-optimized for effective expression in P. pastoris (SEQ ID NO:76) ligated in-frame with a nucleic acid molecule encoding S. cerevisiae MNN2-s signal peptide (53: SEQ ID NO:50) operably linked at the 5′ end to a nucleic acid molecule that has the inducible P. pastoris AOX1 promoter sequence (SEQ ID NO:2) and at the 3′ end to a nucleic acid molecule that has the S. cerevisiae CYC transcription termination sequence (SEQ ID NO:3). For selecting transformants, the plasmid comprises an expression cassette encoding the S. cerevisiae ARR3 ORF in which the nucleic acid molecule encoding the ORF (SEQ ID NO:11) is operably linked at the 5′ end to a nucleic acid molecule having the P. pastoris RPL10 promoter sequence (SEQ ID NO:4) and at the 3′ end to a nucleic acid molecule having the S. cerevisiae CYC transcription termination sequence (SEQ ID NO:3). The expression cassettes are in tandem and are flanked on one side by a nucleic acid molecule comprising a nucleotide sequence from the 5′ region and ORF of the HIS3 gene ending at the stop codon (SEQ ID NO:87) followed by a P. pastoris ALG3 termination sequence and on the other side by a nucleic acid molecule comprising a nucleotide sequence from the 3′ region of the HIS3 gene (SEQ ID NO:88). Plasmid pGLY5755 was linearized with SfiI and the linearized plasmid transformed into strain YGLY10299 to produce a number of strains in which the expression cassettes have been inserted into the HIS3 locus immediately following the HIS3 ORF by double-crossover homologous recombination. The strain YGLY11566 was selected from the strains produced.

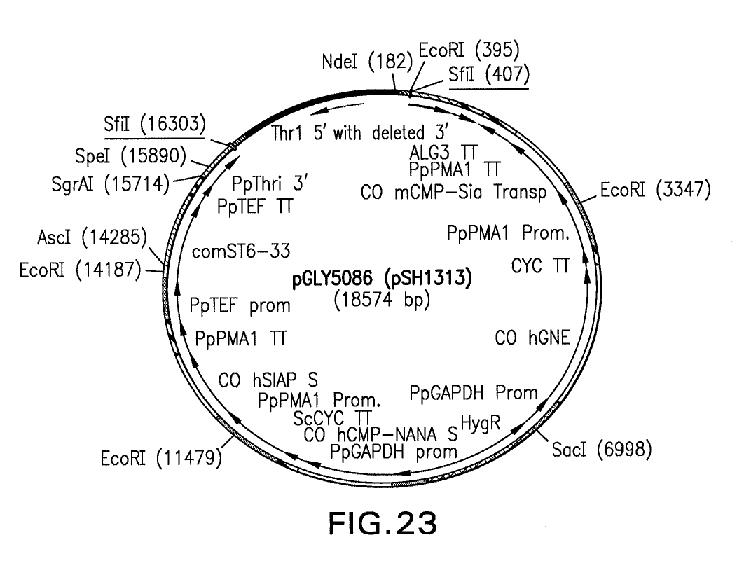

Plasmid pGLY5086 (FIG. 23) is a KINKO plasmid for introducing a second set of the genes involved in producing sialylated N-glycans into P. pastoris. The plasmid is similar to plasmid YGLY5086 except that the plasmid targets the P. pastoris THR1 locus. The expression cassettes are flanked on one side by a nucleic acid molecule comprising a nucleotide sequence from the 5′ region and ORF of the THR1 gene ending at the stop codon (SEQ ID NO:89) followed by a P. pastoris ALG3 termination sequence and on the other side by a nucleic acid molecule comprising a nucleotide sequence from the 3′ region of the THR1 gene (SEQ ID NO:90). Plasmid pGLY5086 was transformed into strain YGLY11566 to produce a number of strains of which strain YGLY12680 was selected.

### Example 3

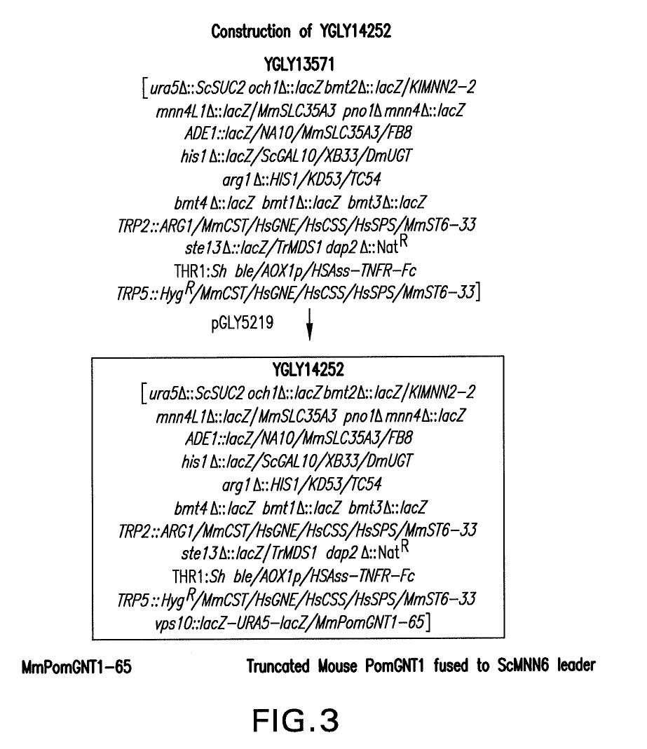

This example shows the construction of Pichia pastoris strain YGLY14252, a GS6.0 strain capable of producing TNFRII-Fc fragment fusion protein with sialylated N-glycans and O-glycans. FIG. 3 provides a flow diagram illustrating construction of the strain. Strain YGLY13571 was transformed as follows to produce strain YGLY14252.

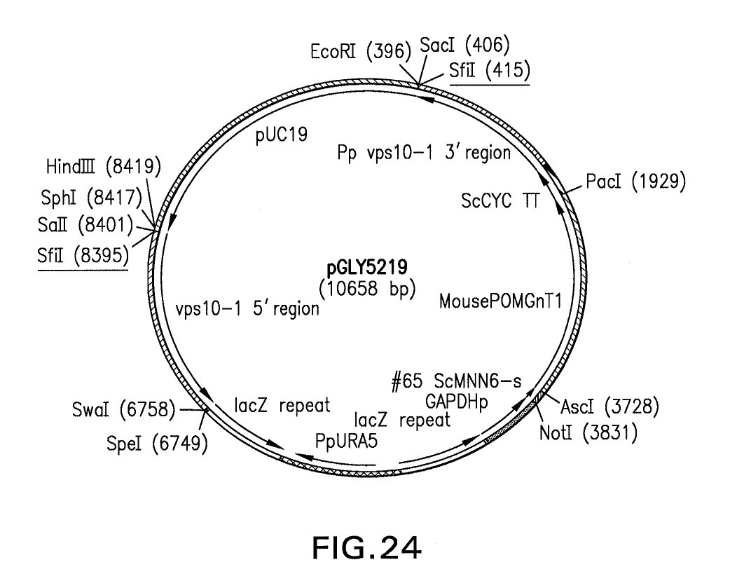

Plasmid pGLY5219 (FIG. 24) is an integration plasmid that encodes a chimeric mouse POMGnT I and targets the VPS10-1 locus in P. pastoris. The expression cassette encoding the chimeric mouse POMGnT I comprises a nucleic acid molecule encoding the catalytic domain of the mouse POMGnT I ORF codon-optimized for effective expression in P. pastoris (SEQ ID NO:76) ligated in-frame with a nucleic acid molecule encoding S. cerevisiae Mnn6-s signal peptide (65: SEQ ID NO:77) operably linked at the 5′ end to a nucleic acid molecule that has the inducible P. pastoris GAPDH promoter sequence (SEQ ID NO:5) and at the 3′ end to a nucleic acid molecule that has the S. cerevisiae CYC transcription termination sequence (SEQ ID NO:3). For selecting transformants, the plasmid comprises an expression cassette comprising the URA5 gene flanked by lacZ repeats as described previously. The expression cassettes are in tandem and are flanked on one side by a nucleic acid molecule comprising a nucleotide sequence from the 5′ region of the VPS10-1 gene (SEQ ID NO:91) and on the other side by a nucleic acid molecule comprising a nucleotide sequence from the 3′ region of the VPS10-1 gene (SEQ ID NO:92). Plasmid pGLY5219 was linearized with SfiI and the linearized plasmid transformed into strain YGLY13571 to produce a number of strains in which the expression cassettes have been inserted into the VPS10-1 locus. The strain YGLY14252 was selected from the strains produced.

### Example 4

This example shows the construction of Pichia pastoris strains YGLY14954 and YGLY14297, each a G56.0 strain capable of producing TNFRII-Fc fragment fusion protein with sialylated N-glycans and O-glycans. FIG. 4 provides a flow diagram illustrating construction of the strains. Strain YGLY13571 was transformed as follows to produce strains YGLY14954 and YGLY14927.

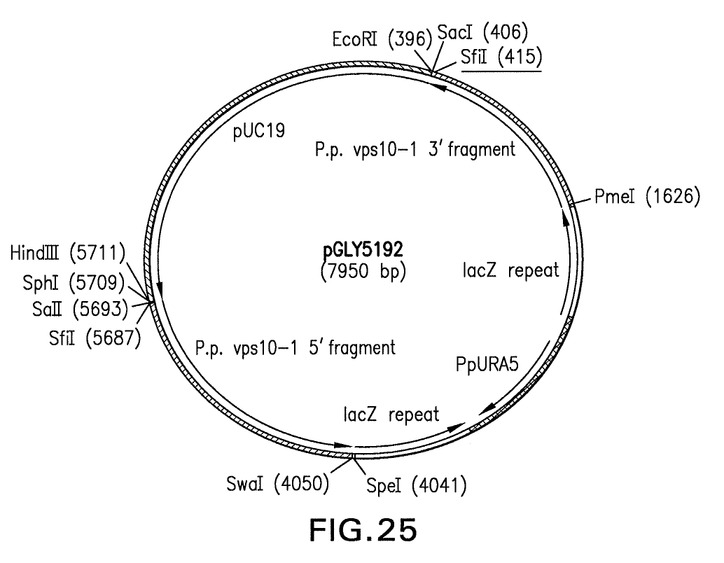

Plasmid pGLY5192 (FIG. 25) is an integration plasmid that targets the VPS10-1 locus. The plasmid comprises an expression cassette comprising the URA5 gene flanked by lacZ repeats as described previously. The expression cassette is flanked on one side by a nucleic acid molecule comprising a nucleotide sequence from the 5′ region of the VPS10-1 gene (SEQ ID NO:91) and on the other side by a nucleic acid molecule comprising a nucleotide sequence from the 3′ region of the VPS10-1 gene (SEQ ID NO:92), Plasmid pGLY5192 was linearized with SfiI and the linearized plasmid transformed into strain YGLY13571 to produce a number of strains in which the expression cassette has been inserted into the VPS10-1 locus. The strain YGLY13663 was selected from the strains produced.

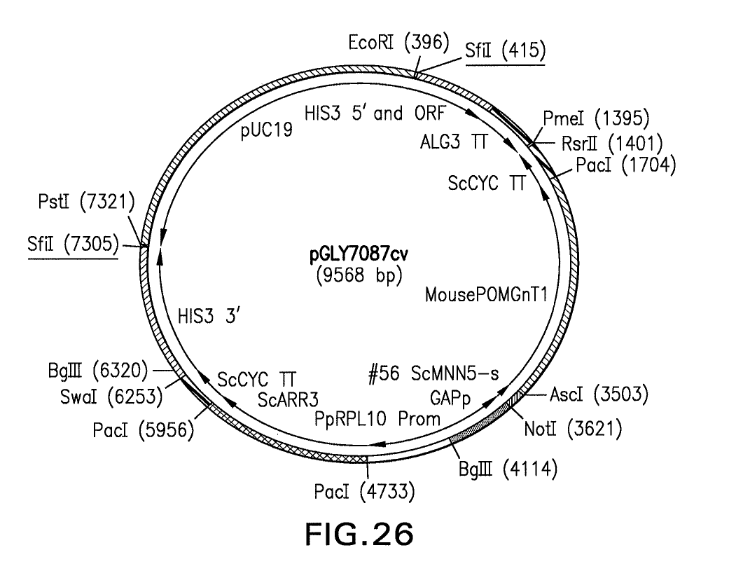

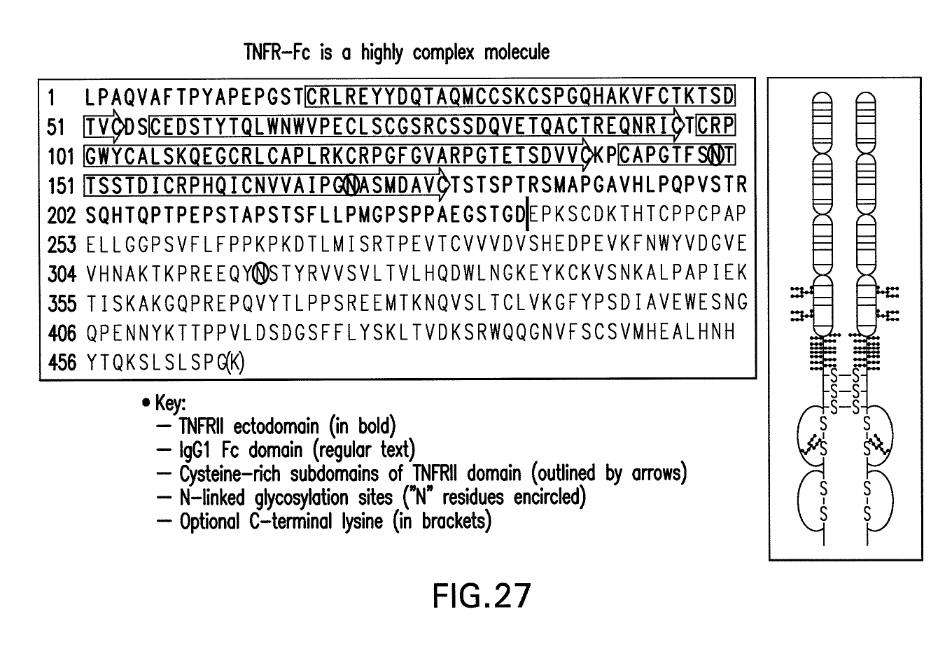

Plasmid pGLY7087 (FIG. 26) is a KINKO integration plasmid that encodes a chimeric mouse POMGnT I and targets the HIS3 locus in P. pastoris. The expression cassette encoding the chimeric mouse POMGnT I comprises a nucleic acid molecule encoding the catalytic domain of the mouse POMGnT I ORF codon-optimized for effective expression in P. pastoris (SEQ ID NO:76) ligated in-frame with a nucleic acid molecule encoding S. cerevisiae Mnn5-s signal peptide (56: SEQ ID NO:78) operably linked at the 5′ end to a nucleic acid molecule that has the inducible P. pastoris GAPDH promoter sequence (SEQ ID NO:5) and at the 3′ end to a nucleic acid molecule that has the S. cerevisiae CYC transcription termination sequence (SEQ ID NO:3). For selecting transformants, the plasmid comprises an expression cassette encoding the S. cerevisiae ARR3 ORF in which the nucleic acid molecule encoding the ORF (SEQ ID NO:11) is operably linked at the 5′ end to a nucleic acid molecule having the P. pastoris RPL10 promoter sequence (SEQ ID NO:4) and at the 3′ end to a nucleic acid molecule having the S. cerevisiae CYC transcription termination sequence (SEQ ID NO:3). The expression cassettes are in tandem and are flanked on one side by a nucleic acid molecule comprising a nucleotide sequence from the 5′ region and ORF of the HIS3 gene ending at the stop codon (SEQ ID NO:87) followed by a P. pastoris ALG3 termination sequence and on the other side by a nucleic acid molecule comprising a nucleotide sequence from the 3′ region of the HIS3 gene (SEQ ID NO:88). Plasmid pGLY7087 was linearized with SfiI and the linearized plasmid transformed into strain YGLY13663 to produce a number of strains in which the expression cassettes have been inserted into the HIS3 locus immediately following the HIS3 ORF by double-crossover homologous recombination. The strains YGLY14954 and YGLY14927 were selected from the strains produced.

### Example 5

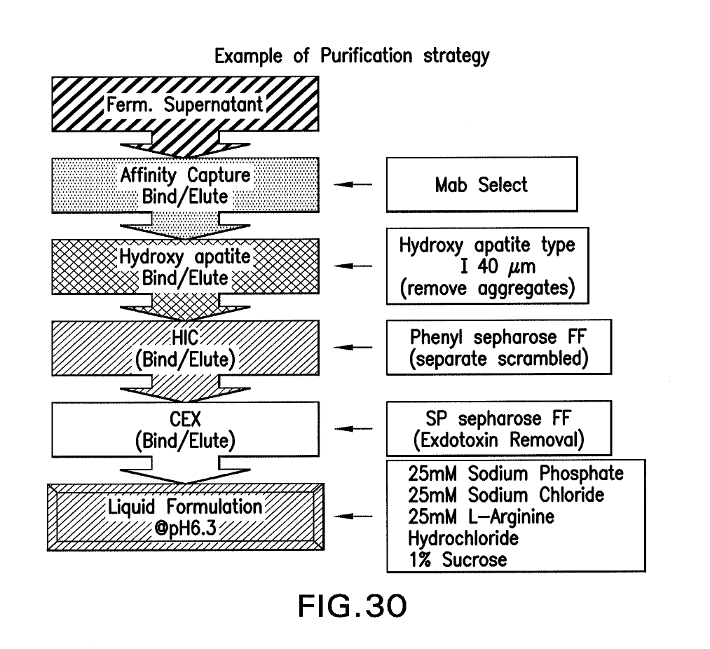

Purification strategy for YGLY10299 (produces Form 1 TNFRII-Fc fragment fusion protein), YGLY11731 (Form 2 TNFRII-Fc fragment fusion protein), and YGLY12680 (Form 3 TNFRII-Fc fragment fusion protein) as shown in FIG. 30.

Form 1 is TNFRII-Fc fragment fusion protein in which the extent of O-glycosylation is reduced and the length of the O-glycans is about one mannose residue. Form 2 is TNFRII-Fc fragment fusion protein in which the extent of O-glycosylation is reduced and the length of the O-glycans is about one mannose residue as for Form 1 but wherein the amount of sialylated N-glycans on the glycoprotein is enhanced. Form 3 is a TNFRII-Fc fragment fusion protein that is similar to Form 2 but further having sialylated O-glycans.

YGLY10299, YGLY11731, and YGLY12680 were grown as follows. The primary culture was prepared by inoculating two 2.8 L baffled Fernbach flasks containing 500 mL of BSGY media with a 2 mL Research Cell Bank of the relevant strain. After 48 hours of incubation, the cells were transferred to inoculate the fermentor. The fermentation batch media contained: 40 g glycerol (Sigma Aldrich, St. Louis, Mo.), 18.2 g sorbitol (Acros Organics, Geel, Belgium), 2.3 g mono-basic potassium phosphate, (Fisher Scientific, Fair Lawn, N.J.) 11.9 g di-basic potassium phosphate (EMD, Gibbstown, N.J.), 10 g Yeast Extract (Sensient, Milwaukee, Wis.), 20 g fly-Soy (Sheffield Bioscience, Norwich, N.Y.), 13.4 g YNB (BD, Franklin Lakes, N.J.), and 4×10⁻³ g biotin (Sigma-Aldrich, St. Louis, Mo.) per liter of medium.

Fermentations were conducted in 3 L & 15 L dished-bottom glass autoclavable and 40 L SIP bioreactors (1.5 L, 8 L & 16 L starting volume respectively) (Applikon, Foster City, Calif.). The fermenters were run in a simple fed-batch mode with the following conditions: temperature of 24±1° C.; pH of 6.5±0.2 maintained by the addition of 30% NH₄OH; airflow of approximately 0.7±0.1 vvm; dissolved oxygen of 20% of saturation was maintained by cascading feedback control of the agitation rate (from 350 to 1200 rpm) followed by supplementation of pure oxygen to the sparged air stream up to 0.1 vvm. After the depletion of the initial charge of glycerol as seen by a sharp increase in dissolved oxygen concentration, a 50% (w/w) glycerol solution containing PTM2 Salts and Biotin was fed at an exponential rate of 5.33 g/L/h increasing at 0.08 l/h for 8 hours to achieve a target cell density of 200 +/−20 g/L (wet cell weight). After a 30 minute Transition period, a 100% methanol solution containing PTM2 Salts and Biotin was initiated. The methanol was fed at an exponential feeding rate of 1.33 g/L/h increasing at 0.01 l/h for 36 hours. At the end of the fermentation, the supernatant was obtained by centrifugation at 13,000×g for 30 minutes and subsequently purified via affinity chromatography.

The purification of TNFRII-Fc fragment fusion protein obtained from the three strains as shown in FIG. 30 was as follows. The TNFRII-Fc fragment fusion protein was captured by affinity chromatography from the culture medium (supernatant medium) of P. pastoris using MABSELECT from GE Healthcare (PolyA-agarose media; Cat. #17-5199-03). The cell free supernatant medium was loaded on to MABSELECT column pre-equilibrated with 3 column volume of 20 mM Tris-HCl pH7.0. The column was washed with 2 column volumes of 20 mM Tris-HCl pH 7.0 and 5 column volume of 20 mM Tris-HCl, 1 M NaCl pH 7.0 to remove the host cell protein contaminants. The TNFRII-Fc fragment fusion protein was eluted with 7 column volumes of 50 mM sodium citrate pH 3.0. The eluted fusion protein was neutralized immediately with 1 M Tris-HCl pH 8.0.

Macro-prep Ceramic Hydroxyapatite type I 40 μm Chromatography (Bio-Rad Laboratories, Cat #157-0040) was used as the first intermediate purification step to remove aggregated forms of TNFRII-Fc fragment fusion protein. The Hydroxyapatite column was equilibrated with 3 column volumes of 5 mM Sodium phosphate pH6.5 and the mabselect pool containing TNFRII-Fc fragment fusion protein that was buffer exchanged into the equilibration buffer was applied on to the column. After loading, the column was washed with 3 column volumes of the equilibration buffer and elution was performed by developing a gradient over 20 column volumes ranging from 0 to 1000 mM sodium chloride. The TNFRII-Fc fragment fusion protein that elutes around 550-650 mM sodium chloride was pooled together.

Hydrophobic Interaction Chromatography (HIC) step was employed as the second intermediate purification step to separate the scrambled or misfolded TNFRII-Fc fragment fusion protein. The Hydroxyapatite pool sample of TNFRII-Fc fragment fusion protein was adjusted to 1 M Ammonium sulfate concentration and loaded on to the Phenyl SEPHAROSE 6 FF (low sub) (GE Healthcare Cat #17-0965-05) column that was pre-equilibrated with 20 mM Sodium phosphate, 1M Ammonium sulfate pH 7.0. After loading, the column was washed with 3 column volumes of the equilibration buffer and elution was performed by developing a gradient over 30 column volumes ranging from 1 M to 0 M ammonium sulfate in 20 mM sodium phosphate pH 7.0. The unscrambled TNFRII-Fc fragment fusion protein that elutes out as a second peak from the HIC column was collected.

Cation Exchange Chromatography (CEX) was employed as the polishing step to clean up the endotoxins and formulate TNFRII-Fc fragment fusion protein into the formulation buffer containing, 25 mM sodium phosphate, 25 mM sodium chloride, 25 mM L-arginine hydrochloride, 1% sucrose pH 6.5±0.2. The HIC peak 2 TNFRII-Fc fragment fusion protein pool that was dialyzed in 25 mM sodium phosphate pH 5.0 was loaded on to the SP SEPHAROSE FF (GE Healthcare Cat #17-0729-01) column that was pre-equilibrated with 25 mM sodium phosphate pH 5.0. After loading, the column was washed with 10 column volumes of 25 mM sodium phosphate pH 5.0 containing 10 mM CHAPS, 10 mM EDTA followed by 10 column volumes wash with 25 mM Sodium phosphate pH 7.0. TNFRII-Fc fragment fusion protein was eluted as a single step elution with the formulation buffer. The peak region containing the TNFRII-Fc fragment fusion protein was pooled and sterile filtered using 0.2 μm PES (PolyEtherSulfone) membrane filter and stored @4° C. until PK/PD studies.

### Example 6

The Glycan composition of TNFRII-Fc fragment fusion protein produced in YGLY10299 (produces Form 1), YGLY11731 (produces Form 2), and YGLY12680 (produces Form 3) was performed as follows.

### O-Glycan Analysis by HPAEC-PAD

Analysis of O-glycans on the TNFRII-Fc fragment fusion protein can use the following protocol.

Yeast strains are grown in shakeflasks containing 100 mL of BMGY for 48 hours, centrifuged, and the cell pellet and washed 1× with BMMY, and then resuspended in 50 mL BMMY and grown an additional 48 hours prior to harvest by centrifugation. Secreted TNFRII-Fc fragment fusion protein is purified from cleared supernatants using protein A chromatography (Li et al. Nat. Biotechnol. 24(2):210-5 (2006)), and the O-glycans released from and separated from protein by alkaline elimination (β-elimination) (Harvey, Mass Spectrometry Reviews 18: 349-451 (1999), Stadheim et al., Nat. Protoc. 3:1026-31 (2006)). This process also reduces the newly formed reducing terminus of the released O-glycan (either oligomannose or mannose) to mannitol. The mannitol group thus serves as a unique indicator of each O-glycan.

About 0.5 nmole or more of protein, contained within a volume of 100 μL PBS buffer, is used for β-elimination. The protein sample is treated with 25 μL alkaline borohydride reagent and incubated at 50° C. for 16 hours. About 20 μL arabitol internal standard is added, followed by 10 μL glacial acetic acid. The sample is then centrifuged through a Millipore filter containing both SEPABEADS and AG 50W-X8 resin and washed with water. The samples, including wash, are transferred to plastic autosampler vials and evaporated to dryness in a centrifugal evaporator. 150 μL 1% AcOH/MeOH is added to the samples and the samples evaporated to dryness in a centrifugal evaporator. This last step is repeated five more times. 200 μL of water is added and 100 μL of the sample is analyzed by high pH anion-exchange chromatography coupled with pulsed electrochemical detection-HPLC (HPAEC-PAD) according to the manufacturer (Dionex, Sunnyvale, Calif.).

### N-Glycan Analysis

To quantify the relative amount of each glycoform, the N-glycosidase F released glycans were labeled with 2-aminobenzidine (2-AB) and analyzed by HPLC as described in Choi et al., Proc. Natl. Acad. Sci. USA 100: 5022-5027 (2003) and Hamilton et al., Science 313: 1441-1443 (2006).

### Total Sialic Acid Determination

The following assay detects total sialic acid content on glycoproteins as a ratio of moles sialic acid/mole protein. Sialic acid was released from glycoprotein samples by acid hydrolysis and analysed by HPAEC-PAD using the following method: About 10-15 μg of protein sample were buffer-exchanged into phosphate buffered saline. Four hundred μL of 0.1M hydrochloric acid was added, and the sample heated at 80° C. for 1 hour. After drying in a SpeedVac (Savant), the samples were reconstituted with 500 μL of water. One hundred uL was then subjected to HPAEC-PAD analysis.

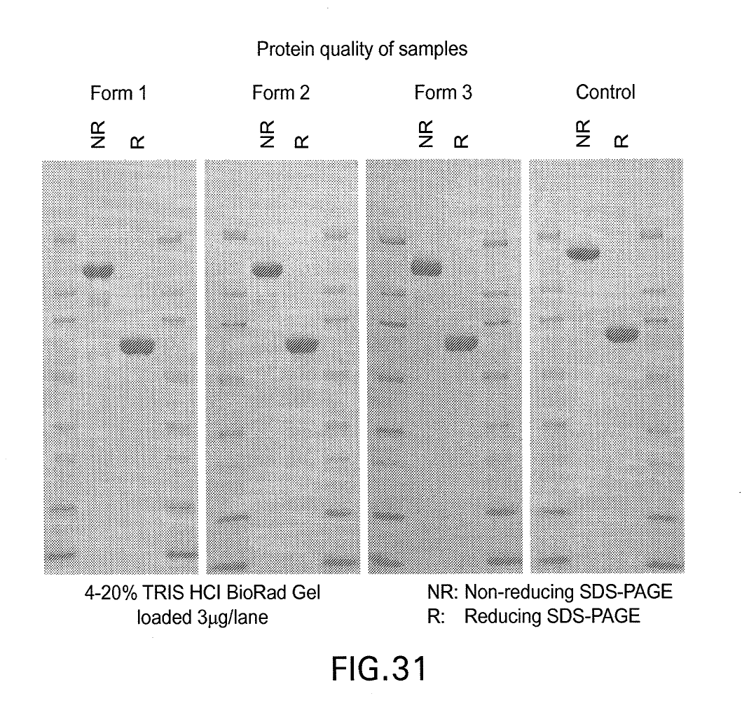

Purified TNFRII-Fc fragment fusion protein was electrophoresed on Tris-buffered 4-20% gradient SDS-polyacrylamide gels obtained from BioRad Laboratories (Hercules, Calif.). About 3 μg of protein prepared in either reducing or non-reducing loading buffer was applied to a lane. A control consisted of commercially-available ENBREL. FIG. 31 shows that all three forms of TNFRII-Fc fragment fusion protein appeared to be similar in size to commercial ENBREL.

The Glycan compositions of the three forms of TNFRII-Fc fragment fusion protein were determined and the results presented in FIG. 32. The figure shows that the glycan composition of the TNFRII-Fc fragment fusion protein was distinguishable from the glycan composition of ENBREL.

### Example 7

TNFRII-Fc fragment fusion protein produced in YGLY10299 (produces Form 1), YGLY11731 (produces Form 2), and YGLY12680 (produces Form 3) was analyzed to assess and compare the bioactivity of the forms of TNFRII-Fc fragment fusion protein. The assays that used were (1) an in vitro assay to measure the effect sialylation of TNFRII-Fc fragment fusion protein has on its ability to inhibit TNFα-induced cell killing of L929 cells, (2) an in vitro assay to measure the effect sialylation of TNFRII-Fc fragment fusion protein has on its ability to inhibit TNFα-stimulated release of IL-6 in A549 cells, and (3) an in vivo assay in rat to measure the effect sialylation of TNFRII-fc fusion protein has on pharmacokinetics.

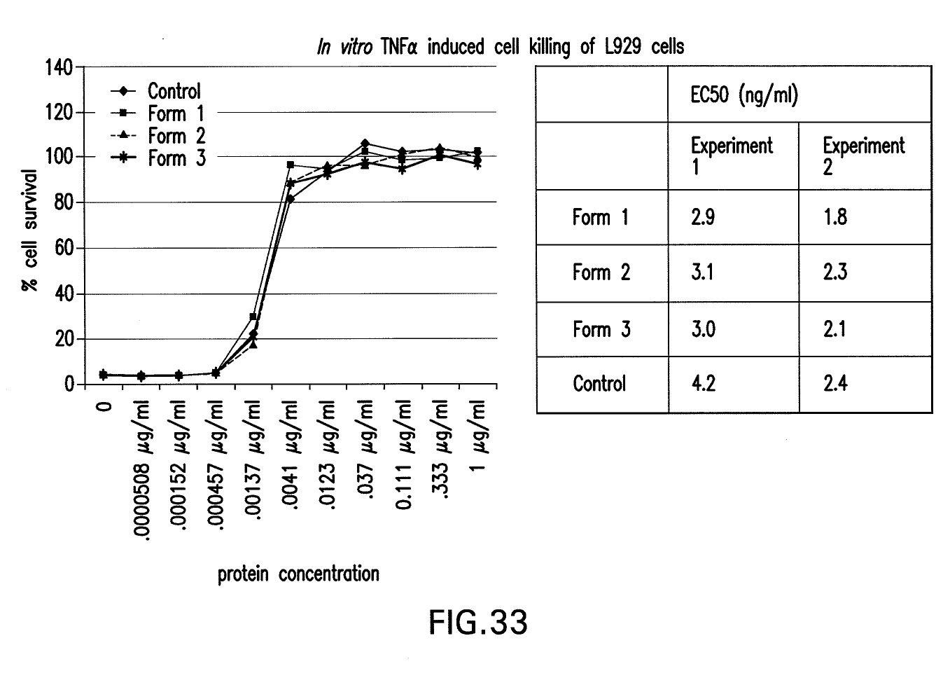

The three forms were compared to commercial ENBREL for ability to inhibit TNFα-induced cell killing of L929 cells. L929 cells were seeded overnight in 96-well plates at about 10,000 cells/well in Eagle's Minimum Essential Medium (ATCC Cat No. 30-2003) supplemented with 10% Fetal Bovine Serum at 37° C. and 5% CO₂. Cells were then treated with human recombinant TNFα at 25 ng/mL with or without TNFRII-Fc fragment fusion protein or commercial ENBREL and then incubated for 24 hours under the same conditions. Then cell viability was measured by ATPlite (luminescence readout from Perkin-Elmer, Waltham, Mass., see also U.S. Pat. No. 6,503,723), The results are shown in FIG. 33 and show that the three forms of TNFRII-Fc fragment fusion protein were comparable to commercial ENBREL in inhibiting cell killing.

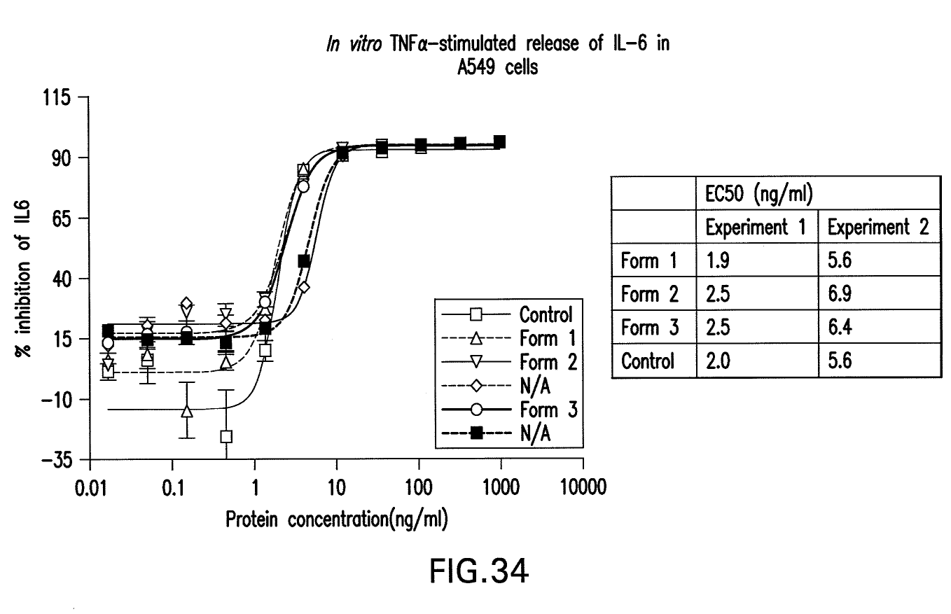

The three forms were compared to commercial ENBREL for ability to inhibit TNFα-stimulated release of IL-6 in A549 cells. A549 cells were seeded overnight in 96-well plates at about 50,000 cells/well in F-12K Medium (ATCC Cat No. 30-2009) medium supplemented with 10% Fetal Bovine Serum at 37° C. and 5% CO₂. Cells were then treated in triplicate with one of the three forms of TNFRII-Fc fragment fusion protein or commercial ENBREL and then stimulated with 3 ng/mL human recombinant TNFα and then incubated overnight under the same conditions. Then IL6 production was determined by AlphaLISA assay (Perkin-Elmer, Waltham, Mass.). The results are shown in FIG. 34 and show that the three forms of TNFRII-Fc fragment fusion protein were comparable to commercial ENBREL in inhibiting TNFα-stimulated release of IL-6.

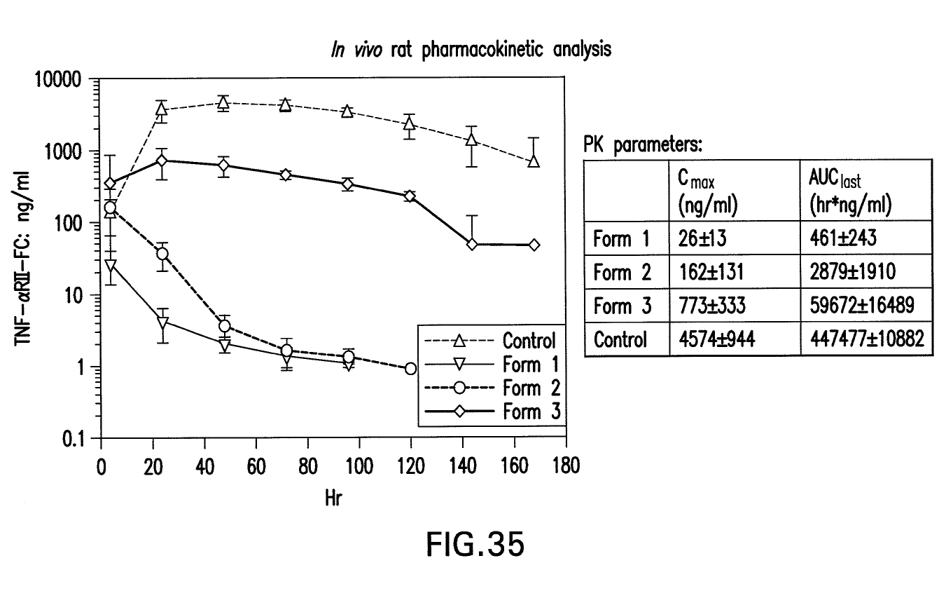

The in vivo pharmacokinetics for each of the three forms was compared to that of commercial ENBREL. Sprague Dawley (SD) rats were dosed subcutaneously (SC) at 1 mg/kg with one of the three forms or commercial ENBREL and serum samples collected at 4, 24, 48, 72, 96, 120, 144, and 168 hour time points following administration. Serum concentration of the TNFRII-Fc fragment fusion protein or commercial ENBREL was determined with a Gyro immunoassay (Gyros US Inc., Monmouth Junction, N.J.) using anti-TNFRII antibody as the capture antibody and labeled anti-Fc antibody for detection. The results are shown in FIG. 35 and show that Forms 1 and 2 of the TNFRII-Fc fragment fusion protein exhibited about 155-900 fold lower exposure than commercial ENBREL following SC administration and Form 3 TNFRII-Fc fragment fusion protein exhibited about 9-10 fold lower exposure than commercial ENBREL following SC administration. The results show that there is an apparent correlation between the extent of sialylation and increased in vivo pharmacokinetics.

Although this example demonstrates that the O-sialylated form of TNFRII-Fc (Form 3) has more activity in vivo compared to the O-mannose reduced glycan forms (Forms 1 and 2), all three forms demonstrated similar activity in in vitro assays. As such, it is foreseeable that one skilled in the art could increase the bioavailability and/or half-life of the O-mannose reduced glycan forms, to provide a therapeutic molecule with similar in vivo characteristics to the O-sialylated form or commercial ENBREL. One such strategy would be to increase the bioavailability of the molecule by formulation buffer optimization. An alternative strategy would be to increase the half-life of the molecule by conjugation to a carrier molecule to increase its physical size, for example, covalent linkage to polyethylene glycol.

### Example 8

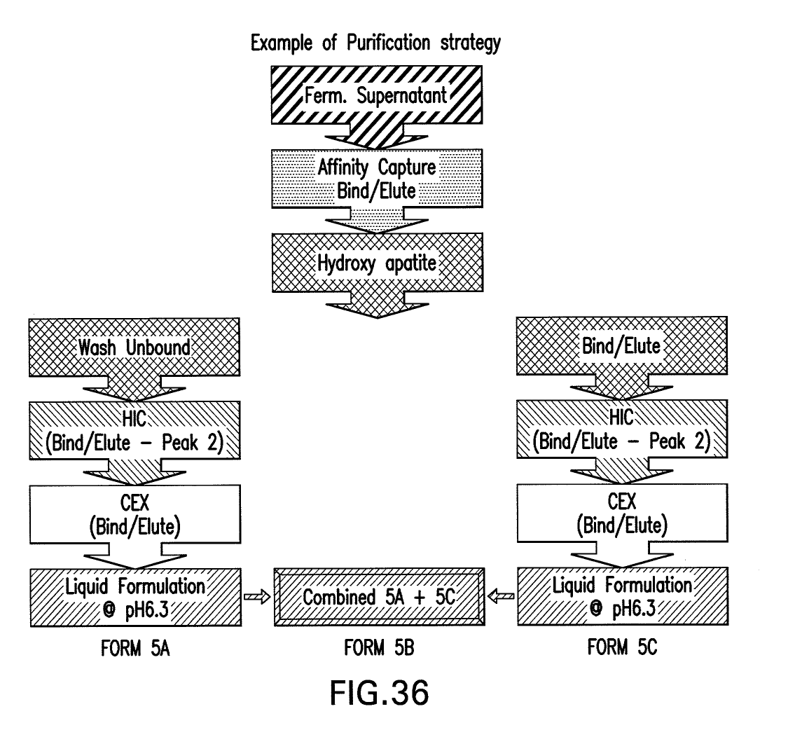

Purification strategy for TNFRII-Fc fragment fusion protein produced in strain YGLY14252 as shown in FIG. 36. The purification strategy enabled isolation of three forms of TNFRII-Fc fragment fusion protein: Form 5A, which has high relative total sialic acid (TSA) content; Form 513, which has medium TSA content; and, Form 5C, which has low TSA content.

YGLY14252 was grown as described in Example 5 above. The purification of Forms 5A, 513, and 5C of TNFRII-Fc fragment fusion protein obtained from YGLY14252 as shown in FIG. 36 was as follows.

Briefly, the same strategy as described in Example 5 was used with the following changes in the first intermediate purification step using Macro-Prep Ceramic Hydroxyapatite type I 40 μm resin. This step was not only used to remove the aggregated forms of TNFRII-Fc fragment fusion protein, but also to separate highly sialylated N- and O-Glycan containing fractions of TNFRII-Fc fragment fusion protein.

The Hydroxyapatite column was equilibrated with 3 column volumes of 5 mM sodium phosphate pH 6.5 and the mabselect pool containing TNFRII-Fc fragment fusion protein that was buffer exchanged into the equilibration buffer was applied on to the column. After loading, the column was washed with 3 column volumes of the equilibration buffer. The TNFRII-Fc fragment fusion protein that was present in the flowthrough and wash-unbound were collected together as one pool and used for generating Form 5A which contains highly sialylated N- and O-glycans. Elution was performed by developing a gradient over 20 column volume ranging from 0 to 1000 mM Sodium chloride. TNFRII-Fc fragment fusion protein that elutes around 550-650 mM Sodium chloride was pooled together and used for Form 5C generation.

The final formulated TNFRII-Fc fragment fusion protein of Forms 5A and 5C were mixed 1:1 protein ratio to generate Form 5B. All the three Forms 5A, 5B and 5C final formulated samples were stored @4° C. until PK/PD studies.

### Example 9

The three forms of TNFRII-Fc fragment fusion protein obtained as shown in FIG. 36 were analyzed to assess and compare the bioactivity of the 5A, 5B, and 5C forms of TNFRII-Fc fragment fusion protein. The assays that used were (1) an in vitro assay to measure the effect sialylation of TNFRII-Fc fragment fusion protein has on its ability to inhibit TNFα-induced cell killing of L929 cells, (2) an in vitro assay to measure the effect sialylation of TNFRII-Fc fragment fusion protein has on its ability to inhibit TNFα-stimulated release of IL-6 in A549 cells, and (3) an in vivo assay in rat and mouse to measure the effect sialylation of TNFRII-fc fusion protein has on pharmacokinetics.

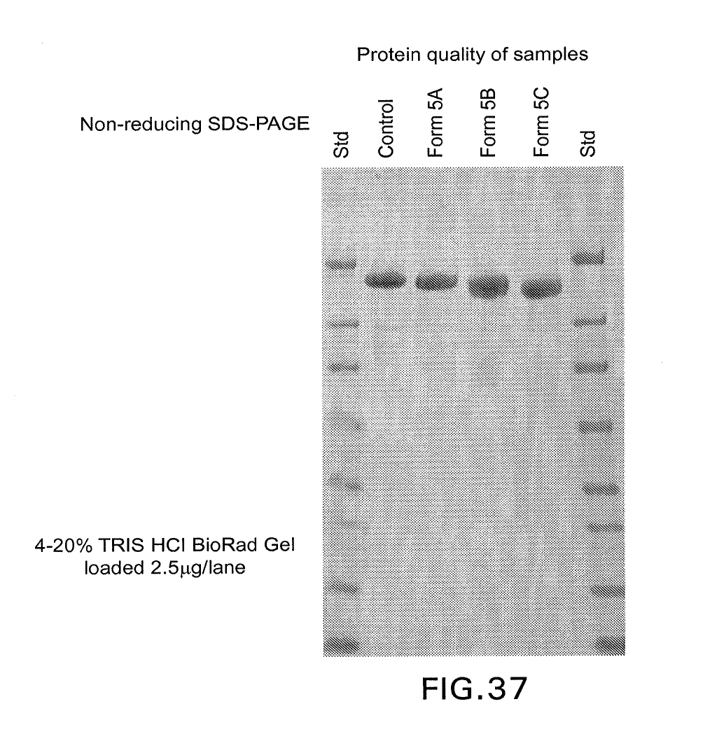

Purified 5A, 5B, and 5C forms of TNFRII-Fc fragment fusion protein were electrophoresed on Tris-buffered 4-20% gradient SDS-polyacrylamide gels obtained from BioRad Laboratories (Hercules, Calif.). About 3 μg of non-reduced protein was applied to a lane. A control consisted of commercially-available ENBREL. FIG. 37 shows that the Form 5A of TNFRII-Fc fragment fusion protein appeared to be similar in size to commercial ENBREL.

The glycan compositions of the three forms of TNFRII-Fc fragment fusion protein were determined as in Example 6 and the results presented in FIG. 38. The figure shows that the glycan composition of each of the three fractions of TNFRII-Fc fragment fusion protein was distinguishable from the glycan composition of ENBREL.

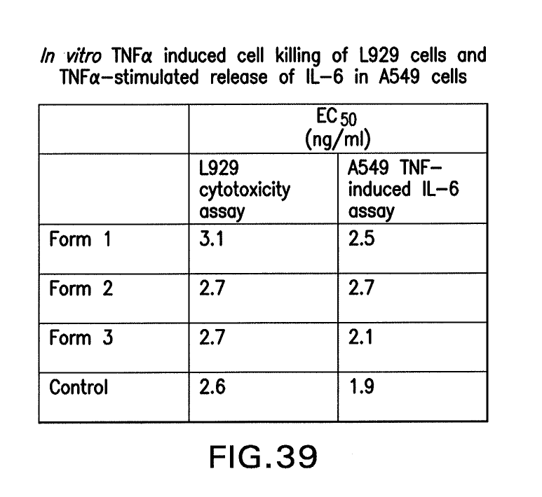

FIG. 39 shows the results of an in vitro assay to measure the effect sialylation of TNFRII-Fc fragment fusion protein has on its ability to inhibit TNFα-induced cell killing of L929 cells or inhibit TNFα-stimulated release of IL-6 in A549 cells. No significant difference was observed between Merck TNFRII-Fc samples and commercial ENBREL.

TNFRII-Fc fragment fusion protein Form 5A had a similar PK profile to commercial ENBREL following SC administration in both rat and mouse models (FIG. 40 and FIG. 41, respectively). In contrast, TNFRII-Fc fragment fusion protein Forms 5B and 5C, each possessing a lower TSA content to Form 5A, had markedly lower in vivo PK when compared to both commercial ENBREL and Form 5A (FIG. 40 and FIG. 41). The results show that there is a direct correlation between the extent of sialylation and increased in vivo pharmacokinetics.

### Example 10

Pichia TNFRII-Fc was tested together with ENBREL for efficacy in a chronic mouse model of rheumatoid arthritis. The Tg197 genetically engineered mice overexpress a human TNF transgene and develop progressive arthritis (Keffer et al., EMBO J. (13): 4025-4031 (1991)). The primary intent of the study was to verify whether the ability of Pichia TNFRII-Fc to neutralize TNF bioactivity translates into an ability to block the chronic effects of overexpressed TNF; the secondary purpose of the study was to compare the chronic effects of Pichia TNFRII-Fc to those of ENBREL. Transgenic mice were separated into 7 groups consisting of 8 gender and age-matched mice each, which received intraperitoneally 10 μl of test compounds per gram of body weight, twice weekly. The groups received different test materials and dose levels, as follows: Vehicle, Pichia TNFRII-Fc at 30, 10 and 3 mg/kg; commercial ENBREL at 30, 10 and 3 mg/kg. Treatment was initiated at the onset of arthritis (three weeks of age) and continued over 8 weeks; the study was concluded at 10 weeks of age.

The assessment indicates (FIG. 42) that Pichia TNFRII-Fc has in vivo potency and target efficacy. Its effectiveness shows a dose effect relationship, with higher doses increasing the anti-arthritic effect. The effects that Pichia TNFRII-Fc and commercial Enbrel have on the arthritic scores are similar at 30, 10 and 3 mg/kg dose levels.

### Example 11

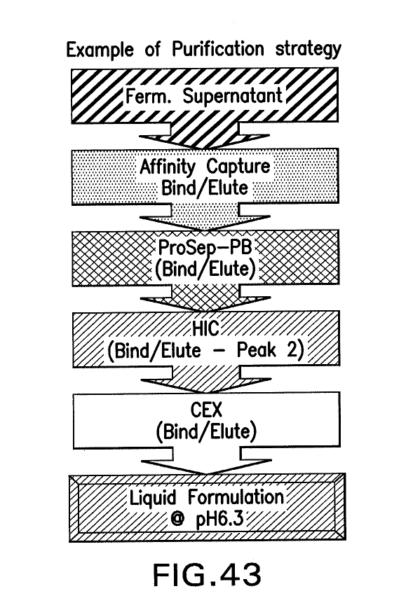

An alternative purification strategy for enrichment of highly sialylated glycoforms of TNFRII-Fc was developed using phenyl borate chromatography instead of hydroxyapatite chromatography as shown by the scheme in FIG. 43. This strategy was similar to the strategy as described in EXAMPLE 8 above except with the following changes in the first intermediate purification step in which PROSEP-PB chromatography media (non-compressible media comprising m-aminophenylborate ligands attached to glass beads; Millipore Corp. Cat #113247327) was used instead of Macro-Prep Ceramic Hydroxyapatite type I 40 μm resin to enrich for highly sialylated N and O-linked glycan containing fractions of TNFRII-Fc fragment fusion protein.

The PROSEP-PB column was equilibrated with 3 column volumes of 50 mM HEPES (N′-2-hydroxyethylpiperazine-N′-2 ethanesulphonic acid) pH 8.0 and the mabselect pool containing TNFRII-Fc fragment fusion protein that was previously buffer exchanged into the equilibration buffer was applied on to the column. After loading, the column was washed with 3 column volumes of the equilibration buffer. Elution was performed by developing a linear gradient over 30 column volumes ranging from 0 to 125 mM sorbitol in 50 mM HEPES pH8.0. Highly sialylated forms of TNFRII-Fc fragment fusion protein that elutes earlier in the gradient ranging between 7 mM to 20 mM sorbitol were collected and further processed through second intermediate step purification utilizing Hydrophobic Interaction Chromatography.

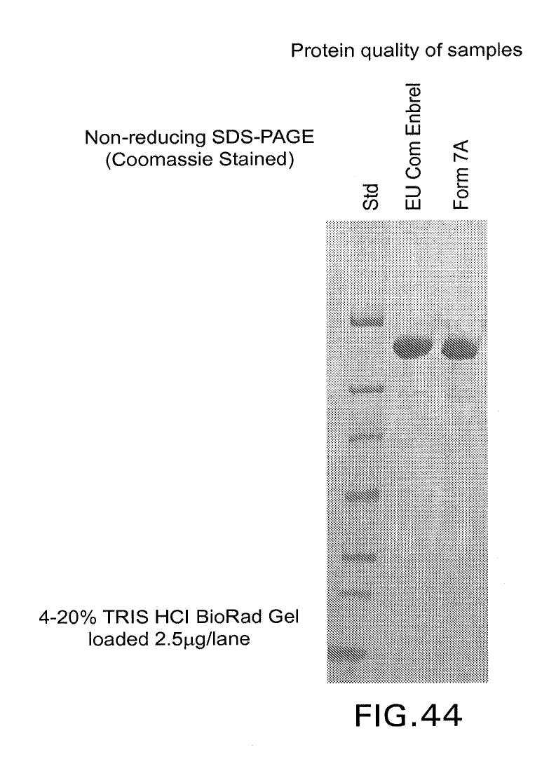

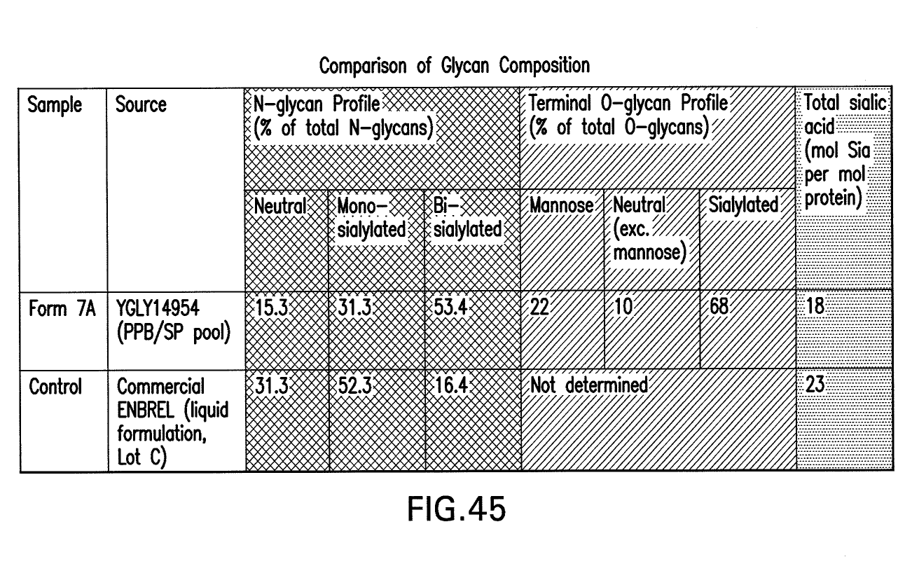

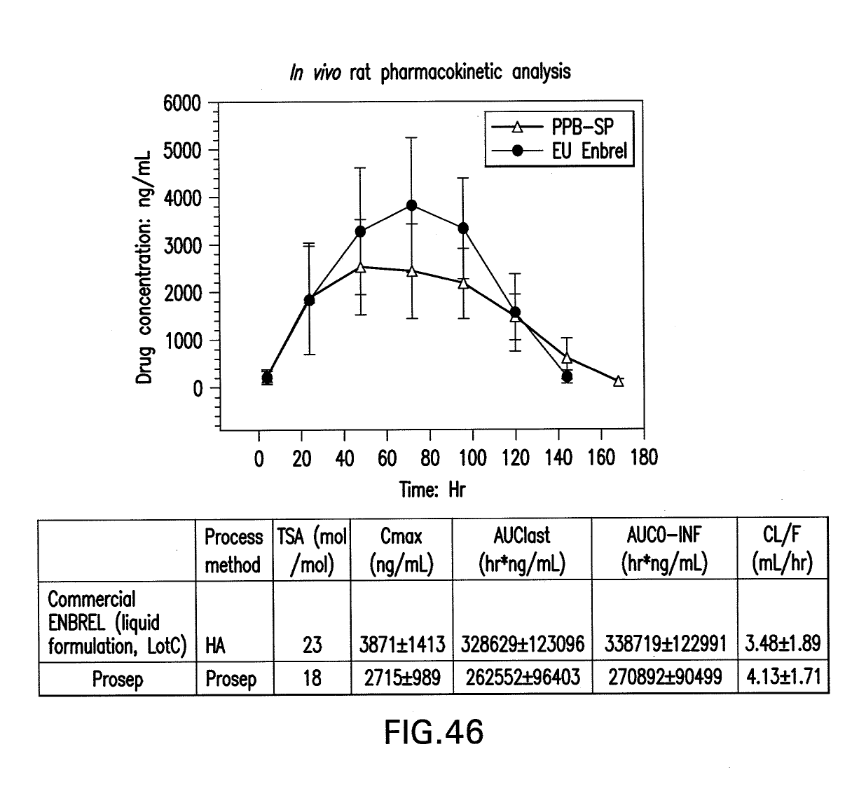

FIG. 44 demonstrates that the protein quality of the material isolated (Form 7A) using this purification strategy was of similar quality to that of the commercial ENBREL control. Characterization of the glycan quality of Form 7A material (FIG. 45) indicates that the TSA content compared to the commercial Enbrel lot used is similar to that highlighted in FIG. 37, when comparing Form 5A to a different lot of commercial ENBREL. The in vivo comparison of the material purified using the Prosep-PB purification strategy in a rat pharmacokinetic study (FIG. 46) indicates that the Form 7A material was comparable to commercial ENBREL.

While the various expression cassettes were integrated into particular loci of the Pichia pastoris genome in the examples herein, it is understood that the operation of the invention is independent of the loci used for integration. Loci other than those disclosed herein can be used for integration of the expression cassettes. Suitable integration sites include those enumerated in U.S. Published Application No. 20070072262 and include homologs to loci known for Saccharomyces cerevisiae and other yeast or fungi.

TABLE OF SEQUENCES

| | Description | |
|---|---|---|
| | Pp = Pichia | |
| | pastoris | |
| SEQ | Sc = | |
| ID | Saccharomyces | |
| NO: | cerevisiae | Sequence |
| 1 | Sequence of the | AAATGCGTACCTCTTCTACGAGATTCAAGCGAATGAG |
| | PpPMA1 | AATAATGTAATATGCAAGATCAGAAAGAATGAAAGG |
| | promoter: | AGTTGAAAAAAAAAACCGTTGCGTTTTGACCTTGAAT |
| | | GGGGTGGAGGTTTCCATTCAAAGTAAAGCCTGTGTCT |
| | | TGGTATTTTCGGCGGCACAAGAAATCGTAATTTTCATC |
| | | TTCTAAACGATGAAGATCGCAGCCCAACCTGTATGTA |
| | | GTTAACCGGTCGGAATTATAAGAAAGATTTTCGATCA |
| | | ACAAACCCTAGCAAATAGAAAGCAGGGTTACAACTTT |
| | | AAACCGAAGTCACAAACGATAAACCACTCAGCTCCCA |
| | | CCCAAATTCATTCCCACTAGCAGAAAGGAATTATTTA |
| | | ATCCCTCAGGAAACCTCGATGATTCTCCCGTTCTTCCA |
| | | TGGGCGGGTATCGCAAAATGAGGAATTTTTCAAATTT |
| | | CTCTATTGTCAAGACTGTTTATTATCTAAGAAATAGCC |
| | | CAATCCGAAGCTCAGTTTTGAAAAAATCACTTCCGCG |
| | | TTTCTTTTTTACAGCCCGATGAATATCCAAATTTGGAA |
| | | TATGGATTACTCTATCGGGACTGCAGATAATATGACA |
| | | ACAACGCAGATTACATTTTAGGTAAGGCATAAACACC |
| | | AGCCAGAAATGAAACGCCCACTAGCCATGGTCGAATA |
| | | GTCCAATGAATTCAGATAGCTATGGTCTAAAAGCTGA |
| | | TGTTTTTTATTGGGTAATGGCGAAGAGTCCAGTACGAC |
| | | TTCCAGCAGAGCTGAGATGGCCATTTTTGGGGGTATT |
| | | AGTAACTTTTTGAGCTCTTTTCACTTCGATGAAGTGTC |
| | | CCATTCGGGATATAATCGGATCGCGTCGTTTTCTCGAA |
| | | AATACAGCTTAGCGTCGTCCGCTTGTTGTAAAAGCAG |
| | | CACCACATTCCTAATCTCTTATATAAACAAAACAACCC |
| | | AAATTATCAGTGCTGTTTTCCCACCAGATATAAGTTTC |
| | | TTTTCTCTTCCGCTTTTTGATTTTTTATCTCTTTCCTTTA |
| | | AAAACTTCTTTACCTTAAAGGGCGGCC |
| 2 | Pp AOX1 | AACATCCAAAGACGAAAGGTTGAATGAAACCTTTTTG |
| | promoter | CCATCCGACATCCACAGGTCCATTCTCACACATAAGT |
| | | GCCAAACGCAACAGGAGGGGATACACTAGCAGCAGA |
| | | CCGTTGCAAACGCAGGACCTCCACTCCTCTTCTCCTCA |
| | | ACACCCACTTTTGCCATCGAAAAACCAGCCCAGTTATT |
| | | GGGCTTGATTGGAGCTCGCTCATTCCAATTCCTTCTAT |
| | | TAGGCTACTAACACCATGACTTTATTAGCCTGTCTATC |
| | | CTGGCCCCCCTGGCGAGGTTCATGTTTGTTTATTTCCG |
| | | AATGCAACAAGCTCCGCATTACACCCGAACATCACTC |
| | | CAGATGAGGGCTTTCTGAGTGTGGGGTCAAATAGTTT |
| | | CATGTTCCCCAAATGGCCCAAAACTGACAGTTTAAAC |
| | | GCTGTCTTGGAACCTAATATGACAAAAGCGTGATCTC |
| | | ATCCAAGATGAACTAAGTTTGGTTCGTTGAAATGCTA |
| | | ACGGCCAGTTGGTCAAAAAGAAACTTCCAAAAGTCGG |
| | | CATACCGTTTGTCTTGTTTGGTATTGATTGACGAATGC |
| | | TCAAAAATAATCTCATTAATGCTTAGCGCAGTCTCTCT |
| | | ATCGCTTCTGAACCCCGGTGCACCTGTGCCGAAACGC |
| | | AAATGGGGAAACACCCGCTTTTTGGATGATTATGCAT |
| | | TGTCTCCACATTGTATGCTTCCAAGATTCTGGTGGGAA |
| | | TACTGCTGATAGCCTAACGTTCATGATCAAAATTTAAC |
| | | TGTTCTAACCCCTACTTGACAGCAATATATAAACAGA |
| | | AGGAAGCTGCCCTGTCTTAAACCTTTTTTTTTATCATC |
| | | ATTATTAGCTTACTTTCATAATTGCGACTGGTTCCAAT |
| | | TGACAAGCTTTTGATTTTAACGACTTTTAACGACAACT |
| | | TGAGAAGATCAAAAAACAACTAATTATTCGAAACG |
| 3 | SeCYC TT | ACAGGCCCCTTTTCCTTTGTCGATATCATGTAATTAGT |
| | | TATGTCACGCTTACATTCACGCCCTCCTCCCACATCCG |
| | | CTCTAACCGAAAAGGAAGGAGTTAGACAACCTGAAGT |
| | | CTAGGTCCCTATTTATTTTTTTTAATAGTTATGTTAGTA |
| | | TTAAGAACGTTATTTATATTTCAAATTTTTCTTTTTTTT |
| | | CTGTACAAACGCGTGTACGCATGTAACATTATACTGA |
| | | AAACCTTGCTTGAGAAGGTTTTGGGACGCTCGAAGGC |
| | | TTTAATTTGCAAGCTGCCGGCTCTTAAG |
| 4 | PpRPL10 | GTTCTTCGCTTGGTCTTGTATCTCCTTACACTGTATCTT |
| | promoter | CCCATTTGCGTTTAGGTGGTTATCAAAAACTAAAAGG |
| | | AAAAATTTCAGATGTTTATCTCTAAGGTTTTTTCTTTTT |
| | | ACAGTATAACACGTGATGCGTCACGTGGTACTAGATT |
| | | ACGTAAGTTATTTTGGTCCGGTGGGTAAGTGGGTAAG |
| | | AATAGAAAGCATGAAGGTTTACAAAAACGCAGTCACG |
| | | AATTATTGCTACTTCGAGCTTGGAACCACCCCAAAGA |
| | | TTATATTGTACTGATGCACTACCTTCTCGATTTTGCTCC |
| | | TCCAAGAACCTACGAAAAACATTTCTTGAGCCTTTTCA |
| | | ACCTAGACTACACATCAAGTTATTTAAGGTATGTTCCG |
| | | TTAACATGTAAGAAAAGGAGAGGATAGATCGTTTATG |
| | | GGGTACGTCGCCTGATTCAAGCGTGACCATTCGAAGA |
| | | ATAGGCCTTCGAAAGCTGAATAAAGCAAATGTCAGTT |
| | | GCGATTGGTATGCTGACAAATTAGCATAAAAAGCAAT |
| | | AGACTTTCTAACCACCTGTTTTTTTCCTTTTACTTTATT |
| | | TATATTTTGCCACCGTACTAACAAGTTCAGACAAA |
| 5 | PpGAPDH | TTTTTGTAGAAATGTCTTGGTGTCCTCGTCCAATCAGG |
| | promoter | TAGCCATCTCTGAAATATCTGGCTCCGTTGCAACTCCG |
| | | AACGACCTGCTGGCAACGTAAAATTCTCCGGGGTAAA |
| | | ACTTAAATGTGGAGTAATGGAACCAGAAACGTCTCTT |
| | | CCCTTCTCTCTCCTTCCACCGCCCGTTACCGTCCCTAG |
| | | GAAATTTTACTCTGCTGGAGAGCTTCTTCTACGGCCCC |
| | | CTTGCAGCAATGCTCTTCCCAGCATTACGTTGCGGGTA |
| | | AAACGGAGGTCGTGTACCCGACCTAGCAGCCCAGGGA |
| | | TGGAAAAGTCCCGGCCGTCGCTGGCAATAATAGCGGG |
| | | CGGACGCATGTCATGAGATTATTGGAAACCACCAGAA |
| | | TCGAATATAAAAGGCGAACACCTTTCCCAATTTTGGTT |
| | | TCTCCTGACCCAAAGACTTTAAATTTAATTTATTTGTC |
| | | CCTATTTCAATCAATTGAACAACTATCAAAACACA |
| 6 | PpTEF1 | TTAAGGTTTGGAACAACACTAAACTACCTTGCGGTAC |
| | promoter | TACCATTGACACTACACATCCTTAATTCCAATCCTGTC |
| | | TGGCCTCCTTCACCTTTTAACCATCTTGCCCATTCCAA |
| | | CTCGTGTCAGATTGCGTATCAAGTGAAAAAAAAAAAA |
| | | TTTTAAATCTTTAACCCAATCAGGTAATAACTGTCGCC |
| | | TCTTTTATCTGCCGCACTGCATGAGGTGTCCCCTTAGT |
| | | GGGAAAGAGTACTGAGCCAACCCTGGAGGACAGCAA |
| | | GGGAAAAATACCTACAACTTGCTTCATAATGGTCGTA |
| | | AAAACAATCCTTGTCGGATATAAGTGTTGTAGACTGT |
| | | CCCTTATCCTCTGCGATGTTCTTCCTCTCAAAGTTTGC |
| | | GATTTCTCTCTATCAGAATTGCCATCAAGAGACTCAGG |
| | | ACTAATTTCGCAGTCCCACACGCACTCGTACATGATTG |
| | | GCTGAAATTTCCCTAAAGAATTTCTTTTTCACGAAAAT |
| | | TTTTTTTTTACACAAGATTTTCAGCAGATATAAAATGG |
| | | AGAGCAGGACCTCCGCTGTGACTCTTCTTTTTTTTCTTT |
| | | TATTCTCACTACATACATTTTAGTTATTCGCCAAC |
| 7 | PpTEF1 TT | ATTGCTTGAAGCTTTAATTTATTTTATTAACATAATAA |
| | | TAATACAAGCATGATATATTTGTATTTTGTTCGTTAAC |
| | | ATTGATGTTTTCTTCATTTACTGTTATTGTTTGTAACTT |
| | | TGATCGATTTATCTTTTCTACTTTACTGTAATATGGCTG |
| | | GCGGGTGAGCCTTGAACTCCCTGTATTACTTTACCTTG |
| | | CTATTACTTAATCTATTGACTAGCAGCGACCTCTTCAA |
| | | CCGAAGGGCAAGTACACAGCAAGTTCATGTCTCCGTA |
| | | AGTGTCATCAACCCTGGAAACAGTGGGCCATGTC |
| 8 | PpALG3 TT | ATTTACAATTAGTAATATTAAGGTGGTAAAAACATTC |
| | | GTAGAATTGAAATGAATTAATATAGTATGACAATGGT |
| | | TCATGTCTATAAATCTCCGGCTTCGGTACCTTCTCCCC |
| | | AATTGAATACATTGTCAAAATGAATGGTTGAACTATT |
| | | AGGTTCGCCAGTTTCGTTATTAAGAAAACTGTTAAAAT |
| | | CAAATTCCATATCATCGGTTCCAGTGGGAGGACCAGT |
| | | TCCATCGCCAAAATCCTGTAAGAATCCATTGTCAGAA |
| | | CCTGTAAAGTCAGTTTGAGATGAAATTTTTCCGGTCTT |
| | | TGTTGACTTGGAAGCTTCGTTAAGGTTAGGTGAAACA |
| | | GTTTGATCAACCAGCGGCTCCCGTTTTCGTCGCTTAGT |
| | | AG |
| 9 | PpTRP1 5′ | GCGGAAACGGCAGTAAACAATGGAGCTTCATTAGTGG |
| | region and ORF | GTGTTATTATGGTCCCTGGCCGGGAACGAACGGTGAA |
| | | ACAAGAGGTTGCGAGGGAAATTTCGCAGATGGTGCGG |
| | | GAAAAGAGAATTTCAAAGGGCTCAAAATACTTGGATT |
| | | CCAGACAACTGAGGAAAGAGTGGGACGACTGTCCTCT |
| | | GGAAGACTGGTTTGAGTACAACGTGAAAGAAATAAAC |
| | | AGCAGTGGTCCATTTTTAGTTGGAGTTTTTCGTAATCA |
| | | AAGTATAGATGAAATCCAGCAAGCTATCCACACTCAT |
| | | GGTTTGGATTTCGTCCAACTACATGGGTCTGAGGATTT |
| | | TGATTCGTATATACGCAATATCCCAGTTCCTGTGATTA |
| | | CCAGATACACAGATAATGCCGTCGATGGTCTTACCGG |
| | | AGAAGACCTCGCTATAAATAGGGCCCTGGTGCTACTG |
| | | GACAGCGAGCAAGGAGGTGAAGGAAAAACCATCGAT |
| | | TGGGCTCGTGCACAAAAATTTGGAGAACGTAGAGGAA |
| | | AATATTTACTAGCCGGAGGTTTGACACCTGATAATGTT |
| | | GCTCATGCTCGATCTCATACTGGCTGTATTGGTGTTGA |
| | | CGTCTCTGGTGGGGTAGAAACAAATGCCTCAAAAGAT |
| | | ATGGACAAGATCACACAATTTATCAGAAACGCTACAT |
| | | AA |
| 10 | PpTRP1 3′ | AAGTCAATTAAATACACGCTTGAAAGGACATTACATA |
| | region | GCTTTCGATTTAAGCAGAACCAGAAATGTAGAACCAC |
| | | TTGTCAATAGATTGGTCAATCTTAGCAGGAGCGGCTG |
| | | GGCTAGCAGTTGGAACAGCAGAGGTTGCTGAAGGTGA |
| | | GAAGGATGGAGTGGATTGCAAAGTGGTGTTGGTTAAG |
| | | TCAATCTCACCAGGGCTGGTTTTGCCAAAAATCAACTT |
| | | CTCCCAGGCTTCACGGCATTCTTGAATGACCTCTTCTG |
| | | CATACTTCTTGTTCTTGCATTCACCAGAGAAAGCAAAC |
| | | TGGTTCTCAGGTTTTCCATCAGGGATCTTGTAAATTCT |
| | | GAACCATTCGTTGGTAGCTCTCAACAAGCCCGGCATG |
| | | TGCTTTTCAACATCCTCGATGTCATTGAGCTTAGGAGC |
| | | CAATGGGTCGTTGATGTCGATGACGATGACCTTCCAG |
| | | TCAGTCTCTCCCTCATCCAACAAAGCCATAACACCGA |
| | | GGACCTTGACTTGCTTGACCTGTCCAGTGTAACCTACG |
| | | GCTTCACCAATTTCGCAAACGTCCAATGGATCATTGTC |
| | | ACCCTTGGCCTTGGTCTCTGGATGAGTGACGTTAGGGT |
| | | CTTCCCATGTCTGAGGGAAGGCACCGTAGTTGTGAAT |
| | | GTATCCGTGGTGAGGGAAACAGTTACGAACGAAACGA |
| | | AGTTTTCCCTTCTTTGTGTCCTGAAGAATTGGGTTCAG |
| | | TTTCTCCTCCTTGGAAATCTCCAACTTGGCGTTGGTCC |
| | | AACGGGGGACTTCAACAACCATGTTGAGAACCTTCTT |
| | | GGATTCGTCAGCATAAAGTGGGATGTCGTGGAAAGGA |
| | | GATACGACTT |
| 11 | ScARR3 ORF | ATGTCAGAAGATCAAAAAAGTGAAAATTCCGTACCTT |
| | | CTAAGGTTAATATGGTGAATCGCACCGATATACTGAC |
| | | TACGATCAAGTCATTGTCATGGCTTGACTTGATGTTGC |
| | | CATTTACTATAATTCTCTCCATAATCATTGCAGTAATA |
| | | ATTTCTGTCTATGTGCCTTCTTCCCGTCACACTTTTGAC |
| | | GCTGAAGGTCATCCCAATCTAATGGGAGTGTCCATTC |
| | | CTTTGACTGTTGGTATGATTGTAATGATGATTCCCCCG |
| | | ATCTGCAAAGTTTCCTGGGAGTCTATTCACAAGTACTT |
| | | CTACAGGAGCTATATAAGGAAGCAACTAGCCCTCTCG |
| | | TTATTTTTGAATTGGGTCATCGGTCCTTTGTTGATGAC |
| | | AGCATTGGCGTGGATGGCGCTATTCGATTATAAGGAA |
| | | TACCGTCAAGGCATTATTATGATCGGAGTAGCTAGAT |
| | | GCATTGCCATGGTGCTAATTTGGAATCAGATTGCTGG |
| | | AGGAGACAATGATCTCTGCGTCGTGCTTGTTATTACAA |
| | | ACTCGCTTTTACAGATGGTATTATATGCACCATTGCAG |
| | | ATATTTTACTGTTATGTTATTTCTCATGACCACCTGAA |
| | | TACTTCAAATAGGGTATTATTCGAAGAGGTTGCAAAG |
| | | TCTGTCGGAGTTTTTCTCGGCATACCACTGGGAATTGG |
| | | CATTATCATACGTTTGGGAAGTCTTACCATAGCTGGTA |
| | | AAAGTAATTATGAAAAATACATTTTGAGATTTATTTCT |
| | | CCATGGGCAATGATCGGATTTCATTACACTTTATTTGT |
| | | TATTTTTATTAGTAGAGGTTATCAATTTATCCACGAAA |
| | | TTGGTTCTGCAATATTGTGCTTTGTCCCATTGGTGCTTT |
| | | ACTTCTTTATTGCATGGTTTTTGACCTTCGCATTAATG |
| | | AGGTACTTATCAATATCTAGGAGTGATACACAAAGAG |
| | | AATGTAGCTGTGACCAAGAACTACTTTTAAAGAGGGT |
| | | CTGGGGAAGAAAGTCTTGTGAAGCTAGCTTTTCTATTA |
| | | CGATGACGCAATGTTTCACTATGGCTTCAAATAATTTT |
| | | GAACTATCCCTGGCAATTGCTATTTCCTTATATGGTAA |
| | | CAATAGCAAGCAAGCAATAGCTGCAACATTTGGGCCG |
| | | TTGCTAGAAGTTCCAATTTTATTGATTTTGGCAATAGT |
| | | CGCGAGAATCCTTAAACCATATTATATATGGAACAAT |
| | | AGAAATTAA |
| 12 | PpURA6 region | CAAATGCAAGAGGACATTAGAAATGTGTTTGGTAAGA |
| | | ACATGAAGCCGGAGGCATACAAACGATTCACAGATTT |
| | | GAAGGAGGAAAACAAACTGCATCCACCGGAAGTGCC |
| | | AGCAGCCGTGTATGCCAACCTTGCTCTCAAAGGCATT |
| | | CCTACGGATCTGAGTGGGAAATATCTGAGATTCACAG |
| | | ACCCACTATTGGAACAGTACCAAACCTAGTTTGGCCG |
| | | ATCCATGATTATGTAATGCATATAGTTTTTGTCGATGC |
| | | TCACCCGTTTCGAGTCTGTCTCGTATCGTCTTACGTAT |
| | | AAGTTCAAGCATGTTTACCAGGTCTGTTAGAAACTCCT |
| | | TTGTGAGGGCAGGACCTATTCGTCTCGGTCCCGTTGTT |
| | | TCTAAGAGACTGTACAGCCAAGCGCAGAATGGTGGCA |
| | | TTAACCATAAGAGGATTCTGATCGGACTTGGTCTATTG |
| | | GCTATTGGAACCACCCTTTACGGGACAACCAACCCTA |
| | | CCAAGACTCCTATTGCATTTGTGGAACCAGCCACGGA |
| | | AAGAGCGTTTAAGGACGGAGACGTCTCTGTGATTTTT |
| | | GTTCTCGGAGGTCCAGGAGCTGGAAAAGGTACCCAAT |
| | | GTGCCAAACTAGTGAGTAATTACGGATTTGTTCACCTG |
| | | TCAGCTGGAGACTTGTTACGTGCAGAACAGAAGAGGG |
| | | AGGGGTCTAAGTATGGAGAGATGATTTCCCAGTATAT |
| | | CAGAGATGGACTGATAGTACCTCAAGAGGTCACCATT |
| | | GCGCTCTTGGAGCAGGCCATGAAGGAAAACTTCGAGA |
| | | AAGGGAAGACACGGTTCTTGATTGATGGATTCCCTCG |
| | | TAAGATGGACCAGGCCAAAACTTTTGAGGAAAAAGTC |
| | | GCAAAGTCCAAGGTGACACTTTTCTTTGATTGTCCCGA |
| | | ATCAGTGCTCCTTGAGAGATTACTTAAAAGAGGACAG |
| | | ACAAGCGGAAGAGAGGATGATAATGCGGAGAGTATC |
| | | AAAAAAAGATTCAAAACATTCGTGGAAACTTCGATGC |
| | | CTGTGGTGGACTATTTCGGGAAGCAAGGACGCGTTTT |
| | | GAAGGTATCTTGTGACCACCCTGTGGATCAAGTGTATT |
| | | CACAGGTTGTGTCGGTGCTAAAAGAGAAGGGGATCTT |
| | | TGCCGATAACGAGACGGAGAATAAATAA |
| 13 | NatR expression | TGTTTAGCTTGCCTCGTCCCCGCCGGGTCACCCGGCCA |
| | cassette (CDS | GCGACATGGAGGCCCAGAATACCCTCCTTGACAGTCT |
| | 385-954, | TGACGTGCGCAGCTCAGGGGCATGATGTGACTGTCGC |
| | represented in | CCGTACATTTAGCCCATACATCCCCATGTATAATCATT |
| | bold) | TGCATCCATACATTTTGATGGCCGCACGGCGCGAAGC |
| | | AAAAATTACGGCTCCTCGCTGCAGACCTGCGAGCAGG |
| | | GAAACGCTCCCCTCACAGACGCGTTGAATTGTCCCCA |
| | | CGCCGCGCCCCTGTAGAGAAATATAAAAGGTTAGGAT |
| | | TTGCCACTGAGGTTCTTCTTTCATATACTTCCTTTTAAA |
| | | ATCTTGCTAGGATACAGTTCTCACATCACATCCGAACA |
| | | TAAACAACCATGGGTACCACTCTTGACGACACGGCT |
| | | TACCGGTACCGCACCAGTGTCCCGGGGGACGCCGA |
| | | GGCCATCGAGGCACTGGATGGGTCCTTCACCACCG |
| | | ACACCGTCTTCCGCGTCACCGCCACCGGGGACGGC |
| | | TTCACCCTGCGGGAGGTGCCGGTGGACCCGCCCCT |
| | | GACCAAGGTGTTCCCCGACGACGAATCGGACGACG |
| | | AATCGGACGACGGGGAGGACGGCGACCCGGACTC |
| | | CCGGACGTTCGTCGCGTACGGGGACGACGGCGACC |
| | | TGGCGGGCTTCGTGGTCGTCTCGTACTCCGGCTGG |
| | | AACCGCCGGCTGACCGTCGAGGACATCGAGGTCGC |
| | | CCCGGAGCACCGGGGGCACGGGGTCGGGCGCGCG |
| | | TTGATGGGGCTCGCGACGGAGTTCGCCCGCGAGCG |
| | | GGGCGCCGGGCACCTCTGGCTGGAGGTCACCAACG |
| | | TCAACGCACCGGCGATCCACGCGTACCGGCGGATG |
| | | GGGTTCACCCTCTGCGGCCTGGACACCGCCCTGTA |
| | | CGACGGCACCGCCTCGGACGGCGAGCAGGCGCTCT |
| | | ACATGAGCATGCCCTGCCCCTAATCAGTACTGACAA |
| | | TAAAAAGATTCTTGTTTTCAAGAACTTGTCATTTGTAT |
| | | AGTTTTTTTATATTGTAGTTGTTCTATTTTAATCAAATG |
| | | TTAGCGTGATTTATATTTTTTTTCGCCTCGACATCATCT |
| | | GCCCAGATGCGAAGTTAAGTGCGCAGAAAGTAATATC |
| | | ATGCGTCAATCGTATGTGAATGCTGGTCGCTATACTGC |
| | | TGTCGATTCGATACTAACGCCGCCATCCAGTGTCGAA |
| | | AAC |
| 14 | Sequence of the | ATGGCCAAGTTGACCAGTGCCGTTCCGGTGCTCACCG |
| | Sh ble ORF | CGCGCGACGTCGCCGGAGCGGTCGAGTTCTGGACCGA |
| | (Zeocin | CCGGCTCGGGTTCTCCCGGGACTTCGTGGAGGACGAC |
| | resistance | TTCGCCGGTGTGGTCCGGGACGACGTGACCCTGTTCAT |
| | marker): | CAGCGCGGTCCAGGACCAGGTGGTGCCGGACAACACC |
| | | CTGGCCTGGGTGTGGGTGCGCGGCCTGGACGAGCTGT |
| | | ACGCCGAGTGGTCGGAGGTCGTGTCCACGAACTTCCG |
| | | GGACGCCTCCGGGCCGGCCATGACCGAGATCGGCGAG |
| | | CAGCCGTGGGGGCGGGAGTTCGCCCTGCGCGACCCGG |
| | | CCGGCAACTGCGTGCACTTCGTGGCCGAGGAGCAGGA |
| | | CTGA |
| 15 | PpAOX1 TT | TCAAGAGGATGTCAGAATGCCATTTGCCTGAGAGATG |
| | | CAGGCTTCATTTTGATACTTTTTTATTTGTAACCTATAT |
| | | AGTATAGGATTTTTTTTGTCATTTTGTTTCTTCTCGTAC |
| | | GAGCTTGCTCCTGATCAGCCTATCTCGCAGCTGATGAA |
| | | TATCTTGTGGTAGGGGTTTGGGAAAATCATTCGAGTTT |
| | | GATGTTTTTCTTGGTATTTCCCACTCCTCTTCAGAGTAC |
| | | AGAAGATTAAGTGAGACGTTCGTTTGTGCA |
| 16 | SeTEF1 | GATCCCCCACACACCATAGCTTCAAAATGTTTCTACTC |
| | promoter | CTTTTTTACTCTTCCAGATTTTCTCGGACTCCGCGCATC |
| | | GCCGTACCACTTCAAAACACCCAAGCACAGCATACTA |
| | | AATTTCCCCTCTTTCTTCCTCTAGGGTGTCGTTAATTAC |
| | | CCGTACTAAAGGTTTGGAAAAGAAAAAAGAGACCGC |
| | | CTCGTTTCTTTTTCTTCGTCGAAAAAGGCAATAAAAAT |
| | | TTTTATCACGTTTCTTTTTCTTGAAAATTTTTTTTTTTG |
| | | ATTTTTTTCTCTTTCGATGACCTCCCATTGATATTTAAG |
| | | TTAATAAACGGTCTTCAATTTCTCAAGTTTCAGTTTCA |
| | | TTTTTCTTGTTCTATTACAACTTTTTTTACTTCTTGCTC |
| | | ATTAGAAAGAAAGCATAGCAATCTAATCTAAGTTTTA |
| | | ATTACAAA |
| 17 | S. cerevisiae | AGGCCTCGCAACAACCTATAATTGAGTTAAGTGCCTTT |
| | invertase gene | CCAAGCTAAAAAGTTTGAGGTTATAGGGGCTTAGCAT |
| | (ScSUC2) ORF | CCACACGTCACAATCTCGGGTATCGAGTATAGTATGT |
| | underlined | AGAATTACGGCAGGAGGTTTCCCAATGAACAAAGGAC |
| | | AGGGGCACGGTGAGCTGTCGAAGGTATCCATTTTATC |
| | | ATGTTTCGTTTGTACAAGCACGACATACTAAGACATTT |
| | | ACCGTATGGGAGTTGTTGTCCTAGCGTAGTTCTCGCTC |
| | | CCCCAGCAAAGCTCAAAAAAGTACGTCATTTAGAATA |
| | | GTTTGTGAGCAAATTACCAGTCGGTATGCTACGTTAG |
| | | AAAGGCCCACAGTATTCTTCTACCAAAGGCGTGCCTTT |
| | | GTTGAACTCGATCCATTATGAGGGCTTCCATTATTCCC |
| | | CGCATTTTATTACTCTGAACAGGAATAAAAAGAAAA |
| | | AACCCAGTTTAGGAAATTATCCGGGGGCGAAGAAATA |
| | | CGCGTAGCGTTAATCGACCCCACGTCCAGGGTTTTTCC |
| | | ATGGAGGTTTCTGGAAAAACTGACGAGGAATGTGATT |
| | | ATAAATCCCTTTATGTGATGTCTAAGACTTTTAAGGTA |
| | | CGCCCGATGTTTGCCTATTACCATCATAGAGACGTTTC |
| | | TTTTCGAGGAATGCTTAAACGACTTTGTTTGACAAAAA |
| | | TGTTGCCTAAGGGCTCTATAGTAAACCATTTGGAAGA |
| | | AAGATTTGACGACTTTTTTTTTTTGGATTTCGATCCTAT |
| | | AATCCTTCCTCCTGAAAAGAAACATATAAATAGATAT |
| | | GTATTATTCTTCAAAACATTCTCTTGTTCTTGTGCTTTT |
| | | TTTTTACCATATATCTTACTTTTTTTTTTCTCTCAGAGA |
| | | AACAAGCAAAACAAAAAGCTTTTCTTTTCACTAACGT |
| | | ATATGATGCTTTTGCAAGCTTTCCTTTTCCTTTTGGCTG |
| | | GTTTTGCAGCCAAAATATCTGCATCAATGACAAACGA |
| | | AACTAGCGATAGACCTTTGGTCCACTTCACACCCAAC |
| | | AAGGGCTGGATGAATGACCCAAATGGGTTGTGGTACG |
| | | ATGAAAAAGATGCCAAATGGCATCTGTACTTTCAATA |
| | | CAACCCAAATGACACCGTATGGGGTACGCCATTGTTT |
| | | TGGGGCCATGCTACTTCCGATGATTTGACTAATTGGGA |
| | | AGATCAACCCATTGCTATCGCTCCCAAGCGTAACGAT |
| | | TCAGGTGCTTTCTCTGGCTCCATGGTGGTTGATTACAA |
| | | CAACACGAGTGGGTTTTTCAATGATACTATTGATCCAA |
| | | GACAAAGATGCGTTGCGATTTGGACTTATAACACTCC |
| | | TGAAAGTGAAGAGCAATACATTAGCTATTCTCTTGAT |
| | | GGTGGTTACACTTTTACTGAATACCAAAAGAACCCTG |
| | | TTTTAGCTGCCAACTCCACTCAATTCAGAGATCCAAAG |
| | | GTGTTCTGGTATGAACCTTCTCAAAAATGGATTATGAC |
| | | GGCTGCCAAATCACAAGACTACAAAATTGAAATTTAC |
| | | TCCTCTGATGACTTGAAGTCCTGGAAGCTAGAATCTGC |
| | | ATTTGCCAATGAAGGTTTCTTAGGCTACCAATACGAAT |
| | | GTCCAGGTTTGATTGAAGTCCCAACTGAGCAAGATCC |
| | | TTCCAAATCTTATTGGGTCATGTTTATTTCTATCAACC |
| | | CAGGTGCACCTGCTGGCGGTTCCTTCAACCAATATTTT |
| | | GTTGGATCCTTCAATGGTACTCATTTTGAAGCGTTTGA |
| | | CAATCAATCTAGAGTGGTAGATTTTGGTAAGGACTAC |
| | | TATGCCTTGCAAACTTTCTTCAACACTGACCCAACCTA |
| | | CGGTTCAGCATTAGGTATTGCCTGGGCTTCAAACTGG |
| | | GAGTACAGTGCCTTTGTCCCAACTAACCCATGGAGAT |
| | | CATCCATGTCTTTGGTCCGCAAGTTTTCTTTGAACACT |
| | | GAATATCAAGCTAATCCAGAGACTGAATTGATCAATT |
| | | TGAAAGCCGAACCAATATTGAACATTAGTAATGCTGG |
| | | TCCCTGGTCTCGTTTTGCTACTAACACAACTCTAACTA |
| | | AGGCCAATTCTTACAATGTCGATTTGAGCAACTCGACT |
| | | GGTACCCTAGAGTTTGAGTTGGTTTACGCTGTTAACAC |
| | | CACACAAACCATATCCAAATCCGTCTTTGCCGACTTAT |
| | | CACTTTGGTTCAAGGGTTTAGAAGATCCTGAAGAATA |
| | | TTTGAGAATGGGTTTTGAAGTCAGTGCTTCTTCCTTCT |
| | | TTTTGGACCGTGGTAACTCTAAGGTCAAGTTTGTCAAG |
| | | GAGAACCCATATTTCACAAACAGAATGTCTGTCAACA |
| | | ACCAACCATTCAAGTCTGAGAACGACCTAAGTTACTA |
| | | TAAAGTGTACGGCCTACTGGATCAAAACATCTTGGAA |
| | | TTGTACTTCAACGATGGAGATGTGGTTTCTACAAATAC |
| | | CTACTTCATGACCACCGGTAACGCTCTAGGATCTGTGA |
| | | ACATGACCACTGGTGTCGATAATTTGTTCTACATTGAC |
| | | AAGTTCCAAGTAAGGGAAGTAAAATAGAGGTTATAA |
| | | AACTTATTGTCTTTTTTATTTTTTTCAAAAGCCATTCTA |
| | | AAGGGCTTTAGCTAACGAGTGACGAATGTAAAACTTT |
| | | ATGATTTCAAAGAATACCTCCAAACCATTGAAAATGT |
| | | ATTTTTATTTTTATTTTCTCCCGACCCCAGTTACCTGGA |
| | | ATTTGTTCTTTATGTACTTTATATAAGTATAATTCTCTT |
| | | AAAAATTTTTACTACTTTGCAATAGACATCATTTTTTC |
| | | ACGTAATAAACCCACAATCGTAATGTAGTTGCCTTAC |
| | | ACTACTAGGATGGACCTTTTTGCCTTTATCTGTTTTGT |
| | | ACTGACACAATGAAACCGGGTAAAGTATTAGTTATGT |
| | | GAAAATTTAAAAGCATTAAGTAGAAGTATACCATATT |
| | | GTAAAAAAAAAAAGCGTTGTCTTCTACGTAAAAGTGT |
| | | TCTCAAAAAGAAGTAGTGAGGGAAATGGATACCAAGC |
| | | TATCTGTAACAGGAGCTAAAAAATCTCAGGGAAAAGC |
| | | TTCTGGTTTGGGAAACGGTCGAC |
| 18 | Sequence of the | ATCGGCCTTTGTTGATGCAAGTTTTACGTGGATCATGG |
| | 5′-Region used | ACTAAGGAGTTTTATTTGGACCAAGTTCATCGTCCTAG |
| | for knock out of | ACATTACGGAAAGGGTTCTGCTCCTCTTTTTGGAAACT |
| | PpURA5: | TTTTGGAACCTCTGAGTATGACAGCTTGGTGGATTGTA |
| | | CCCATGGTATGGCTTCCTGTGAATTTCTATTTTTTCTAC |
| | | ATTGGATTCACCAATCAAAACAAATTAGTCGCCATGG |
| | | CTTTTTGGCTTTTGGGTCTATTTGTTTGGACCTTCTTGG |
| | | AATATGCTTTGCATAGATTTTTGTTCCACTTGGACTAC |
| | | TATCTTCCAGAGAATCAAATTGCATTTACCATTCATTT |
| | | CTTATTGCATGGGATACACCACTATTTACCAATGGATA |
| | | AATACAGATTGGTGATGCCACCTACACTTTTCATTGTA |
| | | CTTTGCTACCCAATCAAGACGCTCGTCTTTTCTGTTCT |
| | | ACCATATTACATGGCTTGTTCTGGATTTGCAGGTGGAT |
| | | TCCTGGGCTATATCATGTATGATGTCACTCATTACGTT |
| | | CTGCATCACTCCAAGCTGCCTCGTTATTTCCAAGAGTT |
| | | GAAGAAATATCATTTGGAACATCACTACAAGAATTAC |
| | | GAGTTAGGCTTTGGTGTCACTTCCAAATTCTGGGACAA |
| | | AGTCTTTGGGACTTATCTGGGTCCAGACGATGTGTATC |
| | | AAAAGACAAATTAGAGTATTTATAAAGTTATGTAAGC |
| | | AAATAGGGGCTAATAGGGAAAGAAAAATTTTGGTTCT |
| | | TTATCAGAGCTGGCTCGCGCGCAGTGTTTTTCGTGCTC |
| | | CTTTGTAATAGTCATTTTTGACTACTGTTCAGATTGAA |
| | | ATCACATTGAAGATGTCACTCGAGGGGTACCAAAAAA |
| | | GGTTTTTGGATGCTGCAGTGGCTTCGC |
| 19 | Sequence of the | GGTCTTTTCAACAAAGCTCCATTAGTGAGTCAGCTGGC |
| | 3′-Region used | TGAATCTTATGCACAGGCCATCATTAACAGCAACCTG |
| | for knock out of | GAGATAGACGTTGTATTTGGACCAGCTTATAAAGGTA |
| | PpURA5: | TTCCTTTGGCTGCTATTACCGTGTTGAAGTTGTACGAG |
| | | CTCGGCGGCAAAAAATACGAAAATGTCGGATATGCGT |
| | | TCAATAGAAAAGAAAAGAAAGACCACGGAGAAGGTG |
| | | GAAGCATCGTTGGAGAAAGTCTAAAGAATAAAAGAGT |
| | | ACTGATTATCGATGATGTGATGACTGCAGGTACTGCT |
| | | ATCAACGAAGCATTTGCTATAATTGGAGCTGAAGGTG |
| | | GGAGAGTTGAAGGTAGTATTATTGCCCTAGATAGAAT |
| | | GGAGACTACAGGAGATGACTCAAATACCAGTGCTACC |
| | | CAGGCTGTTAGTCAGAGATATGGTACCCCTGTCTTGA |
| | | GTATAGTGACATTGGACCATATTGTGGCCCATTTGGGC |
| | | GAAACTTTCACAGCAGACGAGAAATCTCAAATGGAAA |
| | | CGTATAGAAAAAAGTATTTGCCCAAATAAGTATGAAT |
| | | CTGCTTCGAATGAATGAATTAATCCAATTATCTTCTCA |
| | | CCATTATTTTCTTCTGTTTCGGAGCTTTGGGCACGGCG |
| | | GCGGGTGGTGCGGGCTCAGGTTCCCTTTCATAAACAG |
| | | ATTTAGTACTTGGATGCTTAATAGTGAATGGCGAATGC |
| | | AAAGGAACAATTTCGTTCATCTTTAACCCTTTCACTCG |
| | | GGGTACACGTTCTGGAATGTACCCGCCCTGTTGCAACT |
| | | CAGGTGGACCGGGCAATTCTTGAACTTTCTGTAACGTT |
| | | GTTGGATGTTCAACCAGAAATTGTCCTACCAACTGTAT |
| | | TAGTTTCCTTTTGGTCTTATATTGTTCATCGAGATACTT |
| | | CCCACTCTCCTTGATAGCCACTCTCACTCTTCCTGGAT |
| | | TACCAAAATCTTGAGGATGAGTCTTTTCAGGCTCCAG |
| | | GATGCAAGGTATATCCAAGTACCTGCAAGCATCTAAT |
| | | ATTGTCTTTGCCAGGGGGTTCTCCACACCATACTCCTT |
| | | TTGGCGCATGC |
| 20 | Sequence of the | TCTAGAGGGACTTATCTGGGTCCAGACGATGTGTATC |
| | PpURA5 | AAAAGACAAATTAGAGTATTTATAAAGTTATGTAAGC |
| | auxotrophic | AAATAGGGGCTAATAGGGAAAGAAAAATTTTGGTTCT |
| | marker: | TTATCAGAGCTGGCTCGCGCGCAGTGTTTTTCGTGCTC |
| | | CTTTGTAATAGTCATTTTTGACTACTGTTCAGATTGAA |
| | | ATCACATTGAAGATGTCACTGGAGGGGTACCAAAAAA |
| | | GGTTTTTGGATGCTGCAGTGGCTTCGCAGGCCTTGAAG |
| | | TTTGGAACTTTCACCTTGAAAAGTGGAAGACAGTCTC |
| | | CATACTTCTTTAACATGGGTCTTTTCAACAAAGCTCCA |
| | | TTAGTGAGTCAGCTGGCTGAATCTTATGCTCAGGCCAT |
| | | CATTAACAGCAACCTGGAGATAGACGTTGTATTTGGA |
| | | CCAGCTTATAAAGGTATTCCTTTGGCTGCTATTACCGT |
| | | GTTGAAGTTGTACGAGCTGGGCGGCAAAAAATACGAA |
| | | AATGTCGGATATGCGTTCAATAGAAAAGAAAAGAAAG |
| | | ACCACGGAGAAGGTGGAAGCATCGTTGGAGAAAGTCT |
| | | AAAGAATAAAAGAGTACTGATTATCGATGATGTGATG |
| | | ACTGCAGGTACTGCTATCAACGAAGCATTTGCTATAA |
| | | TTGGAGCTGAAGGTGGGAGAGTTGAAGGTTGTATTAT |
| | | TGCCCTAGATAGAATGGAGACTACAGGAGATGACTCA |
| | | AATACCAGTGCTACCCAGGCTGTTAGTCAGAGATATG |
| | | GTACCCCTGTCTTGAGTATAGTGACATTGGACCATATT |
| | | GTGGCCCATTTGGGCGAAACTTTCACAGCAGACGAGA |
| | | AATCTCAAATGGAAACGTATAGAAAAAAGTATTTGCC |
| | | CAAATAAGTATGAATCTGCTTCGAATGAATGAATTAA |
| | | TCCAATTATCTTCTCACCATTATTTTCTTCTGTTTCGGA |
| | | GCTTTGGGCACGGCGGCGGATCC |
| 21 | Sequence of the | CCTGCACTGGATGGTGGCGCTGGATGGTAAGCCGCTG |
| | part of the Ec | GCAAGCGGTGAAGTGCCTCTGGATGTCGCTCCACAAG |
| | lacZ gene that | GTAAACAGTTGATTGAACTGCCTGAACTACCGCAGCC |
| | was used to | GGAGAGCGCCGGGCAACTCTGGCTCACAGTACGCGTA |
| | construct the | GTGCAACCGAACGCGACCGCATGGTCAGAAGCCGGGC |
| | PpURA5 blaster | ACATCAGCGCCTGGCAGCAGTGGCGTCTGGCGGAAAA |
| | (recyclable | CCTCAGTGTGACGCTCCCCGCCGCGTCCCACGCCATCC |
| | auxotrophic | CGCATCTGACCACCAGCGAAATGGATTTTTGCATCGA |
| | marker) | GCTGGGTAATAAGCGTTGGCAATTTAACCGCCAGTCA |
| | | GGCTTTCTTTCACAGATGTGGATTGGCGATAAAAAAC |
| | | AACTGCTGACGCCGCTGCGCGATCAGTTCACCCGTGC |
| | | ACCGCTGGATAACGACATTGGCGTAAGTGAAGCGACC |
| | | CGCATTGACCCTAACGCCTGGGTCGAACGCTGGAAGG |
| | | CGGCGGGCCATTACCAGGCCGAAGCAGCGTTGTTGCA |
| | | GTGCACGGCAGATACACTTGCTGATGCGGTGCTGATT |
| | | ACGACCGCTCACGCGTGGCAGCATCAGGGGAAAACCT |
| | | TATTTATCAGCCGGAAAACCTACCGGATTGATGGTAG |
| | | TGGTCAAATGGCGATTACCGTTGATGTTGAAGTGGCG |
| | | AGCGATACACCGCATCCGGCGCGGATTGGCCTGAACT |
| | | GCCAG |
| 22 | Sequence of the | AAAACCTTTTTTCCTATTCAAACACAAGGCATTGCTTC |
| | 5′-Region used | AACACGTGTGCGTATCCTTAACACAGATACTCCATACT |
| | for knock out of | TCTAATAATGTGATAGACGAATACAAAGATGTTCACT |
| | PpOCH1: | CTGTGTTGTGTCTACAAGCATTTCTTATTCTGATTGGG |
| | | GATATTCTAGTTACAGCACTAAACAACTGGCGATACA |
| | | AACTTAAATTAAATAATCCGAATCTAGAAAATGAACT |
| | | TTTGGATGGTCCGCCTGTTGGTTGGATAAATCAATACC |
| | | GATTAAATGGATTCTATTCCAATGAGAGAGTAATCCA |
| | | AGACACTCTGATGTCAATAATCATTTGCTTGCAACAAC |
| | | AAACCCGTCATCTAATCAAAGGGTTTGATGAGGCTTA |
| | | CCTTCAATTGCAGATAAACTCATTGCTGTCCACTGCTG |
| | | TATTATGTGAGAATATGGGTGATGAATCTGGTCTTCTC |
| | | CACTCAGCTAACATGGCTGTTTGGGCAAAGGTGGTAC |
| | | AATTATACGGAGATCAGGCAATAGTGAAATTGTTGAA |
| | | TATGGCTACTGGACGATGCTTCAAGGATGTACGTCTA |
| | | GTAGGAGCCGTGGGAAGATTGCTGGCAGAACCAGTTG |
| | | GCACGTCGCAACAATCCCCAAGAAATGAAATAAGTGA |
| | | AAACGTAACGTCAAAGACAGCAATGGAGTCAATATTG |
| | | ATAACACCACTGGCAGAGCGGTTCGTACGTCGTTTTG |
| | | GAGCCGATATGAGGCTCAGCGTGCTAACAGCACGATT |
| | | GACAAGAAGACTCTCGAGTGACAGTAGGTTGAGTAAA |
| | | GTATTCGCTTAGATTCCCAACCTTCGTTTTATTCTTTCG |
| | | TAGACAAAGAAGCTGCATGCGAACATAGGGACAACTT |
| | | TTATAAATCCAATTGTCAAACCAACGTAAAACCCTCT |
| | | GGCACCATTTTCAACATATATTTGTGAAGCAGTACGC |
| | | AATATCGATAAATACTCACCGTTGTTTGTAACAGCCCC |
| | | AACTTGCATACGCCTTCTAATGACCTCAAATGGATAA |
| | | GCCGCAGCTTGTGCTAACATACCAGCAGCACCGCCCG |
| | | CGGTCAGCTGCGCCCACACATATAAAGGCAATCTACG |
| | | ATCATGGGAGGAATTAGTTTTGACCGTCAGGTCTTCA |
| | | AGAGTTTTGAACTCTTCTTCTTGAACTGTGTAACCTTT |
| | | TAAATGACGGGATCTAAATACGTCATGGATGAGATCA |
| | | TGTGTGTAAAAACTGACTCCAGCATATGGAATCATTC |
| | | CAAAGATTGTAGGAGCGAACCCACGATAAAAGTTTCC |
| | | CAACCTTGCCAAAGTGTCTAATGCTGTGACTTGAAATC |
| | | TGGGTTCCTCGTTGAAGACCCTGCGTACTATGCCCAAA |
| | | AACTTTCCTCCACGAGCCCTATTAACTTCTCTATGAGT |
| | | TTCAAATGCCAAACGGACACGGATTAGGTCCAATGGG |
| | | TAAGTGAAAAACACAGAGCAAACCCCAGCTAATGAG |
| | | CCGGCCAGTAACCGTCTTGGAGCTGTTTCATAAGAGT |
| | | CATTAGGGATCAATAACGTTCTAATCTGTTCATAACAT |
| | | ACAAATTTTATGGCTGCATAGGGAAAAATTCTCAACA |
| | | GGGTAGCCGAATGACCCTGATATAGACCTGCGACACC |
| | | ATCATACCCATAGATCTGCCTGACAGCCTTAAAGAGC |
| | | CCGCTAAAAGACCCGGAAAACCGAGAGAACTCTGGAT |
| | | TAGCAGTCTGAAAAAGAATCTTCACTCTGTCTAGTGG |
| | | AGCAATTAATGTCTTAGCGGCACTTCCTGCTACTCCGC |
| | | CAGCTACTCCTGAATAGATCACATACTGCAAAGACTG |
| | | CTTGTCGATGACCTTGGGGTTATTTAGCTTCAAGGGCA |
| | | ATTTTTGGGACATTTTGGACACAGGAGACTCAGAAAC |
| | | AGACACAGAGCGTTCTGAGTCCTGGTGCTCCTGACGT |
| | | AGGCCTAGAACAGGAATTATTGGCTTTATTTGTTTGTC |
| | | CATTTCATAGGCTTGGGGTAATAGATAGATGACAGAG |
| | | AAATAGAGAAGACCTAATATTTTTTGTTCATGGCAAAT |
| | | CGCGGGTTCGCGGTCGGGTCACACACGGAGAAGTAAT |
| | | GAGAAGAGCTGGTAATCTGGGGTAAAAGGGTTCAAAA |
| | | GAAGGTCGCCTGGTAGGGATGCAATACAAGGTTGTCT |
| | | TGGAGTTTACATTGACCAGATGATTTGGCTTTTTCTCT |
| | | GTTCAATTCACATTTTTCAGCGAGAATCGGATTGACGG |
| | | AGAAATGGCGGGGTGTGGGGTGGATAGATGGCAGAA |
| | | ATGCTCGCAATCACCGCGAAAGAAAGACTTTATGGAA |
| | | TAGAACTACTGGGTGGTGTAAGGATTACATAGCTAGT |
| | | CCAATGGAGTCCGTTGGAAAGGTAAGAAGAAGCTAAA |
| | | ACCGGCTAAGTAACTAGGGAAGAATGATCAGACTTTG |
| | | ATTTGATGAGGTCTGAAAATACTCTGCTGCTTTTTCAG |
| | | TTGCTTTTTCCCTGCAACCTATCATTTTCCTTTTCATAA |
| | | GCCTGCCTTTTCTGTTTTCACTTATATGAGTTCCGCCG |
| | | AGACTTCCCCAAATTCTCTCCTGGAACATTCTCTATCG |
| | | CTCTCCTTCCAAGTTGCGCCCCCTGGCACTGCCTAGTA |
| | | ATATTACCACGCGACTTATATTCAGTTCCACAATTTCC |
| | | AGTGTTCGTAGCAAATATCATCAGCCATGGCGAAGGC |
| | | AGATGGCAGTTTGCTCTACTATAATCCTCACAATCCAC |
| | | CCAGAAGGTATTACTTCTACATGGCTATATTCGCCGTT |
| | | TCTGTCATTTGCGTTTTGTACGGACCCTCACAACAATT |
| | | ATCATCTCCAAAAATAGACTATGATCCATTGACGCTCC |
| | | GATCACTTGATTTGAAGACTTTGGAAGCTCCTTCACAG |
| | | TTGAGTCCAGGCACCGTAGAAGATAATCTTCG |
| 23 | Sequence of the | AAAGCTAGAGTAAAATAGATATAGCGAGATTAGAGA |
| | 3′-Region used | ATGAATACCTTCTTCTAAGCGATCGTCCGTCATCATAG |
| | for knock out of | AATATCATGGACTGTATAGTTTTTTTTTTGTACATATA |
| | PpOCH1 | ATGATTAAACGGTCATCCAACATCTCGTTGACAGATCT |
| | | CTCAGTACGCGAAATCCCTGACTATCAAAGCAAGAAC |
| | | CGATGAAGAAAAAAACAACAGTAACCCAAACACCAC |
| | | AACAAACACTTTATCTTCTCCCCCCCAACACCAATCAT |
| | | CAAAGAGATGTCGGAACCAAACACCAAGAAGCAAAA |
| | | ACTAACCCCATATAAAAACATCCTGGTAGATAATGCT |
| | | GGTAACCCGCTCTCCTTCCATATTCTGGGCTACTTCAC |
| | | GAAGTCTGACCGGTCTCAGTTGATCAACATGATCCTC |
| | | GAAATGGGTGGCAAGATCGTTCCAGACCTGCCTCCTC |
| | | TGGTAGATGGAGTGTTGTTTTTGACAGGGGATTACAA |
| | | GTCTATTGATGAAGATACCCTAAAGCAACTGGGGGAC |
| | | GTTCCAATATACAGAGACTCCTTCATCTACCAGTGTTT |
| | | TGTGCACAAGACATCTCTTCCCATTGACACTTTCCGAA |
| | | TTGACAAGAACGTCGACTTGGCTCAAGATTTGATCAA |
| | | TAGGGCCCTTCAAGAGTCTGTGGATCATGTCACTTCTG |
| | | CCAGCACAGCTGCAGCTGCTGCTGTTGTTGTCGCTACC |
| | | AACGGCCTGTCTTCTAAACCAGACGCTCGTACTAGCA |
| | | AAATACAGTTCACTCCCGAAGAAGATCGTTTTATTCTT |
| | | GACTTTGTTAGGAGAAATCCTAAACGAAGAAACACAC |
| | | ATCAACTGTACACTGAGCTCGCTCAGCACATGAAAAA |
| | | CCATACGAATCATTCTATCCGCCACAGATTTCGTCGTA |
| | | ATCTTTCCGCTCAACTTGATTGGGTTTATGATATCGAT |
| | | CCATTGACCAACCAACCTCGAAAAGATGAAAACGGGA |
| | | ACTACATCAAGGTACAAGGCCTTCCA |
| 24 | K. lactis UDP- | AAACGTAACGCCTGGCACTCTATTTTCTCAAACTTCTG |
| | GlcNAc | GGACGGAAGAGCTAAATATTGTGTTGCTTGAACAAAC |
| | transporter gene | CCAAAAAAACAAAAAAATGAACAAACTAAAACTACA |
| | (KIMNN2-2) | CCTAAATAAACCGTGTGTAAAACGTAGTACCATATTA |
| | ORF underlined | CTAGAAAAGATCACAAGTGTATCACACATGTGCATCT |
| | | CATATTACATCTTTTATCCAATCCATTCTCTCTATCCCG |
| | | TCTGTTCCTGTCAGATTCTTTTTCCATAAAAAGAAGAA |
| | | GACCCCGAATCTCACCGGTACAATGCAAAACTGCTGA |
| | | AAAAAAAAGAAAGTTCACTGGATACGGGAACAGTGC |
| | | CAGTAGGCTTCACCACATGGACAAAACAATTGACGAT |
| | | AAAATAAGCAGGTGAGCTTCTTTTTCAAGTCACGATC |
| | | CCTTTATGTCTCAGAAACAATATATACAAGCTAAACC |
| | | CTTTTGAACCAGTTCTCTCTTCATAGTTATGTTCACAT |
| | | AAATTGCGGGAACAAGACTCCGCTGGCTGTCAGGTAC |
| | | ACGTTGTAACGTTTTCGTCCGCCCAATTATTAGCACAA |
| | | CATTGGCAAAAAGAAAAACTGCTCGTTTTCTCTACAG |
| | | GTAAATTACAATTTTTTTCAGTAATTTTCGCTGAAAAA |
| | | TTTAAAGGGCAGGAAAAAAAGACGATCTCGACTTTGC |
| | | ATAGATGCAAGAACTGTGGTCAAAACTTGAAATAGTA |
| | | ATTTTGCTGTGCGTGAACTAATAAATATATATATATAT |
| | | ATATATATATATTTGTGTATTTTGTATATGTAATTGTGC |
| | | ACGTCTTGGCTATTGGATATAAGATTTTCGCGGGTTGA |
| | | TGACATAGAGCGTGTACTACTGTAATAGTTGTATATTC |
| | | AAAAGCTGCTGCGTGGAGAAAGACTAAAATAGATAA |
| | | AAAGCACACATTTTGACTTCGGTACCGTCAACTTAGTG |
| | | GGACAGTCTTTTATATTTGGTGTAAGCTCATTTCTGGT |
| | | ACTATTCGAAACAGAACAGTGTTTTCTGTATTACCGTC |
| | | CAATCGTTTGTCATGAGTTTTGTATTGATTTTGTCGTT |
| | | AGTGTTCGGAGGATGTTGTTCCAATGTGATTAGTTTCG |
| | | AGCACATGGTGCAAGGCAGCAATATAAATTTGGGAAA |
| | | TATTGTTACATTCACTCAATTCGTGTCTGTGACGCTAA |
| | | TTCAGTTGCCCAATGCTTTGGACTTCTCTCACTTTCCGT |
| | | TTAGGTTGCGACCTAGACACATTCCTCTTAAGATCCAT |
| | | ATGTTAGCTGTGTTTTTGTTCTTTACCAGTTCAGTCGCC |
| | | AATAACAGTGTGTTTAAATTTGACATTTCCGTTCCGAT |
| | | TCATATTATCATTAGATTTTCAGGTACCACTTTGACGA |
| | | TGATAATAGGTTGGGCTGTTTGTAATAAGAGGTACTCC |
| | | AAACTTCAGGTGCAATCTGCCATCATTATGACGCTTGG |
| | | TGCGATTGTCGCATCATTATACCGTGACAAAGAATTTT |
| | | CAATGGACAGTTTAAAGTTGAATACGGATTCAGTGGG |
| | | TATGACCCAAAAATCTATGTTTGGTATCTTTGTTGTGC |
| | | TAGTGGCCACTGCCTTGATGTCATTGTTGTCGTTGCTC |
| | | AACGAATGGACGTATAACAAGTACGGGAAACATTGGA |
| | | AAGAAACTTTGTTCTATTCGCATTTCTTGGCTCTACCG |
| | | TTGTTTATGTTGGGGTACACAAGGCTCAGAGACGAAT |
| | | TCAGAGACCTCTTAATTTCCTCAGACTCAATGGATATT |
| | | CCTATTGTTAAATTACCAATTGCTACGAAACTTTTCAT |
| | | GCTAATAGCAAATAACGTGACCCAGTTCATTTGTATC |
| | | AAAGGTGTTAACATGCTAGCTAGTAACACGGATGCTT |
| | | TGACACTTTCTGTCGTGCTTCTAGTGCGTAAATTTGTT |
| | | AGTCTTTTACTCAGTGTCTACATCTACAAGAACGTCCT |
| | | ATCCGTGACTGCATACCTAGGGACCATCACCGTGTTCC |
| | | TGGGAGCTGGTTTGTATTCATATGGTTCGGTCAAAACT |
| | | GCACTGCCTCGCTGAAACAATCCACGTCTGTATGATA |
| | | CTCGTTTCAGAATTTTTTTGATTTTCTGCCGGATATGGT |
| | | TTCTCATCTTTACAATCGCATTCTTAATTATACCAGAA |
| | | CGTAATTCAATGATCCCAGTGACTCGTAACTCTTATAT |
| | | GTCAATTTAAGC |
| 25 | Sequence of the | GGCCGAGCGGGCCTAGATTTTCACTACAAATTTCAAA |
| | 5′-Region used | ACTACGCGGATTTATTGTCTCAGAGAGCAATTTGGCAT |
| | for knock out of | TTCTGAGCGTAGCAGGAGGCTTCATAAGATTGTATAG |
| | PpBMT2 | GACCGTACCAACAAATTGCCGAGGCACAACACGGTAT |
| | | GCTGTGCACTTATGTGGCTACTTCCCTACAACGGAATG |
| | | AAACCTTCCTCTTTCCGCTTAAACGAGAAAGTGTGTCG |
| | | CAATTGAATGCAGGTGCCTGTGCGCCTTGGTGTATTGT |
| | | TTTTGAGGGCCCAATTTATCAGGCGCCTTTTTTCTTGG |
| | | TTGTTTTCCCTTAGCCTCAAGCAAGGTTGGTCTATTTC |
| | | ATCTCCGCTTCTATACCGTGCCTGATACTGTTGGATGA |
| | | GAACACGACTCAACTTCCTGCTGCTCTGTATTGCCAGT |
| | | GTTTTGTCTGTGATTTGGATCGGAGTCCTCCTTACTTG |
| | | GAATGATAATAATCTTGGCGGAATCTCCCTAAACGGA |
| | | GGCAAGGATTCTGCCTATGATGATCTGCTATCATTGGG |
| | | AAGCTTCAACGACATGGAGGTCGACTCCTATGTCACC |
| | | AACATCTACGACAATGCTCCAGTGCTAGGATGTACGG |
| | | ATTTGTCTTATCATGGATTGTTGAAAGTCACCCCAAAG |
| | | CATGACTTAGCTTGCGATTTGGAGTTCATAAGAGCTCA |
| | | GATTTTGGACATTGACGTTTACTCCGCCATAAAAGACT |
| | | TAGAAGATAAAGCCTTGACTGTAAAACAAAAGGTTGA |
| | | AAAACACTGGTTTACGTTTTATGGTAGTTCAGTCTTTC |
| | | TGCCCGAACACGATGTGCATTACCTGGTTAGACGAGT |
| | | CATCTTTTCGGCTGAAGGAAAGGCGAACTCTCCAGTA |
| | | ACATC |
| 26 | Sequence of the | CCATATGATGGGTGTTTGCTCACTCGTATGGATCAAAA |
| | 3′-Region used | TTCCATGGTTTCTTCTGTACAACTTGTACACTTATTTGG |
| | for knock out of | ACTTTTCTAACGGTTTTTCTGGTGATTTGAGAAGTCCT |
| | PpBMT2 | TATTTTGGTGTTCGCAGCTTATCCGTGATTGAACCATC |
| | | AGAAATACTGCAGCTCGTTATCTAGTTTCAGAATGTGT |
| | | TGTAGAATACAATCAATTCTGAGTCTAGTTTGGGTGGG |
| | | TCTTGGCGACGGGACCGTTATATGCATCTATGCAGTGT |
| | | TAAGGTACATAGAATGAAAATGTAGGGGTTAATCGAA |
| | | AGCATCGTTAATTTCAGTAGAACGTAGTTCTATTCCCT |
| | | ACCCAAATAATTTGCCAAGAATGCTTCGTATCCACAT |
| | | ACGCAGTGGACGTAGCAAATTTCACTTTGGACTGTGA |
| | | CCTCAAGTCGTTATCTTCTACTTGGACATTGATGGTCA |
| | | TTACGTAATCCACAAAGAATTGGATAGCCTCTCGTTTT |
| | | ATCTAGTGCACAGCCTAATAGCACTTAAGTAAGAGCA |
| | | ATGGACAAATTTGCATAGACATTGAGCTAGATACGTA |
| | | ACTCAGATCTTGTTCACTCATGGTGTACTCGAAGTACT |
| | | GCTGGAACCGTTACCTCTTATCATTTCGCTACTGGCTC |
| | | GTGAAACTACTGGATGAAAAAAAAAAAAGAGCTGAA |
| | | AGCGAGATCATCCCATTTTGTCATCATACAAATTCACG |
| | | CTTGCAGTTTTGCTTCGTTAACAAGACAAGATGTCTTT |
| | | ATCAAAGACCCGTTTTTTCTTCTTGAAGAATACTTCCC |
| | | TGTTGAGCACATGCAAACCATATTTATCTCAGATTTCA |
| | | CTCAACTTGGGTGCTTCCAAGAGAAGTAAAATTCTTCC |
| | | CACTGCATCAACTTCCAAGAAACCCGTAGACCAGTTT |
| | | CTCTTCAGCCAAAAGAAGTTGCTCGCCGATCACCGCG |
| | | GTAACAGAGGAGTCAGAAGGTTTCACACCCTTCCATC |
| | | CCGATTTCAAAGTCAAAGTGCTGCGTTGAACCAAGGT |
| | | TTTCAGGTTGCCAAAGCCCAGTCTGCAAAAACTAGTT |
| | | CCAAATGGCCTATTAATTCCCATAAAAGTGTTGGCTAC |
| | | GTATGTATCGGTACCTCCATTCTGGTATTTGCTATTGT |
| | | TGTCGTTGGTGGGTTGACTAGACTGACCGAATCCGGT |
| | | CTTTCCATAACGGAGTGGAAACCTATCACTGGTTCGGT |
| | | TCCCCCACTGACTGAGGAAGACTGGAAGTTGGAATTT |
| | | GAAAAATACAAACAAAGCCCTGAGTTTCAGGAACTAA |
| | | ATTCTCACATAACATTGGAAGAGTTCAAGTTTATATTT |
| | | TCCATGGAATGGGGACATAGATTGTTGGGAAGGGTCA |
| | | TCGGCCTGTCGTTTGTTCTTCCCACGTTTTACTTCATTG |
| | | CCCGTCGAAAGTGTTCCAAAGATGTTGCATTGAAACT |
| | | GCTTGCAATATGCTCTATGATAGGATTCCAAGGTTTCA |
| | | TCGGCTGGTGGATGGTGTATTCCGGATTGGACAAACA |
| | | GCAATTGGCTGAACGTAACTCCAAACCAACTGTGTCT |
| | | CCATATCGCTTAACTACCCATCTTGGAACTGCATTTGT |
| | | TATTTACTGTTACATGATTTACACAGGGCTTCAAGTTT |
| | | TGAAGAACTATAAGATCATGAAACAGCCTGAAGCGTA |
| | | TGTTCAAATTTTCAAGCAAATTGCGTCTCCAAAATTGA |
| | | AAACTTTCAAGAGACTCTCTTCAGTTCTATTAGGCCTG |
| | | GTG |
| 27 | DNA encodes | ATGTCTGCCAACCTAAAATATCTTTCCTTGGGAATTTT |
| | MmSLC35A3 | GGTGTTTCAGACTACCAGTCTGGTTCTAACGATGCGGT |
| | UDP-GlcNAc | ATTCTAGGACTTTAAAAGAGGAGGGGCCTCGTTATCT |
| | transporter | GTCTTCTACAGCAGTGGTTGTGGCTGAATTTTTGAAGA |
| | | TAATGGCCTGCATCTTTTTAGTCTACAAAGACAGTAAG |
| | | TGTAGTGTGAGAGCACTGAATAGAGTACTGCATGATG |
| | | AAATTCTTAATAAGCCCATGGAAACCCTGAAGCTCGC |
| | | TATCCCGTCAGGGATATATACTCTTCAGAACAACTTAC |
| | | TCTATGTGGCACTGTCAAACCTAGATGCAGCCACTTAC |
| | | CAGGTTACATATCAGTTGAAAATACTTACAACAGCAT |
| | | TATTTTCTGTGTCTATGCTTGGTAAAAAATTAGGTGTG |
| | | TACCAGTGGCTCTCCCTAGTAATTCTGATGGCAGGAGT |
| | | TGCTTTTGTACAGTGGCCTTCAGATTCTCAAGAGCTGA |
| | | ACTCTAAGGACCTTTCAACAGGCTCACAGTTTGTAGG |
| | | CCTCATGGCAGTTCTCACAGCCTGTTTTTCAAGTGGCT |
| | | TTGCTGGAGTTTATTTTGAGAAAATCTTAAAAGAAAC |
| | | AAAACAGTCAGTATGGATAAGGAACATTCAACTTGGT |
| | | TTCTTTGGAAGTATATTTGGATTAATGGGTGTATACGT |
| | | TTATGATGGAGAATTGGTCTCAAAGAATGGATTTTTTC |
| | | AGGGATATAATCAACTGACGTGGATAGTTGTTGCTCT |
| | | GCAGGCACTTGGAGGCCTTGTAATAGCTGCTGTCATC |
| | | AAATATGCAGATAACATTTTAAAAGGATTTGCGACCT |
| | | CCTTATCCATAATATTGTCAACAATAATATCTTATTTT |
| | | TGGTTGCAAGATTTTGTGCCAACCAGTGTCTTTTTCCT |
| | | TGGAGCCATCCTTGTAATAGCAGCTACTTTCTTGTATG |
| | | GTTACGATCCCAAACCTGCAGGAAATCCCACTAAAGC |
| | | ATAG |
| 28 | Sequence of the | GATCTGGCCATTGTGAAACTTGACACTAAAGACAAAA |
| | 5′-Region used | CTCTTAGAGTTTCCAATCACTTAGGAGACGATGTTTCC |
| | for knock out of | TACAACGAGTACGATCCCTCATTGATCATGAGCAATTT |
| | PpMNN4L1 | GTATGTGAAAAAAGTCATCGACCTTGACACCTTGGAT |
| | | AAAAGGGCTGGAGGAGGTGGAACCACCTGTGCAGGC |
| | | GGTCTGAAAGTGTTCAAGTACGGATCTACTACCAAAT |
| | | ATACATCTGGTAACCTGAACGGCGTCAGGTTAGTATA |
| | | CTGGAACGAAGGAAAGTTGCAAAGCTCCAAATTTGTG |
| | | GTTCGATCCTCTAATTACTCTCAAAAGCTTGGAGGAA |
| | | ACAGCAACGCCGAATCAATTGACAACAATGGTGTGGG |
| | | TTTTGCCTCAGCTGGAGACTCAGGCGCATGGATTCTTT |
| | | CCAAGCTACAAGATGTTAGGGAGTACCAGTCATTCAC |
| | | TGAAAAGCTAGGTGAAGCTACGATGAGCATTTTCGAT |
| | | TTCCACGGTCTTAAACAGGAGACTTCTACTACAGGGC |
| | | TTGGGGTAGTTGGTATGATTCATTCTTACGACGGTGAG |
| | | TTCAAACAGTTTGGTTTGTTCACTCCAATGACATCTAT |
| | | TCTACAAAGACTTCAACGAGTGACCAATGTAGAATGG |
| | | TGTGTAGCGGGTTGCGAAGATGGGGATGTGGACACTG |
| | | AAGGAGAACACGAATTGAGTGATTTGGAACAACTGCA |
| | | TATGCATAGTGATTCCGACTAGTCAGGCAAGAGAGAG |
| | | CCCTCAAATTTACCTCTCTGCCCCTCCTCACTCCTTTTG |
| | | GTACGCATAATTGCAGTATAAAGAACTTGCTGCCAGC |
| | | CAGTAATCTTATTTCATACGCAGTTCTATATAGCACAT |
| | | AATCTTGCTTGTATGTATGAAATTTACCGCGTTTTAGT |
| | | TGAAATTGTTTATGTTGTGTGCCTTGCATGAAATCTCT |
| | | CGTTAGCCCTATCCTTACATTTAACTGGTCTCAAAACC |
| | | TCTACCAATTCCATTGCTGTACAACAATATGAGGCGG |
| | | CATTACTGTAGGGTTGGAAAAAAATTGTCATTCCAGC |
| | | TAGAGATCACACGACTTCATCACGCTTATTGCTCCTCA |
| | | TTGCTAAATCATTTACTCTTGACTTCGACCCAGAAAAG |
| | | TTCGCC |
| 29 | Sequence of the | GCATGTCAAACTTGAACACAACGACTAGATAGTTGTT |
| | 3′-Region used | TTTTCTATATAAAACGAAACGTTATCATCTTTAATAAT |
| | for knock out of | CATTGAGGTTTACCCTTATAGTTCCGTATTTTCGTTTCC |
| | PpMNN4L1 | AAACTTAGTAATCTTTTGGAAATATCATCAAAGCTGGT |
| | | GCCAATCTTCTTGTTTGAAGTTTCAAACTGCTCCACCA |
| | | AGCTACTTAGAGACTGTTCTAGGTCTGAAGCAACTTC |
| | | GAACACAGAGACAGCTGCCGCCGATTGTTCTTTTTTGT |
| | | GTTTTTCTTCTGGAAGAGGGGCATCATCTTGTATGTCC |
| | | AATGCCCGTATCCTTTCTGAGTTGTCCGACACATTGTC |
| | | CTTCGAAGAGTTTCCTGACATTGGGCTTCTTCTATCCG |
| | | TGTATTAATTTTGGGTTAAGTTCCTCGTTTGCATAGCA |
| | | GTGGATACCTCGATTTTTTTGGCTCCTATTTACCTGAC |
| | | ATAATATTCTACTATAATCCAACTTGGACGCGTCATCT |
| | | ATGATAACTAGGCTCTCCTTTGTTCAAAGGGGACGTCT |
| | | TCATAATCCACTGGCACGAAGTAAGTCTGCAACGAGG |
| | | CGGCTTTTGCAACAGAACGATAGTGTCGTTTCGTACTT |
| | | GGACTATGCTAAACAAAAGGATCTGTCAAACATTTCA |
| | | ACCGTGTTTCAAGGCACTCTTTACGAATTATCGACCAA |
| | | GACCTTCCTAGACGAACATTTCAACATATCCAGGCTA |
| | | CTGCTTCAAGGTGGTGCAAATGATAAAGGTATAGATA |
| | | TTAGATGTGTTTGGGACCTAAAACAGTTCTTGCCTGAA |
| | | GATTCCCTTGAGCAACAGGCTTCAATAGCCAAGTTAG |
| | | AGAAGCAGTACCAAATCGGTAACAAAAGGGGGAAGC |
| | | ATATAAAACCTTTACTATTGCGACAAAATCCATCCTTG |
| | | AAAGTAAAGCTGTTTGTTCAATGTAAAGCATACGAAA |
| | | CGAAGGAGGTAGATCCTAAGATGGTTAGAGAACTTAA |
| | | CGGGACATACTCCAGCTGCATCCCATATTACGATCGCT |
| | | GGAAGACTTTTTTCATGTACGTATCGCCCACCAACCTT |
| | | TCAAAGCAAGCTAGGTATGATTTTGACAGTTCTCACA |
| | | ATCCATTGGTTTTCATGCAACTTGAAAAAACCCAACTC |
| | | AAACTTCATGGGGATCCATACAATGTAAATCATTACG |
| | | AGAGGGCGAGGTTGAAAAGTTTCCATTGCAATCACGT |
| | | CGCATCATGGCTACTGAAAGGCCTTAAC |
| 30 | Sequence of the | TCATTCTATATGTTCAAGAAAAGGGTAGTGAAAGGAA |
| | 5′-Region used | AGAAAAGGCATATAGGCGAGGGAGAGTTAGCTAGCA |
| | for knock out of | TACAAGATAATGAAGGATCAATAGCGGTAGTTAAAGT |
| | PpPNO1 and | GCACAAGAAAAGAGCACCTGTTGAGGCTGATGATAAA |
| | PpMNN4 | GCTCCAATTACATTGCCACAGAGAAACACAGTAACAG |
| | | AAATAGGAGGGGATGCACCACGAGAAGAGCATTCAG |
| | | TGAACAACTTTGCCAAATTCATAACCCCAAGCGCTAA |
| | | TAAGCCAATGTCAAAGTCGGCTACTAACATTAATAGT |
| | | ACAACAACTATCGATTTTCAACCAGATGTTTGCAAGG |
| | | ACTACAAACAGACAGGTTACTGCGGATATGGTGACAC |
| | | TTGTAAGTTTTTGCACCTGAGGGATGATTTCAAACAGG |
| | | GATGGAAATTAGATAGGGAGTGGGAAAATGTCCAAA |
| | | AGAAGAAGCATAATACTCTCAAAGGGGTTAAGGAGAT |
| | | CCAAATGTTTAATGAAGATGAGCTCAAAGATATCCCG |
| | | TTTAAATGCATTATATGCAAAGGAGATTACAAATCAC |
| | | CCGTGAAAACTTCTTGCAATCATTATTTTTGCGAACAA |
| | | TGTTTCCTGCAACGGTCAAGAAGAAAACCAAATTGTA |
| | | TTATATGTGGCAGAGACACTTTAGGAGTTGCTTTACCA |
| | | GCAAAGAAGTTGTCCCAATTTCTGGCTAAGATACATA |
| | | ATAATGAAAGTAATAAAGTTTAGTAATTGCATTGCGTT |
| | | GACTATTGATTGCATTGATGTCGTGTGATACTTTCACC |
| | | GAAAAAAAACACGAAGCGCAATAGGAGCGGTTGCAT |
| | | ATTAGTCCCCAAAGCTATTTAATTGTGCCTGAAACTGT |
| | | TTTTTAAGCTCATCAAGCATAATTGTATGCATTGCGAC |
| | | GTAACCAACGTTTAGGCGCAGTTTAATCATAGCCCAC |
| | | TGCTAAGCC |
| 31 | Sequence of the | CGGAGGAATGCAAATAATAATCTCCTTAATTACCCAC |
| | 3′-Region used | TGATAAGCTCAAGAGACGCGGTTTGAAAACGATATAA |
| | for knock out of | TGAATCATTTGGATTTTATAATAAACCCTGACAGTTTT |
| | PpPNO1 and | TCCACTGTATTGTTTTAACACTCATTGGAAGCTGTATT |
| | PpMNN4 | GATTCTAAGAAGCTAGAAATCAATACGGCCATACAAA |
| | | AGATGACATTGAATAAGCACCGGCTTTTTTGATTAGC |
| | | ATATACCTTAAAGCATGCATTCATGGCTACATAGTTGT |
| | | TAAAGGGCTTCTTCCATTATCAGTATAATGAATTACAT |
| | | AATCATGCACTTATATTTGCCCATCTCTGTTCTCTCACT |
| | | CTTGCCTGGGTATATTCTATGAAATTGCGTATAGCGTG |
| | | TCTCCAGTTGAACCCCAAGCTTGGCGAGTTTGAAGAG |
| | | AATGCTAACCTTGCGTATTCCTTGCTTCAGGAAACATT |
| | | CAAGGAGAAACAGGTCAAGAAGCCAAACATTTTGATC |
| | | CTTCCCGAGTTAGCATTGACTGGCTACAATTTTCAAAG |
| | | CCAGCAGCGGATAGAGCCTTTTTTGGAGGAAACAACC |
| | | AAGGGAGCTAGTACCCAATGGGCTCAAAAAGTATCCA |
| | | AGACGTGGGATTGCTTTACTTTAATAGGATACCCAGA |
| | | AAAAAGTTTAGAGAGCCCTCCCCGTATTTACAACAGT |
| | | GCGGTACTTGTATCGCCTCAGGGAAAAGTAATGAACA |
| | | ACTACAGAAAGTCCTTCTTGTATGAAGCTGATGAACA |
| | | TTGGGGATGTTCGGAATCTTCTGATGGGTTTCAAACAG |
| | | TAGATTTATTAATTGAAGGAAAGACTGTAAAGACATC |
| | | ATTTGGAATTTGCATGGATTTGAATCCTTATAAATTTG |
| | | AAGCTCCATTCACAGACTTCGAGTTCAGTGGCCATTGC |
| | | TTGAAAACCGGTACAAGACTCATTTTGTGCCCAATGG |
| | | CCTGGTTGTCCCCTCTATCGCCTTCCATTAAAAAGGAT |
| | | CTTAGTGATATAGAGAAAAGCAGACTTCAAAAGTTCT |
| | | ACCTTGAAAAAATAGATACCCCGGAATTTGACGTTAA |
| | | TTACGAATTGAAAAAAGATGAAGTATTGCCCACCCGT |
| | | ATGAATGAAACGTTGGAAACAATTGACTTTGAGCCTT |
| | | CAAAACCGGACTACTCTAATATAAATTATTGGATACT |
| | | AAGGTTTTTTCCCTTTCTGACTCATGTCTATAAACGAG |
| | | ATGTGCTCAAAGAGAATGCAGTTGCAGTCTTATGCAA |
| | | CCGAGTTGGCATTGAGAGTGATGTCTTGTACGGAGGA |
| | | TCAACCACGATTCTAAACTTCAATGGTAAGTTAGCATC |
| | | GACACAAGAGGAGCTGGAGTTGTACGGGCAGACTAAT |
| | | AGTCTCAACCCCAGTGTGGAAGTATTGGGGGCCCTTG |
| | | GCATGGGTCAACAGGGAATTCTAGTACGAGACATTGA |
| | | ATTAACATAATATACAATATACAATAAACACAAATAA |
| | | AGAATACAAGCCTGACAAAAATTCACAAATTATTGCC |
| | | TAGACTTGTCGTTATCAGCAGCGACCTTTTTCCAATGC |
| | | TCAATTTCACGATATGCCTTTTCTAGCTCTGCTTTAAG |
| | | CTTCTCATTGGAATTGGCTAACTCGTTGACTGCTTGGT |
| | | CAGTGATGAGTTTCTCCAAGGTCCATTTCTCGATGTTG |
| | | TTGTTTTCGTTTTCCTTTAATCTCTTGATATAATCAACA |
| | | GCCTTCTTTAATATCTGAGCCTTGTTCGAGTCCCCTGT |
| | | TGGCAACAGAGCGGCCAGTTCCTTTATTCCGTGGTTTA |
| | | TATTTTCTCTTCTACGCCTTTCTACTTCTTTGTGATTCT |
| | | CTTTACGCATCTTATGCCATTCTTCAGAACCAGTGGCT |
| | | GGCTTAACCGAATAGCCAGAGCCTGAAGAAGCCGCAC |
| | | TAGAAGAAGCAGTGGCATTGTTGACTATGG |
| 32 | DNA encodes | TCAGTCAGTGCTCTTGATGGTGACCCAGCAAGTTTGAC |
| | human GnTI | CAGAGAAGTGATTAGATTGGCCCAAGACGCAGAGGTG |
| | catalytic domain | GAGTTGGAGAGACAACGTGGACTGCTGCAGCAAATCG |
| | (NA) | GAGATGCATTGTCTAGTCAAAGAGGTAGGGTGCCTAC |
| | Codon- | CGCAGCTCCTCCAGCACAGCCTAGAGTGCATGTGACC |
| | optimized | CCTGCACCAGCTGTGATTCCTATCTTGGTCATCGCCTG |
| | | TGACAGATCTACTGTTAGAAGATGTCTGGACAAGCTG |
| | | TTGCATTACAGACCATCTGCTGAGTTGTTCCCTATCAT |
| | | CGTTAGTCAAGACTGTGGTCACGAGGAGACTGCCCAA |
| | | GCCATCGCCTCCTACGGATCTGCTGTCACTCACATCAG |
| | | ACAGCCTGACCTGTCATCTATTGCTGTGCCACCAGACC |
| | | ACAGAAAGTTCCAAGGTTACTACAAGATCGCTAGACA |
| | | CTACAGATGGGCATTGGGTCAAGTCTTCAGACAGTTT |
| | | AGATTCCCTGCTGCTGTGGTGGTGGAGGATGACTTGG |
| | | AGGTGGCTCCTGACTTCTTTGAGTACTTTAGAGCAACC |
| | | TATCCATTGCTGAAGGCAGACCCATCCCTGTGGTGTGT |
| | | CTCTGCCTGGAATGACAACGGTAAGGAGCAAATGGTG |
| | | GACGCTTCTAGGCCTGAGCTGTTGTACAGAACCGACT |
| | | TCTTTCCTGGTCTGGGATGGTTGCTGTTGGCTGAGTTG |
| | | TGGGCTGAGTTGGAGCCTAAGTGGCCAAAGGCATTCT |
| | | GGGACGACTGGATGAGAAGACCTGAGCAAAGACAGG |
| | | GTAGAGCCTGTATCAGACCTGAGATCTCAAGAACCAT |
| | | GACCTTTGGTAGAAAGGGAGTGTCTCACGGTCAATTC |
| | | TTTGACCAACACTTGAAGTTTATCAAGCTGAACCAGC |
| | | AATTTGTGCACTTCACCCAACTGGACCTGTCTTACTTG |
| | | CAGAGAGAGGCCTATGACAGAGATTTCCTAGCTAGAG |
| | | TCTACGGAGCTCCTCAACTGCAAGTGGAGAAAGTGAG |
| | | GACCAATGACAGAAAGGAGTTGGGAGAGGTGAGAGT |
| | | GCAGTACACTGGTAGGGACTCCTTTAAGGCTTTCGCTA |
| | | AGGCTCTGGGTGTCATGGATGACCTTAAGTCTGGAGT |
| | | TCCTAGAGCTGGTTACAGAGGTATTGTCACCTTTCAAT |
| | | TCAGAGGTAGAAGAGTCCACTTGGCTCCTCCACCTAC |
| | | TTGGGAGGGTTATGATCCTTCTTGGAATTAG |
| 33 | DNA encodes | ATGCCCAGAAAAATATTTAACTACTTCATTTTGACTGT |
| | Pp SEC12 (10) | ATTCATGGCAATTCTTGCTATTGTTTTACAATGGTCTA |
| | The last 9 | TAGAGAATGGACATGGGCGCGCC |
| | nucleotides are | |
| | the linker | |
| | containing the | |
| | AscI restriction | |
| | site used for | |
| | fusion to | |
| | proteins of | |
| | interest. | |
| 34 | Sequence of the | GAAGTAAAGTTGGCGAAACTTTGGGAACCTTTGGTTA |
| | PpSEC4 | AAACTTTGTAATTTTTGTCGCTACCCATTAGGCAGAAT |
| | promoter | CTGCATCTTGGGAGGGGGATGTGGTGGCGTTCTGAGA |
| | | TGTACGCGAAGAATGAAGAGCCAGTGGTAACAACAG |
| | | GCCTAGAGAGATACGGGCATAATGGGTATAACCTACA |
| | | AGTTAAGAATGTAGCAGCCCTGGAAACCAGATTGAAA |
| | | CGAAAAACGAAATCATTTAAACTGTAGGATGTTTTGG |
| | | CTCATTGTCTGGAAGGCTGGCTGTTTATTGCCCTGTTC |
| | | TTTGCATGGGAATAAGCTATTATATCCCTCACATAATC |
| | | CCAGAAAATAGATTGAAGCAACGCGAAATCCTTACGT |
| | | ATCGAAGTAGCCTTCTTACACATTCACGTTGTACGGAT |
| | | AAGAAAACTACTCAAACGAACAATC |
| 35 | Sequence of the | AATAGATATAGCGAGATTAGAGAATGAATACCTTCTT |
| | PpOCH1 | CTAAGCGATCGTCCGTCATCATAGAATATCATGGACT |
| | terminator | GTATAGTTTTTTTTTTGTACATATAATGATTAAACGGT |
| | | CATCCAACATCTCGTTGACAGATCTCTCAGTACGCGA |
| | | AATCCCTGACTATCAAAGCAAGAACCGATGAAGAAAA |
| | | AAACAACAGTAACCCAAACACCACAACAAACACTTTA |
| | | TCTTCTCCCCCCCAACACCAATCATCAAAGAGATGTCG |
| | | GAACACAAACACCAAGAAGCAAAAACTAACCCCATA |
| | | TAAAAACATCCTGGTAGATAATGCTGGTAACCCGCTC |
| | | TCCTTCCATATTCTGGGCTACTTCACGAAGTCTGACCG |
| | | GTCTCAGTTGATCAACATGATCCTCGAAATGG |
| 36 | DNA encodes | GAGCCCGCTGACGCCACCATCCGTGAGAAGAGGGCAA |
| | Mm ManI | AGATCAAAGAGATGATGACCCATGCTTGGAATAATTA |
| | catalytic domain | TAAACGCTATGCGTGGGGCTTGAACGAACTGAAACCT |
| | (FB) | ATATCAAAAGAAGGCCATTCAAGCAGTTTGTTTGGCA |
| | | ACATCAAAGGAGCTACAATAGTAGATGCCCTGGATAC |
| | | CCTTTTCATTATGGGCATGAAGACTGAATTTCAAGAA |
| | | GCTAAATCGTGGATTAAAAAATATTTAGATTTTAATGT |
| | | GAATGCTGAAGTTTCTGTTTTTGAAGTCAACATACGCT |
| | | TCGTCGGTGGACTGCTGTCAGCCTACTATTTGTCCGGA |
| | | GAGGAGATATTTCGAAAGAAAGCAGTGGAACTTGGGG |
| | | TAAAATTGCTACCTGCATTTCATACTCCCTCTGGAATA |
| | | CCTTGGGCATTGCTGAATATGAAAAGTGGGATCGGGC |
| | | GGAACTGGCCCTGGGCCTCTGGAGGCAGCAGTATCCT |
| | | GGCCGAATTTGGAACTCTGCATTTAGAGTTTATGCACT |
| | | TGTCCCACTTATCAGGAGACCCAGTCTTTGCCGAAAA |
| | | GGTTATGAAAATTCGAACAGTGTTGAACAAACTGGAC |
| | | AAACCAGAAGGCCTTTATCCTAACTATCTGAACCCCA |
| | | GTAGTGGACAGTGGGGTCAACATCATGTGTCGGTTGG |
| | | AGGACTTGGAGACAGCTTTTATGAATATTTGCTTAAGG |
| | | CGTGGTTAATGTCTGACAAGACAGATCTCGAAGCCAA |
| | | GAAGATGTATTTTGATGCTGTTCAGGCCATCGAGACTC |
| | | ACTTGATCCGCAAGTCAAGTGGGGGACTAACGTACAT |
| | | CGCAGAGTGGAAGGGGGGCCTCCTGGAACACAAGAT |
| | | GGGCCACCTGACGTGCTTTGCAGGAGGCATGTTTGCA |
| | | CTTGGGGCAGATGGAGCTCCGGAAGCCCGGGCCCAAC |
| | | ACTACCTTGAACTCGGAGCTGAAATTGCCCGCACTTGT |
| | | CATGAATCTTATAATCGTACATATGTGAAGTTGGGAC |
| | | CGGAAGCGTTTCGATTTGATGGCGGTGTGGAAGCTAT |
| | | TGCCACGAGGCAAAATGAAAAGTATTACATCTTACGG |
| | | CCCGAGGTCATCGAGACATACATGTACATGTGGCGAC |
| | | TGACTCACGACCCCAAGTACAGGACCTGGGCCTGGGA |
| | | AGCCGTGGAGGCTCTAGAAAGTCACTGCAGAGTGAAC |
| | | GGAGGCTACTCAGGCTTACGGGATGTTTACATTGCCC |
| | | GTGAGAGTTATGACGATGTCCAGCAAAGTTTCTTCCTG |
| | | GCAGAGACACTGAAGTATTTGTACTTGATATTTTCCGA |
| | | TGATGACCTTCTTCCACTAGAACACTGGATCTTCAACA |
| | | CCGAGGCTCATCCTTTCCCTATACTCCGTGAACAGAAG |
| | | AAGGAAATTGATGGCAAAGAGAAATGA |
| 37 | DNA encodes | ATGAACACTATCCACATAATAAAATTACCGCTTAACT |
| | ScSEC12 (8) | ACGCCAACTACACCTCAATGAAACAAAAAATCTCTAA |
| | The last 9 | ATTTTTCACCAACTTCATCCTTATTGTGCTGCTTTCTTA |
| | nucleotides are | CATTTTACAGTTCTCCTATAAGCACAATTTGCATTCCA |
| | the linker | TGCTTTTCAATTACGCGAAGGACAATTTTCTAACGAAA |
| | containing the | AGAGACACCATCTCTTCGCCCTACGTAGTTGATGAAG |
| | AscI restriction | ACTTACATCAAACAACTTTGTTTGGCAACCACGGTAC |
| | site used for | AAAAACATCTGTACCTAGCGTAGATTCCATAAAAGTG |
| | fusion to | CATGGCGTGGGGCGCGCC |
| | proteins of | |
| | interest | |
| 38 | Sequence of the | GAGTCGGCCAAGAGATGATAACTGTTACTAAGCTTCT |
| | 5′-region that | CCGTAATTAGTGGTATTTTGTAACTTTTACCAATAATC |
| | was used to | GTTTATGAATACGGATATTTTTCGACCTTATCCAGTGC |
| | knock into the | CAAATCACGTAACTTAATCATGGTTTAAATACTCCACT |
| | PpADE1 locus | TGAACGATTCATTATTCAGAAAAAAGTCAGGTTGGCA |
| | | GAAACACTTGGGCGCTTTGAAGAGTATAAGAGTATTA |
| | | AGCATTAAACATCTGAACTTTCACCGCCCCAATATACT |
| | | ACTCTAGGAAACTCGAAAAATTCCTTTCCATGTGTCAT |
| | | CGCTTCCAACACACTTTGCTGTATCCTTCCAAGTATGT |
| | | CCATTGTGAACACTGATCTGGACGGAATCCTACCTTTA |
| | | ATCGCCAAAGGAAAGGTTAGAGACATTTATGCAGTCG |
| | | ATGAGAACAACTTGCTGTTCGTCGCAACTGACCGTAT |
| | | CTCCGCTTACGATGTGATTATGACAAACGGTATTCCTG |
| | | ATAAGGGAAAGATTTTGACTCAGCTCTCAGTTTTCTGG |
| | | TTTGATTTTTTGGCACCCTACATAAAGAATCATTTGGT |
| | | TGCTTCTAATGACAAGGAAGTCTTTGCTTTACTACCAT |
| | | CAAAACTGTCTGAAGAAAAaTACAAATCTCAATTAGA |
| | | GGGACGATCCTTGATAGTAAAAAAGCACAGACTGATA |
| | | CCTTTGGAAGCCATTGTCAGAGGTTACATCACTGGAA |
| | | GTGCATGGAAAGAGTACAAGAACTCAAAAACTGTCCA |
| | | TGGAGTCAAGGTTGAAAACGAGAACCTTCAAGAGAGC |
| | | GACGCCTTTCCAACTCCGATTTTCACACCTTCAACGAA |
| | | AGCTGAACAGGGTGAACACGATGAAAACATCTCTATT |
| | | GAACAAGCTGCTGAGATTGTAGGTAAAGACATTTGTG |
| | | AGAAGGTCGCTGTCAAGGCGGTCGAGTTGTATTCTGC |
| | | TGCAAAAAACCTCGCCCTTTTGAAGGGGATCATTATT |
| | | GCTGATACGAAATTCGAATTTGGACTGGACGAAAACA |
| | | ATGAATTGGTACTAGTAGATGAAGTTTTAACTCCAGAT |
| | | TCTTCTAGATTTTGGAATCAAAAGACTTACCAAGTGG |
| | | GTAAATCGCAAGAGAGTTACGATAAGCAGTTTCTCAG |
| | | AGATTGGTTGACGGCCAACGGATTGAATGGCAAAGAG |
| | | GGCGTAGCCATGGATGCAGAAATTGCTATCAAGAGTA |
| | | AAGAAAAGTATATTGAAGCTTATGAAGCAATTACTGG |
| | | CAAGAAATGGGCTTGA |
| 39 | Sequence of the | ATGATTAGTACCCTCCTCGCCTTTTTCAGACATCTGAA |
| | 3′-region that | ATTTCCCTTATTCTTCCAATTCCATATAAAATCCTATTT |
| | was used to | AGGTAATTAGTAAACAATGATCATAAAGTGAAATCAT |
| | knock into the | TCAAGTAACCATTCCGTTTATCGTTGATTTAAAATCAA |
| | PpADE1 locus | TAACGAATGAATGTCGGTCTGAGTAGTCAATTTGTTGC |
| | | CTTGGAGCTCATTGGCAGGGGGTCTTTTGGCTCAGTAT |
| | | GGAAGGTTGAAAGGAAAACAGATGGAAAGTGGTTCG |
| | | TCAGAAAAGAGGTATCCTACATGAAGATGAATGCCAA |
| | | AGAGATATCTCAAGTGATAGCTGAGTTCAGAATTCTT |
| | | AGTGAGTTAAGCCATCCCAACATTGTGAAGTACCTTC |
| | | ATCACGAACATATTTCTGAGAATAAAACTGTCAATTT |
| | | ATACATGGAATACTGTGATGGTGGAGATCTCTCCAAG |
| | | CTGATTCGAACACATAGAAGGAACAAAGAGTACATTT |
| | | CAGAAGAAAAAATATGGAGTATTTTTACGCAGGTTTT |
| | | ATTAGCATTGTATCGTTGTCATTATGGAACTGATTTCA |
| | | CGGCTTCAAAGGAGTTTGAATCGCTCAATAAAGGTAA |
| | | TAGACGAACCCAGAATCCTTCGTGGGTAGACTCGACA |
| | | AGAGTTATTATTCACAGGGATATAAAACCCGACAACA |
| | | TCTTTCTGATGAACAATTCAAACCTTGTCAAACTGGGA |
| | | GATTTTGGATTAGCAAAAATTCTGGACCAAGAAAACG |
| | | ATTTTGCCAAAACATACGTCGGTACGCCGTATTACATG |
| | | TCTCCTGAAGTGCTGTTGGACCAACCCTACTCACCATT |
| | | ATGTGATATATGGTCTCTTGGGTGCGTCATGTATGAGC |
| | | TATGTGCATTGAGGCCTCCTT |
| 40 | DNA encodes | ATGACAGCTCAGTTACAAAGTGAAAGTACTTCTAAAA |
| | ScGAL10 | TTGTTTTGGTTACAGGTGGTGCTGGATACATTGGTTCA |
| | | CACACTGTGGTAGAGCTAATTGAGAATGGATATGACT |
| | | GTGTTGTTGCTGATAACCTGTCGAATTCAACTTATGAT |
| | | TCTGTAGCCAGGTTAGAGGTCTTGACCAAGCATCACA |
| | | TTCCCTTCTATGAGGTTGATTTGTGTGACCGAAAAGGT |
| | | CTGGAAAAGGTTTTCAAAGAATATAAAATTGATTCGG |
| | | TAATTCACTTTGCTGGTTTAAAGGCTGTAGGTGAATCT |
| | | ACACAAATCCCGCTGAGATACTATCACAATAACATTT |
| | | TGGGAACTGTCGTTTTATTAGAGTTAATGCAACAATAC |
| | | AACGTTTCCAAATTTGTTTTTTCATCTTCTGCTACTGTC |
| | | TATGGTGATGCTACGAGATTCCCAAATATGATTCCTAT |
| | | CCCAGAAGAATGTCCCTTAGGGCCTACTAATCCGTAT |
| | | GGTCATACGAAATACGCCATTGAGAATATCTTGAATG |
| | | ATCTTTACAATAGCGACAAAAAAAGTTGGAAGTTTGC |
| | | TATCTTGCGTTATTTTAACCCAATTGGCGCACATCCCT |
| | | CTGGATTAATCGGAGAAGATCCGCTAGGTATACCAAA |
| | | CAATTTGTTGCCATATATGGCTCAAGTAGCTGTTGGTA |
| | | GGCGCGAGAAGCTTTACATCTTCGGAGACGATTATGA |
| | | TTCCAGAGATGGTACCCCGATCAGGGATTATATCCAC |
| | | GTAGTTGATCTAGCAAAAGGTCATATTGCAGCCCTGC |
| | | AATACCTAGAGGCCTACAATGAAAATGAAGGTTTGTG |
| | | TCGTGAGTGGAACTTGGGTTCCGGTAAAGGTTCTACA |
| | | GTTTTTGAAGTTTATCATGCATTCTGCAAAGCTTCTGG |
| | | TATTGATCTTCCATACAAAGTTACGGGCAGAAGAGCA |
| | | GGTGATGTTTTGAACTTGACGGCTAAACCAGATAGGG |
| | | CCAAACGCGAACTGAAATGGCAGACCGAGTTGCAGGT |
| | | TGAAGACTCCTGCAAGGATTTATGGAAATGGACTACT |
| | | GAGAATCCTTTTGGTTACCAGTTAAGGGGTGTCGAGG |
| | | CCAGATTTTCCGCTGAAGATATGCGTTATGACGCAAG |
| | | ATTTGTGACTATTGGTGCCGGCACCAGATTTCAAGCCA |
| | | CGTTTGCCAATTTGGGCGCCAGCATTGTTGACCTGAAA |
| | | GTGAACGGACAATCAGTTGTTCTTGGCTATGAAAATG |
| | | AGGAAGGGTATTTGAATCCTGATAGTGCTTATATAGG |
| | | CGCCACGATCGGCAGGTATGCTAATCGTATTTCGAAG |
| | | GGTAAGTTTAGTTTATGCAACAAAGACTATCAGTTAA |
| | | CCGTTAATAACGGCGTTAATGCGAATCATAGTAGTAT |
| | | CGGTTCTTTCCACAGAAAAAGATTTTTGGGACCCATCA |
| | | TTCAAAATCCTTCAAAGGATGTTTTTACCGCCGAGTAC |
| | | ATGCTGATAGATAATGAGAAGGACACCGAATTTCCAG |
| | | GTGATCTATTGGTAACCATACAGTATACTGTGAACGTT |
| | | GCCCAAAAAAGTTTGGAAATGGTATATAAAGGTAAAT |
| | | TGACTGCTGGTGAAGCGACGCCAATAAATTTAACAAA |
| | | TCATAGTTATTTCAATCTGAACAAGCCATATGGAGAC |
| | | ACTATTGAGGGTACGGAGATTATGGTGCGTTCAAAAA |
| | | AATCTGTTGATGTCGACAAAAACATGATTCCTACGGG |
| | | TAATATCGTCGATAGAGAAATTGCTACCTTTAACTCTA |
| | | CAAAGCCAACGGTCTTAGGCCCCAAAAATCCCCAGTT |
| | | TGATTGTTGTTTTGTGGTGGATGAAAATGCTAAGCCAA |
| | | GTCAAATCAATACTCTAAACAATGAATTGACGCTTATT |
| | | GTCAAGGCTTTTCATCCCGATTCCAATATTACATTAGA |
| | | AGTTTTAAGTACAGAGCCAACTTATCAATTTTATACCG |
| | | GTGATTTCTTGTCTGCTGGTTACGAAGCAAGACAAGG |
| | | TTTTGCAATTGAGCCTGGTAGATACATTGATGCTATCA |
| | | ATCAAGAGAACTGGAAAGATTGTGTAACCTTGAAAAA |
| | | CGGTGAAACTTACGGGTCCAAGATTGTCTACAGATTTT |
| | | CCTGA |
| 41 | Sequence of the | TAAGCTTCACGATTTGTGTTCCAGTTTATCCCCCCTTT |
| | PpPMA1 | ATATACCGTTAACCCTTTCCCTGTTGAGCTGACTGTTG |
| | terminator | TTGTATTACCGCAATTTTTCCAAGTTTGCCATGCTTTTC |
| | | GTGTTATTTGACCGATGTCTTTTTTCCCAAATCAAACT |
| | | ATATTTGTTACCATTTAAACCAAGTTATCTTTTGTATT |
| | | AAGAGTCTAAGTTTGTTCCCAGGCTTCATGTGAGAGT |
| | | GATAACCATCCAGACTATGATTCTTGTTTTTTATTGGG |
| | | TTTGTTTGTGTGATACATCTGAGTTGTGATTCGTAAAG |
| | | TATGTCAGTCTATCTAGATTTTTAATAGTTAATTGGTA |
| | | ATCAATGACTTGTTTGTTTTAACTTTTAAATTGTGGGT |
| | | CGTATCCACGCGTTTAGTATAGCTGTTCATGGCTGTTA |
| | | GAGGAGGGCGATGTTTATATACAGAGGACAAGAATGA |
| | | GGAGGCGGCGTGTATTTTTAAAATGGAGACGCGACTC |
| | | CTGTACACCTTATCGGTTGG |
| 42 | hGalT codon | GGTAGAGATTTGTCTAGATTGCCACAGTTGGTTGGTGT |
| | optimized (XB) | TTCCACTCCATTGCAAGGAGGTTCTAACTCTGCTGCTG |
| | | CTATTGGTCAATCTTCCGGTGAGTTGAGAACTGGTGG |
| | | AGCTAGACCACCTCCACCATTGGGAGCTTCCTCTCAAC |
| | | CAAGACCAGGTGGTGATTCTTCTCCAGTTGTTGACTCT |
| | | GGTCCAGGTCCAGCTTCTAACTTGACTTCCGTTCCAGT |
| | | TCCACACACTACTGCTTTGTCCTTGCCAGCTTGTCCAG |
| | | AAGAATCCCCATTGTTGGTTGGTCCAATGTTGATCGAG |
| | | TTCAACATGCCAGTTGACTTGGAGTTGGTTGCTAAGCA |
| | | GAACCCAAACGTTAAGATGGGTGGTAGATACGCTCCA |
| | | AGAGACTGTGTTTCCCCACACAAAGTTGCTATCATCAT |
| | | CCCATTCAGAAACAGACAGGAGCACTTGAAGTACTGG |
| | | TTGTACTACTTGCACCCAGTTTTGCAAAGACAGCAGTT |
| | | GGACTACGGTATCTACGTTATCAACCAGGCTGGTGAC |
| | | ACTATTTTCAACAGAGCTAAGTTGTTGAATGTTGGTTT |
| | | CCAGGAGGCTTTGAAGGATTACGACTACACTTGTTTC |
| | | GTTTTCTCCGACGTTGACTTGATTCCAATGAACGACCA |
| | | CAACGCTTACAGATGTTTCTCCCAGCCAAGACACATTT |
| | | CTGTTGCTATGGACAAGTTCGGTTTCTCCTTGCCATAC |
| | | GTTCAATACTTCGGTGGTGTTTCCGCTTTGTCCAAGCA |
| | | GCAGTTCTTGACTATCAACGGTTTCCCAAACAATTACT |
| | | GGGGATGGGGTGGTGAAGATGACGACATCTTTAACAG |
| | | ATTGGTTTTCAGAGGAATGTCCATCTCTAGACCAAAC |
| | | GCTGTTGTTGGTAGATGTAGAATGATCAGACACTCCA |
| | | GAGACAAGAAGAACGAGCCAAACCCACAAAGATTCG |
| | | ACAGAATCGCTCACACTAAGGAAACTATGTTGTCCGA |
| | | CGGATTGAACTCCTTGACTTACCAGGTTTTGGACGTTC |
| | | AGAGATACCCATTGTACACTCAGATCACTGTTGACAT |
| | | CGGTACTCCATCCTAG |
| 43 | DNA encodes | ATGGCCCTCTTTCTCAGTAAGAGACTGTTGAGATTTAC |
| | ScMnt1 (Kre2) | CGTCATTGCAGGTGCGGTTATTGTTCTCCTCCTAACAT |
| | (33) | TGAATTCCAACAGTAGAACTCAGCAATATATTCCGAG |
| | | TTCCATCTCCGCTGCATTTGATTTTACCTCAGGATCTA |
| | | TATCCCCTGAACAACAAGTCATCGGGCGCGCC |
| 44 | DNA encodes | ATGAATAGCATACACATGAACGCCAATACGCTGAAGT |
| | DmUGT | ACATCAGCCTGCTGACGCTGACCCTGCAGAATGCCAT |
| | | CCTGGGCCTCAGCATGCGCTACGCCCGCACCCGGCCA |
| | | GGCGACATCTTCCTCAGCTCCACGGCCGTACTCATGGC |
| | | AGAGTTCGCCAAACTGATCACGTGCCTGTTCCTGGTCT |
| | | TCAACGAGGAGGGCAAGGATGCCCAGAAGTTTGTACG |
| | | CTCGCTGCACAAGACCATCATTGCGAATCCCATGGAC |
| | | ACGCTGAAGGTGTGCGTCCCCTCGCTGGTCTATATCGT |
| | | TCAAAACAATCTGCTGTACGTCTCTGCCTCCCATTTGG |
| | | ATGCGGCCACCTACCAGGTGACGTACCAGCTGAAGAT |
| | | TCTCACCACGGCCATGTTCGCGGTTGTCATTCTGCGCC |
| | | GCAAGCTGCTGAACACGCAGTGGGGTGCGCTGCTGCT |
| | | CCTGGTGATGGGCATCGTCCTGGTGCAGTTGGCCCAA |
| | | ACGGAGGGTCCGACGAGTGGCTCAGCCGGTGGTGCCG |
| | | CAGCTGCAGCCACGGCCGCCTCCTCTGGCGGTGCTCC |
| | | CGAGCAGAACAGGATGCTCGGACTGTGGGCCGCACTG |
| | | GGCGCCTGCTTCCTCTCCGGATTCGCGGGCATCTACTT |
| | | TGAGAAGATCCTCAAGGGTGCCGAGATCTCCGTGTGG |
| | | ATGCGGAATGTGCAGTTGAGTCTGCTCAGCATTCCCTT |
| | | CGGCCTGCTCACCTGTTTCGTTAACGACGGCAGTAGG |
| | | ATCTTCGACCAGGGATTCTTCAAGGGCTACGATCTGTT |
| | | TGTCTGGTACCTGGTCCTGCTGCAGGCCGGCGGTGGA |
| | | TTGATCGTTGCCGTGGTGGTCAAGTACGCGGATAACA |
| | | TTCTCAAGGGCTTCGCCACCTCGCTGGCCATCATCATC |
| | | TCGTGCGTGGCCTCCATATACATCTTCGACTTCAATCT |
| | | CACGCTGCAGTTCAGCTTCGGAGCTGGCCTGGTCATC |
| | | GCCTCCATATTTCTCTACGGCTACGATCCGGCCAGGTC |
| | | GGCGCCGAAGCCAACTATGCATGGTCCTGGCGGCGAT |
| | | GAGGAGAAGCTGCTGCCGCGCGTCTAG |
| 45 | Sequence of the | TGGACACAGGAGACTCAGAAACAGACACAGAGCGTT |
| | PpOCH1 | CTGAGTCCTGGTGCTCCTGACGTAGGCCTAGAACAGG |
| | promoter | AATTATTGGCTTTATTTGTTTGTCCATTTCATAGGCTTG |
| | | GGGTAATAGATAGATGACAGAGAAATAGAGAAGACC |
| | | TAATATTTTTTGTTCATGGCAAATCGCGGGTTCGCGGT |
| | | CGGGTCACACACGGAGAAGTAATGAGAAGAGCTGGT |
| | | AATCTGGGGTAAAAGGGTTCAAAAGAAGGTCGCCTGG |
| | | TAGGGATGCAATACAAGGTTGTCTTGGAGTTTACATTG |
| | | ACCAGATGATTTGGCTTTTTCTCTGTTCAATTCACATTT |
| | | TTCAGCGAGAATCGGATTGACGGAGAAATGGCGGGGT |
| | | GTGGGGTGGATAGATGGCAGAAATGCTCGCAATCACC |
| | | GCGAAAGAAAGACTTTATGGAATAGAACTACTGGGTG |
| | | GTGTAAGGATTACATAGCTAGTCCAATGGAGTCCGTT |
| | | GGAAAGGTAAGAAGAAGCTAAAACCGGCTAAGTAAC |
| | | TAGGGAAGAATGATCAGACTTTGATTTGATGAGGTCT |
| | | GAAAATACTCTGCTGCTTTTTCAGTTGCTTTTTCCCTGC |
| | | AACCTATCATTTTCCTTTTCATAAGCCTGCCTTTTCTGT |
| | | TTTCACTTATATGAGTTCCGCCGAGACTTCCCCAAATT |
| | | CTCTCCTGGAACATTCTCTATCGCTCTCCTTCCAAGTT |
| | | GCGCCCCCTGGCACTGCCTAGTAATATTACCACGCGA |
| | | CTTATATTCAGTTCCACAATTTCCAGTGTTCGTAGCAA |
| | | ATATCATCAGCC |
| 46 | Sequence of the | AATATATACCTCATTTGTTCAATTTGGTGTAAAGAGTG |
| | PpALG12 | TGGCGGATAGACTTCTTGTAAATCAGGAAAGCTACAA |
| | terminator | TTCCAATTGCTGCAAAAAATACCAATGCCCATAAACC |
| | | AGTATGAGCGGTGCCTTCGACGGATTGCTTACTTTCCG |
| | | ACCCTTTGTCGTTTGATTCTTCTGCCTTTGGTGAGTCA |
| | | GTTTGTTTCGACTTTATATCTGACTCATCAACTTCCTTT |
| | | ACGGTTGCGTTTTTAATCATAATTTTAGCCGTTGGCTT |
| | | ATTATCCCTTGAGTTGGTAGGAGTTTTGATGATGCTG |
| 47 | Sequence of the | TAACTGGCCCTTTGACGTTTCTGACAATAGTTCTAGAG |
| | 5′-Region used | GAGTCGTCCAAAAACTCAACTCTGACTTGGGTGACAC |
| | for knock out of | CACCACGGGATCCGGTTCTTCCGAGGACCTTGATGAC |
| | PpHIS1 | CTTGGCTAATGTAACTGGAGTTTTAGTATCCATTTTAA |
| | | GATGTGTGTTTCTGTAGGTTCTGGGTTGGAAAAAAATT |
| | | TTAGACACCAGAAGAGAGGAGTGAACTGGTTTGCGTG |
| | | GGTTTAGACTGTGTAAGGCACTACTCTGTCGAAGTTTT |
| | | AGATAGGGGTTACCCGCTCCGATGCATGGGAAGCGAT |
| | | TAGCCCGGCTGTTGCCCGTTTGGTTTTTGAAGGGTAAT |
| | | TTTCAATATCTCTGTTTGAGTCATCAATTTCATATTCA |
| | | AAGATTCAAAAACAAAATCTGGTCCAAGGAGCGCATT |
| | | TAGGATTATGGAGTTGGCGAATCACTTGAACGATAGA |
| | | CTATTATTTGC |
| 48 | Sequence of the | GTGACATTCTTGTCTTTGAGATCAGTAATTGTAGAGCA |
| | 3′-Region used | TAGATAGAATAATATTCAAGACCAACGGCTTCTCTTC |
| | for knock out of | GGAAGCTCCAAGTAGCTTATAGTGATGAGTACCGGCA |
| | PpHIS1 | TATATTTATAGGCTTAAAATTTCGAGGGTTCACTATAT |
| | | TCGTTTAGTGGGAAGAGTTCCTTTCACTCTTGTTATCT |
| | | ATATTGTCAGCGTGGACTGTTTATAACTGTACCAACTT |
| | | AGTTTCTTTCAACTCCAGGTTAAGAGACATAAATGTCC |
| | | TTTGATGCTGACAATAATCAGTGGAATTCAAGGAAGG |
| | | ACAATCCCGACCTCAATCTGTTCATTAATGAAGAGTTC |
| | | GAATCGTCCTTAAATCAAGCGCTAGACTCAATTGTCA |
| | | ATGAGAACCCTTTCTTTGACCAAGAAACTATAAATAG |
| | | ATCGAATGACAAAGTTGGAAATGAGTCCATTAGCTTA |
| | | CATGATATTGAGCAGGCAGACCAAAATAAACCGTCCT |
| | | TTGAGAGCGATATTGATGGTTCGGCGCCGTTGATAAG |
| | | AGACGACAAATTGCCAAAGAAACAAAGCTGGGGGCT |
| | | GAGCAATTTTTTTTCAAGAAGAAATAGCATATGTTTAC |
| | | CACTACATGAAAATGATTCAAGTGTTGTTAAGACCGA |
| | | AAGATCTATTGCAGTGGGAACACCCCATCTTCAATAC |
| | | TGCTTCAATGGAATCTCCAATGCCAAGTACAATGCATT |
| | | TACCTTTTTCCCAGTCATCCTATACGAGCAATTCAAAT |
| | | TTTTTTTCAATTTATACTTTACTTTAGTGGCTCTCTCTC |
| | | AAGCGATACCGCAACTTCGCATTGGATATCTTTCTTCG |
| | | TATGTCGTCCCACTTTTGTTTGTACTCATAGTGACCAT |
| | | GTCAAAAGAGGCGATGGATGATATTCAACGCCGAAGA |
| | | AGGGATAGAGAACAGAACAATGAACCATATGAGGTTC |
| | | TGTCCAGCCCATCACCAGTTTTGTCCAAAAACTTAAAA |
| | | TGTGGTCACTTGGTTCGATTGCATAAGGGAATGAGAG |
| | | TGCCCGCAGATATGGTTCTTGTCCAGTCAAGCGAATCC |
| | | ACCGGAGAGTCATTTATCAAGACAGATCAGCTGGATG |
| | | GTGAGACTGATTGGAAGCTTCGGATTGTTTCTCCAGTT |
| | | ACACAATCGTTACCAATGACTGAACTTCAAAATGTCG |
| | | CCATCACTGCAAGCGCACCCTCAAAATCAATTCACTC |
| | | CTTTCTTGGAAGATTGACCTACAATGGGCAATCATATG |
| | | GTCTTACGATAGACAACACAATGTGGTGTAATACTGT |
| | | ATTAGCTTCTGGTTCAGCAATTGGTTGTATAATTTACA |
| | | CAGGTAAAGATACTCGACAATCGATGAACACAACTCA |
| | | GCCCAAACTGAAAACGGGCTTGTTAGAACTGGAAATC |
| | | AATAGTTTGTCCAAGATCTTATGTGTTTGTGTGTTTGC |
| | | ATTATCTGTCATCTTAGTGCTATTCCAAGGAATAGCTG |
| | | ATGATTGGTACGTCGATATCATGCGGTTTCTCATTCTA |
| | | TTCTCCACTATTATCCCAGTGTCTCTGAGAGTTAACCT |
| | | TGATCTTGGAAAGTCAGTCCATGCTCATCAAATAGAA |
| | | ACTGATAGCTCAATACCTGAAACCGTTGTTAGAACTA |
| | | GTACAATACCGGAAGACCTGGGAAGAATTGAATACCT |
| | | ATTAAGTGACAAAACTGGAACTCTTACTCAAAATGAT |
| | | ATGGAAATGAAAAAACTACACCTAGGAACAGTCTCTT |
| | | ATGCTGGTGATACCATGGATATTATTTCTGATCATGTT |
| | | AAAGGTCTTAATAACGCTAAAACATCGAGGAAAGATC |
| | | TTGGTATGAGAATAAGAGATTTGGTTACAACTCTGGC |
| | | CATCTG |
| 49 | DNA encodes | AGAGACGATCCAATTAGACCTCCATTGAAGGTTGCTA |
| | Drosophila | GATCCCCAAGACCAGGTCAATGTCAAGATGTTGTTCA |
| | melanogaster | GGACGTCCCAAACGTTGATGTCCAGATGTTGGAGTTG |
| | ManII codon- | TACGATAGAATGTCCTTCAAGGACATTGATGGTGGTG |
| | optimized (KD) | TTTGGAAGCAGGGTTGGAACATTAAGTACGATCCATT |
| | | GAAGTACAACGCTCATCACAAGTTGAAGGTCTTCGTT |
| | | GTCCCACACTCCCACAACGATCCTGGTTGGATTCAGA |
| | | CCTTCGAGGAATACTACCAGCACGACACCAAGCACAT |
| | | CTTGTCCAACGCTTTGAGACATTTGCACGACAACCCA |
| | | GAGATGAAGTTCATCTGGGCTGAAATCTCCTACTTCGC |
| | | TAGATTCTACCACGATTTGGGTGAGAACAAGAAGTTG |
| | | CAGATGAAGTCCATCGTCAAGAACGGTCAGTTGGAAT |
| | | TCGTCACTGGTGGATGGGTCATGCCAGACGAGGCTAA |
| | | CTCCCACTGGAGAAACGTTTTGTTGCAGTTGACCGAA |
| | | GGTCAAACTTGGTTGAAGCAATTCATGAACGTCACTC |
| | | CAACTGCTTCCTGGGCTATCGATCCATTCGGACACTCT |
| | | CCAACTATGCCATACATTTTGCAGAAGTCTGGTTTCAA |
| | | GAATATGTTGATCCAGAGAACCCACTACTCCGTTAAG |
| | | AAGGAGTTGGCTCAACAGAGACAGTTGGAGTTCTTGT |
| | | GGAGACAGATCTGGGACAACAAAGGTGACACTGCTTT |
| | | GTTCACCCACATGATGCCATTCTACTCTTACGACATTC |
| | | CTCATACCTGTGGTCCAGATCCAAAGGTTTGTTGTCAG |
| | | TTCGATTTCAAAAGAATGGGTTCCTTCGGTTTGTCTTG |
| | | TCCATGGAAGGTTCCACCTAGAACTATCTCTGATCAA |
| | | AATGTTGCTGCTAGATCCGATTTGTTGGTTGATCAGTG |
| | | GAAGAAGAAGGCTGAGTTGTACAGAACCAACGTCTTG |
| | | TTGATTCCATTGGGTGACGACTTCAGATTCAAGCAGA |
| | | ACACCGAGTGGGATGTTCAGAGAGTCAACTACGAAAG |
| | | ATTGTTCGAACACATCAACTCTCAGGCTCACTTCAATG |
| | | TCCAGGCTCAGTTCGGTACTTTGCAGGAATACTTCGAT |
| | | GCTGTTCACCAGGCTGAAAGAGCTGGACAAGCTGAGT |
| | | TCCCAACCTTGTCTGGTGACTTCTTCACTTACGCTGAT |
| | | AGATCTGATAACTACTGGTCTGGTTACTACACTTCCAG |
| | | ACCATACCATAAGAGAATGGACAGAGTCTTGATGCAC |
| | | TACGTTAGAGCTGCTGAAATGTTGTCCGCTTGGCACTC |
| | | CTGGGACGGTATGGCTAGAATCGAGGAAAGATTGGAG |
| | | CAGGCTAGAAGAGAGTTGTCCTTGTTCCAGCACCACG |
| | | ACGGTATTACTGGTACTGCTAAAACTCACGTTGTCGTC |
| | | GACTACGAGCAAAGAATGCAGGAAGCTTTGAAAGCTT |
| | | GTCAAATGGTCATGCAACAGTCTGTCTACAGATTGTTG |
| | | ACTAAGCCATCCATCTACTCTCCAGACTTCTCCTTCTC |
| | | CTACTTCACTTTGGACGACTCCAGATGGCCAGGTTCTG |
| | | GTGTTGAGGACTCTAGAACTACCATCATCTTGGGTGA |
| | | GGATATCTTGCCATCCAAGCATGTTGTCATGCACAAC |
| | | ACCTTGCCACACTGGAGAGAGCAGTTGGTTGACTTCT |
| | | ACGTCTCCTCTCCATTCGTTTCTGTTACCGACTTGGCT |
| | | AACAATCCAGTTGAGGCTCAGGTTTCTCCAGTTTGGTC |
| | | TTGGCACCACGACACTTTGACTAAGACTATCCACCCA |
| | | CAAGGTTCCACCACCAAGTACAGAATCATCTTCAAGG |
| | | CTAGAGTTCCACCAATGGGTTTGGCTACCTACGTTTTG |
| | | ACCATCTCCGATTCCAAGCCAGAGCACACCTCCTACG |
| | | CTTCCAATTTGTTGCTTAGAAAGAACCCAACTTCCTTG |
| | | CCATTGGGTCAATACCCAGAGGATGTCAAGTTCGGTG |
| | | ATCCAAGAGAGATCTCCTTGAGAGTTGGTAACGGTCC |
| | | AACCTTGGCTTTCTCTGAGCAGGGTTTGTTGAAGTCCA |
| | | TTCAGTTGACTCAGGATTCTCCACATGTTCCAGTTCAC |
| | | TTCAAGTTCTTGAAGTACGGTGTTAGATCTCATGGTGA |
| | | TAGATCTGGTGCTTACTTGTTCTTGCCAAATGGTCCAG |
| | | CTTCTCCAGTCGAGTTGGGTCAGCCAGTTGTCTTGGTC |
| | | ACTAAGGGTAAATTGGAGTCTTCCGTTTCTGTTGGTTT |
| | | GCCATCTGTCGTTCACCAGACCATCATGAGAGGTGGT |
| | | GCTCCAGAGATTAGAAATTTGGTCGATATTGGTTCTTT |
| | | GGACAACACTGAGATCGTCATGAGATTGGAGACTCAT |
| | | ATCGACTCTGGTGATATCTTCTACACTGATTTGAATGG |
| | | ATTGCAATTCATCAAGAGGAGAAGATTGGACAAGTTG |
| | | CCATTGCAGGCTAACTACTACCCAATTCCATCTGGTAT |
| | | GTTCATTGAGGATGCTAATACCAGATTGACTTTGTTGA |
| | | CCGGTCAACCATTGGGTGGATCTTCTTTGGCTTCTGGT |
| | | GAGTTGGAGATTATGCAAGATAGAAGATTGGCTTCTG |
| | | ATGATGAAAGAGGTTTGGGTCAGGGTGTTTTGGACAA |
| | | CAAGCCAGTTTTGCATATTTACAGATTGGTCTTGGAGA |
| | | AGGTTAACAACTGTGTCAGACCATCTAAGTTGCATCC |
| | | AGCTGGTTACTTGACTTCTGCTGCTCACAAAGCTTCTC |
| | | AGTCTTTGTTGGATCCATTGGACAAGTTCATCTTCGCT |
| | | GAAAATGAGTGGATCGGTGCTCAGGGTCAATTCGGTG |
| | | GTGATCATCCATCTGCTAGAGAGGATTTGGATGTCTCT |
| | | GTCATGAGAAGATTGACCAAGTCTTCTGCTAAAACCC |
| | | AGAGAGTTGGTTACGTTTTGCACAGAACCAATTTGAT |
| | | GCAATGTGGTACTCCAGAGGAGCATACTCAGAAGTTG |
| | | GATGTCTGTCACTTGTTGCCAAATGTTGCTAGATGTGA |
| | | GAGAACTACCTTGACTTTCTTGCAGAATTTGGAGCACT |
| | | TGGATGGTATGGTTGCTCCAGAAGTTTGTCCAATGGA |
| | | AACCGCTGCTTACGTCTCTTCTCACTCTTCTTGA |
| 50 | DNA encodes | ATGCTGCTTACCAAAAGGTTTTCAAAGCTGTTCAAGCT |
| | ScMNN2-s | GACGTTCATAGTTTTGATATTGTGCGGGCTGTTCGTCA |
| | leader (53) | TTACAAACAAATACATGGATGAGAACACGTCG |
| 51 | Sequence of the | CAAGTTGCGTCCGGTATACGTAACGTCTCACGATGAT |
| | PpHIS1 | CAAAGATAATACTTAATCTTCATGGTCTACTGAATAAC |
| | auxotrophic | TCATTTAAACAATTGACTAATTGTACATTATATTGAAC |
| | marker | TTATGCATCCTATTAACGTAATCTTCTGGCTTCTCTCTC |
| | | AGACTCCATCAGACACAGAATATCGTTCTCTCTAACTG |
| | | GTCCTTTGACGTTTCTGACAATAGTTCTAGAGGAGTCG |
| | | TCCAAAAACTCAACTCTGACTTGGGTGACACCACCAC |
| | | GGGATCCGGTTCTTCCGAGGACCTTGATGACCTTGGCT |
| | | AATGTAACTGGAGTTTTAGTATCCATTTTAAGATGTGT |
| | | GTTTCTGTAGGTTCTGGGTTGGAAAAAAATTTTAGACA |
| | | CCAGAAGAGAGGAGTGAACTGGTTTGCGTGGGTTTAG |
| | | ACTGTGTAAGGCACTACTCTGTCGAAGTTTTAGATAG |
| | | GGGTTACCCGCTCCGATGCATGGGAAGCGATTAGCCC |
| | | GGCTGTTGCCCGTTTGGTTTTTGAAGGGTAATTTTCAA |
| | | TATCTCTGTTTGAGTCATCAATTTCATATTCAAAGATT |
| | | CAAAAACAAAATCTGGTCCAAGGAGCGCATTTAGGAT |
| | | TATGGAGTTGGCGAATCACTTGAACGATAGACTATTA |
| | | TTTGCTGTTCCTAAAGAGGGCAGATTGTATGAGAAAT |
| | | GCGTTGAATTACTTAGGGGATCAGATATTCAGTTTCGA |
| | | AGATCCAGTAGATTGGATATAGCTTTGTGCACTAACCT |
| | | GCCCCTGGCATTGGTTTTCCTTCCAGCTGCTGACATTC |
| | | CCACGTTTGTAGGAGAGGGTAAATGTGATTTGGGTAT |
| | | AACTGGTATTGACCAGGTTCAGGAAAGTGACGTAGAT |
| | | GTCATACCTTTATTAGACTTGAATTTCGGTAAGTGCAA |
| | | GTTGCAGATTCAAGTTCCCGAGAATGGTGACTTGAAA |
| | | GAACCTAAACAGCTAATTGGTAAAGAAATTGTTTCCT |
| | | CCTTTACTAGCTTAACCACCAGGTACTTTGAACAACTG |
| | | GAAGGAGTTAAGCCTGGTGAGCCACTAAAGACAAAA |
| | | ATCAAATATGTTGGAGGGTCTGTTGAGGCCTCTTGTGC |
| | | CCTAGGAGTTGCCGATGCTATTGTGGATCTTGTTGAGA |
| | | GTGGAGAAACCATGAAAGCGGCAGGGCTGATCGATAT |
| | | TGAAACTGTTCTTTCTACTTCCGCTTACCTGATCTCTTC |
| | | GAAGCATCCTCAACACCCAGAACTGATGGATACTATC |
| | | AAGGAGAGAATTGAAGGTGTACTGACTGCTCAGAAGT |
| | | ATGTCTTGTGTAATTACAACGCACCTAGAGGTAACCTT |
| | | CCTCAGCTGCTAAAACTGACTCCAGGCAAGAGAGCTG |
| | | CTACCGTTTCTCCATTAGATGAAGAAGATTGGGTGGG |
| | | AGTGTCCTCGATGGTAGAGAAGAAAGATGTTGGAAGA |
| | | ATCATGGACGAATTAAAGAAACAAGGTGCCAGTGACA |
| | | TTCTTGTCTTTGAGATCAGTAATTGTAGAGCATAGATA |
| | | GAATAATATTCAAGACCAACGGCTTCTCTTCGGAAGC |
| | | TCCAAGTAGCTTATAGTGATGAGTACCGGCATATATTT |
| | | ATAGGCTTAAAATTTCGAGGGTTCACTATATTCGTTTA |
| | | GTGGGAAGAGTTCCTTTCACTCTTGTTATCTATATTGT |
| | | CAGCGTGGACTGTTTATAACTGTACCAACTTAGTTTCT |
| | | TTCAACTCCAGGTTAAGAGACATAAATGTCCTTTGATGC |
| 52 | DNA encodes | TCCTTGGTTTACCAATTGAACTTCGACCAGATGTTGAG |
| | Rat GnT II | AAACGTTGACAAGGACGGTACTTGGTCTCCTGGTGAG |
| | (TC) | TTGGTTTTGGTTGTTCAGGTTCACAACAGACCAGAGTA |
| | Codon- | CTTGAGATTGTTGATCGACTCCTTGAGAAAGGCTCAA |
| | optimized | GGTATCAGAGAGGTTTTGGTTATCTTCTCCCACGATTT |
| | | CTGGTCTGCTGAGATCAACTCCTTGATCTCCTCCGTTG |
| | | ACTTCTGTCCAGTTTTGCAGGTTTTCTTCCCATTCTCCA |
| | | TCCAATTGTACCCATCTGAGTTCCCAGGTTCTGATCCA |
| | | AGAGACTGTCCAAGAGACTTGAAGAAGAACGCTGCTT |
| | | TGAAGTTGGGTTGTATCAACGCTGAATACCCAGATTCT |
| | | TTCGGTCACTACAGAGAGGCTAAGTTCTCCCAAACTA |
| | | AGCATCATTGGTGGTGGAAGTTGCACTTTGTTTGGGAG |
| | | AGAGTTAAGGTTTTGCAGGACTACACTGGATTGATCTT |
| | | GTTCTTGGAGGAGGATCATTACTTGGCTCCAGACTTCT |
| | | ACCACGTTTTCAAGAAGATGTGGAAGTTGAAGCAACA |
| | | AGAGTGTCCAGGTTGTGACGTTTTGTCCTTGGGAACTT |
| | | ACACTACTATCAGATCCTTCTACGGTATCGCTGACAAG |
| | | GTTGACGTTAAGACTTGGAAGTCCACTGAACACAACA |
| | | TGGGATTGGCTTTGACTAGAGATGCTTACCAGAAGTT |
| | | GATCGAGTGTACTGACACTTTCTGTACTTACGACGACT |
| | | ACAACTGGGACTGGACTTTGCAGTACTTGACTTTGGCT |
| | | TGTTTGCCAAAAGTTTGGAAGGTTTTGGTTCCACAGGC |
| | | TCCAAGAATTTTCCACGCTGGTGACTGTGGAATGCAC |
| | | CACAAGAAAACTTGTAGACCATCCACTCAGTCCGCTC |
| | | AAATTGAGTCCTTGTTGAACAACAACAAGCAGTACTT |
| | | GTTCCCAGAGACTTTGGTTATCGGAGAGAAGTTTCCA |
| | | ATGGCTGCTATTTCCCCACCAAGAAAGAATGGTGGAT |
| | | GGGGTGATATTAGAGACCACGAGTTGTGTAAATCCTA |
| | | CAGAAGATTGCAGTAG |
| 53 | DNA encodes | ATGCTGCTTACCAAAAGGTTTTCAAAGCTGTTCAAGCT |
| | ScMNN2 leader | GACGTTCATAGTTTTGATATTGTGCGGGCTGTTCGTCA |
| | (54) | TTACAAACAAATACATGGATGAGAACACGTCGGTCAA |
| | The last 9 | GGAGTACAAGGAGTACTTAGACAGATATGTCCAGAGT |
| | nucleotides are | TACTCCAATAAGTATTCATCTTCCTCAGACGCCGCCAG |
| | the linker | CGCTGACGATTCAACCCCATTGAGGGACAATGATGAG |
| | containing the | GCAGGCAATGAAAAGTTGAAAAGCTTCTACAACAACG |
| | AscI restriction | TTTTCAACTTTCTAATGGTTGATTCGCCCGGGCGCGCC |
| | site | |
| 54 | Sequence of the | GATCTGGCCTTCCCTGAATTTTTACGTCCAGCTATACG |
| | 5′-Region used | ATCCGTTGTGACTGTATTTCCTGAAATGAAGTTTCAAC |
| | for knock out of | CTAAAGTTTTGGTTGTACTTGCTCCACCTACCACGGAA |
| | PpARG1 | ACTAATATCGAAACCAATGAAAAAGTAGAACTGGAAT |
| | | CGTCAATCGAAATTCGCAACCAAGTGGAACCCAAAGA |
| | | CTTGAATCTTTCTAAAGTCTATTCTAGTGACACTAATG |
| | | GCAACAGAAGATTTGAGCTGACTTTTCAAATGAATCT |
| | | CAATAATGCAATATCAACATCAGACAATCAATGGGCT |
| | | TTGTCTAGTGACACAGGATCAATTATAGTAGTGTCTTC |
| | | TGCAGGAAGAATAACTTCCCCGATCCTAGAAGTCGGG |
| | | GCATCCGTCTGTGTCTTAAGATCGTACAACGAACACCT |
| | | TTTGGCAATAACTTGTGAAGGAACATGCTTTTCATGGA |
| | | ATTTAAAGAAGCAAGAATGTGTTCTAAACAGCATTTC |
| | | ATTAGCACCTATAGTCAATTCACACATGCTAGTTAAG |
| | | AAAGTTGGAGATGCAAGGAACTATTCTATTGTATCTG |
| | | CCGAAGGAGACAACAATCCGTTACCCCAGATTCTAGA |
| | | CTGCGAACTTTCCAAAAATGGCGCTCCAATTGTGGCTC |
| | | TTAGCACGAAAGACATCTACTCTTATTCAAAGAAAAT |
| | | GAAATGCTGGATCCATTTGATTGATTCGAAATACTTTG |
| | | AATTGTTGGGTGCTGACAATGCACTGTTTGAGTGTGTG |
| | | GAAGCGCTAGAAGGTCCAATTGGAATGCTAATTCATA |
| | | GATTGGTAGATGAGTTCTTCCATGAAAACACTGCCGG |
| | | TAAAAAACTCAAACTTTACAACAAGCGAGTACTGGAG |
| | | GACCTTTCAAATTCACTTGAAGAACTAGGTGAAAATG |
| | | CGTCTCAATTAAGAGAGAAACTTGACAAACTCTATGG |
| | | TGATGAGGTTGAGGCTTCTTGACCTCTTCTCTCTATCT |
| | | GCGTTTCTTTTTTTTTTTTTTTTTTTTTTTTTTTCAGTTG |
| | | AGCCAGACCGCGCTAAACGCATACCAATTGCCAAATC |
| | | AGGCAATTGTGAGACAGTGGTAAAAAAGATGCCTGCA |
| | | AAGTTAGATTCACACAGTAAGAGAGATCCTACTCATA |
| | | AATGAGGCGCTTATTTAGTAGCTAGTGATAGCCACTG |
| | | CGGTTCTGCTTTATGCTATTTGTTGTATGCCTTACTATC |
| | | TTTGTTTGGCTCCTTTTTCTTGACGTTTTCCGTTGGAGG |
| | | GACTCCCTATTCTGAGTCATGAGCCGCACAGATTATCG |
| | | CCCAAAATTGACAAAATCTTCTGGCGAAAAAAGTATA |
| | | AAAGGAGAAAAAAGCTCACCCTTTTCCAGCGTAGAAA |
| | | GTATATATCAGTCATTGAAGAC |
| 55 | Sequence of the | GGGACTTTAACTCAAGTAAAAGGATAGTTGTACAATT |
| | 3′-Region used | ATATATACGAAGAATAAATCATTACAAAAAGTATTCG |
| | for knock out of | TTTCTTTGATTCTTAACAGGATTCATTTTCTGGGTGTCA |
| | PpARG1 | TCAGGTACAGCGCTGAATATCTTGAAGTTAACATCGA |
| | | GCTCATCATCGACGTTCATCACACTAGCCACGTTTCCG |
| | | CAACGGTAGCAATAATTAGGAGCGGACCACACAGTGA |
| | | CGACATCTTTCTCTTTGAAATGGTATCTGAAGCCTTCC |
| | | ATGACCAATTGATGGGCTCTAGCGATGAGTTGCAAGT |
| | | TATTAATGTGGTTGAACTCACGTGCTACTCGAGCACCG |
| | | AATAACCAGCCAGCTCCACGAGGAGAAACAGCCCAA |
| | | CTGTCGACTTCATCTGGGTCAGACCAAACCAAGTCAC |
| | | AAAATCCTCCTTCATGAGGGACCTCTTGCGCTCGGCTG |
| | | AGAACTCTGATTTGATCTAACATGCGAATATCGGGAG |
| | | AGAGACCACCATGGATACATAATATTTTACCATCAAT |
| | | GATGGCACTAAGGGTTAAAAAGTCGAACACCTGGCAA |
| | | CAGTACTTCCAGACAGTGGTGGAACCATATTTATTGA |
| | | GACATTCCTCATAAAATCCATAAACCTGAGTGATCTGT |
| | | CTGGATTCATGATTTCCCCTTACCAATGTGATATGTTG |
| | | AGGAAACTTAATTTTTAAAATCATGAGTAACGTGAAC |
| | | GTCTCCAACGAGAAATAGCCTCTATCCACATAGTCTCC |
| | | TAGGAAGATATAGTTCTGTTTTATTCCATTAGAGGAGG |
| | | ATCCGGGAAACCCACCACTAATCTTGAAAAGTTCCAG |
| | | TAGATCGTGAAATTGGCCGTGAATATCTCCGCATACT |
| | | GTCACTGGACTCTGCACTGGCTGTATATTGGATTCCTC |
| | | CATCAGCAAATCCTTCACCCGTTCGCAAAGATGCTTCA |
| | | TATCATTTTCACTTAAAGCCTTGCAGCTTTTGACTTCTT |
| | | CAAACCACTGATCTGGTCCTCTTTCTGGCATGATTAAG |
| | | GTCTATAATATTTCTGAGCTGAGATGTAAAAAAAAAT |
| | | AATAAAAATGGGGAGTGAAAAAGTGTGTAGCTTTTAG |
| | | GAGTTTGGGATTGATACCCCAAAATGATCTTTATGAG |
| | | AATTAAAAGGTAGATACGCTTTTAATAAGAACACCTA |
| | | TCTATAGTACTTTGTGGTCTTGAGTAATTGAGATGTTC |
| | | AGCTTCTGAGGTTTGCCGTTATTCTGGGATAGTAGTGC |
| | | GCGACCAAACAACCCGCCAGGCAAAGTGTGTTGTGCT |
| | | CGAAGACGATTGCCAGAAGAGTAAGTCCGTCCTGCCT |
| | | CAGATGTTACACACTTTCTTCCCTAGACAGTCGATGCA |
| | | TCATCGGATTTAAACCTGAAACTTTGATGCCATGATAC |
| | | GCCTAGTCACGTCGACTGAGATTTTAGATAAGCCCCG |
| | | ATCCCTTTAGTACATTCCTGTTATCCATGGATGGAATG |
| | | GCCTGATA |
| 56 | Sequence of the | AAGCTTGTTCACCGTTGGGACTTTTCCGTGGACAATGT |
| | 5′-Region used | TGACTACTCCAGGAGGGATTCCAGCTTTCTCTACTAGC |
| | for knock out of | TCAGCAATAATCAATGCAGCCCCAGGCGCCCGTTCTG |
| | PpBMT4 | ATGGCTTGATGACCGTTGTATTGCCTGTCACTATAGCC |
| | | AGGGGTAGGGTCCATAAAGGAATCATAGCAGGGAAA |
| | | TTAAAAGGGCATATTGATGCAATCACTCCCAATGGCT |
| | | CTCTTGCCATTGAAGTCTCCATATCAGCACTAACTTCC |
| | | AAGAAGGACCCCTTCAAGTCTGACGTGATAGAGCACG |
| | | CTTGCTCTGCCACCTGTAGTCCTCTCAAAACGTCACCT |
| | | TGTGCATCAGCAAAGACTTTACCTTGCTCCAATACTAT |
| | | GACGGAGGCAATTCTGTCAAAATTCTCTCTCAGCAATT |
| | | CAACCAACTTGAAAGCAAATTGCTGTCTCTTGATGAT |
| | | GGAGACTTTTTTCCAAGATTGAAATGCAATGTGGGAC |
| | | GACTCAATTGCTTCTTCCAGCTCCTCTTCGGTTGATTG |
| | | AGGAACTTTTGAAACCACAAAATTGGTCGTTGGGTCA |
| | | TGTACATCAAACCATTCTGTAGATTTAGATTCGACGAA |
| | | AGCGTTGTTGATGAAGGAAAAGGTTGGATACGGTTTG |
| | | TCGGTCTCTTTGGTATGGCCGGTGGGGTATGCAATTGC |
| | | AGTAGAAGATAATTGGACAGCCATTGTTGAAGGTAGA |
| | | GAAAAGGTCAGGGAACTTGGGGGTTATTTATACCATT |
| | | TTACCCCACAAATAACAACTGAAAAGTACCCATTCCA |
| | | TAGTGAGAGGTAACCGACGGAAAAAGACGGGCCCAT |
| | | GTTCTGGGACCAATAGAACTGTGTAATCCATTGGGAC |
| | | TAATCAACAGACGATTGGCAATATAATGAAATAGTTC |
| | | GTTGAAAAGCCACGTCAGCTGTCTTTTCATTAACTTTG |
| | | GTCGGACACAACATTTTCTACTGTTGTATCTGTCCTAC |
| | | TTTGCTTATCATCTGCCACAGGGCAAGTGGATTTCCTT |
| | | CTCGCGCGGCTGGGTGAAAACGGTTAACGTGAA |
| 57 | Sequence of the | GCCTTGGGGGACTTCAAGTCTTTGCTAGAAACTAGAT |
| | 3′-Region used | GAGGTCAGGCCCTCTTATGGTTGTGTCCCAATTGGGCA |
| | for knock out of | ATTTCACTCACCTAAAAAGCATGACAATTATTTAGCG |
| | PpBMT4 | AAATAGGTAGTATATTTTCCCTCATCTCCCAAGCAGTT |
| | | TCGTTTTTGCATCCATATCTCTCAAATGAGCAGCTACG |
| | | ACTCATTAGAACCAGAGTCAAGTAGGGGTGAGCTCAG |
| | | TCATCAGCCTTCGTTTCTAAAACGATTGAGTTCTTTTG |
| | | TTGCTACAGGAAGCGCCCTAGGGAACTTTCGCACTTT |
| | | GGAAATAGATTTTGATGACCAAGAGCGGGAGTTGATA |
| | | TTAGAGAGGCTGTCCAAAGTACATGGGATCAGGCCGG |
| | | CCAAATTGATTGGTGTGACTAAACCATTGTGTACTTGG |
| | | ACACTCTATTACAAAAGCGAAGATGATTTGAAGTATT |
| | | ACAAGTCCCGAAGTGTTAGAGGATTCTATCGAGCCCA |
| | | GAATGAAATCATCAACCGTTATCAGCAGATTGATAAA |
| | | CTCTTGGAAAGCGGTATCCCATTTTCATTATTGAAGAA |
| | | CTACGATAATGAAGATGTGAGAGACGGCGACCCTCTG |
| | | AACGTAGACGAAGAAACAAATCTACTTTTGGGGTACA |
| | | ATAGAGAAAGTGAATCAAGGGAGGTATTTGTGGCCAT |
| | | AATACTCAACTCTATCATTAATG |
| 58 | Sequence of the | CATATGGTGAGAGCCGTTCTGCACAACTAGATGTTTTC |
| | 5′-Region used | GAGCTTCGCATTGTTTCCTGCAGCTCGACTATTGAATT |
| | for knock out of | AAGATTTCCGGATATCTCCAATCTCACAAAAACTTATG |
| | PpBMT1 | TTGACCACGTGCTTTCCTGAGGCGAGGTGTTTTATATG |
| | | CAAGCTGCCAAAAATGGAAAACGAATGGCCATTTTTC |
| | | GCCCAGGCAAATTATTCGATTACTGCTGTCATAAAGA |
| | | CAGTGTTGCAAGGCTCACATTTTTTTTTAGGATCCGAG |
| | | ATAAAGTGAATACAGGACAGCTTATCTCTATATCTTGT |
| | | ACCATTCGTGAATCTTAAGAGTTCGGTTAGGGGGACT |
| | | CTAGTTGAGGGTTGGCACTCACGTATGGCTGGGCGCA |
| | | GAAATAAAATTCAGGCGCAGCAGCACTTATCGATG |
| 59 | Sequence of the | GAATTCACAGTTATAAATAAAAACAAAAACTCAAAAA |
| | 3′-Region used | GTTTGGGCTCCACAAAATAACTTAATTTAAATTTTTGT |
| | for knock out of | CTAATAAATGAATGTAATTCCAAGATTATGTGATGCA |
| | PpBMT1 | AGCACAGTATGCTTCAGCCCTATGCAGCTACTAATGTC |
| | | AATCTCGCCTGCGAGCGGGCCTAGATTTTCACTACAA |
| | | ATTTCAAAACTACGCGGATTTATTGTCTCAGAGAGCA |
| | | ATTTGGCATTTCTGAGCGTAGCAGGAGGCTTCATAAG |
| | | ATTGTATAGGACCGTACCAACAAATTGCCGAGGCACA |
| | | ACACGGTATGCTGTGCACTTATGTGGCTACTTCCCTAC |
| | | AACGGAATGAAACCTTCCTCTTTCCGCTTAAACGAGA |
| | | AAGTGTGTCGCAATTGAATGCAGGTGCCTGTGCGCCT |
| | | TGGTGTATTGTTTTTGAGGGCCCAATTTATCAGGCGCC |
| | | TTTTTTCTTGGTTGTTTTCCCTTAGCCTCAAGCAAGGTT |
| | | GGTCTATTTCATCTCCGCTTCTATACCGTGCCTGATAC |
| | | TGTTGGATGAGAACACGACTCAACTTCCTGCTGCTCTG |
| | | TATTGCCAGTGTTTTGTCTGTGATTTGGATCGGAGTCC |
| | | TCCTTACTTGGAATGATAATAATCTTGGCGGAATCTCC |
| | | CTAAACGGAGGCAAGGATTCTGCCTATGATGATCTGC |
| | | TATCATTGGGAAGCTT |
| 60 | Sequence of the | GATATCTCCCTGGGGACAATATGTGTTGCAACTGTTCG |
| | 5′-Region used | TTGTTGGTGCCCCAGTCCCCCAACCGGTACTAATCGGT |
| | for knock out of | CTATGTTCCCGTAACTCATATTCGGTTAGAACTAGAAC |
| | PpBMT3 | AATAAGTGCATCATTGTTCAACATTGTGGTTCAATTGT |
| | | CGAACATTGCTGGTGCTTATATCTACAGGGAAGACGA |
| | | TAAGCCTTTGTACAAGAGAGGTAACAGACAGTTAATT |
| | | GGTATTTCTTTGGGAGTCGTTGCCCTCTACGTTGTCTC |
| | | CAAGACATACTACATTCTGAGAAACAGATGGAAGACT |
| | | CAAAAATGGGAGAAGCTTAGTGAAGAAGAGAAAGTT |
| | | GCCTACTTGGACAGAGCTGAGAAGGAGAACCTGGGTT |
| | | CTAAGAGGCTGGACTTTTTGTTCGAGAGTTAAACTGC |
| | | ATAATTTTTTCTAAGTAAATTTCATAGTTATGAAATTT |
| | | CTGCAGCTTAGTGTTTACTGCATCGTTTACTGCATCAC |
| | | CCTGTAAATAATGTGAGCTTTTTTCCTTCCATTGCTTG |
| | | GTATCTTCCTTGCTGCTGTTT |
| 61 | Sequence of the | ACAAAACAGTCATGTACAGAACTAACGCCTTTAAGAT |
| | 3′-Region used | GCAGACCACTGAAAAGAATTGGGTCCCATTTTTCTTG |
| | for knock out of | AAAGACGACCAGGAATCTGTCCATTTTGTTTACTCGTT |
| | PpBMT3 | CAATCCTCTGAGAGTACTCAACTGCAGTCTTGATAAC |
| | | GGTGCATGTGATGTTCTATTTGAGTTACCACATGATTT |
| | | TGGCATGTCTTCCGAGCTACGTGGTGCCACTCCTATGC |
| | | TCAATCTTCCTCAGGCAATCCCGATGGCAGACGACAA |
| | | AGAAATTTGGGTTTCATTCCCAAGAACGAGAATATCA |
| | | GATTGCGGGTGTTCTGAAACAATGTACAGGCCAATGT |
| | | TAATGCTTTTTGTTAGAGAAGGAACAAACTTTTTTGCT |
| | | GAGC |
| 62 | PpTRP2: 5′ and | ACTGGGCCTTTAGAGGGTGCTGAAGTTGACCCCTTGG |
| | ORF | TGCTTCTGGAAAAAGAACTGAAGGGCACCAGACAAGC |
| | | GCAACTTCCTGGTATTCCTCGTCTAAGTGGTGGTGCCA |
| | | TAGGATACATCTCGTACGATTGTATTAAGTACTTTGAA |
| | | CCAAAAACTGAAAGAAAACTGAAAGATGTTTTGCAAC |
| | | TTCCGGAAGCAGCTTTGATGTTGTTCGACACGATCGTG |
| | | GCTTTTGACAATGTTTATCAAAGATTCCAGGTAATTGG |
| | | AAACGTTTCTCTATCCGTTGATGACTCGGACGAAGCTA |
| | | TTCTTGAGAAATATTATAAGACAAGAGAAGAAGTGGA |
| | | AAAGATCAGTAAAGTGGTATTTGACAATAAAACTGTT |
| | | CCCTACTATGAACAGAAAGATATTATTCAAGGCCAAA |
| | | CGTTCACCTCTAATATTGGTCAGGAAGGGTATGAAAA |
| | | CCATGTTCGCAAGCTGAAAGAACATATTCTGAAAGGA |
| | | GACATCTTCCAAGCTGTTCCCTCTCAAAGGGTAGCCA |
| | | GGCCGACCTCATTGCACCCTTTCAACATCTATCGTCAT |
| | | TTGAGAACTGTCAATCCTTCTCCATACATGTTCTATAT |
| | | TGACTATCTAGACTTCCAAGTTGTTGGTGCTTCACCTG |
| | | AATTACTAGTTAAATCCGACAACAACAACAAAATCAT |
| | | CACACATCCTATTGCTGGAACTCTTCCCAGAGGTAAA |
| | | ACTATCGAAGAGGACGACAATTATGCTAAGCAATTGA |
| | | AGTCGTCTTTGAAAGACAGGGCCGAGCACGTCATGCT |
| | | GGTAGATTTGGCCAGAAATGATATTAACCGTGTGTGT |
| | | GAGCCCACCAGTACCACGGTTGATCGTTTATTGACTGT |
| | | GGAGAGATTTTCTCATGTGATGCATCTTGTGTCAGAAG |
| | | TCAGTGGAACATTGAGACCAAACAAGACTCGCTTCGA |
| | | TGCTTTCAGATCCATTTTCCCAGCAGGTACCGTCTCCG |
| | | GTGCTCCGAAGGTAAGAGCAATGCAACTCATAGGAGA |
| | | ATTGGAAGGAGAAAAGAGAGGTGTTTATGCGGGGGCC |
| | | GTAGGACACTGGTCGTACGATGGAAAATCGATGGACA |
| | | CATGTATTGCCTTAAGAACAATGGTCGTCAAGGACGG |
| | | TGTCGCTTACCTTCAAGCCGGAGGTGGAATTGTCTACG |
| | | ATTCTGACCCCTATGACGAGTACATCGAAACCATGAA |
| | | CAAAATGAGATCCAACAATAACACCATCTTGGAGGCT |
| | | GAGAAAATCTGGACCGATAGGTTGGCCAGAGACGAG |
| | | AATCAAAGTGAATCCGAAGAAAACGATCAATGA |
| 63 | PpTRP2 3′ | ACGGAGGACGTAAGTAGGAATTTATGTAATCATGCCA |
| | region | ATACATCTTTAGATTTCTTCCTCTTCTTTTTAACGAAAG |
| | | ACCTCCAGTTTTGCACTCTCGACTCTCTAGTATCTTCC |
| | | CATTTCTGTTGCTGCAACCTCTTGCCTTCTGTTTCCTTC |
| | | AATTGTTCTTCTTTCTTCTGTTGCACTTGGCCTTCTTCC |
| | | TCCATCTTTCGTTTTTTTTCAAGCCTTTTCAGCAGTTCT |
| | | TCTTCCAAGAGCAGTTCTTTGATTTTCTCTCTCCAATCC |
| | | ACCAAAAAACTGGATGAATTCAACCGGGCATCATCAA |
| | | TGTTCCACTTTCTTTCTCTTATCAATAATCTACGTGCTT |
| | | CGGCATACGAGGAATCCAGTTGCTCCCTAATCGAGTC |
| | | ATCCACAAGGTTAGCATGGGCCTTTTTCAGGGTGTCA |
| | | AAAGCATCTGGAGCTCGTTTATTCGGAGTCTTGTCTGG |
| | | ATGGATCAGCAAAGACTTTTTGCGGAAAGTCTTTCTTA |
| | | TATCTTCCGGAGAACAACCTGGTTTCAAATCCAAGAT |
| | | GGCATAGCTGTCCAATTTGAAAGTGGAAAGAATCCTG |
| | | CCAATTTCCTTCTCTCGTGTCAGCTCGTTCTCCTCCTTT |
| | | TGCAACAGGTCCACTTCATCTGGCATTTTTCTTTATGT |
| | | TAACTTTAATTATTATTAATTATAAAGTTGATTATCGT |
| | | TATCAAAATAATCATATTCGAGAAATAATCCGTCCAT |
| | | GCAATATATAAATAAGAATTCATAATAATGTAATGAT |
| | | AACAGTACCTCTGATGACCTTTGATGAACCGCAATTTT |
| | | CTTTCCAATGACAAGACATCCCTATAATACAATTATAC |
| | | AGTTTATATATCACAAATAATCACCTTTTTATAAGAAA |
| | | ACCGTCCTCTCCGTAACAGAACTTATTATCCGCACGTT |
| | | ATGGTTAACACACTACTAATACCGATATAGTGTATGA |
| | | AGTCGCTACGAGATAGCCATCCAGGAAACTTACCAAT |
| | | TCATCAGCACTTTCATGATCCGATTGTTGGCTTTATTC |
| | | TTTGCGAGACAGATACTTGCCAATGAAATAACTGATC |
| | | CCACAGATGAGAATCCGGTGCTCGT |
| 64 | Mouse CMP- | ATGGCTCCAGCTAGAGAAAACGTTTCCTTGTTCTTCAA |
| | sialic acid | GTTGTACTGTTTGGCTGTTATGACTTTGGTTGCTGCTG |
| | transporter | CTTACACTGTTGCTTTGAGATACACTAGAACTACTGCT |
| | (MmCST) | GAGGAGTTGTACTTCTCCACTACTGCTGTTTGTATCAC |
| | Codon | TGAGGTTATCAAGTTGTTGATCTCCGTTGGTTTGTTGG |
| | optimized | CTAAGGAGACTGGTTCTTTGGGAAGATTCAAGGCTTC |
| | | CTTGTCCGAAAACGTTTTGGGTTCCCCAAAGGAGTTG |
| | | GCTAAGTTGTCTGTTCCATCCTTGGTTTACGCTGTTCA |
| | | GAACAACATGGCTTTCTTGGCTTTGTCTAACTTGGACG |
| | | CTGCTGTTTACCAAGTTACTTACCAGTTGAAGATCCCA |
| | | TGTACTGCTTTGTGTACTGTTTTGATGTTGAACAGAAC |
| | | ATTGTCCAAGTTGCAGTGGATCTCCGTTTTCATGTTGT |
| | | GTGGTGGTGTTACTTTGGTTCAGTGGAAGCCAGCTCA |
| | | AGCTTCCAAAGTTGTTGTTGCTCAGAACCCATTGTTGG |
| | | GTTTCGGTGCTATTGCTATCGCTGTTTTGTGTTCCGGTT |
| | | TCGCTGGTGTTTACTTCGAGAAGGTTTTGAAGTCCTCC |
| | | GACACTTCTTTGTGGGTTAGAAACATCCAGATGTACTT |
| | | GTCCGGTATCGTTGTTACTTTGGCTGGTACTTACTTGT |
| | | CTGACGGTGCTGAGATTCAAGAGAAGGGATTCTTCTA |
| | | CGGTTACACTTACTATGTTTGGTTCGTTATCTTCTTGGC |
| | | TTCCGTTGGTGGTTTGTACACTTCCGTTGTTGTTAAGT |
| | | ACACTGACAACATCATGAAGGGATTCTCTGCTGCTGC |
| | | TGCTATTGTTTTGTCCACTATCGCTTCCGTTTTGTTGTT |
| | | CGGATTGCAGATCACATTGTCCTTTGCTTTGGGAGCTT |
| | | TGTTGGTTTGTGTTTCCATCTACTTGTACGGATTGCCA |
| | | AGACAAGACACTACTTCCATTCAGCAAGAGGCTACTT |
| | | CCAAGGAGAGAATCATCGGTGTTTAGTAG |
| 65 | Human UDP- | ATGGAAAAGAACGGTAACAACAGAAAGTTGAGAGTTT |
| | GlcNAc 2- | GTGTTGCTACTTGTAACAGAGCTGACTACTCCAAGTTG |
| | epimerase/N- | GCTCCAATCATGTTCGGTATCAAGACTGAGCCAGAGT |
| | acetylmanno- | TCTTCGAGTTGGACGTTGTTGTTTTGGGTTCCCACTTG |
| | samine kinase | ATTGATGACTACGGTAACACTTACAGAATGATCGAGC |
| | (HsGNE) | AGGACGACTTCGACATCAACACTAGATTGCACACTAT |
| | codon | TGTTAGAGGAGAGGACGAAGCTGCTATGGTTGAATCT |
| | opitimized | GTTGGATTGGCTTTGGTTAAGTTGCCAGACGTTTTGAA |
| | | CAGATTGAAGCCAGACATCATGATTGTTCACGGTGAC |
| | | AGATTCGATGCTTTGGCTTTGGCTACTTCCGCTGCTTT |
| | | GATGAACATTAGAATCTTGCACATCGAGGGTGGTGAA |
| | | GTTTCTGGTACTATCGACGACTCCATCAGACACGCTAT |
| | | CACTAAGTTGGCTCACTACCATGTTTGTTGTACTAGAT |
| | | CCGCTGAGCAACACTTGATTTCCATGTGTGAGGACCA |
| | | CGACAGAATTTTGTTGGCTGGTTGTCCATCTTACGACA |
| | | AGTTGTTGTCCGCTAAGAACAAGGACTACATGTCCAT |
| | | CATCAGAATGTGGTTGGGTGACGACGTTAAGTCTAAG |
| | | GACTACATCGTTGCTTTGCAGCACCCAGTTACTACTGA |
| | | CATCAAGCACTCCATCAAGATGTTCGAGTTGACTTTGG |
| | | ACGCTTTGATCTCCTTCAACAAGAGAACTTTGGTTTTG |
| | | TTCCCAAACATTGACGCTGGTTCCAAAGAGATGGTTA |
| | | GAGTTATGAGAAAGAAGGGTATCGAACACCACCCAA |
| | | ACTTCAGAGCTGTTAAGCACGTTCCATTCGACCAATTC |
| | | ATCCAGTTGGTTGCTCATGCTGGTTGTATGATCGGTAA |
| | | CTCCTCCTGTGGTGTTAGAGAAGTTGGTGCTTTCGGTA |
| | | CTCCAGTTATCAACTTGGGTACTAGACAGATCGGTAG |
| | | AGAGACTGGAGAAAACGTTTTGCATGTTAGAGATGCT |
| | | GACACTCAGGACAAGATTTTGCAGGCTTTGCACTTGC |
| | | AATTCGGAAAGCAGTACCCATGTTCCAAAATCTACGG |
| | | TGACGGTAACGCTGTTCCAAGAATCTTGAAGTTTTTGA |
| | | AGTCCATCGACTTGCAAGAGCCATTGCAGAAGAAGTT |
| | | CTGTTTCCCACCAGTTAAGGAGAACATCTCCCAGGAC |
| | | ATTGACCACATCTTGGAGACATTGTCCGCTTTGGCTGT |
| | | TGATTTGGGTGGAACTAACTTGAGAGTTGCTATCGTTT |
| | | CCATGAAGGGAGAGATCGTTAAGAAGTACACTCAGTT |
| | | CAACCCAAAGACTTACGAGGAGAGAATCAACTTGATC |
| | | TTGCAGATGTGTGTTGAAGCTGCTGCTGAGGCTGTTAA |
| | | GTTGAACTGTAGAATCTTGGGTGTTGGTATCTCTACTG |
| | | GTGGTAGAGTTAATCCAAGAGAGGGTATCGTTTTGCA |
| | | CTCCACTAAGTTGATTCAGGAGTGGAACTCCGTTGATT |
| | | TGAGAACTCCATTGTCCGACACATTGCACTTGCCAGTT |
| | | TGGGTTGACAACGACGGTAATTGTGCTGCTTTGGCTG |
| | | AGAGAAAGTTCGGTCAAGGAAAGGGATTGGAGAACTT |
| | | CGTTACTTTGATCACTGGTACTGGTATTGGTGGTGGTA |
| | | TCATTCACCAGCACGAGTTGATTCACGGTTCTTCCTTC |
| | | TGTGCTGCTGAATTGGGACACTTGGTTGTTTCTTTGGA |
| | | CGGTCCAGACTGTTCTTGTGGTTCCCACGGTTGTATTG |
| | | AAGCTTACGCATCAGGAATGGCATTGCAGAGAGAGGC |
| | | TAAGAAGTTGCACGACGAGGACTTGTTGTTGGTTGAG |
| | | GGAATGTCTGTTCCAAAGGACGAGGCTGTTGGTGCTT |
| | | TGCATTTGATCCAGGCTGCTAAGTTGGGTAATGCTAA |
| | | GGCTCAGTCCATCTTGAGAACTGCTGGTACTGCTTTGG |
| | | GATTGGGTGTTGTTAATATCTTGCACACTATGAACCCA |
| | | TCCTTGGTTATCTTGTCCGGTGTTTTGGCTTCTCACTAC |
| | | ATCCACATCGTTAAGGACGTTATCAGACAGCAAGCTT |
| | | TGTCCTCCGTTCAAGACGTTGATGTTGTTGTTTCCGAC |
| | | TTGGTTGACCCAGCTTTGTTGGGTGCTGCTTCCATGGT |
| | | TTTGGACTACACTACTAGAAGAATCTACTAATAG |
| 66 | Sequence of the | CAGTTGAGCCAGACCGCGCTAAACGCATACCAATTGC |
| | PpARG1 | CAAATCAGGCAATTGTGAGACAGTGGTAAAAAAGATG |
| | auxotrophic | CCTGCAAAGTTAGATTCACACAGTAAGAGAGATCCTA |
| | marker | CTCATAAATGAGGCGCTTATTTAGTAGCTAGTGATAG |
| | | CCACTGCGGTTCTGCTTTATGCTATTTGTTGTATGCCTT |
| | | ACTATCTTTGTTTGGCTCCTTTTTCTTGACGTTTTCCGT |
| | | TGGAGGGACTCCCTATTCTGAGTCATGAGCCGCACAG |
| | | ATTATCGCCCAAAATTGACAAAATCTTCTGGCGAAAA |
| | | AAGTATAAAAGGAGAAAAAAGCTCACCCTTTTCCAGC |
| | | GTAGAAAGTATATATCAGTCATTGAAGACTATTATTTA |
| | | AATAACACAATGTCTAAAGGAAAAGTTTGTTTGGCCT |
| | | ACTCCGGTGGTTTGGATACCTCCATCATCCTAGCTTGG |
| | | TTGTTGGAGCAGGGATACGAAGTCGTTGCCTTTTTAGC |
| | | CAACATTGGTCAAGAGGAAGACTTTGAGGCTGCTAGA |
| | | GAGAAAGCTCTGAAGATCGGTGCTACCAAGTTTATCG |
| | | TCAGTGACGTTAGGAAGGAATTTGTTGAGGAAGTTTT |
| | | GTTCCCAGCAGTCCAAGTTAACGCTATCTACGAGAAC |
| | | GTCTACTTACTGGGTACCTCTTTGGCCAGACCAGTCAT |
| | | TGCCAAGGCCCAAATAGAGGTTGCTGAACAAGAAGGT |
| | | TGTTTTGCTGTTGCCCACGGTTGTACCGGAAAGGGTAA |
| | | CGATCAGGTTAGATTTGAGCTTTCCTTTTATGCTCTGA |
| | | AGCCTGACGTTGTCTGTATCGCCCCATGGAGAGACCC |
| | | AGAATTCTTCGAAAGATTCGCTGGTAGAAATGACTTG |
| | | CTGAATTACGCTGCTGAGAAGGATATTCCAGTTGCTC |
| | | AGACTAAAGCCAAGCCATGGTCTACTGATGAGAACAT |
| | | GGCTCACATCTCCTTCGAGGCTGGTATTCTAGAAGATC |
| | | CAAACACTACTCCTCCAAAGGACATGTGGAAGCTCAC |
| | | TGTTGACCCAGAAGATGCACCAGACAAGCCAGAGTTC |
| | | TTTGACGTCCACTTTGAGAAGGGTAAGCCAGTTAAAT |
| | | TAGTTCTCGAGAACAAAACTGAGGTCACCGATCCGGT |
| | | TGAGATCTTTTTGACTGCTAACGCCATTGCTAGAAGAA |
| | | ACGGTGTTGGTAGAATTGACATTGTCGAGAACAGATT |
| | | CATCGGAATCAAGTCCAGAGGTTGTTATGAAACTCCA |
| | | GGTTTGACTCTACTGAGAACCACTCACATCGACTTGG |
| | | AAGGTCTTACCGTTGACCGTGAAGTTAGATCGATCAG |
| | | AGACACTTTTGTTACCCCAACCTACTCTAAGTTGTTAT |
| | | ACAACGGGTTGTACTTTACCCCAGAAGGTGAGTACGT |
| | | CAGAACTATGATTCAGCCTTCTCAAAACACCGTCAAC |
| | | GGTGTTGTTAGAGCCAAGGCCTACAAAGGTAATGTGT |
| | | ATAACCTAGGAAGATACTCTGAAACCGAGAAATTGTA |
| | | CGATGCTACCGAATCTTCCATGGATGAGTTGACCGGA |
| | | TTCCACCCTCAAGAAGCTGGAGGATTTATCACAACAC |
| | | AAGCCATCAGAATCAAGAAGTACGGAGAAAGTGTCA |
| | | GAGAGAAGGGAAAGTTTTTGGGACTTTAACTCAAGTA |
| | | AAAGGATAGTTGTACAATTATATATACGAAGAATAAA |
| | | TCATTACAAAAAGTATTCGTTTCTTTGATTCTTAACAG |
| | | GATTCATTTTCTGGGTGTCATCAGGTACAGCGCTGAAT |
| | | ATCTTGAAGTTAACATCGAGCTCATCATCGACGTTCAT |
| | | CACACTAGCCACGTTTCCGCAACGGTAGCAATAATTA |
| | | GGAGCGGACCACACAGTGACGACATC |
| 67 | Human CMP- | ATGGACTCTGTTGAAAAGGGTGCTGCTACTTCTGTTTC |
| | sialic acid | CAACCCAAGAGGTAGACCATCCAGAGGTAGACCTCCT |
| | synthase | AAGTTGCAGAGAAACTCCAGAGGTGGTCAAGGTAGAG |
| | (HsCSS) codon | GTGTTGAAAAGCCACCACACTTGGCTGCTTTGATCTTG |
| | optimized | GCTAGAGGAGGTTCTAAGGGTATCCCATTGAAGAACA |
| | | TCAAGCACTTGGCTGGTGTTCCATTGATTGGATGGGTT |
| | | TTGAGAGCTGCTTTGGACTCTGGTGCTTTCCAATCTGT |
| | | TTGGGTTTCCACTGACCACGACGAGATTGAGAACGTT |
| | | GCTAAGCAATTCGGTGCTCAGGTTCACAGAAGATCCT |
| | | CTGAGGTTTCCAAGGACTCTTCTACTTCCTTGGACGCT |
| | | ATCATCGAGTTCTTGAACTACCACAACGAGGTTGACA |
| | | TCGTTGGTAACATCCAAGCTACTTCCCCATGTTTGCAC |
| | | CCAACTGACTTGCAAAAAGTTGCTGAGATGATCAGAG |
| | | AAGAGGGTTACGACTCCGTTTTCTCCGTTGTTAGAAGG |
| | | CACCAGTTCAGATGGTCCGAGATTCAGAAGGGTGTTA |
| | | GAGAGGTTACAGAGCCATTGAACTTGAACCCAGCTAA |
| | | AAGACCAAGAAGGCAGGATTGGGACGGTGAATTGTAC |
| | | GAAAACGGTTCCTTCTACTTCGCTAAGAGACACTTGAT |
| | | CGAGATGGGATACTTGCAAGGTGGAAAGATGGCTTAC |
| | | TACGAGATGAGAGCTGAACACTCCGTTGACATCGACG |
| | | TTGATATCGACTGGCCAATTGCTGAGCAGAGAGTTTT |
| | | GAGATACGGTTACTTCGGAAAGGAGAAGTTGAAGGAG |
| | | ATCAAGTTGTTGGTTTGTAACATCGACGGTTGTTTGAC |
| | | TAACGGTCACATCTACGTTTCTGGTGACCAGAAGGAG |
| | | ATTATCTCCTACGACGTTAAGGACGCTATTGGTATCTC |
| | | CTTGTTGAAGAAGTCCGGTATCGAAGTTAGATTGATCT |
| | | CCGAGAGAGCTTGTTCCAAGCAAACATTGTCCTCTTTG |
| | | AAGTTGGACTGTAAGATGGAGGTTTCCGTTTCTGACA |
| | | AGTTGGCTGTTGTTGACGAATGGAGAAAGGAGATGGG |
| | | TTTGTGTTGGAAGGAAGTTGCTTACTTGGGTAACGAA |
| | | GTTTCTGACGAGGAGTGTTTGAAGAGAGTTGGTTTGTC |
| | | TGGTGCTCCAGCTGATGCTTGTTCCACTGCTCAAAAGG |
| | | CTGTTGGTTACATCTGTAAGTGTAACGGTGGTAGAGGT |
| | | GCTATTAGAGAGTTCGCTGAGCACATCTGTTTGTTGAT |
| | | GGAGAAAGTTAATAACTCCTGTCAGAAGTAGTAG |
| 68 | Human N- | ATGCCATTGGAATTGGAGTTGTGTCCTGGTAGATGGGT |
| | acetylneuraminate- | TGGTGGTCAACACCCATGTTTCATCATCGCTGAGATCG |
| | 9-phosphate | GTCAAAACCACCAAGGAGACTTGGACGTTGCTAAGAG |
| | synthase | AATGATCAGAATGGCTAAGGAATGTGGTGCTGACTGT |
| | (HsSPS) codon | GCTAAGTTCCAGAAGTCCGAGTTGGAGTTCAAGTTCA |
| | optimized | ACAGAAAGGCTTTGGAAAGACCATACACTTCCAAGCA |
| | | CTCTTGGGGAAAGACTTACGGAGAACACAAGAGACAC |
| | | TTGGAGTTCTCTCACGACCAATACAGAGAGTTGCAGA |
| | | GATACGCTGAGGAAGTTGGTATCTTCTTCACTGCTTCT |
| | | GGAATGGACGAAATGGCTGTTGAGTTCTTGCACGAGT |
| | | TGAACGTTCCATTCTTCAAAGTTGGTTCCGGTGACACT |
| | | AACAACTTCCCATACTTGGAAAAGACTGCTAAGAAAG |
| | | GTAGACCAATGGTTATCTCCTCTGGAATGCAGTCTATG |
| | | GACACTATGAAGCAGGTTTACCAGATCGTTAAGCCAT |
| | | TGAACCCAAACTTTTGTTTCTTGCAGTGTACTTCCGCT |
| | | TACCCATTGCAACCAGAGGACGTTAATTTGAGAGTTA |
| | | TCTCCGAGTACCAGAAGTTGTTCCCAGACATCCCAATT |
| | | GGTTACTCTGGTCACGAGACTGGTATTGCTATTTCCGT |
| | | TGCTGCTGTTGCTTTGGGTGCTAAGGTTTTGGAGAGAC |
| | | ACATCACTTTGGACAAGACTTGGAAGGGTTCTGATCA |
| | | CTCTGCTTCTTTGGAACCTGGTGAGTTGGCTGAACTTG |
| | | TTAGATCAGTTAGATTGGTTGAGAGAGCTTTGGGTTCC |
| | | CCAACTAAGCAATTGTTGCCATGTGAGATGGCTTGTA |
| | | ACGAGAAGTTGGGAAAGTCCGTTGTTGCTAAGGTTAA |
| | | GATCCCAGAGGGTACTATCTTGACTATGGACATGTTG |
| | | ACTGTTAAAGTTGGAGAGCCAAAGGGTTACCCACCAG |
| | | AGGACATCTTTAACTTGGTTGGTAAAAAGGTTTTGGTT |
| | | ACTGTTGAGGAGGACGACACTATTATGGAGGAGTTGG |
| | | TTGACAACCACGGAAAGAAGATCAAGTCCTAG |
| 69 | Mouse alpha- | GTTTTTCAAATGCCAAAGTCCCAGGAGAAAGTTGCTG |
| | 2,6-sialyl | TTGGTCCAGCTCCACAAGCTGTTTTCTCCAACTCCAAG |
| | transferase | CAAGATCCAAAGGAGGGTGTTCAAATCTTGTCCTACC |
| | catalytic domain | CAAGAGTTACTGCTAAGGTTAAGCCACAACCATCCTT |
| | (MmmST6) | GCAAGTTTGGGACAAGGACTCCACTTACTCCAAGTTG |
| | codon optimized | AACCCAAGATTGTTGAAGATTTGGAGAAACTACTTGA |
| | | ACATGAACAAGTACAAGGTTTCCTACAAGGGTCCAGG |
| | | TCCAGGTGTTAAGTTCTCCGTTGAGGCTTTGAGATGTC |
| | | ACTTGAGAGACCACGTTAACGTTTCCATGATCGAGGC |
| | | TACTGACTTCCCATTCAACACTACTGAATGGGAGGGA |
| | | TACTTGCCAAAGGAGAACTTCAGAACTAAGGCTGGTC |
| | | CATGGCATAAGTGTGCTGTTGTTTCTTCTGCTGGTTCC |
| | | TTGAAGAACTCCCAGTTGGGTAGAGAAATTGACAACC |
| | | ACGACGCTGTTTTGAGATTCAACGGTGCTCCAACTGA |
| | | CAACTTCCAGCAGGATGTTGGTACTAAGACTACTATC |
| | | AGATTGGTTAACTCCCAATTGGTTACTACTGAGAAGA |
| | | GATTCTTGAAGGACTCCTTGTACACTGAGGGAATCTTG |
| | | ATTTTGTGGGACCCATCTGTTTACCACGCTGACATTCC |
| | | ACAATGGTATCAGAAGCCAGACTACAACTTCTTCGAG |
| | | ACTTACAAGTCCTACAGAAGATTGCACCCATCCCAGC |
| | | CATTCTACATCTTGAAGCCACAAATGCCATGGGAATT |
| | | GTGGGACATCATCCAGGAAATTTCCCCAGACTTGATC |
| | | CAACCAAACCCACCATCTTCTGGAATGTTGGGTATCAT |
| | | CATCATGATGACTTTGTGTGACCAGGTTGACATCTACG |
| | | AGTTCTTGCCATCCAAGAGAAAGACTGATGTTTGTTAC |
| | | TACCACCAGAAGTTCTTCGACTCCGCTTGTACTATGGG |
| | | AGCTTACCACCCATTGTTGTTCGAGAAGAACATGGTT |
| | | AAGCACTTGAACGAAGGTACTGACGAGGACATCTACT |
| | | TGTTCGGAAAGGCTACTTTGTCCGGTTTCAGAAACAA |
| | | CAGATGTTAG |
| 70 | HSA signal | ATGAAGTGGGTTACCTTTATCTCTTTGTTGTTTCTTTTC |
| | peptide DNA | TCTTCTGCTTACTCT |
| 71 | HSA signal | MKWVTFISLLFLFSSAYS |
| | peptide | |
| 72 | TNFRII-Fc | CTGCCAGCTCAAGTTGCTTTTACTCCATACGCTCCAGA |
| | fragment fusion | ACCAGGTTCTACTTGTAGATTGAGAGAGTACTACGAC |
| | protein (C- | CAAACTGCTCAGATGTGTTGTTCCAAGTGTTCTCCAGG |
| | terminal K-less) | TCAACACGCTAAGGTTTTCTGTACTAAGACTTCCGACA |
| | 1-705 encodes | CTGTTTGTGACTCTTGTGAGGACTCCACTTACACTCAA |
| | TNFRII | TTGTGGAACTGGGTTCCAGAATGTTTGTCCTGTGGTTC |
| | (underlined) | CAGATGTTCTTCCGACCAAGTTGAGACTCAGGCTTGTA |
| | | CTAGAGAGCAGAACAGAATCTGTACTTGTAGACCTGG |
| | | TTGGTACTGTGCTTTGTCCAAGCAAGAGGGTTGTAGAT |
| | | TGTGTGCTCCATTGAGAAAGTGTAGACCAGGTTTCGG |
| | | TGTTGCTAGACCAGGTACAGAAACTTCCGACGTTGTTT |
| | | GTAAGCCATGTGCTCCAGGAACTTTCTCCAACACTACT |
| | | TCCTCCACTGACATCTGTAGACCACACCAAATCTGTAA |
| | | CGTTGTTGCTATCCCAGGTAACGCTTCTATGGACGCTG |
| | | TTTGTACTTCTACTTCCCCAACTAGATCCATGGCTCCA |
| | | GGTGCTGTTCATTTGCCACAGCCAGTTTCCACTAGATC |
| | | CCAACACACTCAACCAACTCCAGAACCATCTACTGCT |
| | | CCATCCACTTCCTTTTTGTTGCCAATGGGACCATCTCC |
| | | ACCTGCTGAAGGTTCTACTGGTGACGAGCCAAAGTCC |
| | | TGTGACAAGACACATACTTGTCCACCATGTCCAGCTCC |
| | | AGAATTGTTGGGTGGTCCATCCGTTTTCTTGTTCCCAC |
| | | CAAAGCCAAAGGACACTTTGATGATCTCCAGAACTCC |
| | | AGAGGTTACATGTGTTGTTGTTGACGTTTCTCACGAGG |
| | | ACCCAGAGGTTAAGTTCAACTGGTACGTTGACGGTGT |
| | | TGAAGTTCACAACGCTAAGACTAAGCCAAGAGAAGA |
| | | GCAGTACAACTCCACTTACAGAGTTGTTTCCGTTTTGA |
| | | CTGTTTTGCACCAGGATTGGTTGAACGGTAAAGAATA |
| | | CAAGTGTAAGGTTTCCAACAAGGCTTTGCCAGCTCCA |
| | | ATCGAAAAGACAATCTCCAAGGCTAAGGGTCAACCAA |
| | | GAGAGCCACAGGTTTACACTTTGCCACCATCCAGAGA |
| | | AGAGATGACTAAGAACCAGGTTTCCTTGACTTGTTTG |
| | | GTTAAAGGATTCTACCCATCCGACATTGCTGTTGAATG |
| | | GGAATCTAACGGTCAACCAGAGAACAACTACAAGACT |
| | | ACTCCACCAGTTTTGGATTCTGACGGTTCCTTCTTCTT |
| | | GTACTCCAAGTTGACTGTTGACAAGTCCAGATGGCAA |
| | | CAGGGTAACGTTTTCTCCTGTTCCGTTATGCATGAGGC |
| | | TTTGCACAACCACTACACTCAAAAGTCCTTGTCTTTGT |
| | | CCCCAGGTTAG |
| 73 | TNFRII-Fc | LPAQVAFTPYAPEPGSTCRLREYYDQTAQMCCSKCSPGQ |
| | fragment fusion | HAKVFCTKTSDTVCDSCEDSTYTQLWNWVPECLSCGSR |
| | protein (C- | CSSDQVETQACTREQNRICTCRPGWYCALSKQEGCRLC |
| | terminal K-less) | APLRKCRPGFGVARPGTETSDVVCKPCAPGTFSNTTSST |
| | 1-235 receptor | DICRPHQICNVVAIPGNASMDAVCTSTSPTRSMAPGAVH |
| | domain | LPQPVSTRSQHTQPTPEPSTAPSTSFLLPMGPSPPAEGSTG |
| | (underlined) | DEPKSCDKTHTCPPCPAPELLGGPSVFLFPPKPKDTLMIS |
| | | RTPEVTCVVVDVSHEDPEVKFNWYVDGVEVHNAKTKP |
| | | REEQYNSTYRVVSVLTVLHQDWLNGKEYKCKVSNKALP |
| | | APIEKTISKAKGQPREPQVYTLPPSREEMTKNQVSLTCLV |
| | | KGFYPSDIAVEWESNGQPENNYKTTPPVLDSDGSFFLYS |
| | | KLTVDKSRWQQGNVFSCSVMHEALHNHYTQKSLSLSPG |
| 74 | TNFRII-Fc | CTGCCAGCTCAAGTTGCTTTTACTCCATACGCTCCAGA |
| | fragment fusion | ACCAGGTTCTACTTGTAGATTGAGAGAGTACTACGAC |
| | protein (with C- | CAAACTGCTCAGATGTGTTGTTCCAAGTGTTCTCCAGG |
| | terminal K) | TCAACACGCTAAGGTTTTCTGTACTAAGACTTCCGACA |
| | 1-705 encode | CTGTTTGTGACTCTTGTGAGGACTCCACTTACACTCAA |
| | TNFRII | TTGTGGAACTGGGTTCCAGAATGTTTGTCCTGTGGTTC |
| | (underlined) | CAGATGTTCTTCCGACCAAGTTGAGACTCAGGCTTGTA |
| | | CTAGAGAGCAGAACAGAATCTGTACTTGTAGACCTGG |
| | | TTGGTACTGTGCTTTGTCCAAGCAAGAGGGTTGTAGAT |
| | | TGTGTGCTCCATTGAGAAAGTGTAGACCAGGTTTCGG |
| | | TGTTGCTAGACCAGGTACAGAAACTTCCGACGTTGTTT |
| | | GTAAGCCATGTGCTCCAGGAACTTTCTCCAACACTACT |
| | | TCCTCCACTGACATCTGTAGACCACACCAAATCTGTAA |
| | | CGTTGTTGCTATCCCAGGTAACGCTTCTATGGACGCTG |
| | | TTTGTACTTCTACTTCCCCAACTAGATCCATGGCTCCA |
| | | GGTGCTGTTCATTTGCCACAGCCAGTTTCCACTAGATC |
| | | CCAACACACTCAACCAACTCCAGAACCATCTACTGCT |
| | | CCATCCACTTCCTTTTTGTTGCCAATGGGACCATCTCC |
| | | ACCTGCTGAAGGTTCTACTGGTGACGAGCCAAAGTCC |
| | | TGTGACAAGACACATACTTGTCCACCATGTCCAGCTCC |
| | | AGAATTGTTGGGTGGTCCATCCGTTTTCTTGTTCCCAC |
| | | CAAAGCCAAAGGACACTTTGATGATCTCCAGAACTCC |
| | | AGAGGTTACATGTGTTGTTGTTGACGTTTCTCACGAGG |
| | | ACCCAGAGGTTAAGTTCAACTGGTACGTTGACGGTGT |
| | | TGAAGTTCACAACGCTAAGACTAAGCCAAGAGAAGA |
| | | GCAGTACAACTCCACTTACAGAGTTGTTTCCGTTTTGA |
| | | CTGTTTTGCACCAGGATTGGTTGAACGGTAAAGAATA |
| | | CAAGTGTAAGGTTTCCAACAAGGCTTTGCCAGCTCCA |
| | | ATCGAAAAGACAATCTCCAAGGCTAAGGGTCAACCAA |
| | | GAGAGCCACAGGTTTACACTTTGCCACCATCCAGAGA |
| | | AGAGATGACTAAGAACCAGGTTTCCTTGACTTGTTTG |
| | | GTTAAAGGATTCTACCCATCCGACATTGCTGTTGAATG |
| | | GGAATCTAACGGTCAACCAGAGAACAACTACAAGACT |
| | | ACTCCACCAGTTTTGGATTCTGACGGTTCCTTCTTCTT |
| | | GTACTCCAAGTTGACTGTTGACAAGTCCAGATGGCAA |
| | | CAGGGTAACGTTTTCTCCTGTTCCGTTATGCATGAGGC |
| | | TTTGCACAACCACTACACTCAAAAGTCCTTGTCTTTGT |
| | | CCCCAGGTAAGTAG |
| 75 | TNFRII-Fc | LPAQVAFTPYAPEPGSTCRLREYYDQTAQMCCSKCSPGQ |
| | fragment fusion | HAKVFCTKTSDTVCDSCEDSTYTQLWNWVPECLSCGSR |
| | protein (with C- | CSSDQVETQACTREQNRICTCRPGWYCALSKQEGCRLC |
| | terminal K) | APLRKCRPGFGVARPGTETSDVVCKPCAPGTFSNTTSST |
| | 1-235 receptor | DICRPHQICNVVAIPGNASMDAVCTSTSPTRSMAPGAVH |
| | domain | LPQPVSTRSQHTQPTPEPSTAPSTSFLLPMGPSPPAEGSTG |
| | (underlined) | DEPKSCDKTHTCPPCPAPELLGGPSVFLFPPKPKDTLMIS |
| | | RTPEVTCVVVDVSHEDPEVKFNWYVDGVEVPHNAKTKP |
| | | REEQYNSTYRVVSVLTVLHQDWLNGKEYKCKVSNKALP |
| | | APIEKTISKAKGQPREPQVYTLPPSREEMTKNQVSLTCLV |
| | | KGFYPSDIAVEWESNGQPENNYKTTPPVLDSDGSFFLYS |
| | | KLTVDKSRWQQGNVFSCSVMHEALHNHYTQKSLSLSPGK |
| 76 | Mouse | CGCGCCATTTCTGAAGCTAACGAGGACCCTGAACCAG |
| | POMGnTI | AACAAGATTACGACGAGGCTTTGGGAAGATTGGAATC |
| | | CCCAAGAAGAAGAGGATCCTCCCCTAGAAGAGTTTTG |
| | | GACGTTGAGGTTTACTCTTCCAGATCCAAGGTTTACGT |
| | | TGCTGTTGACGGTACTACTGTTTTGGAGGACGAGGCT |
| | | AGAGAACAAGGTAGAGGTATCCACGTTATCGTTTTGA |
| | | ACCAGGCTACTGGTCATGTTATGGCTAAGAGAGTTTTC |
| | | GACACTTACTCTCCACACGAAGATGAGGCTATGGTTTT |
| | | GTTCTTGAACATGGTTGCTCCAGGTAGAGTTTTGATTT |
| | | GTACTGTTAAGGACGAGGGATCCTTCCATTTGAAGGA |
| | | CACTGCTAAGGCTTTGTTGAGATCCTTGGGTTCTCAAG |
| | | CTGGTCCAGCTTTGGGATGGAGAGATACTTGGGCTTTC |
| | | GTTGGTAGAAAGGGTGGTCCAGTTTTGGGTGAAAAGC |
| | | ACTCTAAGTCCCCAGCTTTGTCCTCTTGGGGTGACCCA |
| | | GTTTTGTTGAAAACTGACGTTCCATTGTCCTCTGCTGA |
| | | AGAGGCTGAATGTCACTGGGCTGACACTGAGTTGAAC |
| | | AGAAGAAGAAGAAGATTCTGTTCCAAGGTTGAGGGTT |
| | | ACGGTTCTGTTTGTTCCTGTAAGGACCCAACTCCAATT |
| | | GAATTCTCCCCAGACCCATTGCCAGATAACAAGGTTTT |
| | | GAACGTTCCAGTTGCTGTTATCGCTGGTAACAGACCA |
| | | AACTACTTGTACAGAATGTTGAGATCTTTGTTGTCCGC |
| | | TCAGGGAGTTTCTCCACAGATGATCACTGTTTTCATCG |
| | | ACGGTTACTACGAAGAACCAATGGACGTTGTTGCTTT |
| | | GTTCGGATTGAGAGGTATTCAGCACACTCCAATCTCC |
| | | ATCAAGAACGCTAGAGTTTCCCAACACTACAAGGCTT |
| | | CCTTGACTGCTACTTTCAACTTGTTCCCAGAGGCTAAG |
| | | TTCGCTGTTGTTTTGGAAGAGGACTTGGACATTGCTGT |
| | | TGATTTCTTCTCCTTCTTGTCCCAATCCATCCACTTGTT |
| | | GGAAGAGGATGACTCCTTGTACTGTATCTCTGCTTGGA |
| | | ACGACCAAGGTTACGAACACACTGCTGAGGATCCAGC |
| | | TTTGTTGTACAGAGTTGAGACTATGCCAGGATTGGGAT |
| | | GGGTTTTGAGAAAGTCCTTGTACAAAGAGGAGTTGGA |
| | | GCCAAAGTGGCCAACTCCAGAAAAGTTGTGGGATTGG |
| | | GACATGTGGATGAGAATGCCAGAGCAGAGAAGAGGT |
| | | AGAGAGTGTATCATCCCAGACGTTTCCAGATCTTACC |
| | | ACTTCGGTATTGTTGGATTGAACATGAACGGTTACTTC |
| | | CACGAGGCTTACTTCAAGAAGCACAAGTTCAACACTG |
| | | TTCCAGGTGTTCAGTTGAGAAACGTTGACTCCTTGAAG |
| | | AAAGAGGCTTACGAGGTTGAGATCCACAGATTGTTGT |
| | | CTGAGGCTGAGGTTTTGGATCACTCCAAGGATCCATG |
| | | TGAGGACTCATTCTTGCCAGATACTGAGGGTCATACTT |
| | | ACGTTGCTTTCATCAGAATGGAAACTGACGACGACTT |
| | | TGCTACTTGGACTCAGTTGGCTAAGTGTTTGCACATTT |
| | | GGGACTTGGATGTTAGAGGTAACCACAGAGGATTGTG |
| | | GAGATTGTTCAGAAAGAAGAACCACTTCTTGGTTGTT |
| | | GGTGTTCCAGCTTCTCCATACTCCGTTAAGAAGCCACC |
| | | ATCCGTTACTCCAATTTTCTTGGAGCCACCACCAAAGG |
| | | AAGAAGGTGCTCCTGGAGCTGCTGAACAAACTTAGTA |
| | | GTTAA |
| 77 | DNA encodes | ATGCACGTACTGCTGAGCAAAAAAATAGCACGCTTTC |
| | Mnn6-s leader | TGTTGATTTCGTTTGTTTTCGTGCTTGCGCTAATGGTG |
| | (65) | ACAATAAATCATCCAGGGCGCGCC |
| 78 | DNA encodes | ATGCTGATTAGGTTAAAGAAGAGAAAAATCCTGCAGG |
| | Mnn5-s leader | TCATCGTGAGCGCAGTAGTGCTAATTTTATTTTTTTGT |
| | (56) | TCTGTGCATAATGATGTGTCTTCTAGTTGGGGGCGCGCC |
| 79 | HYG^{R} resistance | GATCTGTTTAGCTTGCCTCGTCCCCGCCGGGTCACCCG |
| | cassette | GCCAGCGACATGGAGGCCCAGAATACCCTCCTTGACA |
| | | GTCTTGACGTGCGCAGCTCAGGGGCATGATGTGACTG |
| | | TCGCCCGTACATTTAGCCCATACATCCCCATGTATAAT |
| | | CATTTGCATCCATACATTTTGATGGCCGCACGGCGCGA |
| | | AGCAAAAATTACGGCTCCTCGCTGCGGACCTGCGAGC |
| | | AGGGAAACGCTCCCCTCACAGACGCGTTGAATTGTCC |
| | | CCACGCCGCGCCCCTGTAGAGAAATATAAAAGGTTAG |
| | | GATTTGCCACTGAGGTTCTTCTTTCATATACTTCCTTTT |
| | | AAAATCTTGCTAGGATACAGTTCTCACATCACATCCG |
| | | AACATAAACAACCATGGGTAAAAAGCCTGAACTCACC |
| | | GCGACGTCTGTCGAGAAGTTTCTGATCGAAAAGTTCG |
| | | ACAGCGTCTCCGACCTGATGCAGCTCTCGGAGGGCGA |
| | | AGAATCTCGTGCTTTCAGCTTCGATGTAGGAGGGCGT |
| | | GGATATGTCCTGCGGGTAAATAGCTGCGCCGATGGTT |
| | | TCTACAAAGATCGTTATGTTTATCGGCACTTTGCATCG |
| | | GCCGCGCTCCCGATTCCGGAAGTGCTTGACATTGGGG |
| | | AATTCAGCGAGAGCCTGACCTATTGCATCTCCCGCCGT |
| | | GCACAGGGTGTCACGTTGCAAGACCTGCCTGAAACCG |
| | | AACTGCCCGCTGTTCTGCAGCCGGTCGCGGAGGCCAT |
| | | GGATGCGATCGCTGCGGCCGATCTTAGCCAGACGAGC |
| | | GGGTTCGGCCCATTCGGACCGCAAGGAATCGGTCAAT |
| | | ACACTACATGGCGTGATTTCATATGCGCGATTGCTGAT |
| | | CCCCATGTGTATCACTGGCAAACTGTGATGGACGACA |
| | | CCGTCAGTGCGTCCGTCGCGCAGGCTCTCGATGAGCT |
| | | GATGCTTTGGGCCGAGGACTGCCCCGAAGTCCGGCAC |
| | | CTCGTGCACGCGGATTTCGGCTCCAACAATGTCCTGAC |
| | | GGACAATGGCCGCATAACAGCGGTCATTGACTGGAGC |
| | | GAGGCGATGTTCGGGGATTCCCAATACGAGGTCGCCA |
| | | ACATCTTCTTCTGGAGGCCGTGGTTGGCTTGTATGGAG |
| | | CAGCAGACGCGCTACTTCGAGCGGAGGCATCCGGAGC |
| | | TTGCAGGATCGCCGCGGCTCCGGGCGTATATGCTCCG |
| | | CATTGGTCTTGACCAACTCTATCAGAGCTTGGTTGACG |
| | | GCAATTTCGATGATGCAGCTTGGGCGCAGGGTCGATG |
| | | CGACGCAATCGTCCGATCCGGAGCCGGGACTGTCGGG |
| | | CGTACACAAATCGCCCGCAGAAGCGCGGCCGTCTGGA |
| | | CCGATGGCTGTGTAGAAGTACTCGCCGATAGTGGAAA |
| | | CCGACGCCCCAGCACTCGTCCGAGGGCAAAGGAATAA |
| | | TCAGTACTGACAATAAAAAGATTCTTGTTTTCAAGAA |
| | | CTTGTCATTTGTATAGTTTTTTTATATTGTAGTTGTTCT |
| | | ATTTTAATCAAATGTTAGCGTGATTTATATTTTTTTTCG |
| | | CCTCGACATCATCTGCCCAGATGCGAAGTTAAGTGCG |
| | | CAGAAAGTAATATCATGCGTCAATCGTATGTGAATGC |
| | | TGGTCGCTATACTGCTGTCGATTCGATACTAACGCCGC |
| | | CATCCAGTGTCGAAAACGAGCT |
| 80 | DNA encodes S. cerevisiae | ATG AGA TTC CCA TCC ATC TTC ACT GCT GTT TTG |
| | Mating Factor | TTC GCT GCT TCT TCT GCT TTG GCT |
| | pre signal | |
| | sequence | |
| 81 | DNA encodes Tr | CGCGCCGGATCTCCCAACCCTACGAGGGCGGCAGCAG |
| | ManI catalytic | TCAAGGCCGCATTCCAGACGTCGTGGAACGCTTACCA |
| | domain | CCATTTTGCCTTTCCCCATGACGACCTCCACCCGGTCA |
| | | GCAACAGCTTTGATGATGAGAGAAACGGCTGGGGCTC |
| | | GTCGGCAATCGATGGCTTGGACACGGCTATCCTCATG |
| | | GGGGATGCCGACATTGTGAACACGATCCTTCAGTATG |
| | | TACCGCAGATCAACTTCACCACGACTGCGGTTGCCAA |
| | | CCAAGGCATCTCCGTGTTCGAGACCAACATTCGGTAC |
| | | CTCGGTGGCCTGCTTTCTGCCTATGACCTGTTGCGAGG |
| | | TCCTTTCAGCTCCTTGGCGACAAACCAGACCCTGGTAA |
| | | ACAGCCTTCTGAGGCAGGCTCAAACACTGGCCAACGG |
| | | CCTCAAGGTTGCGTTCACCACTCCCAGCGGTGTCCCGG |
| | | ACCCTACCGTCTTCTTCAACCCTACTGTCCGGAGAAGT |
| | | GGTGCATCTAGCAACAACGTCGCTGAAATTGGAAGCC |
| | | TGGTGCTCGAGTGGACACGGTTGAGCGACCTGACGGG |
| | | AAACCCGCAGTATGCCCAGCTTGCGCAGAAGGGCGAG |
| | | TCGTATCTCCTGAATCCAAAGGGAAGCCCGGAGGCAT |
| | | GGCCTGGCCTGATTGGAACGTTTGTCAGCACGAGCAA |
| | | CGGTACCTTTCAGGATAGCAGCGGCAGCTGGTCCGGC |
| | | CTCATGGACAGCTTCTACGAGTACCTGATCAAGATGT |
| | | ACCTGTACGACCCGGTTGCGTTTGCACACTACAAGGA |
| | | TCGCTGGGTCCTTGCTGCCGACTCGACCATTGCGCATC |
| | | TCGCCTCTCACCCGTCGACGCGCAAGGACTTGACCTTT |
| | | TTGTCTTCGTACAACGGACAGTCTACGTCGCCAAACTC |
| | | AGGACATTTGGCCAGTTTTGCCGGTGGCAACTTCATCT |
| | | TGGGAGGCATTCTCCTGAACGAGCAAAAGTACATTGA |
| | | CTTTGGAATCAAGCTTGCCAGCTCGTACTTTGCCACGT |
| | | ACAACCAGACGGCTTCTGGAATCGGCCCCGAAGGCTT |
| | | CGCGTGGGTGGACAGCGTGACGGGCGCCGGCGGCTCG |
| | | CCGCCCTCGTCCCAGTCCGGGTTCTACTCGTCGGCAGG |
| | | ATTCTGGGTGACGGCACCGTATTACATCCTGCGGCCG |
| | | GAGACGCTGGAGAGCTTGTACTACGCATACCGCGTCA |
| | | CGGGCGACTCCAAGTGGCAGGACCTGGCGTGGGAAGC |
| | | GTTCAGTGCCATTGAGGACGCATGCCGCGCCGGCAGC |
| | | GCGTACTCGTCCATCAACGACGTGACGCAGGCCAACG |
| | | GCGGGGGTGCCTCTGACGATATGGAGAGCTTCTGGTT |
| | | TGCCGAGGCGCTCAAGTATGCGTACCTGATCTTTGCG |
| | | GAGGAGTCGGATGTGCAGGTGCAGGCCAACGGCGGG |
| | | AACAAATTTGTCTTTAACACGGAGGCGCACCCCTTTA |
| | | GCATCCGTTCATCATCACGACGGGGCGGCCACCTTGC |
| | | TTAA |
| 82 | Sequence of the | TTGGGGGCCTCCAGGACTTGCTGAAATTTGCTGACTCA |
| | 5′-Region used | TCTTCGCCATCCAAGGATAATGAGTTAGCTAATGTGA |
| | for knock out of | CAGTTAATGAGTCGTCTTGACTAACGGGGAACATTTC |
| | PpSTE13 | ATTATTTATATCCAGAGTCAATTTGATAGCAGAGTTTG |
| | | TGGTTGAAATACCTATGATTCGGGAGACTTTGTTGTAA |
| | | CGACCATTATCCACAGTTTGGACCGTGAAAATGTCAT |
| | | CGAAGAGAGCAGACGACATATTATCTATTGTGGTAAG |
| | | TGATAGTTGGAAGTCCGACTAAGGCATGAAAATGAGA |
| | | AGACTGAAAATTTAAAGTTTTTGAAAACACTAATCGG |
| | | GTAATAACTTGGAAATTACGTTTACGTGCCTTTAGCTC |
| | | TTGTCCTTACCCCTGATAATCTATCCATTTCCCGAGAG |
| | | ACAATGACATCTCGGACAGCTGAGAACCCGTTCGATA |
| | | TAGAGCTTCAAGAGAATCTAAGTCCACGTTCTTCCAAT |
| | | TCGTCCATATTGGAAAACATTAATGAGTATGCTAGAA |
| | | GACATCGCAATGATTCGCTTTCCCAAGAATGTGATAA |
| | | TGAAGATGAGAACGAAAATCTCAATTATACTGATAAC |
| | | TTGGCCAAGTTTTCAAAGTCTGGAGTATCAAGAAAGA |
| | | GCTGTATGCTAATATTTGGTATTTGCTTTGTTATCTGG |
| | | CTGTTTCTCTTTGCCTTGTATGCGAGGGACAATCGATT |
| | | TTCCAATTTGAACGAGTACGTTCCAGATTCAAACAG |
| 83 | Sequence of the | CTACTGGGAACCACGAGACATCACTGCAGTAGTTTCC |
| | 3′-Region used | AAGTGGATTTCAGATCACTCATTTGTGAATCCTGACAA |
| | for knock out of | AACTGCGATATGGGGGTGGTCTTACGGTGGGTTCACT |
| | PpSTE13 | ACGCTTAAGACATTGGAATATGATTCTGGAGAGGTTTT |
| | | CAAATATGGTATGGCTGTTGCTCCAGTAACTAATTGGC |
| | | TTTTGTATGACTCCATCTACACTGAAAGATACATGAAC |
| | | CTTCCAAAGGACAATGTTGAAGGCTACAGTGAACACA |
| | | GCGTCATTAAGAAGGTTTCCAATTTTAAGAATGTAAA |
| | | CCGATTCTTGGTTTGTCACGGGACTACTGATGATAACG |
| | | TGCATTTTCAGAACACACTAACCTTACTGGACCAGTTC |
| | | AATATTAATGGTGTTGTGAATTACGATCTTCAGGTGTA |
| | | TCCCGACAGTGAACATAGCATTGCCCATCACAACGCA |
| | | AATAAAGTGATCTACGAGAGGTTATTCAAGTGGTTAG |
| | | AGCGGGCATTTAACGATAGATTTTTGTAACATTCCGTA |
| | | CTTCATGCCATACTATATATCCTGCAAGGTTTCCCTTT |
| | | CAGACACAATAATTGCTTTGCAATTTTACATACCACCA |
| | | ATTGGCAAAAATAATCTCTTCAGTAAGTTGAATGCTTT |
| | | TCAAGCCAGCACCGTGAGAAATTGCTACAGCGCGCAT |
| | | TCTAACATCACTTTAAAATTCCCTCGCCGGTGCTCACT |
| | | GGAGTTTCCAACCCTTAGCTTATCAAAATCGGGTGAT |
| | | AACTCTGAGTTTTTTTTTTCACTTCTATTCCTAAACCTT |
| | | CGCCCAATGCTACCACCTCCAATCAACATCCCGAAAT |
| | | GGATAGAAGAGAATGGACATCTCTTGCAACCTCCGGT |
| | | TAATAATTACTGTCTCCACAGAGGAGGATTTACGGTA |
| | | ATGATTGTAGGTGGGCCTAATG |
| 84 | Sequence of the | CACCTGGGCCTGTTGCTGCTGGTACTGCTGTTGGAACT |
| | 5′-Region used | GTTGGTATTGTTGCTGATCTAAGGCCGCCTGTTCCACA |
| | for knock out of | CCGTGTGTATCGAATGCTTGGGCAAAATCATCGCCTG |
| | PpDAP2 | CCGGAGGCCCCACTACCGCTTGTTCCTCCTGCTCTTGT |
| | | TTGTTTTGCTCATTGATGATATCGGCGTCAATGAATTG |
| | | ATCCTCAATCGTGTGGTGGTGGTGTCGTGATTCCTCTT |
| | | CTTTCTTGAGTGCCTTATCCATATTCCTATCTTAGTGTA |
| | | CCAATAATTTTGTTAAACACACGCTGTTGTTTATGAAA |
| | | AGTCGTCAAAAGGTTAAAAATTCTACTTGGTGTGTGTC |
| | | AGAGAAAGTAGTGCAGACCCCCAGTTTGTTGACTAGT |
| | | TGAGAAGGCGGCTCACTATTGCGCGAATAGCATGAGA |
| | | AATTTGCAAACATCTGGCAAAGTGGTCAATACCTGCC |
| | | AACCTGCCAATCTTCGCGACGGAGGCTGTTAAGCGGG |
| | | TTGGGTTCCCAAAGTGAATGGATATTACGGGCAGGAA |
| | | AAACAGCCCCTTCCACACTAGTCTTTGCTACTGACATC |
| | | TTCCCTCTCATGTATCCCGAACACAAGTATCGGGAGTA |
| | | TCAACGGAGGGTGCCCTTATGGCAGTACTCCCTGTTG |
| | | GTGATTGTACTGCTATACGGGTCTCATTTGCTTATCAG |
| | | CACCATCAACTTGATACACTATAACCACAAAAATTAT |
| | | CATGCACACCCAGTCAATAGTGGTATCGTTCTTAATGA |
| | | GTTTGCTGATGACGATTCATTCTCTTTGAATGGCACTC |
| | | TGAACTTGGAGAACTGGAGAAATGGTACCTTTTCCCC |
| | | TAAATTTCATTCCATTCAGTGGACCGAAATAGGTCAG |
| | | GAAGATGACCAGGGATATTACATTCTCTCTTCCAATTC |
| | | CTCTTACATAGTAAAGTCTTTATCCGACCCAGACTTTG |
| | | AATCTGTTCTATTCAACGAGTCTACAATCACTTACAACG |
| 85 | Sequence of the | GGCAGCAAAGCCTTACGTTGATGAGAATAGACTGGCC |
| | 3′-Region used | ATTTGGGGTTGGTCTTATGGAGGTTACATGACGCTAAA |
| | for knock out of | GGTTTTAGAACAGGATAAAGGTGAAACATTCAAATAT |
| | PpDAP2 | GGAATGTCTGTTGCCCCTGTGACGAATTGGAAATTCTA |
| | | TGATTCTATCTACACAGAAAGATACATGCACACTCCTC |
| | | AGGACAATCCAAACTATTATAATTCGTCAATCCATGA |
| | | GATTGATAATTTGAAGGGAGTGAAGAGGTTCTTGCTA |
| | | ATGCACGGAACTGGTGACGACAATGTTCACTTCCAAA |
| | | ATACACTCAAAGTTCTAGATTTATTTGATTTACATGGT |
| | | CTTGAAAACTATGATATCCACGTGTTCCCTGATAGTGA |
| | | TCACAGTATTAGATATCACAACGGTAATGTTATAGTGT |
| | | ATGATAAGCTATTCCATTGGATTAGGCGTGCATTCAA |
| | | GGCTGGCAAATAAATAGGTGCAAAAATATTATTAGAC |
| | | TTTTTTTTTCGTTCGCAAGTTATTACTGTGTACCATACC |
| | | GATCCAATCCGTATTGTAATTCATGTTCTAGATCCAAA |
| | | ATTTGGGACTCTAATTCATGAGGTCTAGGAAGATGAT |
| | | CATCTCTATAGTTTTCAGCGGGGGGCTCGATTTGCGGT |
| | | TGGTCAAAGCTAACATCAAAATGTTTGTCAGGTTCAG |
| | | TGAATGGTAACTGCTGCTCTTGAATTGGTCGTCTGACA |
| | | AATTCTCTAAGTGATAGCACTTCATCTACAATCATTTG |
| | | CTTCATCGTTTCTATATCGTCCACGACCTCAAACGAGA |
| | | AATCGAATTTGGAAGAACAGACGGGCTCATCGTTAGG |
| | | ATCATGCCAAACCTTGAGATATGGATGCTCTAAAGCC |
| | | TCAGTAACTGTAATTCTGTGAGTGGGATCTACCGTGA |
| | | GCATTCGATCCAGTAAGTCTATCGCTTCAGGGTTGGCA |
| | | CCGGGAAATAACTGGCTGAATGGGATCTTGGGCATGA |
| | | ATGGCAGGGAGCGAACATAATCCTGGGCACGCTCTGA |
| | | TCTGATAGACTGAAGTGTCTCTTCCGAAACAGTACCC |
| | | AGCGTACTCAAAATCAAGTTCAATTGATCCACATAGT |
| | | CTCTTCCTCTAAAAATGGGTCGGCCACCTA |
| 86 | Sequence of the | GGCCAGCCCATCACCATGAATGCTTAAAACGCCAACT |
| | PpTHR1 in loci | CCTTCCATCTCATTTTCGTACCAGATTATGACTCTTAG |
| | | GCGGGGAGAATCCCGTCCAGCATAGCGAACATTTCTT |
| | | TTTTTTTTTTTTTTCGTTTCGCATCTCTCTATCGCATTCA |
| | | GAAAAAAATACATATAATTCTTCCAGTTTCCGTCATTC |
| | | ATTACGTTTAAAACTACGAAAGTTTTAGCTCTCTTTTG |
| | | TTTTTGTTTCCTAGATTCGAAATATTTTCTTTATTGAGT |
| | | TTAATTTGTGTGGCAGACAATGGTTAGATCTTTCACCA |
| | | TCAAAGTGCCTGCTTCCTCAGCAAATATAGGACCGGG |
| | | GTTTGACGTTCTGGGAATTGGTCTCAACCTTTACTTGG |
| | | AACTACAAGTCACCATTGATCCCAAAATTGATACCTC |
| | | AAGCGATCCAGAAAATGTGTTATTGTCGTATGAAGGT |
| | | GAGGGGGCTGATGAGGTGTCATTGAAAAGTGACGAAA |
| | | ACTTGATTACGCGCACAGCTCTCTATGTTCTACGTTGT |
| | | GACGACGTCAGGACTTTCCCTAAGGGAACCAAGATTC |
| | | ACGTCATTAACCCTATTCCTCTAGGAAGAGGCTTGGG |
| | | ATCTTCGGGTGCTGCAGTTGTCGCCGGTGCATTGCTCG |
| | | GAAATTCCATCGGACAGCTTGGATACTCCAAACAACG |
| | | TTTACTGGATTACTGTTTGATGATAGAACGTCATCCAG |
| | | ATAACATCACCGCAGCTATGGTGGGTGGTTTCGTTGG |
| | | ATCTTATCTTAGAGATCTTTCACCAGAAGACACCCAG |
| | | AGAAAAGAGATTCCATTAGCAGAAGTCCTGCCAGAAC |
| | | CTCAAGGTGGTATTAACACCGGTCTCAACCCACCAGT |
| | | GCCTCCAAAAAACATTGGGCACCACATCAAATACGGC |
| | | TGGGCAAAAGAGATCAAATGTATTGCCATTATTCCAG |
| | | ACTTTGAAGTATCAACCGCTTCATCTAGAGGCGTTCTT |
| | | CCAACCACTTACGAGAGACATGACATTATTTTCAACCT |
| | | GCAAAGGATAGCCGTTCTTACCACTGCCCTGACACAA |
| | | TCTCCACCAGATCCAAGCTTGATATACCCAGCTATGCA |
| | | GGACAGGATTCACCAACCTTACAGGAAAACTTTGATC |
| | | CACGGACTGACTGAAATACTGTCTTCATTCACCCCAG |
| | | AATTACACAAAGGTTTGTTGGGAATCTGTCTTTCCGGT |
| | | GCTGGGCCCACAATATTAGCCCTCGCAACTGAAAACT |
| | | TCGATCAGATTGCTAAGGACATCATTGCCAGATTTGCT |
| | | GTCGAAGACATCACCTGTAGTTGGAAACTCTTGACCC |
| | | CAGCTCTTGAAGGTTCTGTTGTTGAGGAGCTTGCTTAA |
| | | TAGAAATTAGAACATCCTCTTTAGATTATGATAATACG |
| | | TTTTTAACTTTTCCCCTAACTGTAGTGATGGTATCTGA |
| | | CCCTCTTAGACCTTAGGTTGGACCTTCTCGAATTTCCT |
| | | GCCTCTATCAAAAATCCGACCCTCGACATCGTTTACGT |
| | | ACTTTGCAACCAATTAACTAGTACCGGCAGACGTTCA |
| | | GTGATCATGGCTCTCTATACAAATACCCTGATAACGTT |
| | | TGCATTCCTGACAGTCGGAGGATGTACGTGCTTATTTT |
| | | CTTGCTAGTCCCAAATGTTTTGAGATTGCTCCAATCGT |
| | | TTTTTCAACAATACTAACTGCCAACAAATAGATCTTTT |
| | | ATTCAACGGAAATGGGGAACAATTCAACGTGGGTGAC |
| | | TTTTTGGAGACTACATCTCCCTATATGTGGGCAAATCT |
| | | GGGTATAGCAAGTTGCATTGGATTCTCGGTCATTGGTG |
| | | CTGCATGGGGAATTTTCATAACAGGTTCTTCGATCATC |
| | | GGTGCAGGTGTCAAAGCTCCCAGAATCACAACAAAAA |
| | | ATTTAATCTCCATCATTTTCTGTGAGGTGGTGGCTATT |
| | | TATGGGCTTATTATGGCC |
| 87 | Sequence of | CCTGTGAGTCTGGCTCAATCACTTTTCAAAGATAAGG |
| | PpHIS3 5′ | ACTATTCTGCAGAACATGCAGCCCAGGCAACATCATC |
| | integration | CCAGTTCATCTCTGTGAACACAGGAATAGGATTCCTG |
| | fragment | GACCATATGTTACACGCACTTGCTAAGCACGGCGGCT |
| | | GGTCTGTCATTATCGAATGTGTAGGTGATTTGCACATT |
| | | GATGACCATCATTCAGCAGAAGATACTGGAATCGCAT |
| | | TGGGGATGGCATTCAAAGAAGCCTTGGGCCATGTTCG |
| | | TGGTATCAAAAGATTCGGGTCCGGATTTGCTCCACTA |
| | | GACGAAGCTCTCAGTCGGGCTGTTATTGATATGTCTAA |
| | | CAGGCCCTATGCTGTTGTCGATCTGGGTTTGAAAAGA |
| | | GAGAAGATTGGAGACCTATCGTGTGAGATGATTCCCC |
| | | ATGTTTTGGAAAGTTTTGCCCAAGGAGCCCATGTAAC |
| | | CATGCACGTAGATTGTTTGCGAGGTTTCAACGACCATC |
| | | ATCGTGCCGAGAGTGCATTCAAAGCTTTGGCTATAGC |
| | | TATCAAAGAGGCCATTTCAAGCAACGGCACGGACGAC |
| | | ATTCCAAGTACGAAGGGTGTTCTTTTCTGA |
| 88 | Sequence of | GTCTGGAAGGTGTCTACATCTGTGAAATCCGTATTTAT |
| | PpHIS3 3′ | TTAAGTAAAACAATCAGTAATATAAGATCTTAGTTGG |
| | integration | TTTACCACATAGTCGGTACCGGTCGTGTGAACAATAG |
| | fragment | TTCAATGCCTCCGATTGTGCCTTATTGTTGTGGTCTGC |
| | | ATTTTCGCGGCGAAATTTCTACTTCAGATCGGGGCTGA |
| | | GATGACCTTAGTACTCACATCAACCAGCTCGTTGAAA |
| | | GTTCCCACATGACCACTCAATGTTTAATAGCTTGGCAC |
| | | CCATGAGGTTGAAGAAACTACTTAAGGTGTTTTGTGC |
| | | CTCAGTAGTGCTGTTAGCGGCGACATCTGTGGTGTTAT |
| | | TTTTCCACTTTGGAGGTCAGATCATAATCCCCATACCG |
| | | GAACGCACTGTGACCTTAAGTACTCCTCCCGCAAACG |
| | | ATACTTGGCAGTTTCAACAGTTCTTCAACGGCTATTTA |
| | | GACGCCCTGTTAGAGAATAACCTGTCGTATCCGATAC |
| | | CAGAAAGGTGGAATCATGAAGTTACAAATGTAAGATT |
| | | CTTCAATCGCATAGGTGAATTGCTCTCGGAGAGTAGG |
| | | CTACAGGAGCTGATTCATTTTAGTCCTGAGTTCATAGA |
| | | GGATACCAGTGACAAATTCGACAATATTGTTGAACAA |
| | | ATTCCAGCAAAATGGCCTTACGAAAACATGTACAGAG |
| | | GAGATGGATACGTTATTGTTGGTGGTGGCAGACACAC |
| | | CTTTTTGGCACTGCTGAATATCAACGCTTTGAGAAGAG |
| | | CAGGCAATAAACTGCCAGTTGAGGTCGTGTTGCCAAC |
| | | TTACGACGACTATGAGGAAGATTTCTGTGAAAATCAT |
| | | TTTCCACTTTTGAATGCAAGATGCGTAATCTTAGAAGA |
| | | ACGATTTGGTGACCAAGTTTATCCCCGGTTACAACTAG |
| | | GAGGCTACCAGTTTAAAATATTTGCGATAGCAGCAAG |
| | | TTCATTCAAAAACTGCTTTTTGTTAGATTCAGATAATA |
| | | TACCCTTGCGAAAGATGGATAAGATATTCTCAAGCGA |
| | | ACTATACAAGAATAAGACAATGATTACTTGGCCAGACT |
| 89 | Sequence of | CGAGTCGGCCAGCCCATCACCATGAATGCTTAAAACG |
| | PpTHR1 5′ | CCAACTCCTTCCATCTCATTTTCGTACCAGATTATGAC |
| | integration | TCTTAGGCGGGGAGAATCCCGTCCAGCATAGCGAACA |
| | fragment | TTTCTTTTTTTTTTTTTTTTCGTTTCGCATCTCTCTATCG |
| | | CATTCAGAAAAAAATACATATAATTCTTCCAGTTTCCG |
| | | TCATTCATTACGTTTAAAACTACGAAAGTTTTAGCTCT |
| | | CTTTTGTTTTTGTTTCCTAGATTCGAAATATTTTCTTTA |
| | | TTGAGTTTAATTTGTGTGGCAGACAATGGTTAGATCTT |
| | | TCACCATCAAAGTGCCTGCTTCCTCAGCAAATATAGG |
| | | ACCGGGGTTTGACGTTCTGGGAATTGGTCTCAACCTTT |
| | | ACTTGGAACTACAAGTCACCATTGATCCCAAAATTGA |
| | | TACCTCAAGCGATCCAGAAAATGTGTTATTGTCGTATG |
| | | AAGGTGAGGGGGCTGATGAGGTGTCATTGAAAAGTGA |
| | | CGAAAACTTGATTACGCGCACAGCTCTCTATGTTCTAC |
| | | GTTGTGACGACGTCAGGACTTTCCCTAAGGGAACCAA |
| | | GATTCACGTCATTAACCCTATTCCTCTAGGAAGAGGCT |
| | | TGGGATCTTCGGGTGCTGCAGTTGTC |
| 90 | Sequence of | TAGAAATTAGAACATCCTCTTTAGATTATGATAATACG |
| | PpTHR1 3′ | TTTTTAACTTTTCCCCTAACTGTAGTGATGGTATCTGA |
| | integration | CCCTCTTAGACCTTAGGTTGGACCTTCTCGAATTTCCT |
| | fragment | GCCTCTATCAAAAATCCGACCCTCGACATCGTTTACGT |
| | | ACTTTGCAACCAATTAACTAGTACCGGCAGACGTTCA |
| | | GTGATCATGGCTCTCTATACAAATACCCTGATAACGTT |
| | | TGCATTCCTGACAGTCGGAGGATGTACGTGCTTATTTT |
| | | CTTGCTAGTCCCAAATGTTTTGAGATTGCTCCAATCGT |
| | | TTTTTCAACAATACTAACTGCCAACAAATAGATCTTTT |
| | | ATTCAACGGAAATGGGGAACAATTCAACGTGGGTGAC |
| | | TTTTTGGAGACTACATCTCCCTATATGTGGGCAAATCT |
| | | GGGTATAGCAAGTTGCATTGGATTCTCGGTCATTGGTG |
| | | CTGCATGGGGAATTTTCATAACAGGTTCTTCGATCATC |
| | | GGTGCAGGTGTCAAAGCTCCCAGAATCACAACAAAAA |
| | | ATTTAATCTCCATCATTTTCTGTGAGGTGGTGGCTATT |
| | | TATGGGCTTATTATGGCCATTGT |
| 91 | Sequence of the | AAGTGGGCCAGATTATATAAATATGGATCAACATGAA |
| | 5′-Region used | GCCTTGAAAGATTTCAAGGACAGGCTTAGGAATTACG |
| | for knock out of | AAAAAGTTTACGAGACTATTGACGACCAGGAGGAAGA |
| | PpVPS10-1 | GGAGAACGAACGGTACAATATTCAGTATCTGAAGATA |
| | | ATCAACGCAGGAAAGAAGATAGTCAGTTATAACATAA |
| | | ATGGGTATTTATCGTCCCACACCGTTTTTTATCTCCTG |
| | | AATTTCAATCTTGCAGAACGTCAAATATGGTTGACGA |
| | | CGAATGGAGAGACAGAGTATAACCTTCAAAATAGGAT |
| | | TGGAGGTGATTCCAAATTAAGCAATGAGGGATGGAAA |
| | | TTTGCCAAAGCATTGCCCAAGTTTATAGCACAGAAAA |
| | | GAAAAGAGTTTCAACTTAGACAGTTGACCAAACACTA |
| | | TATCGAGACTCAAACGCCCATTGAAGACGTACCGTTG |
| | | GAGGAGCACACCAAGCCAGTCAAATATTCTGATCTGC |
| | | ATTTCCATGTTTGGTCATCGGCTTTAAAGAGATCTACT |
| | | CAATCAACAACATTTTTTCCATCGGAAAATTACTCTCT |
| | | GAAGCAATTCAGAACGTTGAATGATCTCTGTTGCGGA |
| | | TCACTGGATGGTTTGACTGAACAAGAGTTCAAAAGTA |
| | | AATACAAAGAAGAATACCAGAATTCTCAGACTGATAA |
| | | ACTGAGTTTCAGTTTCCCTGGTATCGGTGGGGAGTCTT |
| | | ATTTGGACGTGATCAACCGTTTGAGACCACTAATAGTT |
| | | GAACTAGAAAGGTTGCCAGAACATGTCCTGGTCATTA |
| | | CCCACCGGGTCATAGTAAGGATTTTACTAGGATATTTC |
| | | ATGAATTTGGATAGAAATCTGTTGACAGATTTGGAAA |
| | | TTTTGCATGGGTATGTTTATTGTATTGAGCCGAAACCT |
| | | TATGGTTTAGACTTAAAGATCTGGCAGTATGATGAGG |
| | | CGGACAACGAGTTTAATGAAGTTGATAAGCTGGAATT |
| | | CATGAAAAGAAGAAGAAAATCGATCAACGTCAACAC |
| | | GACAGATTTCAGAATGCAGTTAAACAAAGAGTTGCAA |
| | | CAGGACGCTCTCAATAATAGTCCTGGTAATAATAGTC |
| | | CGGGCGTATCATCTCTATCTTCATACTCGTCGTCCTCT |
| | | TCCCTTTCCGCTGACGGGAGCGAGGGAGAAACATTAA |
| | | TACCACAAGTATCCCAGGCGGAGAGCTACAACTTTGA |
| | | ATTTAACTCTCTTTCATCATCAGTTTCATCGTTGAAAA |
| | | GGACGACATCTTCTTCCCAACATTTGAGCTCCAATCCT |
| | | AGTTGTCTGAGCATGCATAATGCCTCATTGGACGAGA |
| | | ATGACGACGAACATTTAATAGACCCGGCTTCTACAGA |
| | | CGACAAGCTAAACATGGTATTACAGGACAAAACGCTA |
| | | ATTAAAAAGCTCAAAAGTTTACTACTTGACGAGGCCG |
| | | AAGGCTAGACAATCCACAGTTAATTTTGATACTGTACT |
| | | TTATAACGAGTAACATACATATCTTATGTAATCATCTA |
| | | TGTCACGTCACGTGCGCGCGACATTATTCCGAGAACTT |
| | | GCGCCCTGCTAGCTCCACTGTCAGAGTGATAACTTCCC |
| | | CAAAATAGGATCCAACTGTTTCCAATTGCTTTTGGAAA |
| | | TGTGGATTGAAAGAAACCTCATAGCGTAA |
| 92 | Sequence of the | GACGACGAGGAGAATATCAATTTTGATTCCCGGTAGA |
| | 3′-Region used | TAGCTCACCCACGGTCACACACACAAACACACATACA |
| | for knock out of | CATTAACACACAGAGTTATTAGTTAACAGAGAAAACT |
| | PpVPS10-1 | CTAACAAAGTATTTATTTTCGTTACGTAATCCGACTTT |
| | | TCTTTTTACCGTTTTCTATTGCTCCTCTCATTTGCCCCT |
| | | AAAAGTTGCTCCTCATTACTAAAATCACCACACCATG |
| | | CTCGAATATGATGTTACTAAATGCAAATTGTAGTCGTG |
| | | CCTCTTGTGGTAATACTATAGGGAATATCTCTCGATTA |
| | | CTCGATTCTGGTTAATTTTTTCTTTTTTTATAGGGGAAG |
| | | TTTTTTTTTCTTCCCCTTTCTCTCCAGTTTATTTATTTAC |
| | | TAAGAAAATCCAACAGATACCAACCACCCAAAAAGAT |
| | | CCTAAACAGCCTGTTTTTGAGGAGTTTTTCAGCAGCTA |
| | | AGCTTCATCAGTTTTTTAATACTTAATTTATTGCCCTTC |
| | | ACTTTGTTTCTTGTGGCTTTTAAGGCTCTCCGGAACAG |
| | | CGGTTTCAAAATCAAATCTCAGTTATTTGTTTGCTCCG |
| | | CTTTGTCAGTTCAAAGATCATGGTTTCCGAAAACAAG |
| | | AATCAATCTTCGATTTTGATGGACAACTCCAAGAAGC |
| | | TCTCTCCGAAGCCCATTTTGAATAACAAGAATGAACC |
| | | GTTTGGCATCGGCGTCGATGGACTTCAACATCCTCAAC |
| | | CGACTTTATGCCGCACAGAATCGGAACTCTTGTTCAAC |
| | | TTGAGCCAAGTCAATAAATCCCAAATAACTTTGGACG |
| | | GTGCAGTTACTCCACCTGCTGATGGTAATGGGAATGA |
| | | AGCAAAAAGAGCAAATCTCATCTCTTTTGATGTTCCAT |
| | | CGTCTCAAGTGAAACATAGAGGGTCTATTAGTGCAAG |
| | | GCCCTCGGCAGTGAATGTGTCCCAAATTACCGGGGCC |
| | | CTTTCTCAATCCGGATCTTCTAGAAATCCCTACGATCA |
| | | AACACAGTCACCTCCACCTAGCACTTACGCCTCCAGG |
| | | CAGAACTCCACCCATGGAAATAATATCGATAGCTTGC |
| | | AATATTTGGCAACAAGAGATCTTAGTGCTTTAAGGCT |
| | | GGAAAGAGATGCTTCCGCACGAGAAGCTACCTCTTCT |
| | | GCAGTGTCCACTCCTGTTCAGTTCGATGTACCCAAACA |
| | | ACATCATCTCCTTCATTTAGAACAAGACCCGACAAGG |
| | | CCCATCC |
| 93 | Sequence of | ACGACGGCCAAATTCATGATACACACTCTGTTTCAGCT |
| | PpTRP5 5′ | GGTTTGGACTACCCTGGAGTTGGTCCTGAATTGGCTGC |
| | integration | CTGGAAAGCAAATGGTAGAGCCCAATTTTCCGCTGTA |
| | fragment | ACTGATGCCCAAGCATTAGAGGGATTCAAAATCCTGT |
| | | CTCAATTGGAAGGGATCATTCCAGCACTAGAGTCTAG |
| | | TCATGCAATCTACGGCGCATTGCAAATTGCAAAGACT |
| | | ATGTCTTCGGACCAGTCCTTAGTTATTAATGTATCTGG |
| | | AAGGGGTGATAAGGACGTCCAGAGTGTAGCTGAGATT |
| | | TTACCTAAATTGGGACCTCAAATTGGATGGGATTTGC |
| | | GTTTCAGCGAAGACATTACTAAAGAGTGA |
| 94 | Sequence of | TCGATAGCACAATATTCAACTTGACTGGGTGTTAAGA |
| | PpTRP5 3′ | ACTAAGAGCTCTGGGAAACTTTGTATTTATTACTACCA |
| | integration | ACACAGTCAAATTATTGGATGTGTTTTTTTTTCCAGTA |
| | fragment | CATTTCACTGAGCAGTTTGTTATACTCGGTCTTTAATC |
| | | TCCATATACATGCAGATTGTAATACAGATCTGAACAG |
| | | TTTGATTCTGATTGATCTTGCCACCAATATTCTATTTTT |
| | | GTATCAAGTAACAGAGTCAATGATCATTGGTAACGTA |
| | | ACGGTTTTCGTGTATAGTAGTTAGAGCCCATCTTGTAA |
| | | CCTCATTTCCTCCCATATTAAAGTATCAGTGATTCGCT |
| | | GGAACGATTAACTAAGAAAAAAAAAATATCTGCACAT |
| | | ACTCATCAGTCTGTAAATCTAAGTCAAAACTGCTGTAT |
| | | CCAATAGAAATCGGGATATACCTGGATGTTTTTTCCAC |
| | | ATAAACAAACGGGAGTTCAGCTTACTTATGGTGTTGA |
| | | TGCAATTCAGTATGATCCTACCAATAAAACGAAACTT |
| | | TGGGATTTTGGCTGTTTGAGGGATCAAAAGCTGCACC |
| | | TTTACAAGATTGACGGATCGACCATTAGACCAAAGCA |
| | | AATGGCCACCAA |

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

Patents, patent applications, Genbank Accession Numbers and publications are cited throughout this application, the disclosures of which, particularly, including all disclosed chemical structures and antibody amino acid sequences therein, are incorporated herein by reference. Citation of the above publications or documents is not intended as an admission that any of the foregoing is pertinent prior art, nor does it constitute any admission as to the contents or date of these publications or documents. All references cited herein are incorporated by reference to the same extent as if each individual publication, patent application, or patent, was specifically and individually indicated to be incorporated by reference.

The foregoing written specification is considered to be sufficient to enable one skilled in the art to practice the invention. Various modifications of the invention in addition to those shown and described herein will become apparent to those skilled in the art from the foregoing description and fall within the scope of the appended claims.

What is claimed:

1. A composition comprising a fragment of recombinant human tumor necrosis factor receptor fused to the constant region of an antibody (TNFRII-Fc) wherein the TNFRII-Fc has N-glycans and O-glycans and wherein the O-glycans are of the dystroglycan- or O-mannose reduced glycans, and pharmaceutically acceptable salts thereof.

2. The composition of claim 1, wherein the N-glycans and O-glycans on the TNFRII-Fc are predominantly sialylated with α2,6 or α2,3 sialic acid residues.

3. The composition of claim 1, wherein the N-glycans on the TNFRII-Fc lack fucose residues.

4. The composition of claim 1, wherein the N-glycans and O-glycans on the TNFRII-Fc which are sialylated comprise N-acetylneuraminic acid (NANA) and no N-glycolylneuraminic acid (NGNA).

5. The composition of claim 1, wherein a ratio of mole sialic acid to mole of the TNFRII-Fc is at least 10.

6. The composition of claim 5, wherein a ratio of mole sialic acid to mole of the TNFRII-Fc is about 10 to 21.

7. The composition of claim 5, wherein a ratio of mole sialic acid to mole of the TNFRII-Fc is greater than 21.

8. The composition of claim 1, wherein the N-glycans on the TNFRII-Fc are predominantly mono-, bi-, tri-, or tetra-sialylated N-glycans N-glycans.

9. The composition of claim 1, wherein the O-glycans on the TNFRII-Fc are predominantly sialylated O-glycans.

10. The composition of claim 1, wherein greater than 40% of the O-glycans on the TNFRII-Fc are sialylated O-glycans.

11. The composition of claim 1, wherein about 20% of the O-glycans on the TNFRII-Fc are of the mannose type or a combination of mannose and mannobiose types.

12. The composition of claim 1, wherein less than 50% of O-glycans on the TNFRII-Fc possess terminal mannose.

13. The composition of claim 1, wherein the TNRFII domain of the TNFRII-Fc has an amino acid sequence with at least 90% identity to the amino acid sequence set forth in SEQ ID NO:73 or 75.

14. A method for producing a recombinant human tumor necrosis factor receptor fused to the constant region of an antibody (TNFRII-Fc) having sialylated N-glycans and O-glycans comprising;
(a) providing a recombinant yeast host cell genetically engineered to produce glycoproteins having sialylated N-glycans and further comprising
(i) a nucleic acid molecule encoding the TNFRII-Fc;
(ii) a nucleic acids molecule encoding an α1,2-mannosidase activity linked to a heterologous targeting or signaling peptide that targets the mannosidase activity to the secretory pathway; and
(iii) a nucleic acid molecule encoding an O-linked mannose β1,2-N-acetylglucosaminyltransferase I (POMGnT I);
(b) culturing the host cell under conditions suitable for producing the TNFRII-Fc; and
(c) recovering the TNFRII-Fc from the culture fluid to produce the TNFRII-Fc having sialylated N-glycans and O-glycans.

15. The method of claim 14, wherein the N-glycans and O-glycans on the TNFRII-Fc are predominantly sialylated with α2,6 or α2,3 sialic acid residues.

16. The method of claim 14, wherein the N-glycans on the TNFRII-Fc lack fucose residues.

17. The method of claim 14, wherein the N-glycans and O-glycans on the TNFRII-Fc which are sialylated comprise N-acetylneuraminic acid (NANA) and no N-glycolylneuraminic acid (NGNA).

18. The method of claim 14, wherein a ratio of mole sialic acid to mole of the TNFRII-Fc is at least 10.

19. The method of claim 18, wherein a ratio of mole sialic acid to mole of the TNFRII-Fc is about 10 to 21.

20. The method of claim 18, wherein a ratio of mole sialic acid to mole of the TNFRII-Fc is greater than 21.

21. The method of claim 14, wherein the N-glycans on the TNFRII-Fc are predominantly mono-, bi-, tri-, or tetra-sialylated N-glycans.

22. The method of claim 14, wherein the O-glycans on the TNFRII-Fc are predominantly sialylated O-glycans.

23. The method of claim 14, wherein greater than 40% of the O-glycans on the TNFRII-Fc are sialylated O-glycans.

24. The method of claim 14, wherein less than 50% of O-glycans on the TNFRII-Fc possess terminal mannose.

25. The method of claim 14, wherein about 20% of the O-glycans on the TNFRII-Fc are of the mannose type or a combination of mannose and mannobiose types.

26. The method of claim 14, wherein the TNFRII domain of the TNFRII-Fc has an amino acid sequence with 90% identity to the amino acid sequence set forth in SEQ ID NO:73 or 75.

27. The method of claim 14, wherein the TNFRII-Fc is recovered from the culture fluid in a process comprising a hydroxyapatite or aminophenyl borate chromatography step.

28. A pharmaceutical composition comprising the polypeptide of any one of claims 1 to 13 and a pharmaceutically suitable carrier.

29. Use of the pharmaceutical composition of claim 27 in the manufacture of a medicament for inflammatory diseases and cancers that display an increased and/or unregulated level of soluble TNFRII or polymorphisms.

30. Use of the pharmaceutical composition of claim 27 in the manufacture of a medicament for treating rheumatoid arthritis.
